(19) United States
(12) Patent Application Publication
(10) Pub. No.: US 2026/0199380 A1
(43) Pub. Date: Jul. 16, 2026

(54) METHODS AND COMPOSITIONS FOR TREATING VIRAL INFECTIONS

(71) Applicant: Gilead Sciences, Inc., Foster City, CA (US)

(72) Inventors: Ana Z. Gonzalez Buenrostro, San Mateo, CA (US); Darryl Kato, San Francisco, CA (US); Gregg M. Schwarzwalder, San Carlos, CA (US); Murali Subramanian, Foster City, CA (US)

(21) Appl. No.: 19/412,138

(22) Filed: Dec. 8, 2025

Related U.S. Application Data
(60) Provisional application No. 63/729,942, filed on Dec. 9, 2024.

(51) Int. Cl. A61K 31/675; A61K 9/20; A61P 31/18

(52) U.S. Cl. CPC A61K 31/675; A61K 9/2018; A61K 9/2027; A61K 9/2031; A61K 9/2054; A61P 31/18

(57) ABSTRACT

This disclosure provides methods of treating HIV infections using Compound 1, or a pharmaceutically acceptable salt thereof, and a compound of Formula I, or a pharmaceutically acceptable salt thereof. The present disclosure further provides pharmaceutical formulations, comprising Compound 1, or a pharmaceutically acceptable salt thereof, and a compound of Formula I, or a pharmaceutically acceptable salt thereof.

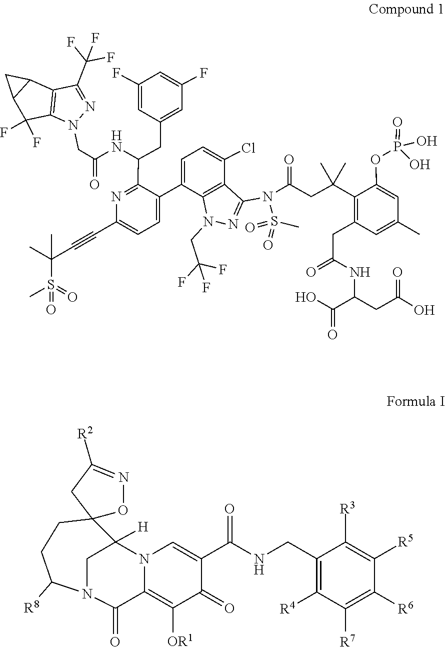

### CROSS-REFERENCE TO RELATED APPLICATIONS

This application claims the benefit of U.S. Provisional Application No. 63/729,942, filed Dec. 9, 2024, the entire contents of which is hereby incorporated by reference in its entirety.

### FIELD

A combination of Compound 1 and compounds of Formula I for treatment (e.g., therapeutic treatment) of viral infections, such as HIV infection, is disclosed. Pharmaceutical formulations of the combination of Compound 1 and compounds of Formula I suitable for treating the viral infections are also disclosed.

### BACKGROUND

Human immunodeficiency virus infection and related diseases are a major public health problem worldwide. Human immunodeficiency virus encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains may limit their usefulness (Palella, et al. N. Engl. J Med. (1998) 338:853-860; Richman, D. D. Nature (2001) 410:995-1001). Accordingly, there is a need for new agents that inhibit the replication of HIV.

A goal of antiretroviral therapy is to achieve viral suppression in the HIV infected patient. Current treatment guidelines published by the United States Department of Health and Human Services provide that achievement of viral suppression requires the use of combination therapies, i.e., several drugs from at least two or more drug classes (Panel on Antiretroviral Guidelines for Adults and Adolescents. Guidelines for the Use of Antiretroviral Agents in Adults and Adolescents Living with HIV. Department of Health and Human Services. Available at http://www.aidsinfo.nih.gov/ContentFiles/AdultandAdolescentGL.pdf. Accessed Feb. 12, 2019). In addition, decisions regarding the treatment of HIV infected patients are complicated when the patient requires treatment for other medical conditions (Id. at F-8). Because the standard of care requires the use of multiple different drugs to suppress HIV, as well as to treat other conditions the patient may be experiencing, the potential for drug interaction is a criterion for selection of a drug regimen. As such, there is a need for antiretroviral therapies having a decreased potential for drug interactions.

In addition, the HIV virus is known to mutate in infected subjects (Tang, et al. Drugs (2012) 72 (9) e1-e25). Because of the proclivity of the HIV virus to mutate, there is a need for anti-HIV drugs to be effective against a range of known HIV variants (Hurt, et al. HIV/AIDS CID (2014) 58, 423-431).

For certain patients, for example, those with difficult or limited access to health care, adherence to daily oral treatment or prophylactic regimens can be challenging. Drugs that offer favorable pharmaceutical properties (for example, improved potency, long-acting pharmacokinetics, low clearance, and/or other properties) are amenable to less frequent administration and provide for better patient compliance. For example, these favorable pharmaceutical properties may facilitate a reduced dose, a reduced pill size and/or a reduced pill count for better patient adherence. Such improvements can, in turn, optimize drug exposure and limit the emergence of drug resistance.

In some cases, compatible combination agents may be needed to modulate PK properties of a drug (such as dose, AUC, Cmax, and oral bioavailability) to achieve the target long-acting dosing regimens. There is thus a need for improved combination therapies.

### SUMMARY

In one aspect, this disclosure provides methods for the therapeutic treatment of an HIV infection in a subject in need thereof, the method comprising administering to the subject a therapeutically effective amount of:
(1) Compound 1, of the formula:

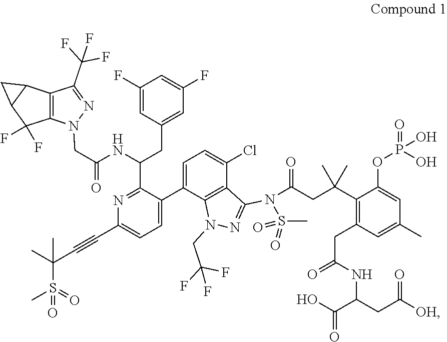 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof; and
(2) a compound of Formula I:

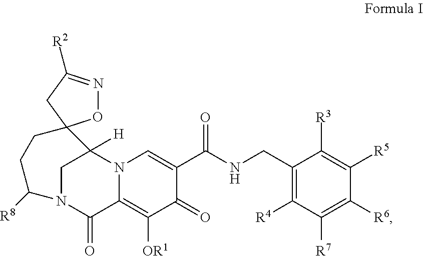

or a pharmaceutically acceptable salt thereof, wherein:
R¹ is —(CR^{1A}R^{1B}O)ₐ(Y)_{b}(CR^{1C}R^{1D})_{d}X;
wherein a is 0 or 1;
   b is 0 or 1;
   d is 0, 1, 2, 3, 4 or 5;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl; or
   optionally R^{1C} and R^{1D} on the same carbon atom are joined to form a spiro cyclopropyl group;
   Y is —C(O)—, —C(O)O—, —C(O)NH— or —C(O)NR^{1H};
   R^{1H} is C₁₋₄alkyl optionally substituted with one or two substituents independently selected from the group consisting of —COOH, —OH, —NH₂, —CONH₂, —P(O)(OH)₂, and —S(O)₂(OH);
   X is selected from the group consisting of:
   (a) —O—P(O)(OR^{1E})₂,
   wherein each R^{1E} is independently H or phenyl;
   (b) —N(R^{1F})₂,
      wherein each R^{1F} is independently H, COO(CR^{1I}R^{1J})ₑOPO(OH)₂, or C₁₋₄alkyl; wherein the C₁₋₄ alkyl is optionally substituted with one or two substituents independently selected from the group consisting of —COOH, —S(O)₂(OH), —P(O)(OH)₂, —OH, —NH₂, and —CONH₂;
      e is 1, 2, or 3;
      each R^{1I} is independently H or C₁₋₃alkyl;
      each R^{1J} is independently H or C₁₋₃alkyl; or
      optionally R^{1I} and R^{1J} on the same carbon atom are joined to form a spiro cyclopropyl group; and
   (c) —N⁺(R^{1G})₃Z⁻,
      wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂;
      Z⁻ is a counterion;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In another aspect, this disclosure provides uses of Compound 1, or a pharmaceutically acceptable salt thereof, and a compound of Formula I, or a pharmaceutically acceptable salt thereof, for the therapeutic treatment of an HIV infection in a subject.

In another aspect, this disclosure provides Compound 1, or a pharmaceutically acceptable salt thereof, and a compound of Formula I, or a pharmaceutically acceptable salt thereof, for use in the therapeutic treatment of an HIV infection in a subject.

In another aspect, this disclosure provides compounds of Formula I, or a pharmaceutically acceptable salt thereof, and Compound 1, or a pharmaceutically acceptable salt thereof, for use in the therapeutic treatment of an HIV infection in a subject.

In another aspect, this disclosure provides pharmaceutical compositions comprising a therapeutically effective amount of (a) Compound 1, or a pharmaceutically acceptable salt thereof, and (b) a therapeutically effective amount of a compound of Formula I, or a pharmaceutically acceptable salt thereof, and a pharmaceutically acceptable excipient.

In another aspect, this disclosure provides kits comprising (a) Compound 1, or a pharmaceutically acceptable salt thereof, and (b) a compound of Formula I, or a pharmaceutically acceptable salt thereof, and instructions for use.

In another aspect, this disclosure provides combinations comprising (a) Compound 1, or a pharmaceutically acceptable salt thereof, and (b) a compound of Formula I, or a pharmaceutically acceptable salt thereof.

Other embodiments, objects, features, and advantages may be set forth in the detailed description of the embodiments that follows, and in part may be apparent from the description, or may be learned by practice, of the claimed embodiments. These objects and advantages may be realized and attained by the processes and compositions particularly pointed out in the description and claims thereof. The foregoing Summary has been made with the understanding that it is to be considered as a brief and general synopsis of some of the embodiments disclosed herein, is provided for the benefit and convenience of the reader, and is not intended to limit in any manner the scope, or range of equivalents, to which the appended claims are lawfully entitled.

### BRIEF DESCRIPTION OF THE DRAWINGS

FIG. 1 shows the plasma concentrations in rats of Compound 1′ and lenacapavir (LEN) following oral administration of Compound 1′ or lenacapavir.

FIG. 2 shows the plasma concentrations in beagle dogs of Compound 1′ and lenacapavir (LEN) following oral administration of Compound 1′ or lenacapavir.

FIG. 3 shows the plasma concentration in dogs of Intermediate B following oral administration of Compound 28 with and without Compound 1′.

FIG. 4 shows the plasma concentration in dogs of Intermediate C following oral administration of Compound 29 with and without Compound 1′.

### DETAILED DESCRIPTION

In the following description, certain specific details are set forth in order to provide a thorough understanding of various embodiments disclosed herein. However, one skilled in the art will understand that the embodiments disclosed herein may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### I. Definitions

Unless the context requires otherwise, throughout the present disclosure and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment disclosed herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ radical.

“Hydroxy” or “hydroxyl” refers to the —OH radical.

The term “C₁₋ₙ alkyl” as used herein, wherein n is an integer, either alone or in combination with another radical, is intended to mean acyclic, straight or branched chain alkyl radicals containing from 1 to n carbon atoms. “C₁₋₆ alkyl” includes, but is not limited to, methyl, ethyl, propyl (n-propyl), butyl (n-butyl), 1-methylethyl (isopropyl), 1-methylpropyl (sec-butyl), 2-methylpropyl (iso-butyl), 1,1-dimethylethyl (tertbutyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

“Alkyl” is hydrocarbon containing normal, secondary or tertiary atoms. For example, an alkyl group can have 1 to 20 carbon atoms (i.e., C₁₋₂₀ alkyl), 1 to 10 carbon atoms (i.e., C₁₋₁₀ alkyl), 1 to 8 carbon atoms (i.e., C₁₋₈ alkyl) or 1 to 6 carbon atoms (i.e., C₁₋₆ alkyl). Examples of suitable alkyl groups include, but are not limited to, methyl (Me, —CH₃), ethyl (Et, CH₂CH₃), 1-propyl (n-Pr, n-propyl, —CH₂CH₂CH₃), 2-propyl (i-Pr, ipropyl, CH(CH₃)₂), 1-butyl (n-Bu, n-butyl, —CH₂CH₂CH₂CH₃), 2-methyl1-propyl (i-Bu, ibutyl, —CH₂CH(CH₃)₂), 2-butyl (s-Bu, s-butyl, CH(CH₃)CH₂CH₃), 2-methyl-2-propyl (t-Bu, tbutyl, —C(CH₃)₃), 1-pentyl (n-pentyl, CH₂CH₂CH₂CH₂CH₃), 2-pentyl (CH(CH₃)CH₂CH₂CH₃), 3-pentyl (CH(CH₂CH₃)₂), 2-methyl-2-butyl (C(CH₃)₂CH₂CH₃), 3-methyl-2-butyl (CH(CH₃)CH(CH₃)₂), 3-methyl1butyl (CH₂CH₂CH(CH₃)₂), 2-methyl-1-butyl (CH₂CH(CH₃)CH₂CH₃), 1-hexyl (CH₂CH₂CH₂CH₂CH₂CH₃), 2-hexyl (CH(CH₃)CH₂CH₂CH₂CH₃), 3-hexyl (CH(CH₂CH₃)(CH₂CH₂CH₃)), 2-methyl-2-pentyl (C(CH₃)₂CH₂CH₂CH₃), 3-methyl-2-pentyl (CH(CH₃)CH(CH₃)CH₂CH₃), 4-methyl-2-pentyl (CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (C(CH₃)₂CH(CH₃)₂), 3,3-dimethyl-2-butyl (—CH(CH₃)C(CH₃)₃, and octyl ((CH₂)₇CH₃). “Alkyl” also refers to a saturated, branched or straight chain hydrocarbon radical having two monovalent radical centers derived by the removal of two hydrogen atoms from the same or two different carbon atoms of a parent alkane. For example, an alkyl group can have 1 to 10 carbon atoms (i.e., C₁₋₁₀alkyl), or 1 to 6 carbon atoms (i.e., C₁₋₆alkyl) or 1-3 carbon atoms (i.e., C₁₋₃ alkyl). Typical alkyl radicals include, but are not limited to, methylene (CH₂), 1,1-ethyl (CH(CH₃)), 1,2-ethyl (CH₂CH₂), 1,1-propyl (CH(CH₂CH₃)), 1,2-propyl (CH₂CH(CH₃)), 1,3-propyl (CH₂CH₂CH₂), 1,4-butyl (CH₂CH₂CH₂CH₂), and the like.

The term “alkenyl” as used herein refers to a straight or branched hydrocarbon containing normal, secondary or tertiary carbon atoms with at least one site of unsaturation, i.e., a carbon-carbon, sp² double bond. For example, an alkenyl group can have 2 to 20 carbon atoms (i.e., C₂-C₂₀ alkenyl, or C₂₋₂₀ alkenyl), 2 to 8 carbon atoms (i.e., C₂-C₈ alkenyl or C₂₋₈), or 2 to 6 carbon atoms (i.e., C₂-C₆ alkenyl, or C₂₋₆ alkenyl). Examples of suitable alkenyl groups include, but are not limited to, ethylene or vinyl (CH═CH₂), allyl (CH₂CH═CH₂), cyclopentenyl (C₁H₇), and 5-hexenyl (CH₂CH₂CH₂CH₂CH═CH₂).

The term “C₂₋ₙalkenyl”, as used herein, wherein n is an integer, either alone or in combination with another radical, is intended to mean an unsaturated, acyclic straight or branched chain radical containing two to n carbon atoms, at least two of which are bonded to each other by a double bond. Examples of such radicals include, but are not limited to, ethenyl (vinyl), 1propenyl, 2propenyl, and 1butenyl. Unless specified otherwise, the term “C₂₋ₙalkenyl” is understood to encompass individual stereoisomers where possible, including but not limited to (E) and (Z) isomers, and mixtures thereof. When a C₂₋ₙalkenyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “halo” or “halogen” as used herein refers to fluoro, chloro, bromo and iodo.

The term “C₃₋ₘ cycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl and cycloheptyl. The term includes fully saturated as well as partially unsaturated rings.

It is to be understood that when a variable is substituted, for example, as described by the phrase “C₁₋₆ alkyl, either alone or as part of a group, is optionally substituted”, the phrase means that the variable C₁₋₆ alkyl can be substituted when it is alone and that it can also be substituted when the variable “C₁₋₆ alkyl” is part of a larger group. Similarly, when stated, other variables can also be substituted “either alone or as part of a group.”

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers or axes of chirality and whose molecules are not mirror images of one another. Diastereomers typically have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “treatment” or “treating,” to the extent it relates to a disease or condition includes inhibiting or ameliorating the disease or condition (e.g., arresting or slowing its development), eliminating the disease or condition (e.g., causing regression or cure of the disease or condition), and/or relieving one or more symptoms of the disease or condition. In the case of HIV, treatment includes reducing the level of HIV viral load in a subject or patient.

In some embodiments, the term “treatment” refers to the administration of a compound or composition according to the present invention to alleviate or eliminate symptoms of HIV infection and/or to reduce viral load in a subject or patient. The term “treatment” also encompasses the administration of a compound or composition according to the present invention before the exposure of the individual to the virus, postexposure of the individual to the virus but before the appearance of symptoms of the disease, and/or prior to the detection of the virus in the blood.

The term “combination therapy” as used herein refers to the use of two or more treatments, such as pharmaceutically active agents (i.e., a “combination”), to treat a single disease or condition. The pharmaceutically active agents can be administered together or separately, as well as simultaneously or sequentially, usually so that their functionalities coincide and have overlapping effects on a subject or patient, resulting in a desired therapeutic effect on the subject or patient. In some embodiments, the pharmaceutically active agents are from at least two or more drug classes.

“Protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

Protecting groups are available, commonly known and used, and are optionally used to prevent side reactions with the protected group during synthetic procedures, i.e., routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. PGs do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene (John Wiley & Sons, Inc., New York, 1991, ISBN 0-471-62301-6) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below.

The term “solvate” refers to a crystalline solid containing amounts of a solvent incorporated within the crystal structure. As used herein, the term “solvate” includes hydrates.

The term “non-solvate” refers to a crystalline solid in which no solvent molecules occupy a specific crystallographic site.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Birge et al., J. Pharm. Sci., 1977, 66, pp. 1-19.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, sulfuric acid, sulfamic acid, nitric acid, phosphoric acid and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like. Particularly preferred are the ammonium, potassium, sodium, calcium, and magnesium salts. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins and the like. Particularly preferred organic nontoxic bases are isopropylamine, diethylamine, ethanolamine, trimethylamine, dicyclohexylamine, choline, and caffeine.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal. The term “antiviral agent” includes, for example, an HIV integrase catalytic site inhibitor selected from the group consisting: raltegravir (ISENTRESS®; Merck); elvitegravir (Gilead); soltegravir (GSK; ViiV); GSK 1265744 (GSK; ViiV) and dolutegravir; an HIV nucleoside reverse transcriptase inhibitor selected from the group consisting of: abacavir (ZIAGEN®; GSK); didanosine (VIDEX®; BMS); tenofovir (VIREAD®; Gilead); emtricitabine (EMTRIVA®; Gilead); lamivudine (EPIVIR®; GSK/Shire); stavudine (ZERIT®; BMS); zidovudine (RETROVIR®; GSK); elvucitabine (Achillion); and festinavir (Oncolys); an HIV non-nucleoside reverse transcriptase inhibitor selected from the group consisting of: nevirapine (VIRAMUNE®; BI); efavirenz (SUSTIVA®; BMS); etravirine (INTELENCE®; J&J); rilpivirine (TMC278, R278474; J&J); fosdevirine (GSK/ViiV); and lersivirine (Pfizer/ViiV); an HIV protease inhibitor selected from the group consisting of: atazanavir (REYATAZ®; BMS); darunavir (PREZISTA®; J&J); indinavir (CRIXIVAN®; Merck); lopinavir (KELETRA®; Abbott); nelfinavir (VIRACEPT®; Pfizer); saquinavir (INVIRASE®; Hoffmann-LaRoche); tipranavir (APTIVUS®; BI); ritonavir (NORVIR®; Abbott); and fosamprenavir (LEXIVA®; GSK/Vertex); an HIV entry inhibitor selected from: maraviroc (SELZENTRY®; Pfizer); enfuvirtide (FUZEON®; Trimeris); and BMS-663068 (BMS); and an HIV maturation inhibitor selected from: bevirimat (Myriad Genetics).

The term “inhibitor of HIV replication” as used herein is intended to mean an agent capable of reducing or eliminating the ability of HIV to replicate in a host cell, whether in vitro, ex vivo or in vivo.

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The term “heteroatom” as used herein is intended to mean 0, S or N.

The terms “O—C₁₋ₙ alkyl” or “C₁₋ₙ alkoxy” as used herein interchangeably, wherein n is an integer, either alone or in combination with another radical, is intended to mean an oxygen atom further bonded to an alkyl radical having 1 to n carbon atoms as defined above. Examples of C₁₋ₙ alkoxy include but are not limited to methoxy (CH₃O—), ethoxy (CH₃CH₂O—), propoxy (CH₃CH₂CH₂O—), 1-methylethoxy (iso-propoxy; (CH₃)₂CHO) and 1,1-dimethylethoxy (tert-butoxy; (CH₃)₃CO). When an C₁₋ₙ alkoxy is substituted, it is understood to be substituted on the alkyl portion thereof, such that the substitution would give rise to a chemically stable compound, such as are recognized by those skilled in the art.

The term “mammal” as used herein is intended to encompass humans, as well as non-human mammals which are susceptible to infection by HIV. Non-human mammals include but are not limited to domestic animals, such as cows, pigs, horses, dogs, cats, rabbits, rats and mice, and non-domestic animals.

The embodiments disclosed herein are also meant to encompass all pharmaceutically acceptable compounds of Formula I, Formula I′, Formula II, or Formula II′ being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. In certain embodiments, these radiolabeled compounds are useful to help determine or measure the effectiveness of the compounds, by characterizing, for example, the site or mode of action, or binding affinity to pharmacologically important site of action. Certain isotopically-labeled compounds of Formula I, Formula I′, Formula II, or Formula II′, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

In certain embodiments, substitution with heavier isotopes such as deuterium, i.e., 2H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵, and ¹³N, can be useful in Positron Emission Topography (PET) studies for examining substrate receptor occupancy. Isotopically-labeled compounds of the compounds disclosed herein can be prepared by techniques known to those skilled in the art or by processes analogous to those described in the Examples as set out below using an appropriate isotopically-labeled reagent in place of the non-labeled reagent previously employed.

The methods, compositions, kits and articles of manufacture provided herein use or include (i) Compound 1, or pharmaceutically acceptable salts thereof, or Compound 1′, or pharmaceutically acceptable salts thereof, and (ii) compounds of Formula I, Formula I′, Formula II, or Formula II′, or pharmaceutically acceptable salts thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds increase resistance to metabolism, and thus are useful for increasing the half-life of compounds or pharmaceutically acceptable salts thereof, when administered to a mammal. See, e.g., Foster, “Deuterium Isotope Effects in Studies of Drug Metabolism”, Trends Pharmacol. Sci., 5(12):524-527 (1984). Such compounds can be synthesized by means known in the art, for example by employing starting materials in which one or more hydrogen atoms have been replaced by deuterium.

The embodiments disclosed herein are also meant to encompass the in vivo metabolic products of the disclosed (i) Compound 1, or pharmaceutically acceptable salts thereof, or Compound 1′, or pharmaceutically acceptable salts thereof, and (ii) compounds of Formula I, or pharmaceutically acceptable salts thereof, Formula I′, or pharmaceutically acceptable salts thereof, Formula II, or pharmaceutically acceptable salts thereof, or Formula II′, or pharmaceutically acceptable salts thereof. Such products may result from, for example, the oxidation, reduction, hydrolysis, amidation, esterification, and the like of the administered compound, primarily due to enzymatic processes. Accordingly, the embodiments disclosed herein include (i) Compound 1, or pharmaceutically acceptable salts thereof, or Compound 1′, or pharmaceutically acceptable salts thereof, and (ii) compounds of Formula I, or pharmaceutically acceptable salts thereof, Formula I′, or pharmaceutically acceptable salts thereof, Formula II, or pharmaceutically acceptable salts thereof, or Formula II′, or pharmaceutically acceptable salts thereof, produced by a process comprising administering a compound according to the embodiments disclosed herein to a mammal for a period of time sufficient to yield a metabolic product thereof. Such products are typically identified by administering a radiolabeled compound according to the embodiments disclosed herein in a detectable dose to an animal, such as rat, mouse, guinea pig, monkey, or to human, allowing sufficient time for metabolism to occur, and isolating its conversion products from the urine, blood or other biological samples.

Compound 1, or pharmaceutically acceptable salts thereof, or Compound 1′, or pharmaceutically acceptable salts thereof, and compounds of Formula I, or pharmaceutically acceptable salts thereof, Formula I′, or pharmaceutically acceptable salts thereof, Formula II, or pharmaceutically acceptable salts thereof, or Formula II′, or pharmaceutically acceptable salts thereof, disclosed herein may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic, scalemic, and optically pure forms. Optically active (+) and (−), (R)- and (S)-, or (D)- and (L)- isomers may be prepared using chiral synthons or chiral reagents, or resolved using methods such as chromatography and fractional crystallization. Techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

“Optional” or “optionally” means that the subsequently described event or circumstances may or may not occur, and that the description includes instances where said event or circumstance occurs and instances in which it does not. For example, “optionally substituted heterocyclyl” means that the heterocyclyl radical may or may not be substituted and that the description includes both substituted heterocyclyl radicals and heterocyclyl radicals having no substitution.

The present disclosure provides methods of treating HIV infections using (i) Compound 1, or a pharmaceutically acceptable salt thereof, or Compound 1′, or pharmaceutically acceptable salts thereof, and (ii) a compound of Formula I, or a pharmaceutically acceptable salt thereof, or a compound of Formula I′, or a pharmaceutically acceptable salt thereof. Also provided herein are pharmaceutical formulations, for example the tablets, comprising the two active pharmaceutical ingredients (APIs), i.e., (i) Compound 1, or a pharmaceutically acceptable salt thereof, or Compound 1′, or pharmaceutically acceptable salts thereof, and (ii) a compound of Formula I, or a pharmaceutically acceptable salt thereof, a compound of Formula I′, or a pharmaceutically acceptable salt thereof, a compound of Formula II, or a pharmaceutically acceptable salt thereof, or a compound of Formula II′, or a pharmaceutically acceptable salt thereof.

### II. Compound 1

Compound 1 is an HIV capsid inhibitor of the formula (see WO 2023/102239):

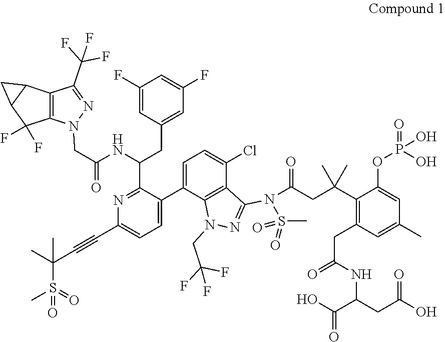 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

The IUPAC name of Compound 1 is (2-(2-(4-(N-(4-chloro-7-(2-(1-(2-(5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl)but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetyl)aspartic acid.

In some embodiments, Compound 1 is Compound 1′, of the formula:

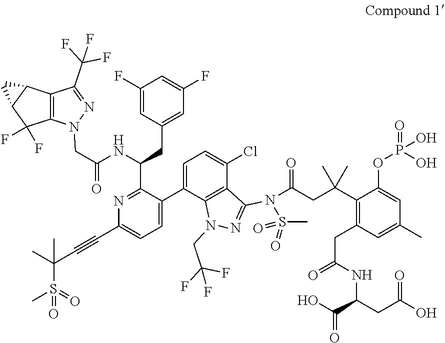 [SMILES: Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof.

Compound 1′ is an HIV capsid inhibitor of the formula (see WO 2023/102239):

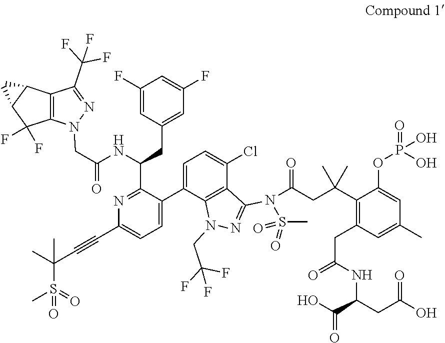 [SMILES: Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

The IUPAC name of Compound 1′ is (2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl)but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetyl)-L-aspartic acid.

In some embodiments, the dosing of Compound 1 or Compound 1′ results in the formation of lenacapavir, which is known to be active against HIV, as disclosed, for example, in U.S. Pat. No. 10,071,985. In some embodiments, Compound 1 or Compound 1′ are converted to lenacapavir in the gastrointestinal tract. In some embodiments, Compound 1 or Compound 1′ are more soluble than lenacapavir and thus are administered orally at a lower effective dose than the required oral effective dose for lenacapavir to achieve the same level of exposure of lenacapavir in vivo.

In some embodiments, the methods of therapeutic treatment comprise administering Compound 1 as a free acid or in the form of a pharmaceutically acceptable salt. The Compound 1 or its pharmaceutically acceptable salts can be present within a tablet in solvated or unsolvated form, and references to “Compound 1” include both of these forms. In particular, references to the Compound 1, or a pharmaceutically acceptable salt thereof, include pharmaceutically acceptable salts of the Compound 1 and pharmaceutically acceptable hydrates of the Compound 1. Additionally, the term “Compound 1” is meant to refer to either or both a single molecule of Compound 1 (or a pharmaceutically acceptable salt thereof), or a preparation of Compound 1 (or a pharmaceutically acceptable salt thereof), where the preparation may be characterized by bulk properties such as purity, particle size, and other characteristics.

As used herein, and in the absence of a specific reference to a particular pharmaceutically acceptable salt and/or solvate of the Compound 1, any dosages, whether expressed in e.g. milligrams or as a % by weight, should be taken as referring to the amount of the Compound 1 free acid, i.e. the amount of the compound of the formula:

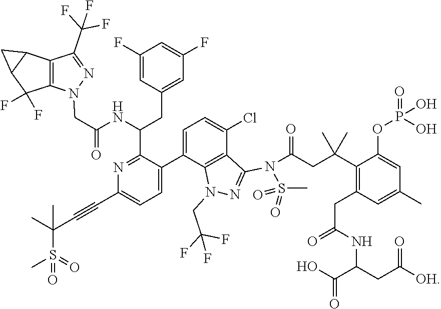 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

The pharmaceutical compositions described herein utilize the Compound 1, wherein the Compound 1 is in the form of a free acid and/or a pharmaceutically acceptable salt thereof. In some embodiments, the pharmaceutical composition comprises the Compound 1. In some embodiments, the pharmaceutical composition comprises a pharmaceutically acceptable salt of the Compound 1.

In some embodiments, the methods of therapeutic treatment comprise administering Compound 1′ as a free acid or in the form of a pharmaceutically acceptable salt. The Compound 1′ or its pharmaceutically acceptable salts can be present within a tablet in solvated or unsolvated form, and references to “Compound 1′” include both of these forms. In particular, references to the Compound 1′, or a pharmaceutically acceptable salt thereof, include pharmaceutically acceptable salts of the Compound 1′ and pharmaceutically acceptable hydrates of the Compound 1′. Additionally, the term “Compound 1′” is meant to refer to either or both a single molecule of Compound 1′ (or a pharmaceutically acceptable salt thereof), or a preparation of Compound 1′ (or a pharmaceutically acceptable salt thereof), where the preparation may be characterized by bulk properties such as purity, particle size, and other characteristics.

As used herein, and in the absence of a specific reference to a particular pharmaceutically acceptable salt and/or solvate of the Compound 1′, any dosages, whether expressed in e.g. milligrams or as a % by weight, should be taken as referring to the amount of the Compound 1′ free acid, i.e. the amount of:

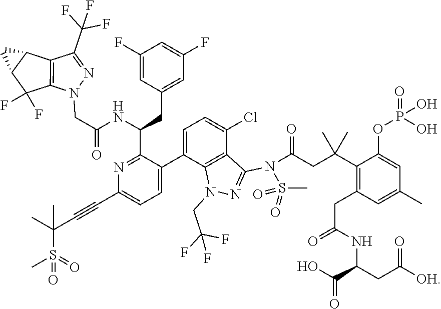 [SMILES: Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

The pharmaceutical compositions described herein utilize the Compound 1′, wherein the Compound 1′ is in the form of a free acid and/or a pharmaceutically acceptable salt thereof. In some embodiments, the pharmaceutical composition comprises the Compound 1′. In some embodiments, the pharmaceutical composition comprises a pharmaceutically acceptable salt of the Compound 1′.

### III. Compounds of Formula I

In some embodiments, the methods, uses, compounds for use, kits, composition, and combinations as disclosed herein comprise a compound of Formula I, or a pharmaceutically acceptable salt thereof, wherein the compound of Formula I is of the formula:

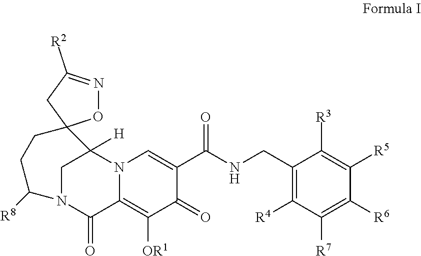

or a pharmaceutically acceptable salt thereof, wherein:
R¹ is —(CR^{1A}R^{1B}O)ₐ(Y)_{b}(CR^{1C}R^{1D})_{d}X;
   wherein a is 0 or 1;
   b is 0 or 1;
   d is 0, 1, 2, 3, 4 or 5;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl; or
   optionally R^{1C} and R^{1D} on the same carbon atom are joined to form a spiro cyclopropyl group;
   Y is —C(O)—, —C(O)O—, —C(O)NH— or —C(O)NR^{1H}—;
   R^{1H} is C₁₋₄alkyl optionally substituted with one or two substituents independently selected from the group consisting of —COOH, —OH, —NH₂, —CONH₂, —P(O)(OH)₂, and —S(O)₂(OH);
   X is selected from the group consisting of:
   (a) —O—P(O)(OR^{1E})₂,
      wherein each R^{1E} is independently H or phenyl;
   (b) —N(R^{1F})₂,
      wherein each R^{1F} is independently H, —COO(CR^{1I}R^{1J})ₑOPO(OH)_{2′} or C₁₋₄alkyl; wherein the C₁₋₄ alkyl is optionally substituted with one or two substituents independently selected from the group consisting of —COOH, —S(O)₂(OH), —P(O)(OH)₂, —OH, —NH₂, and —CONH₂;
      e is 1, 2, or 3;
      each R^{1I} is independently H or C₁₋₃alkyl;
      each R^{1J} is independently H or C₁₋₃alkyl; or
      optionally R¹¹ and R^{1J} on the same carbon atom are joined to form a spiro cyclopropyl group; and
   (c) —N⁺(R^{1G})₃Z⁻,
      wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂;
      Z⁻ is a counterion;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In some embodiments, the compound of Formula I is a compound of Formula I′:

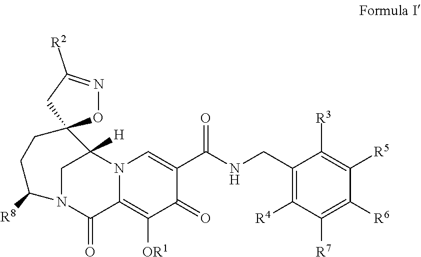

or a pharmaceutically acceptable salt thereof, wherein R¹, R², R³, R⁴, R⁵, R⁶, R⁷, and R⁸ are as defined according to any other embodiment described herein.

In some embodiments, a is 0. In some embodiments, a is 1.

In some embodiments, b is 0. In some embodiments, b is 1.

In some embodiments, d is 1, 2, 3, or 4. In some embodiments, d is 1, 2, or 3. In some embodiments, d is 1. In some embodiments, d is 2. In some embodiments, d is 3. In some embodiments, d is 4.

In some embodiments, each R^{1A} is H. In some embodiments, each R^{1B} is H. In some embodiments, each R^{1A} and R^{1B} is H.

In some embodiments, each R^{1A} is C₁₋₃alkyl and each R^{1B} is H. In some embodiments, each R^{1A} is methyl. In some embodiments, each R^{1A} is methyl and each R^{1B} is H.

In some embodiments, each R^{1C} is H. In some embodiments, each R^{1D} is H. In some embodiments, each R^{1C} and R^{1D} is H. In some embodiments, each R^{1C} is independently H or methyl. In some embodiments, each R^{1D} is independently H or methyl. In some embodiments, each R^{1C} is independently H or methyl and each R^{1D} is H.

In some embodiments, Y is —C(O)—, —C(O)O—, —C(O)NH— or —C(O)NCH₃—. In some embodiments, Y is —C(O)—, —C(O)O— or —C(O)NCH₃—. In some embodiments, Y is —C(O)—. In some embodiments, Y is —C(O)O—. In some embodiments, Y is —C(O)NCH₃—.

In some embodiments, Y is —C(O)NR^{1H}—. In some embodiments, R^{1H} is C₁₋₂alkyl optionally substituted with —COOH, —P(O)(OH)₂, or —S(O)₂(OH).

In some embodiments, X is —O—P(O)(OR^{1E})₂. In some embodiments, each R^{1E} is phenyl. In some embodiments, each R^{1E} is H.

In some embodiments, X is —N(R^{1F})₂. In some embodiments, each R^{1F} is independently —COO(CR^{1I}R^{1J})ₑOPO(OH)₂ or C₁₋₄alkyl; wherein the C₁₋₄ alkyl is optionally substituted with one or two —COOH. In some embodiments, each R^{1F} is independently —COO(CR^{1I}R^{1J})ₑOPO(OH)₂ or C₁₋₄alkyl; wherein the C₁₋₄ alkyl is optionally substituted with one —COOH.

In some embodiments, e is 1 or 2. In some embodiments, e is 1. In some embodiments, e is 2.

In some embodiments, each R^{1I} is H. In some embodiments, each R^{1J} is H. In some embodiments, each R^{1I} and each R^{1I} is H. In some embodiments, each R^{1I} is C₁₋₃alkyl and each R^{1I} is H.

In some embodiments, each R^{1F} is independently —COOCH₂OPO(OH)₂ or C₁₋₄alkyl; wherein the C₁₋₄ alkyl is optionally substituted with one or two —COOH. In some embodiments, each R^{1F} is independently —COOCH₂OPO(OH)₂ or C₁₋₄alkyl; wherein the C₁₋₄ alkyl is optionally substituted with one —COOH.

In some embodiments, each R^{1F} is independently —COO(CH₂)₂OPO(OH)₂, —COOCH₂OPO(OH)₂, —CH₃, or —CH₂COOH. In some embodiments, each R^{1F} is independently —COOCH₂OPO(OH)₂, —CH₃, or —CH₂COOH.

In some embodiments, X is —N⁺(R^{1G})₃Z⁻.

In some embodiments, Z− is selected from the group consisting of acetate, ascorbate, aspartate, besylate, benzoate, bromide, bicarbonate, carbonate, cinnamate, citrate, chloride, formate, fumarate, gluconate, glutamate, glycolate, lactate, malate, maleate, malonate, mandelate, mesylate, nicotinate, nitrate, oxalate, dihydrogen phosphate, hydrogen phosphate, phosphate, propionate, tosylate, pyroglutamate, salicylate, succinate, bisulfate, sulfate, tartrate, thiocyanate, triflate, and trifluoroacetate.

In some embodiments, each R^{1G} is independently C₁₋₄alkyl. In some embodiments, each R^{1G} is —CH₃.

In some embodiments, R¹ is selected from the group consisting of:

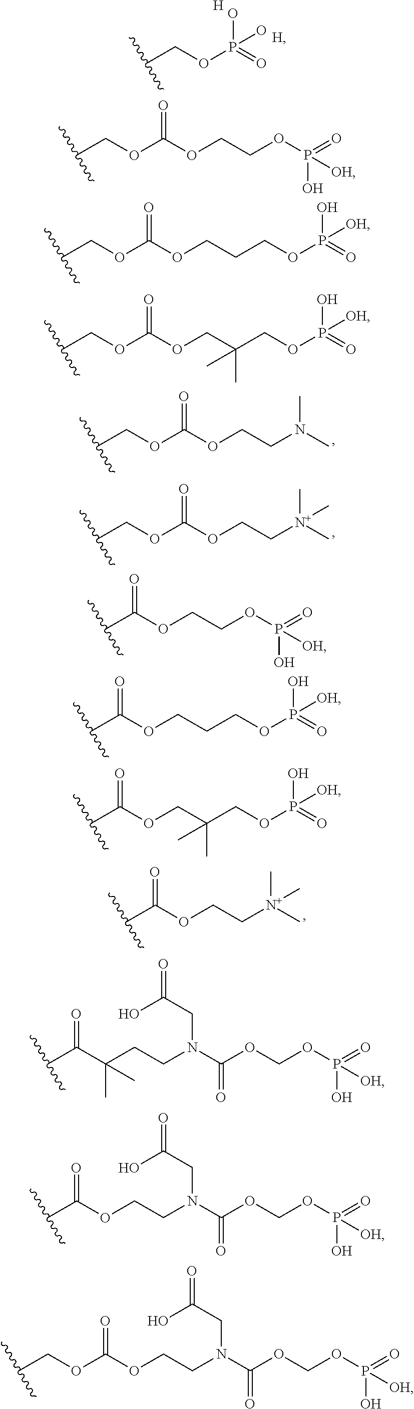

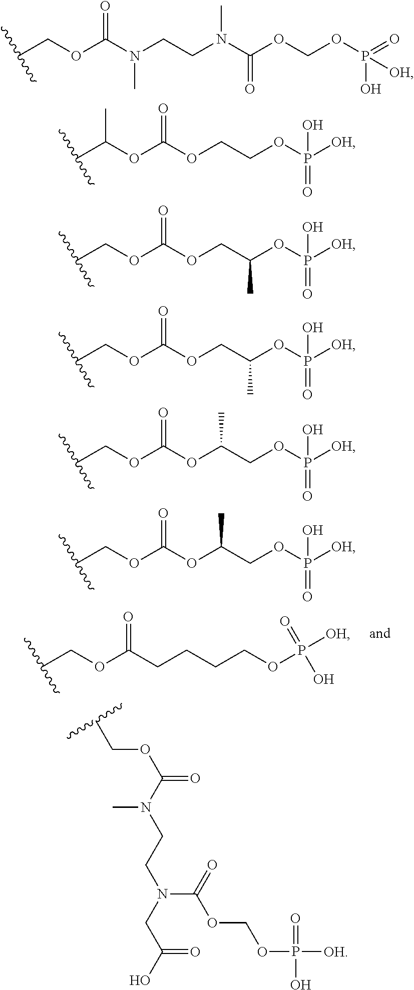

In some embodiments, R¹ is selected from the group consisting of:

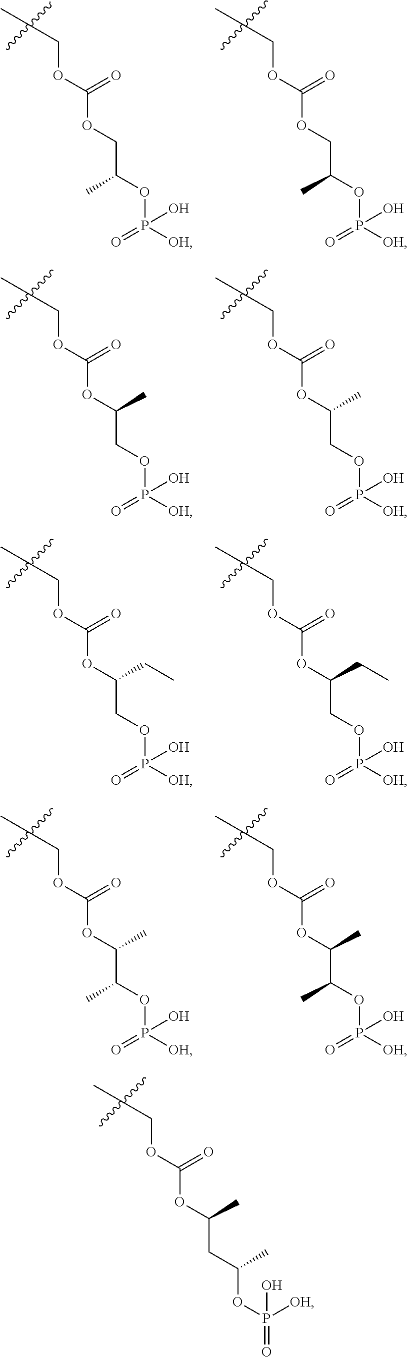

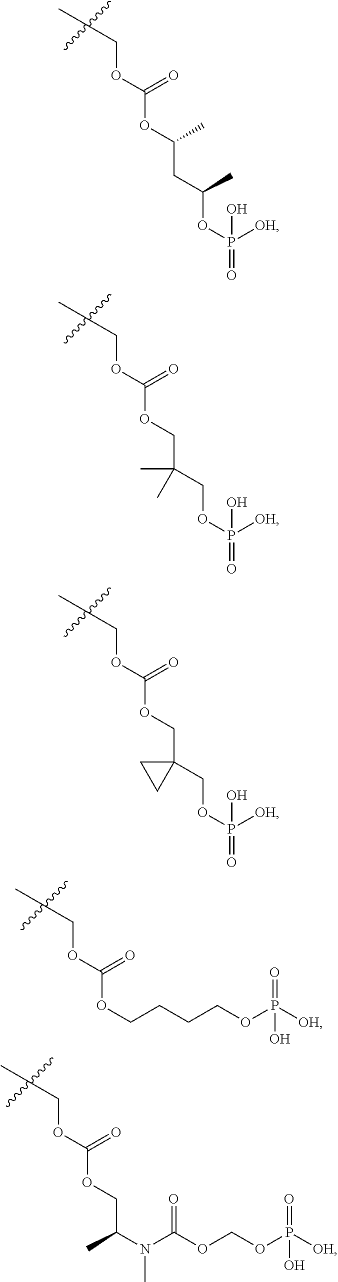 [SMILES: *COC(=O)O[C@H](C)C[C@@H](C)OP(=O)(O)O ; *COC(=O)OCC(C)(C)COP(=O)(O)O ; *COC(=O)OCC1(COP(=O)(O)O)CC1 ; *COC(=O)OCCCCOP(=O)(O)O ; *COC(=O)OC[C@H](C)N(C)C(=O)OCOP(=O)(O)O]

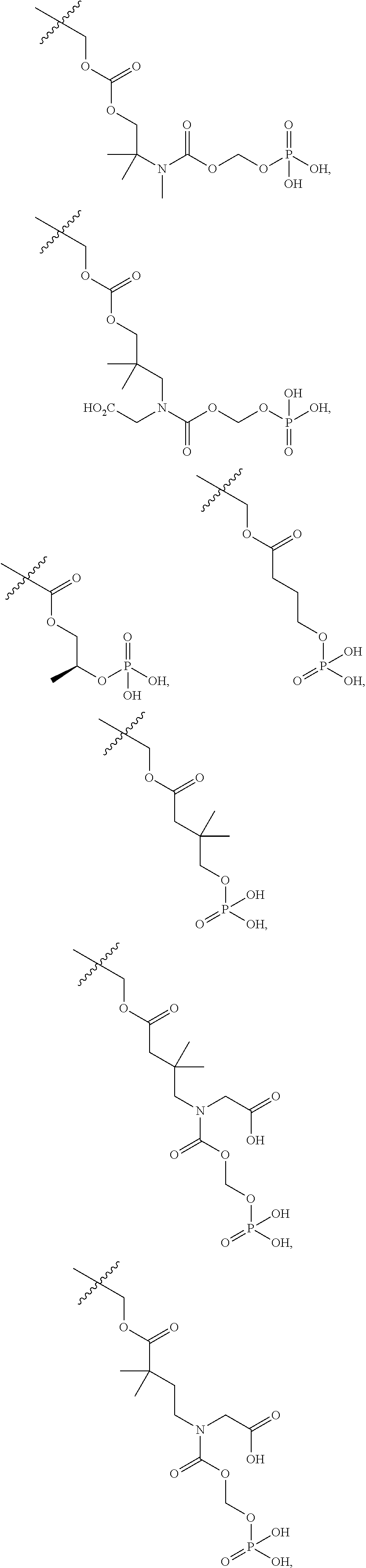

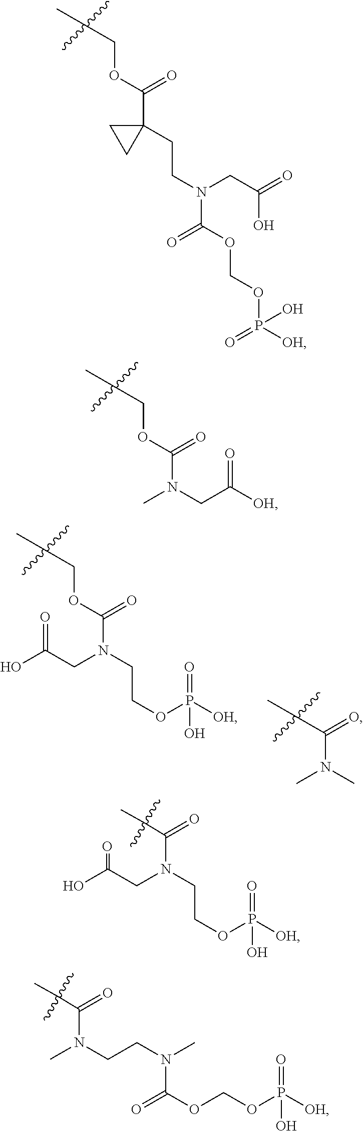

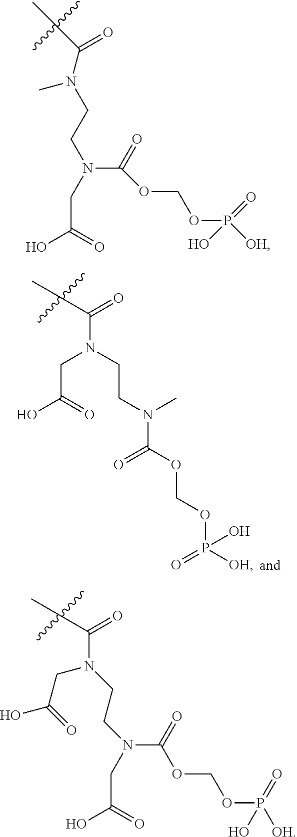 [SMILES: *C(=O)N(C)CCN(CC(=O)O)C(=O)OCOP(=O)(O)O ; *C(=O)N(CCN(C)C(=O)OCOP(=O)(O)O)CC(=O)O ; "and" ; *C(=O)N(CCN(CC(=O)O)C(=O)OCOP(=O)(O)O)CC(=O)O]

In some embodiments, R¹ is selected from the group consisting of:

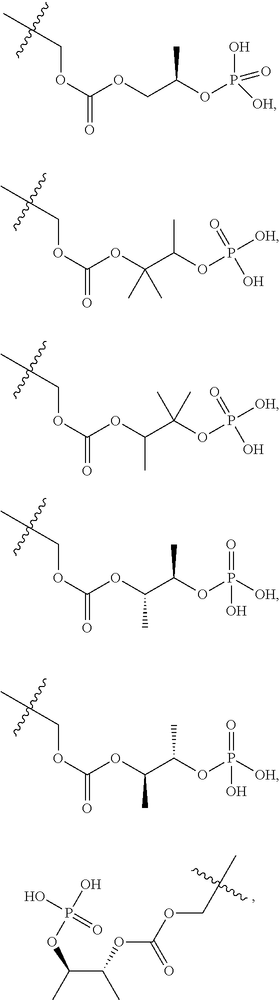 [SMILES: *COC(=O)OC[C@@H](C)OP(=O)(O)O ; *COC(=O)OC(C)(C)C(C)OP(=O)(O)O ; *COC(=O)OC(C)C(C)(C)OP(=O)(O)O ; *COC(=O)O[C@@H](C)[C@@H](C)OP(=O)(O)O ; *COC(=O)O[C@H](C)[C@H](C)OP(=O)(O)O ; *COC(=O)O[C@H](C)[C@@H](C)OP(=O)(O)O]

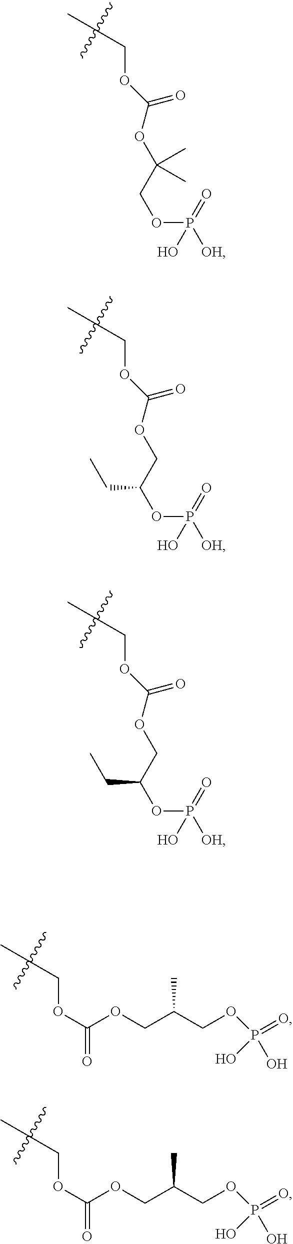 [SMILES: *COC(=O)OC(C)(C)COP(=O)(O)O ; *COC(=O)OC[C@@H](CC)OP(=O)(O)O ; *COC(=O)OC[C@H](CC)OP(=O)(O)O ; *COC(=O)OC[C@H](C)COP(=O)(O)O ; *COC(=O)OC[C@@H](C)COP(=O)(O)O]

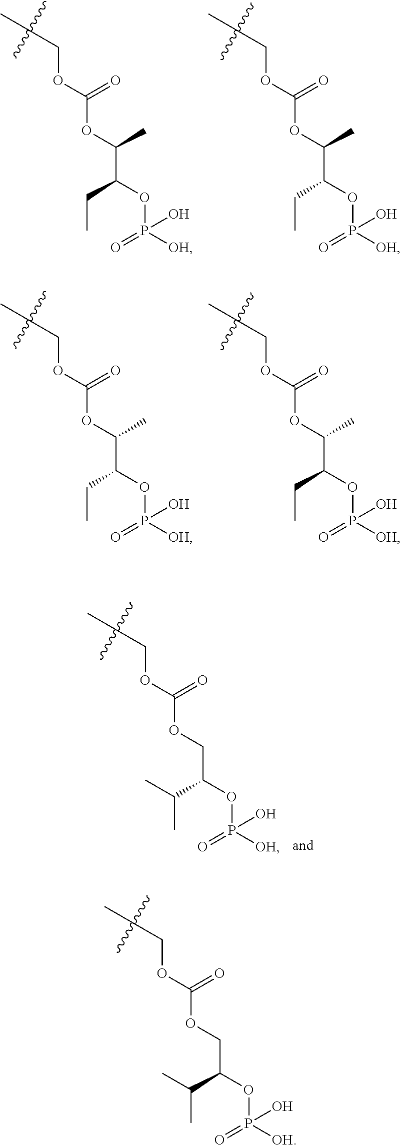 [SMILES: *COC(=O)O[C@@H](C)[C@H](CC)OP(=O)(O)O ; *COC(=O)O[C@@H](C)[C@@H](CC)OP(=O)(O)O ; *COC(=O)O[C@H](C)[C@@H](CC)OP(=O)(O)O ; *COC(=O)O[C@H](C)[C@H](CC)OP(=O)(O)O ; *COC(=O)OC[C@H](OP(=O)(O)O)C(C)C ; *COC(=O)OC[C@@H](OP(=O)(O)O)C(C)C]

In some embodiments, R² is methyl or methoxy. In some embodiments, R² is methyl. In some embodiments, R² is methoxy.

In some embodiments, R³ and R⁶ are each independently a halo. In some embodiments, R³ and R⁶ are each F.

In some embodiments, R⁴, R⁵ and R⁷ are each H.

In some embodiments, R⁸ is C₁₋₃alkyl. In some embodiments, R⁸ is methyl.

In some embodiments, the compound of Formula I or Formula I′ is selected from the group consisting of:

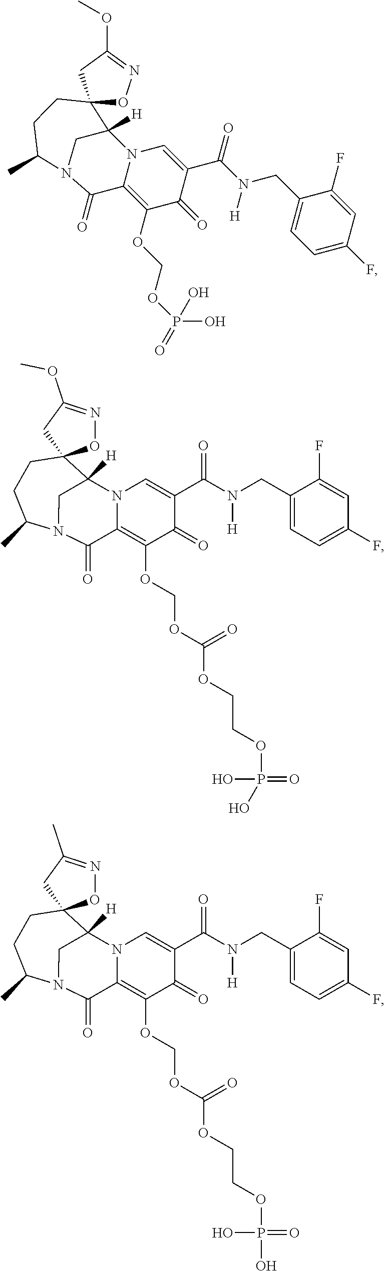 [SMILES: COC1=NO[C@@]2(CC[C@H](C)N3C[C@H]2n2cc(C(=O)NCc4ccc(F)cc4F)c(=O)c(OCOP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1]

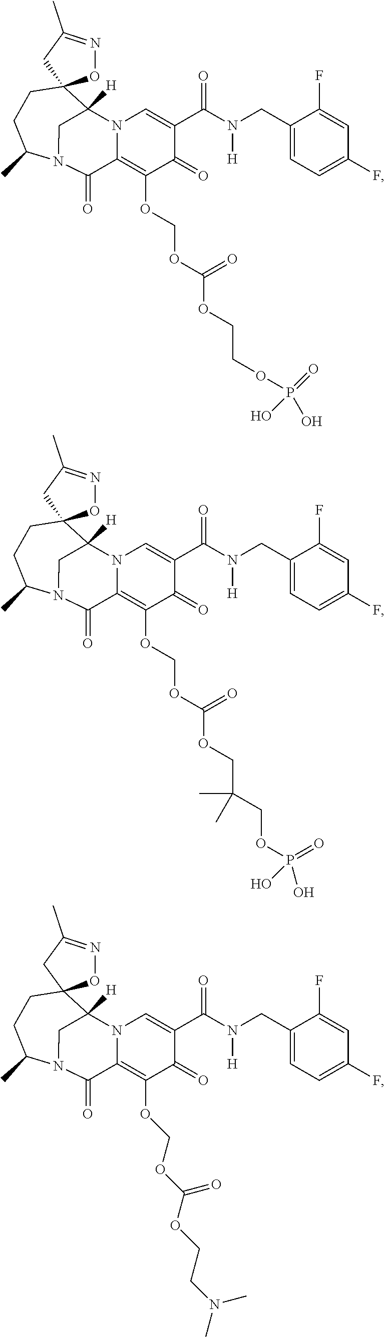 [SMILES: CC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCN(C)C)c2C3=O)C1]

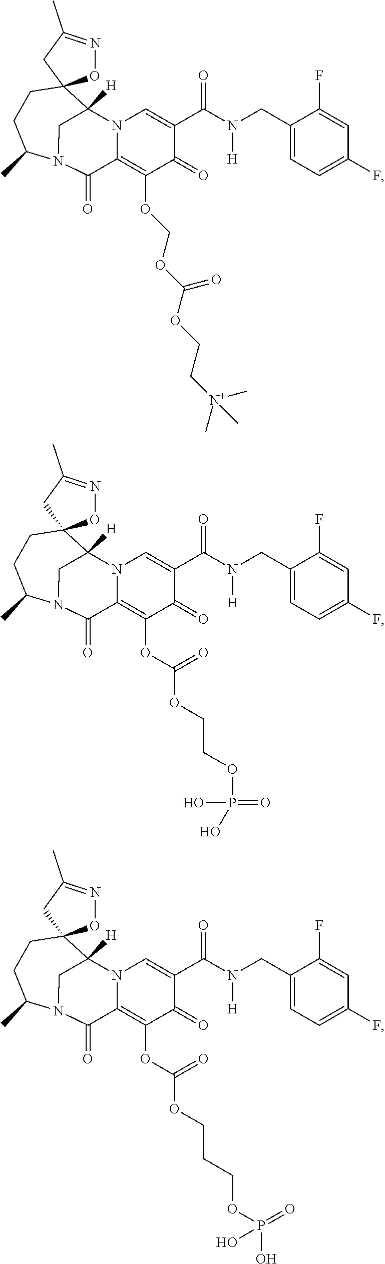 [SMILES: CC1=NO[C@@]2(CC[C@H](C)N3C[C@H]2n2cc(C(=O)NCc4ccc(F)cc4F)c(=O)c(OCOC(=O)OCC[N+](C)(C)C)c2C3=O)C1 ; CC1=NO[C@@]2(CC[C@H](C)N3C[C@H]2n2cc(C(=O)NCc4ccc(F)cc4F)c(=O)c(OC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCCCOP(=O)(O)O)c2C3=O)C1]

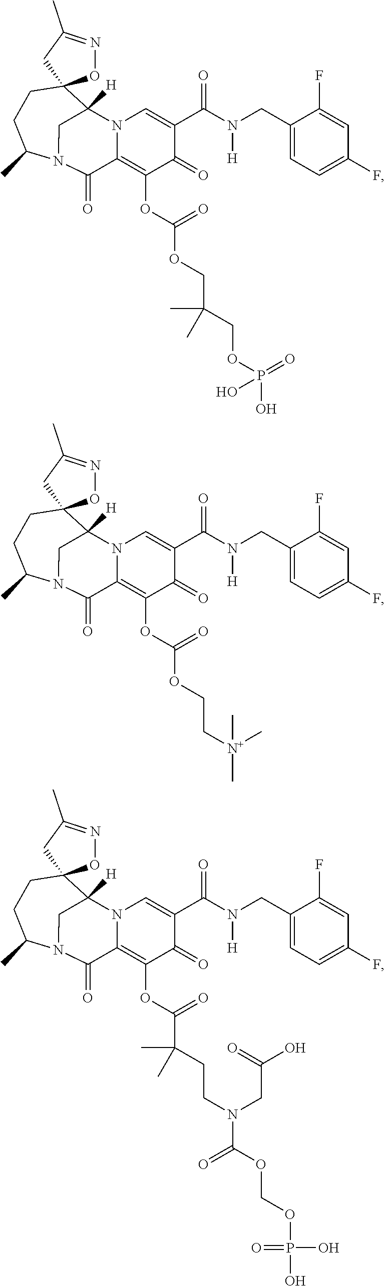 [SMILES: CC1=NO[C@@]2(CC[C@H](C)N3C[C@H]2n2cc(C(=O)NCc4ccc(F)cc4F)c(=O)c(OC(=O)OCC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCC[N+](C)(C)C)c2C3=O)C1 ; CC1=NO[C@@]2(CC[C@H](C)N3C[C@H]2n2cc(C(=O)NCc4ccc(F)cc4F)c(=O)c(OC(=O)C(C)(C)CCN(CC(=O)O)C(=O)OCOP(=O)(O)O)c2C3=O)C1]

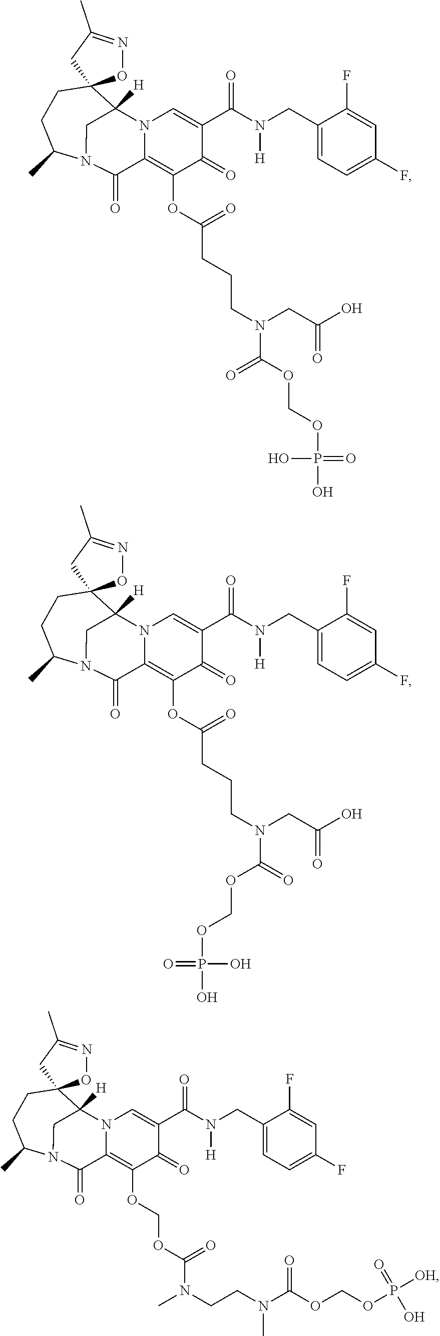 [SMILES: CC1=NO[C@@]2(CC[C@H](C)N3C[C@H]2n2cc(C(=O)NCc4ccc(F)cc4F)c(=O)c(OC(=O)CCCN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)CCCN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)CCN(C)C(=O)OCOP(=O)(O)O)c2C3=O)C1]

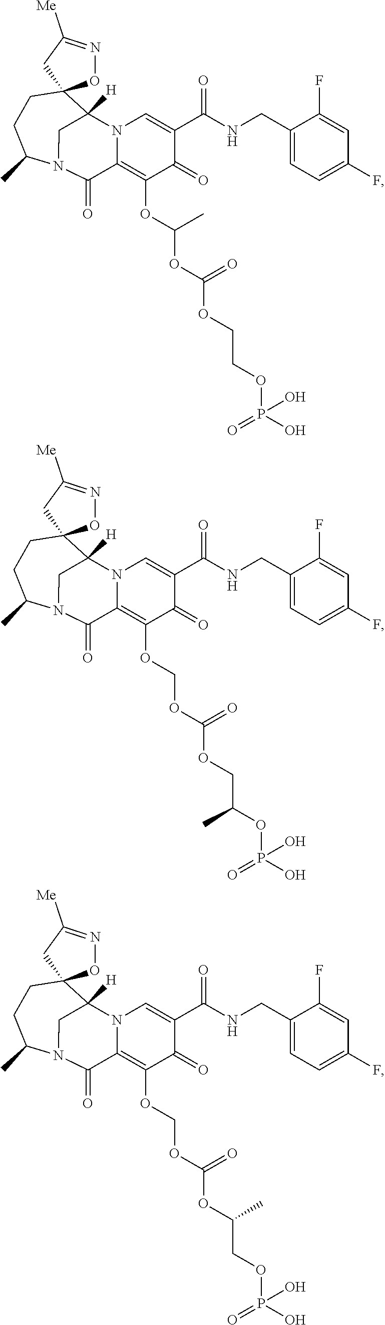 [SMILES: CC1=NO[C@@]2(CC[C@H](C)N3C[C@H]2n2cc(C(=O)NCc4ccc(F)cc4F)c(=O)c(OC(C)OC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1]

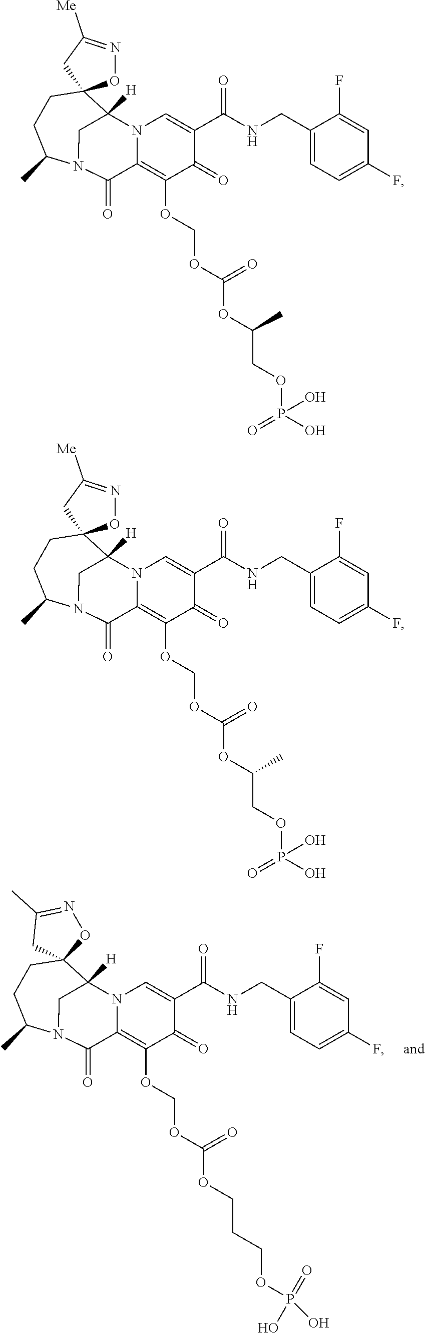 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCCOP(=O)(O)O)c2C3=O)C1]

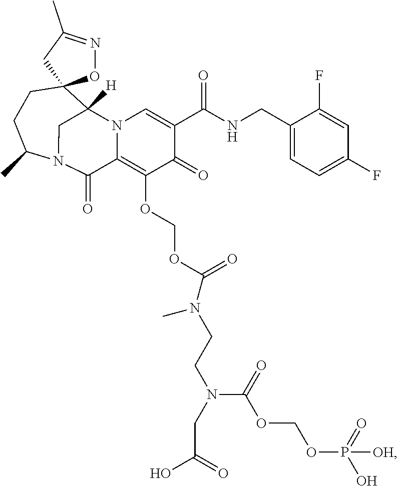 [SMILES: CC1=NO[C@@]2(CC[C@H](C)N3C[C@H]2n2cc(C(=O)NCc4ccc(F)cc4F)c(=O)c(OCOC(=O)N(C)CCN(CC(=O)O)C(=O)OCOP(=O)(O)O)c2C3=O)C1]

pharmaceutically acceptable salt thereof.

In some embodiments, the compound of Formula I or Formula I′ is selected from the group consisting of:

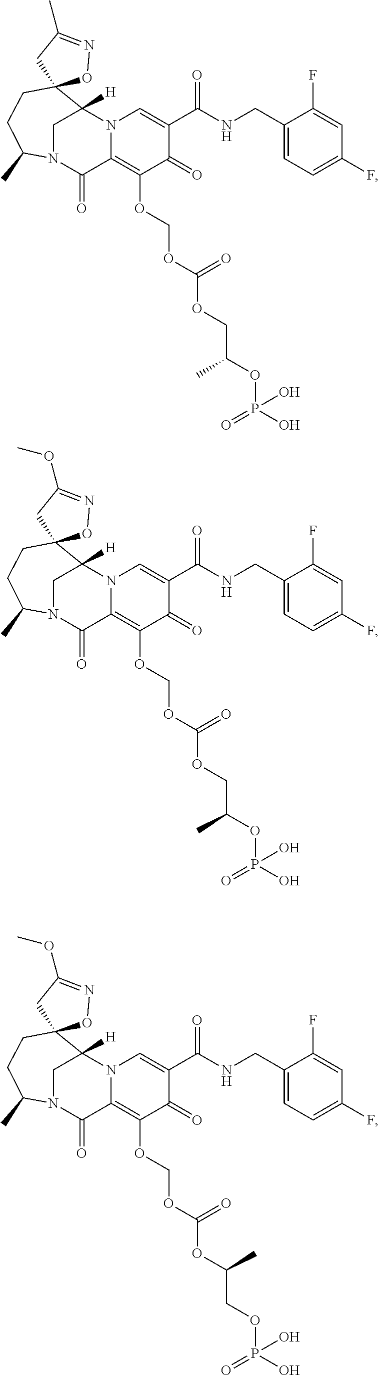 [SMILES: CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

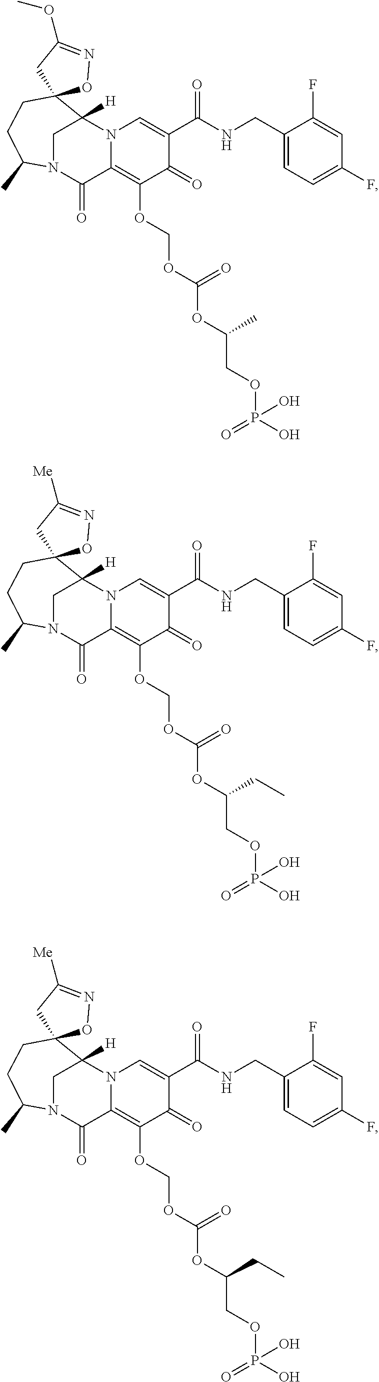 [SMILES: COC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1 ; CC[C@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2]

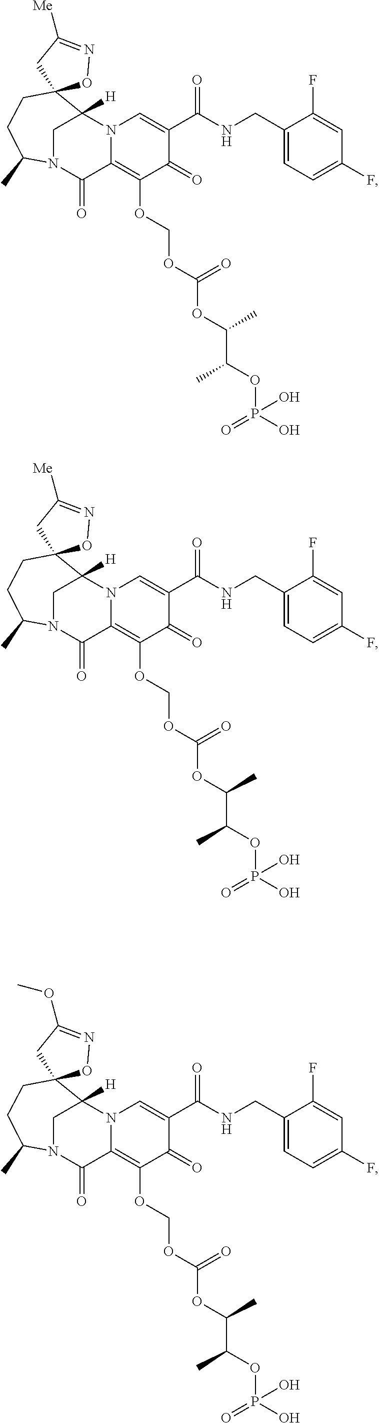 [SMILES: CC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

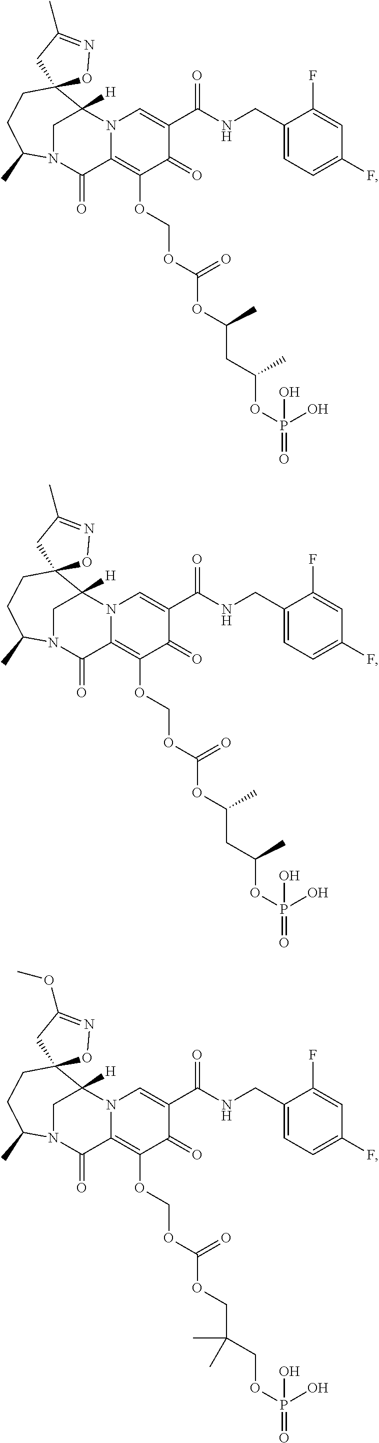 [SMILES: CC1=NO[C@@]2(CC[C@H](C)N3C[C@H]2n2cc(C(=O)NCc4ccc(F)cc4F)c(=O)c(OCOC(=O)O[C@@H](C)C[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)C[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

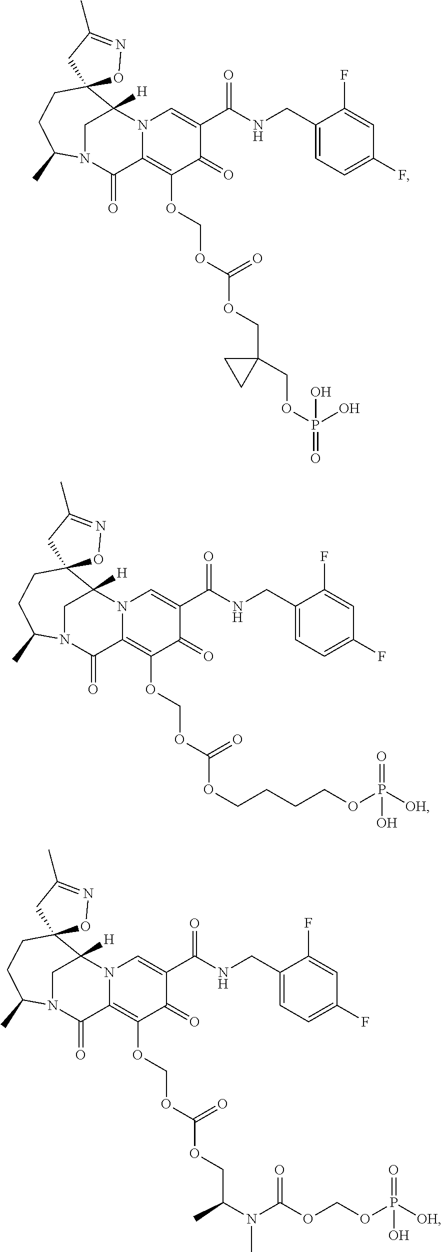 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)CC4)c2C3=O)C1 ; CC1=NO[C@@]2(CC[C@H](C)N3C[C@H]2n2cc(C(=O)NCc4ccc(F)cc4F)c(=O)c(OCOC(=O)OCCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)N(C)C(=O)OCOP(=O)(O)O)c2C3=O)C1]

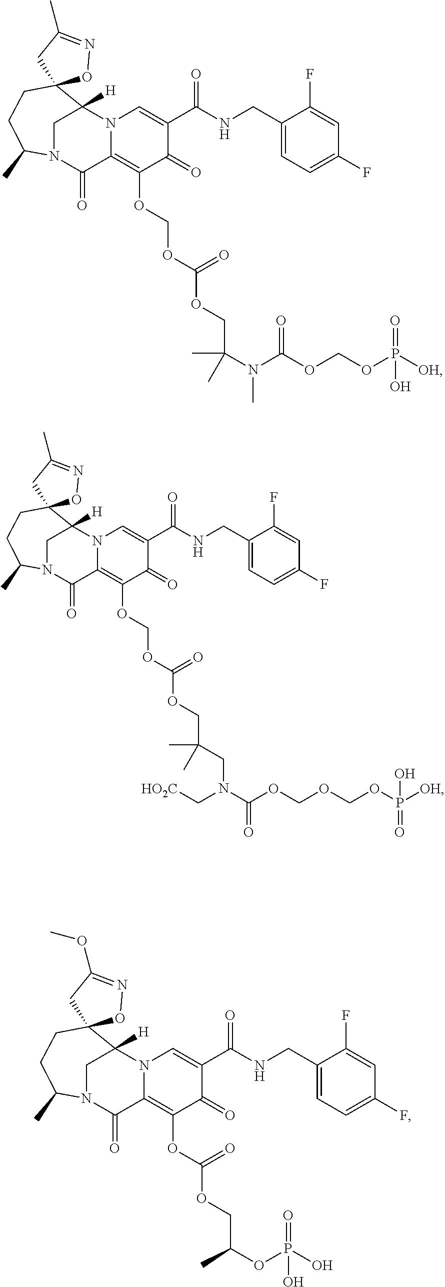 [SMILES: CC1=NO[C@@]2(CC[C@H](C)N3C[C@H]2n2cc(C(=O)NCc4ccc(F)cc4F)c(=O)c(OCOC(=O)OCC(C)(C)N(C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCC(C)(C)CN(CC(=O)O)C(=O)OCOCOP(=O)(O)O)c2C3=O)C1 ; COC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1]

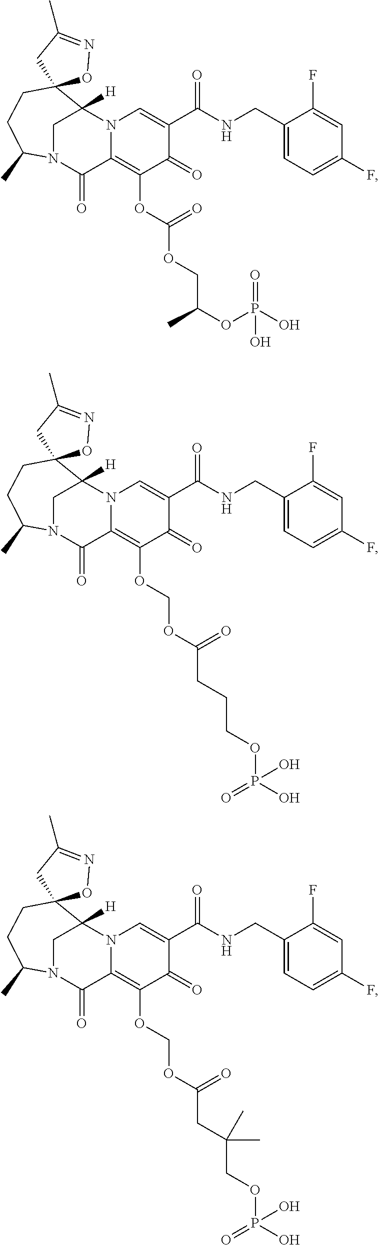 [SMILES: CC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)COP(=O)(O)O)c2C3=O)C1]

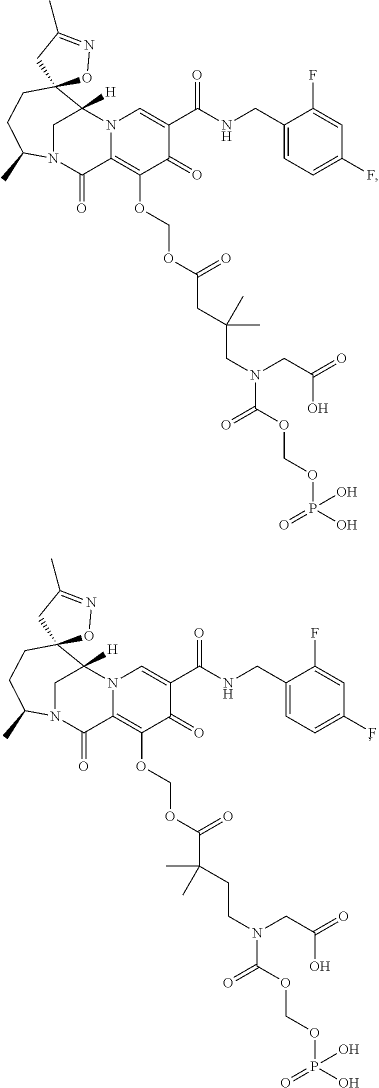 [SMILES: CC1=NO[C@@]2(CC[C@H](C)N3C[C@H]2n2cc(C(=O)NCc4ccc(F)cc4F)c(=O)c(OCOC(=O)CC(C)(C)CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)C(C)(C)CCN(CC(=O)O)C(=O)OCOP(=O)(O)O)c2C3=O)C1]

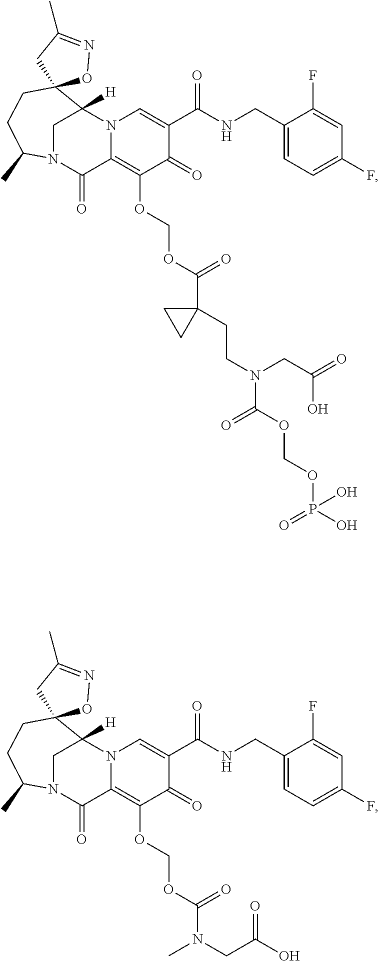 [SMILES: CC1=NO[C@@]2(CC[C@H](C)N3C[C@H]2n2cc(C(=O)NCc4ccc(F)cc4F)c(=O)c(OCOC(=O)C4(CCN(CC(=O)O)C(=O)OCOP(=O)(O)O)CC4)c2C3=O)C1 ; CC1=NO[C@@]2(CC[C@H](C)N3C[C@H]2n2cc(C(=O)NCc4ccc(F)cc4F)c(=O)c(OCOC(=O)N(C)CC(=O)O)c2C3=O)C1]

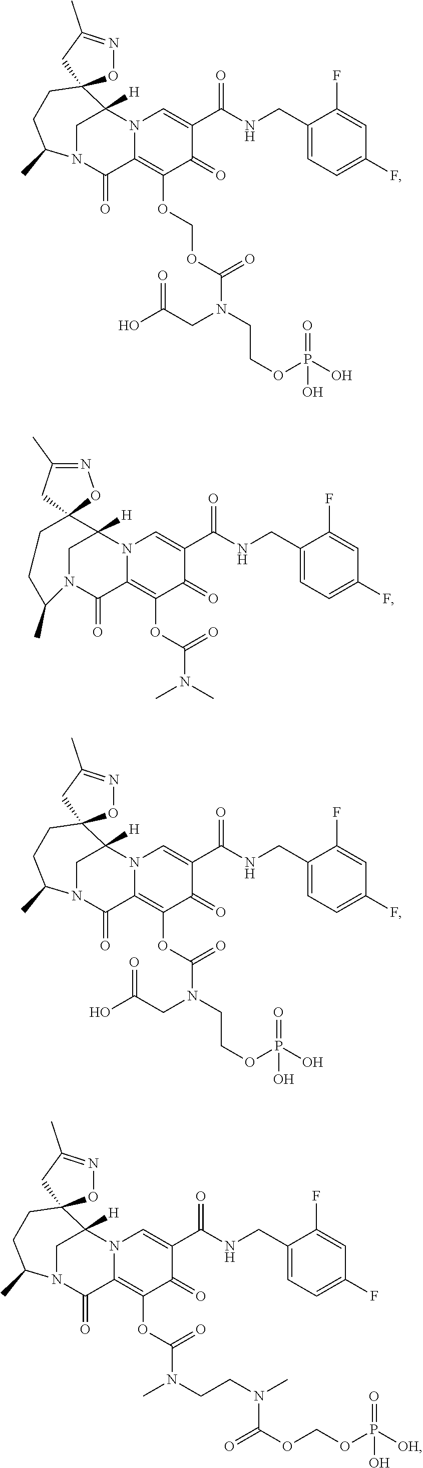 [SMILES: CC1=NO[C@@]2(CC[C@H](C)N3C[C@H]2n2cc(C(=O)NCc4ccc(F)cc4F)c(=O)c(OCOC(=O)N(CCOP(=O)(O)O)CC(=O)O)c2C3=O)C1 ; CC1=NO[C@@]2(CC[C@H](C)N3C[C@H]2n2cc(C(=O)NCc4ccc(F)cc4F)c(=O)c(OC(=O)N(C)C)c2C3=O)C1 ; CC1=NO[C@@]2(CC[C@H](C)N3C[C@H]2n2cc(C(=O)NCc4ccc(F)cc4F)c(=O)c(OC(=O)N(CCOP(=O)(O)O)CC(=O)O)c2C3=O)C1 ; CC1=NO[C@@]2(CC[C@H](C)N3C[C@H]2n2cc(C(=O)NCc4ccc(F)cc4F)c(=O)c(OC(=O)N(C)CCN(C)C(=O)OCOP(=O)(O)O)c2C3=O)C1]

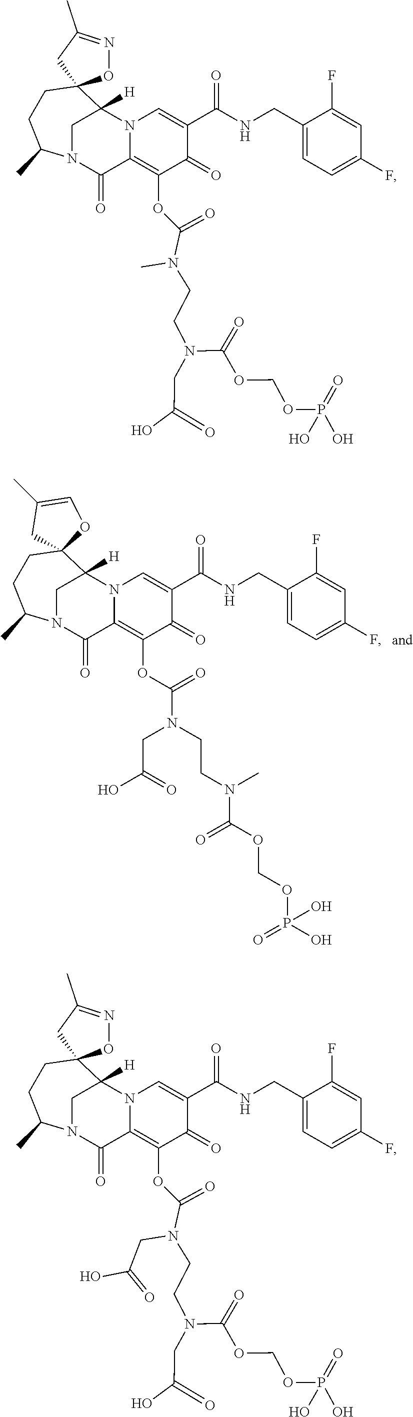 [SMILES: CC1=NO[C@@]2(CC[C@H](C)N3C[C@H]2n2cc(C(=O)NCc4ccc(F)cc4F)c(=O)c(OC(=O)N(C)CCN(CC(=O)O)C(=O)OCOP(=O)(O)O)c2C3=O)C1 ; CC1=CO[C@@]2(CC[C@H](C)N3C[C@H]2n2cc(C(=O)NCc4ccc(F)cc4F)c(=O)c(OC(=O)N(CCN(C)C(=O)OCOP(=O)(O)O)CC(=O)O)c2C3=O)C1 ; "and" ; CC1=NO[C@@]2(CC[C@H](C)N3C[C@H]2n2cc(C(=O)NCc4ccc(F)cc4F)c(=O)c(OC(=O)N(CCN(CC(=O)O)C(=O)OCOP(=O)(O)O)CC(=O)O)c2C3=O)C1]

or a pharmaceutically acceptable salt thereof.

In some embodiments, the compound of Formula I or Formula F is selected from the group consisting of:

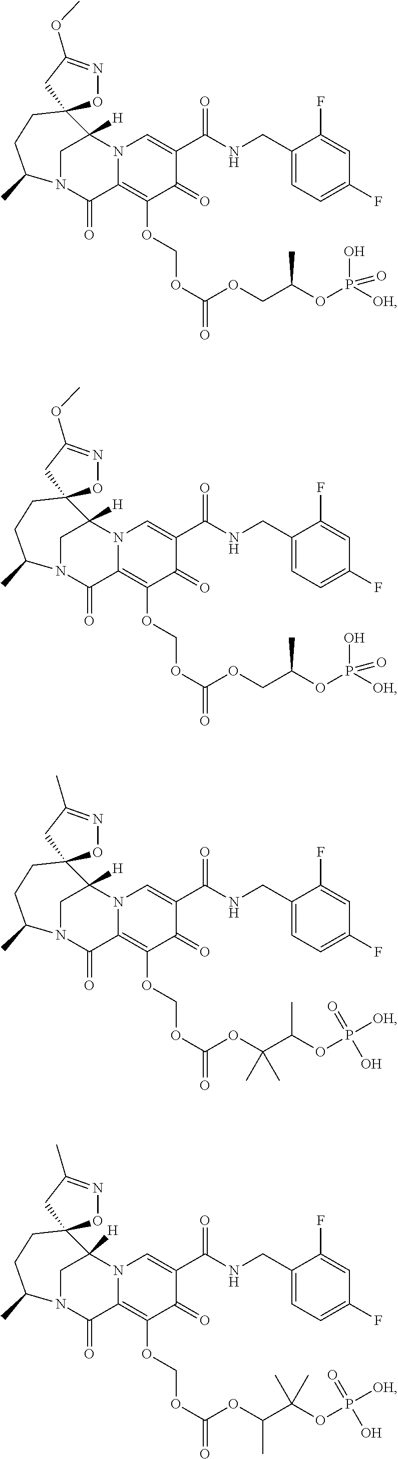 [SMILES: COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC(C)(C)C(C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC(C)C(C)(C)OP(=O)(O)O)c2C3=O)C1]

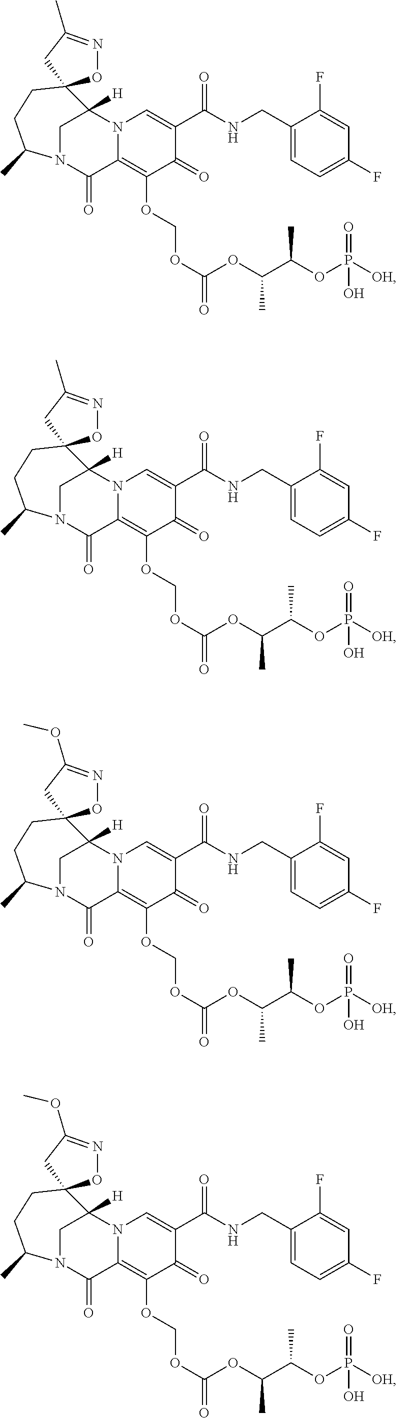 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1]

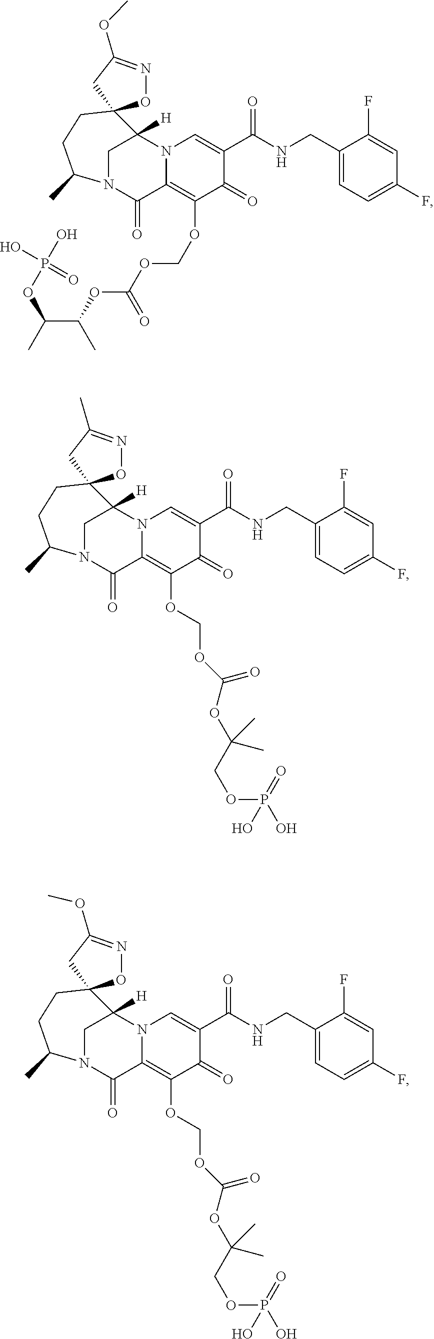 [SMILES: COC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1]

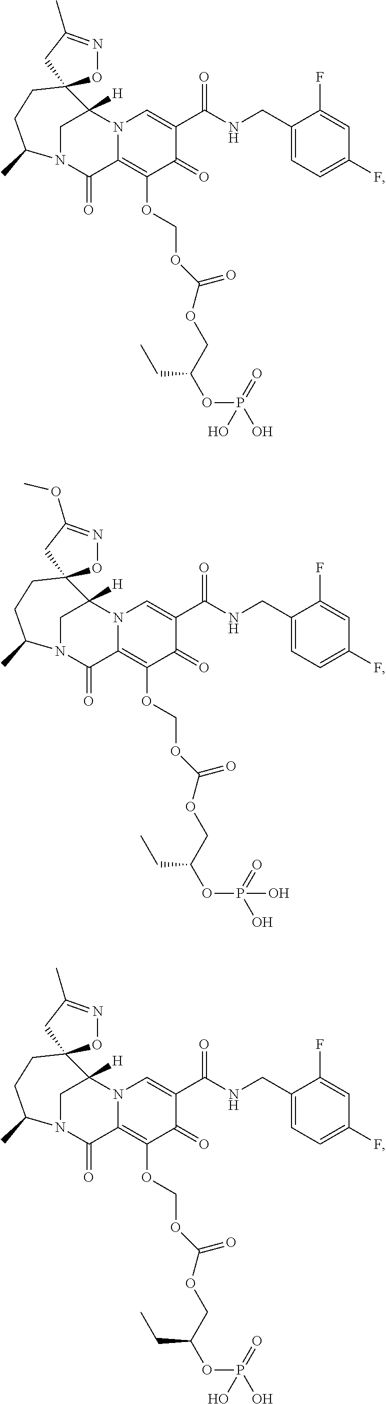 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O ; CC[C@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O ; CC[C@@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O]

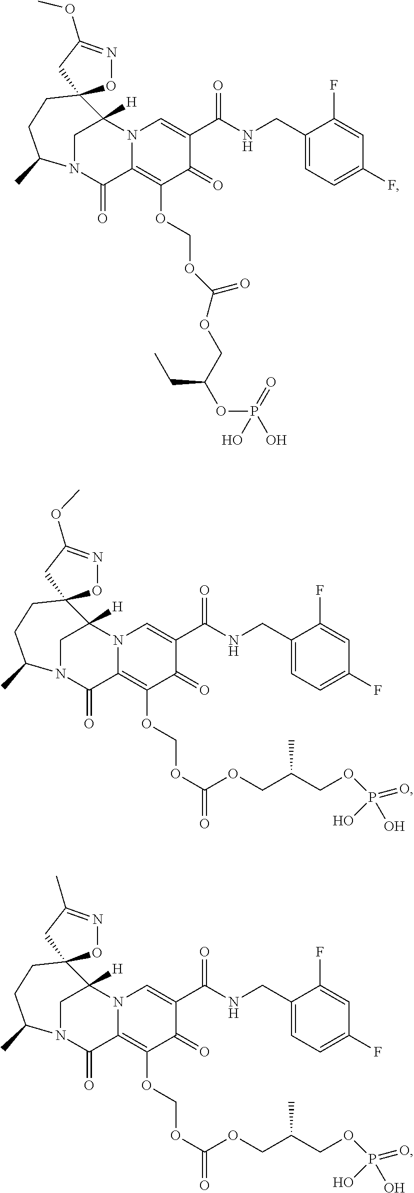 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O ; COC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1]

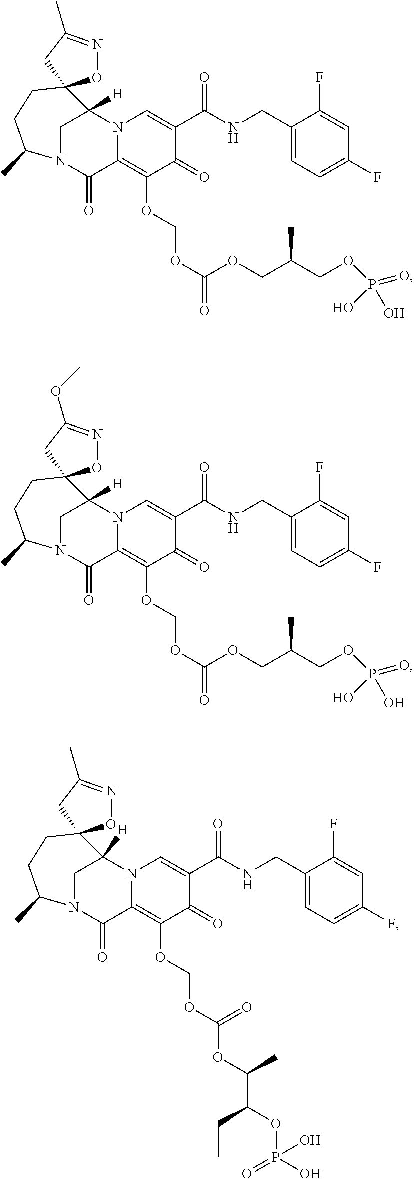 [SMILES: CC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1 ; CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

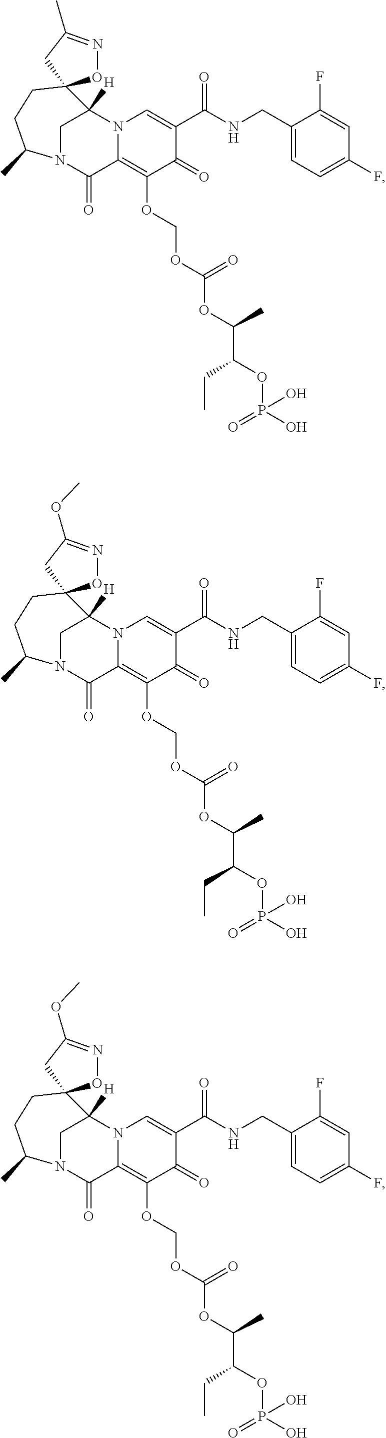 [SMILES: CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

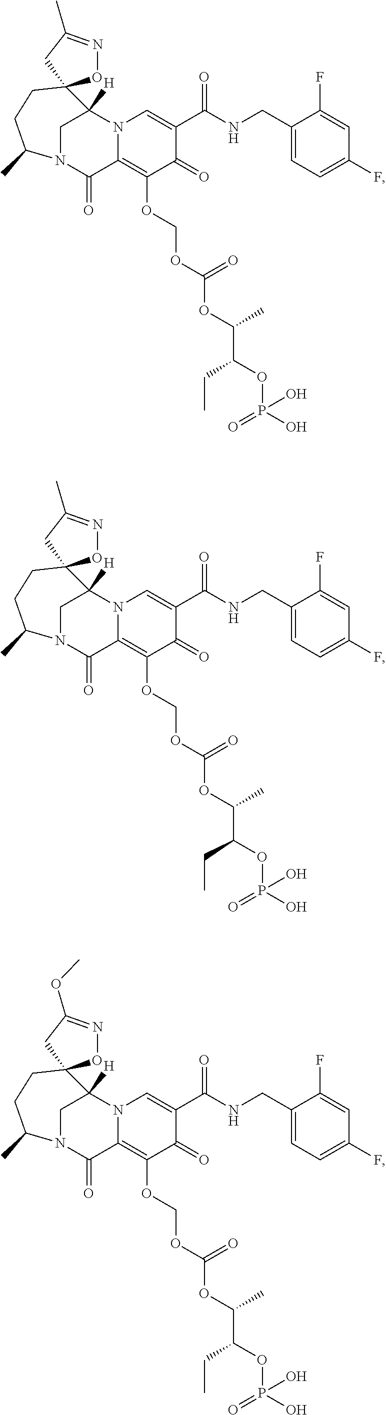 [SMILES: CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

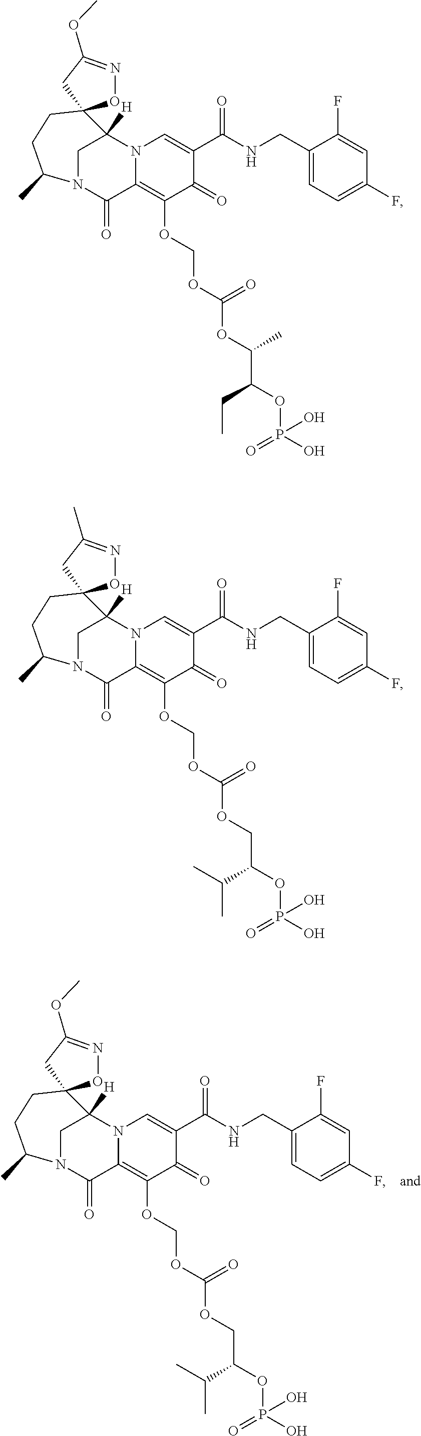 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; CC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1 ; COC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1 ; "and"]

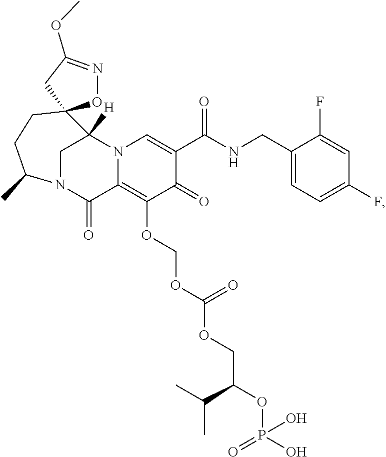 [SMILES: COC1=NO[C@@]2(CC[C@H](C)N3C[C@H]2n2cc(C(=O)NCc4ccc(F)cc4F)c(=O)c(OCOC(=O)OC[C@@H](OP(=O)(O)O)C(C)C)c2C3=O)C1]

or a pharmaceutically acceptable salt thereof.

In some embodiments, the compound of Formula I or Formula I′ is selected from:

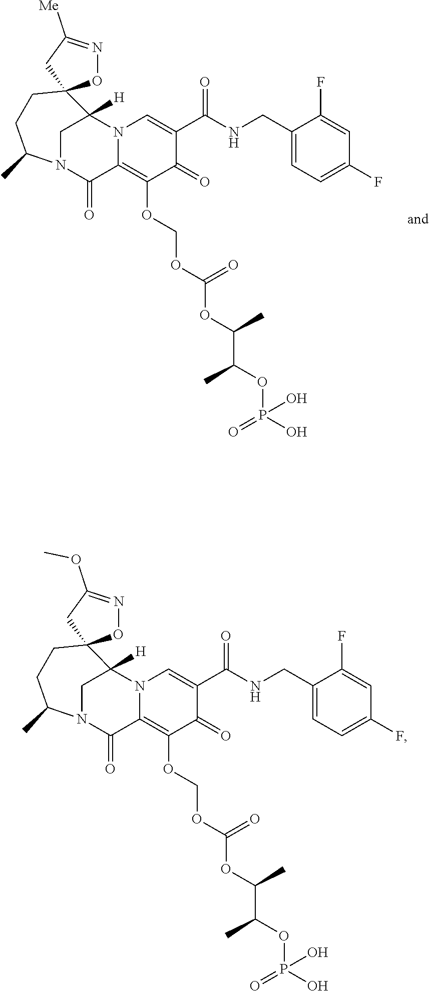 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

pharmaceutically acceptable salt thereof.

In some embodiments, the compound of Formula I or Formula I′ is:

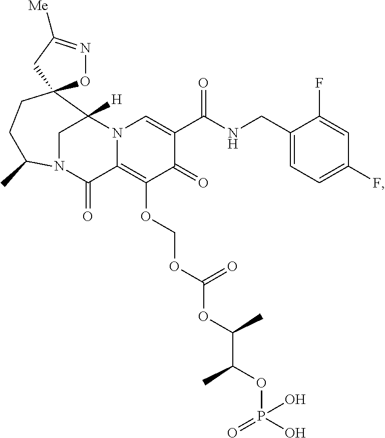 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof.

In some embodiments, the compound of Formula I or Formula I′ is:

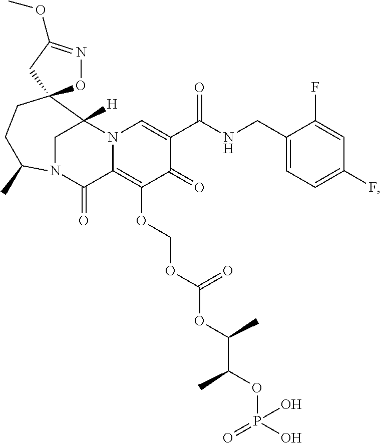 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof.

In some embodiments, the compound of Formula I or Formula I′ is:

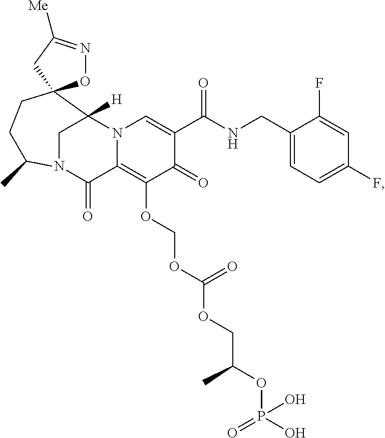 [SMILES: CC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1]

or a pharmaceutically acceptable salt thereof.

In some embodiments, the compound of Formula I or Formula I′ is:

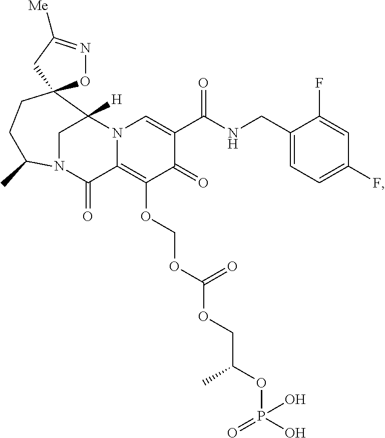 [SMILES: CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1]

or a pharmaceutically acceptable salt thereof.

In some embodiments, the compound of Formula I or Formula I′ is:

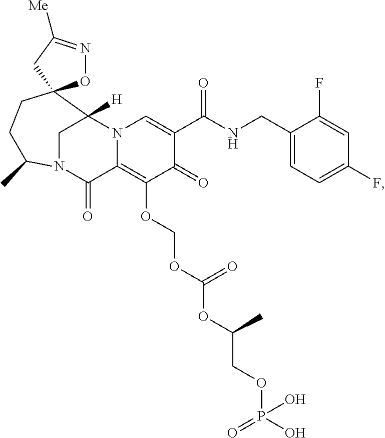 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

or a pharmaceutically acceptable salt thereof.

In some embodiments, the compound of Formula I or Formula I′ is:

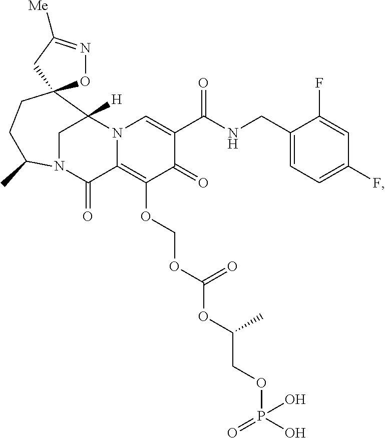 [SMILES: CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1]

or a pharmaceutically acceptable salt thereof.

In some embodiments, the compound of Formula I or Formula I′ is:

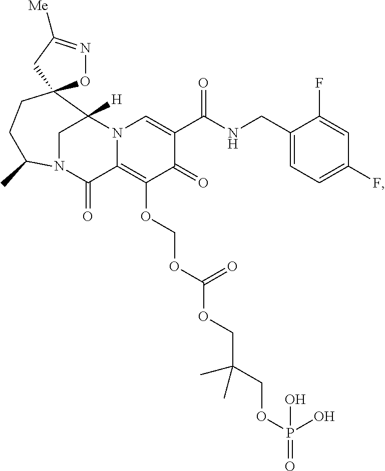 [SMILES: CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

or a pharmaceutically acceptable salt thereof.

In some embodiments, the compound of Formula I or Formula I′ is:

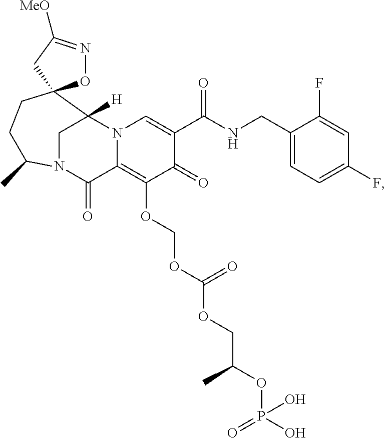 [SMILES: COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1]

or a pharmaceutically acceptable salt thereof.

In some embodiments, the compound of Formula I or Formula I′ is:

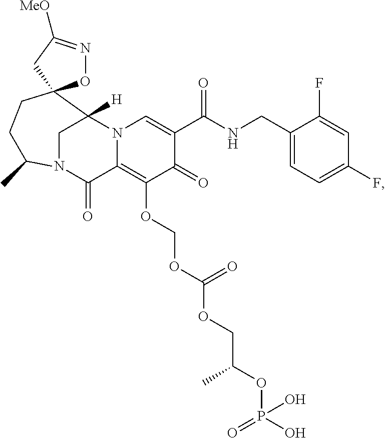 [SMILES: COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1]

or a pharmaceutically acceptable salt thereof.

In some embodiments, the compound of Formula I or Formula I′ is:

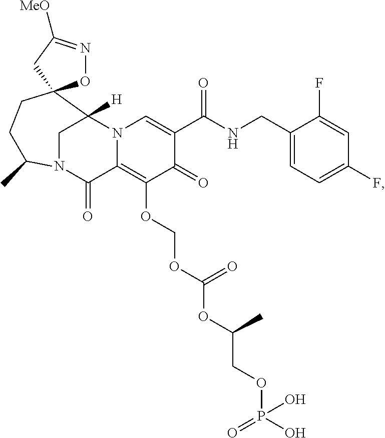 [SMILES: COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

or a pharmaceutically acceptable salt thereof.

In some embodiments, the compound of Formula I or Formula I′ is:

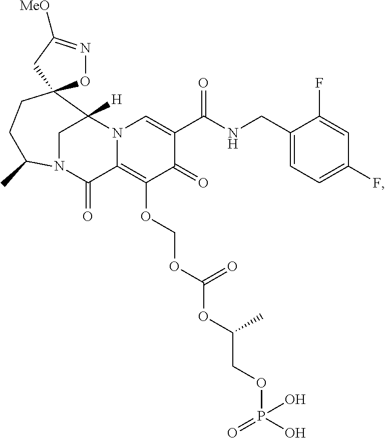 [SMILES: COC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1]

or a pharmaceutically acceptable salt thereof.

In some embodiments, the compound of Formula I or Formula I′ is:

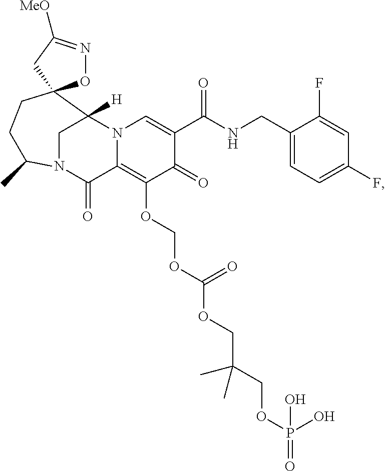 [SMILES: COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

or a pharmaceutically acceptable salt thereof.

In some embodiments, the compound of Formula I or Formula I′ is of the formula of any one of the compounds in Table A below.

TABLE A
Exemplary compounds of the present disclosure.

| Example | Name | Structure |
|---|---|---|
| 1 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl dihydrogen phosphate | 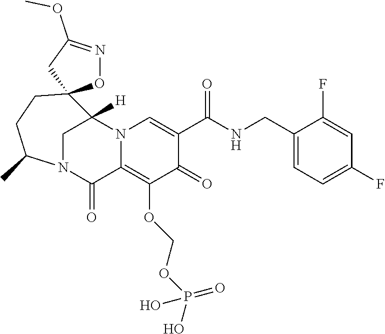 [SMILES: COC1=NO[C@@]2(CC[C@H](C)N3C[C@H]2n2cc(C(=O)NCc4ccc(F)cc4F)c(=O)c(OCOP(=O)(O)O)c2C3=O)C1] |
| 2 | N-(4-(((3′,5S,7′R)-10′-((2,4- difluorobenzyl)carbamoyl)-3,3′-dimethyl-1′,11′- dioxo-1′,4′,5′,11′-tetrahydro-3′H-4H,7′H- spiro[isoxazole-5,6′-[2,7]methanopyrido[1,2- a][1,4]diazonin]-12′-yl)oxy)-3,3-dimethyl-4- oxobutyl)-N- (((phosphonooxy)methoxy)carbonyl)glycine | 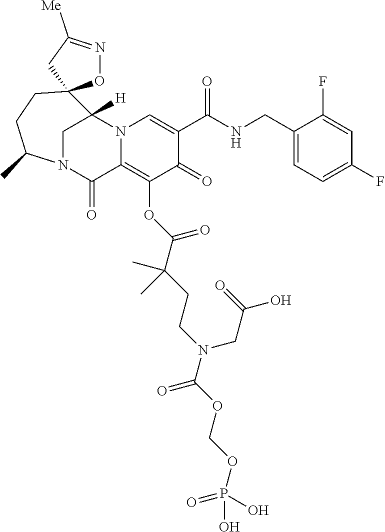 [SMILES: CC1=NO[C@@]2(CC[C@H](C)N3C[C@H]2n2cc(C(=O)NCc4ccc(F)cc4F)c(=O)c(OC(=O)C(C)(C)CCN(CC(=O)O)C(=O)OCOP(=O)(O)O)c2C3=O)C1] |
| 3 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl (2-(phosphonooxy)ethyl) carbonate | 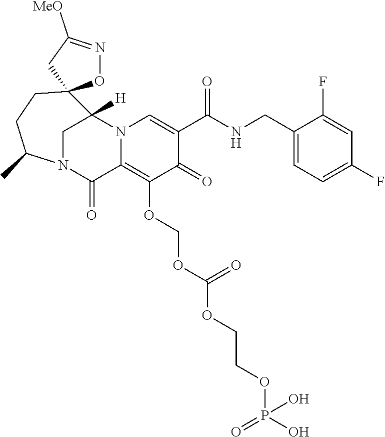 [SMILES: COC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1] |
| 4 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl (2- (phosphonooxy)ethyl) carbonate | 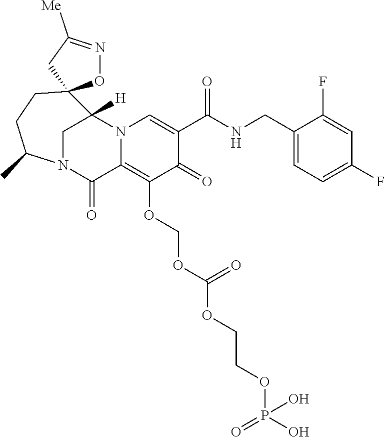 [SMILES: CC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1] |
| 5 | 1-(((3′,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)ethyl (2- (phosphonooxy)ethyl) carbonate | 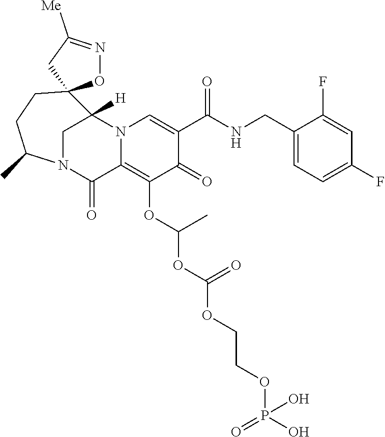 [SMILES: CC1=NO[C@@]2(CC[C@H](C)N3C[C@H]2n2cc(C(=O)NCc4ccc(F)cc4F)c(=O)c(OC(C)OC(=O)OCCOP(=O)(O)O)c2C3=O)C1] |
| 6 | (3′S,5S,7′R)-10′-((2,4- difluorobenzyl)carbamoyl)-3,3′-dimethyl-1′,11′- dioxo-1′,4′,5,11′-tetrahydro-3′H,4H,7′H- spiro[isoxazole-5,6′-[2,7]methanopyrido[1,2- a][1,4]diazonin]-12′-yl (2-(phosphonooxy)ethyl) carbonate | 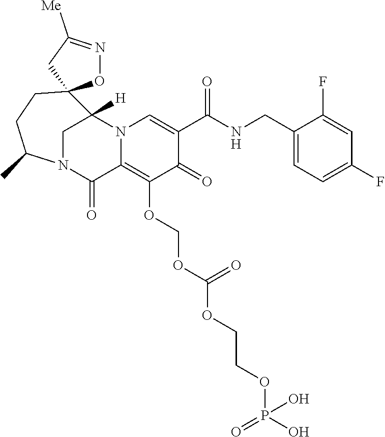 [SMILES: CC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1] |
| 7 | N-(2-(((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′- yl)oxy)carbonyl)oxy)ethyl)-N- (((phosphonooxy)methoxy)carbonyl)glycine | 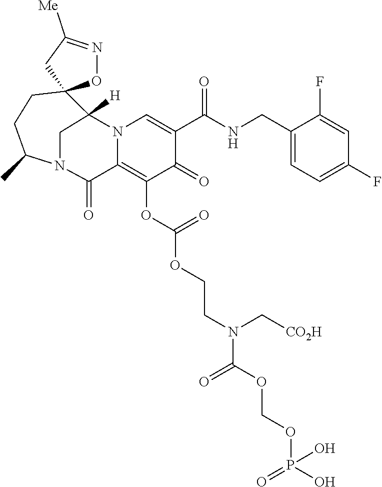 [SMILES: CC1=NO[C@@]2(CC[C@H](C)N3C[C@H]2n2cc(C(=O)NCc4ccc(F)cc4F)c(=O)c(OC(=O)OCCN(CC(=O)O)C(=O)OCOP(=O)(O)O)c2C3=O)C1] |
| 8 | N-(2-((((((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methoxy)carbonyl)oxy)ethyl)-N- (((phosphonooxy)methoxy)carbonyl)glycine | 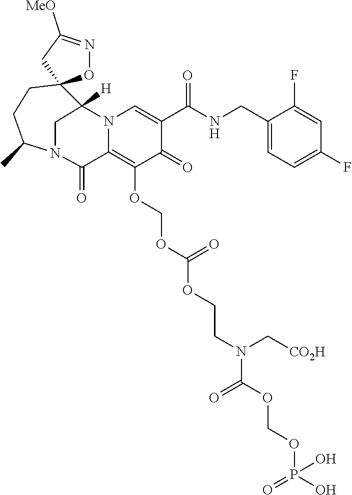 [SMILES: COC1=NO[C@@]2(CC[C@H](C)N3C[C@H]2n2cc(C(=O)NCc4ccc(F)cc4F)c(=O)c(OCOC(=O)OCCN(CC(=O)O)C(=O)OCOP(=O)(O)O)c2C3=O)C1] |
| 9 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((phosphonooxy)methyl)ethane-1,2- diylbis(methylcarbamate) | 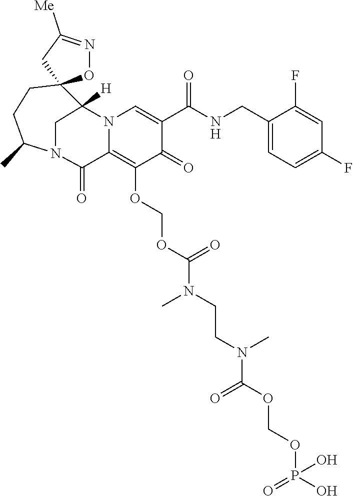 [SMILES: CC1=NO[C@@]2(CC[C@H](C)N3C[C@H]2n2cc(C(=O)NCc4ccc(F)cc4F)c(=O)c(OCOC(=O)N(C)CCN(C)C(=O)OCOP(=O)(O)O)c2C3=O)C1] |
| 10 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl (3- (phosphonooxy)propyl) carbonate | 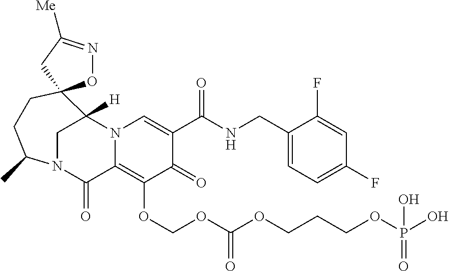 [SMILES: CC1=NO[C@@]2(CC[C@H](C)N3C[C@H]2n2cc(C(=O)NCc4ccc(F)cc4F)c(=O)c(OCOC(=O)OCCCOP(=O)(O)O)c2C3=O)C1] |
| 11 | (3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl (3- (phosphonooxy)propyl) carbonate | 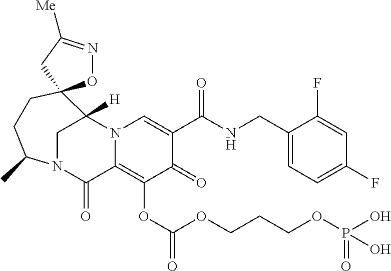 [SMILES: CC1=NO[C@@]2(CC[C@H](C)N3C[C@H]2n2cc(C(=O)NCc4ccc(F)cc4F)c(=O)c(OC(=O)OCCCOP(=O)(O)O)c2C3=O)C1] |
| 12 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl (2,2- dimethyl-3-(phosphonooxy)propyl) carbonate | 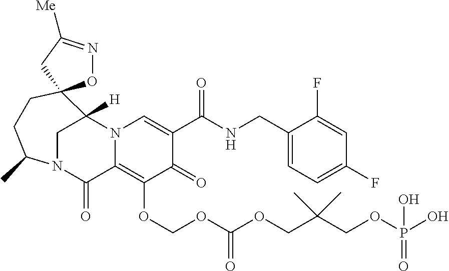 [SMILES: CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1] |
| 13 | (3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl (2,2-dimethyl-3- (phosphonooxy)propyl) carbonate | 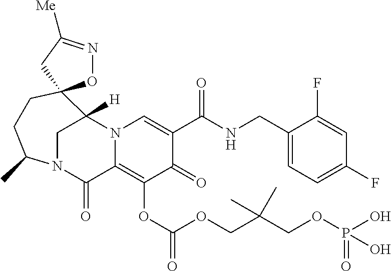 [SMILES: CC1=NO[C@@]2(CC[C@H](C)N3C[C@H]2n2cc(C(=O)NCc4ccc(F)cc4F)c(=O)c(OC(=O)OCC(C)(C)COP(=O)(O)O)c2C3=O)C1] |
| 14 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl (2- (dimethylamino)ethyl) carbonate | 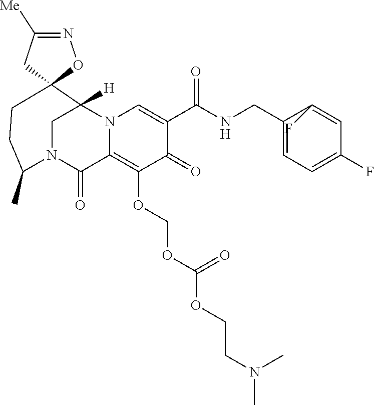 |
| 15 | 2-((((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′- yl)oxy)methoxy)carbonyl)oxy)-N,N,N- trimethylethan-1-aminium trifluoroacetate | 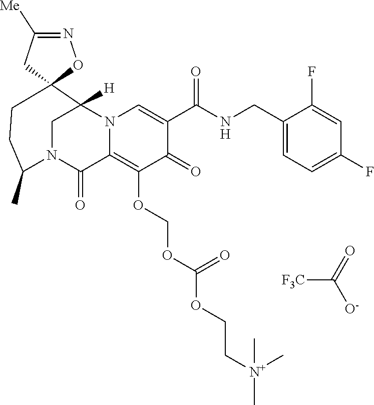 [SMILES: CC1=NO[C@@]2(CC[C@H](C)N3C[C@H]2n2cc(C(=O)NCc4ccc(F)cc4F)c(=O)c(OCOC(=O)OCC[N+](C)(C)C)c2C3=O)C1 ; O=C([O-])C(F)(F)F] |
| 16 | 2-(((((3′,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)carbonyl)oxy)- N,N,N-trimethylethan-1-aminium trifluoroacetic acid | 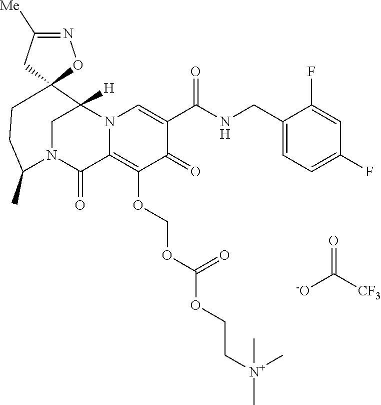 |
| 17 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((S)-2- (phosphonooxy)propyl) carbonate | 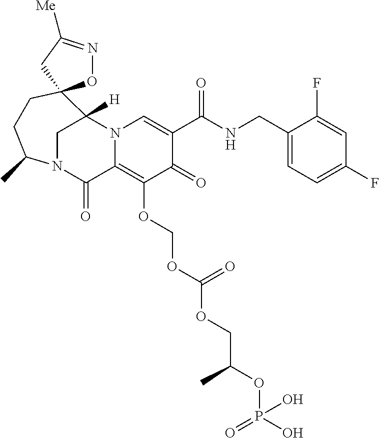 [SMILES: CC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1] |
| 18 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((R)-1- (phosphonooxy)propan-2-yl) carbonate | 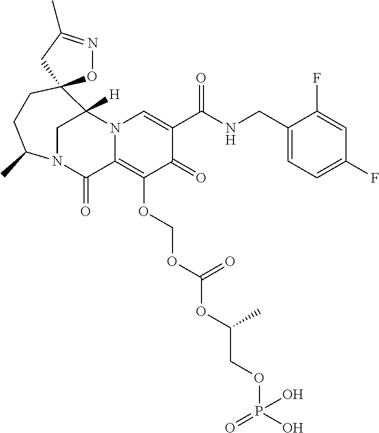 [SMILES: CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1] |
| 19 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((S)-1- (phosphonooxy)propan-2-yl) carbonate | 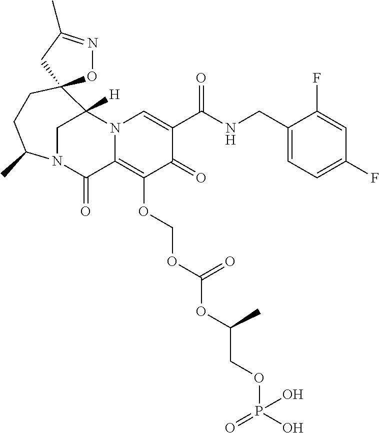 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1] |
| 20 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl 5- (phosphonooxy)pentanoate | 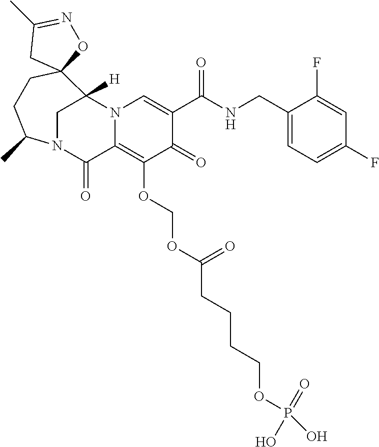 [SMILES: CC1=NO[C@@]2(CC[C@H](C)N3C[C@H]2n2cc(C(=O)NCc4ccc(F)cc4F)c(=O)c(OCOC(=O)CCCCOP(=O)(O)O)c2C3=O)C1] |
| 21 | N-(2-((((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′- yl)oxy)methoxy)carbonyl)(methyl)amino)ethyl)- N-(((phosphonooxy)methoxy)carbonyl)glycine | 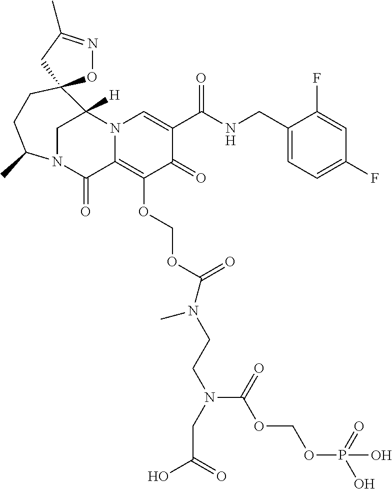 [SMILES: CC1=NO[C@@]2(CC[C@H](C)N3C[C@H]2n2cc(C(=O)NCc4ccc(F)cc4F)c(=O)c(OCOC(=O)N(C)CCN(CC(=O)O)C(=O)OCOP(=O)(O)O)c2C3=O)C1] |
| 22 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((R)-2- (phosphonooxy)propyl) carbonate | 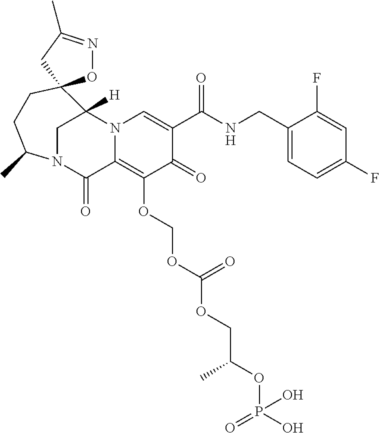 [SMILES: CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1] |
| 23 | (((3′S,5S,7′R)-10′-((2,4- difluorobenzyl)carbamoyl)-3-methoxy-3′- methyl-1′,11′-dioxo-1′,4′,5′,11′-tetrahydro-3′H,4H,7′H- spiro[isoxazole-5,6′-[2,7]methanopyrido[1,2-a][1,4]diazonin]- 12′-yl)oxy)methyl ((S)-2-(phosphonooxy)propyl) carbonate | 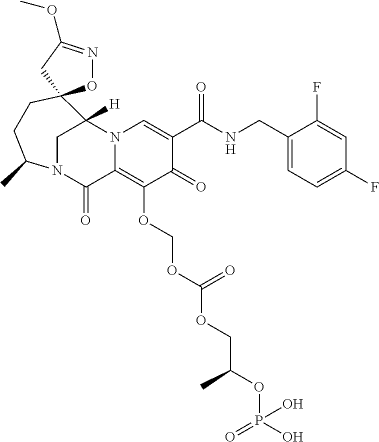 [SMILES: COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1] |
| 24 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((S)-1-(phosphonooxy)propan-2- yl) carbonate | 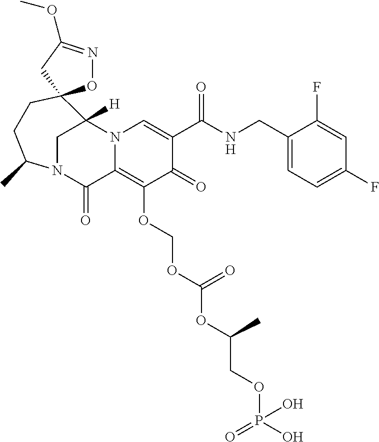 [SMILES: COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1] |
| 25 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((R)-1- (phosphonooxy)butan-2-yl) carbonate | 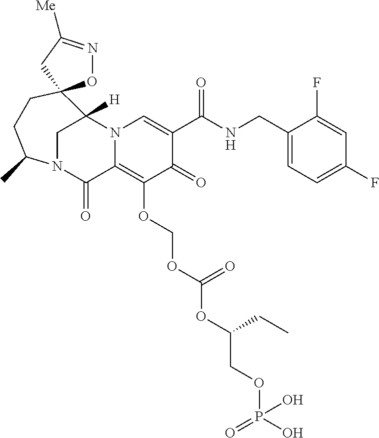 [SMILES: CC[C@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2] |
| 26 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((S)-1- (phosphonooxy)butan-2-yl) carbonate | 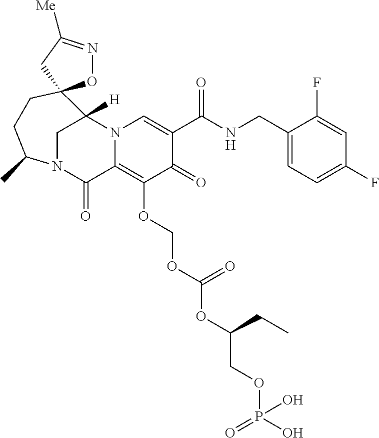 [SMILES: CC[C@@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2] |
| 27 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2R,3R)-3- (phosphonooxy)butan-2-yl) carbonate | 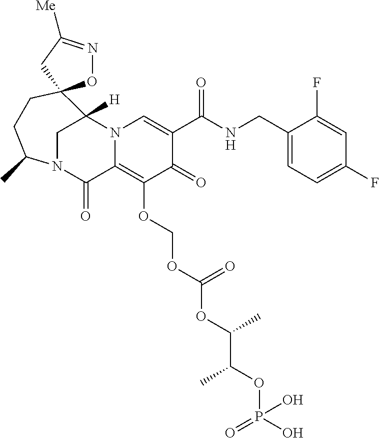 [SMILES: CC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1] |
| 28 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2S,3S)-3- (phosphonooxy)butan-2-yl) carbonate | 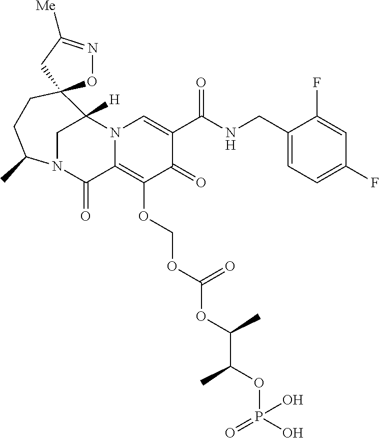 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1] |
| 29 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((2S,3S)-3- (phosphonooxy)butan-2-yl) carbonate | 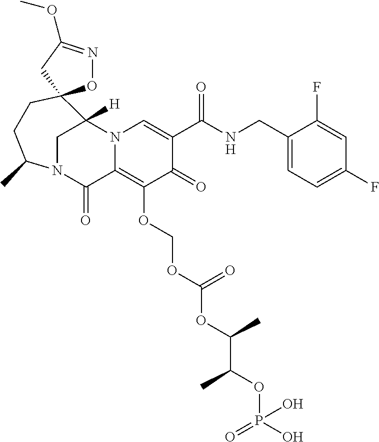 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1] |
| 30 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2S,4S)-4- (phosphonooxy)pentan-2-yl) carbonate | 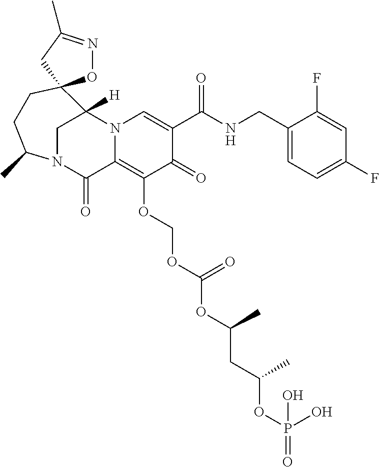 [SMILES: CC1=NO[C@@]2(CC[C@H](C)N3C[C@H]2n2cc(C(=O)NCc4ccc(F)cc4F)c(=O)c(OCOC(=O)O[C@@H](C)C[C@H](C)OP(=O)(O)O)c2C3=O)C1] |
| 31 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2R,4R)-4- (phosphonooxy)pentan-2-yl) carbonate | 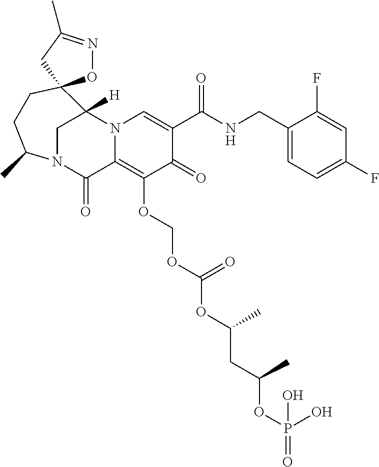 [SMILES: CC1=NO[C@@]2(CC[C@H](C)N3C[C@H]2n2cc(C(=O)NCc4ccc(F)cc4F)c(=O)c(OCOC(=O)O[C@H](C)C[C@@H](C)OP(=O)(O)O)c2C3=O)C1] |
| 32 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl (2,2-dimethyl-3- (phosphonooxy)propyl) carbonate | 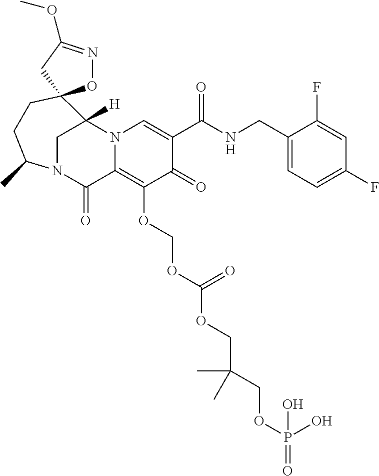 [SMILES: COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1] |
| 33 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((1- ((phosphonooxy)methyl)cyclopropyl)methyl) carbonate | 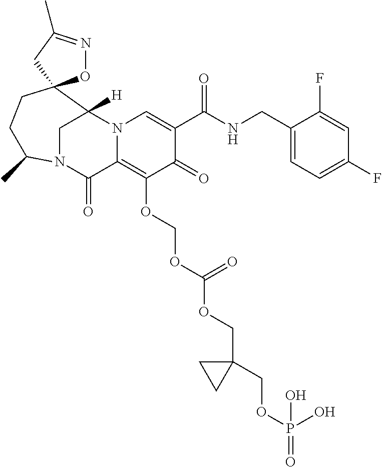 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)CC4)c2C3=O)C1] |
| 34 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl (4- (phosphonooxy)butyl) carbonate | 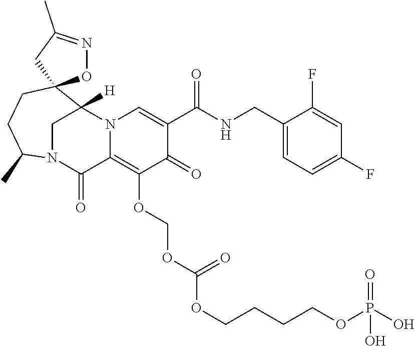 [SMILES: CC1=NO[C@@]2(CC[C@H](C)N3C[C@H]2n2cc(C(=O)NCc4ccc(F)cc4F)c(=O)c(OCOC(=O)OCCCCOP(=O)(O)O)c2C3=O)C1] |
| 35 | (phosphonooxy)methyl ((2S)-1- ((((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′- yl)oxy)methoxy)carbonyl)oxy)propan-2- yl)(methyl)carbamate | 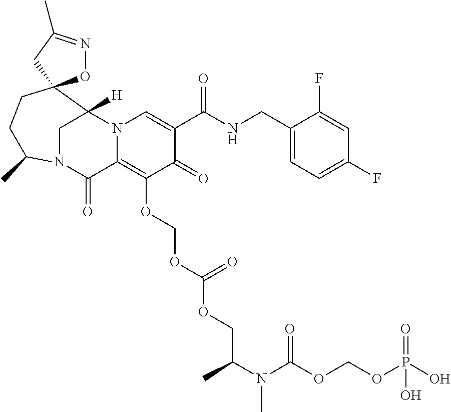 [SMILES: CC1=NO[C@@]2(CC[C@H](C)N3C[C@H]2n2cc(C(=O)NCc4ccc(F)cc4F)c(=O)c(OCOC(=O)OC[C@H](C)N(C)C(=O)OCOP(=O)(O)O)c2C3=O)C1] |
| 36 | (phosphonooxy)methyl (1-((((((3′S,5S,7′R)-10′- ((2,4-difluorobenzyl)carbamoyl)-3,3′-dimethyl- 1′,11′-dioxo-1′,4′,5′,11′-tetrahydro-3′H,4H,7′H- spiro[isoxazole-5,6′-[2,7]methanopyrido[1,2- a][1,4]diazonin]-12′- yl)oxy)methoxy)carbonyl)oxy)-2-methylpropan- 2-yl)(methyl)carbamate | 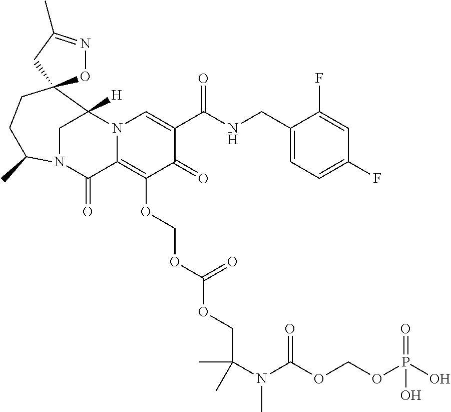 [SMILES: CC1=NO[C@@]2(CC[C@H](C)N3C[C@H]2n2cc(C(=O)NCc4ccc(F)cc4F)c(=O)c(OCOC(=O)OCC(C)(C)N(C)C(=O)OCOP(=O)(O)O)c2C3=O)C1] |
| 37 | N-(3-((((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′- yl)oxy)methoxy)carbonyl)oxy)-2,2- dimethylpropyl)-N- (((phosphonooxy)methoxy)carbonyl)glycine | 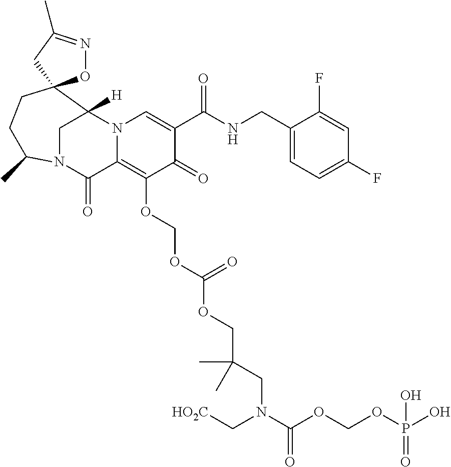 [SMILES: CC1=NO[C@@]2(CC[C@H](C)N3C[C@H]2n2cc(C(=O)NCc4ccc(F)cc4F)c(=O)c(OCOC(=O)OCC(C)(C)CN(CC(=O)O)C(=O)OCOP(=O)(O)O)c2C3=O)C1] |
| 38 | (3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido1,2-a][1,4]diazonin]-12′-yl ((S)-2-(phosphonooxy)propyl) carbonate | 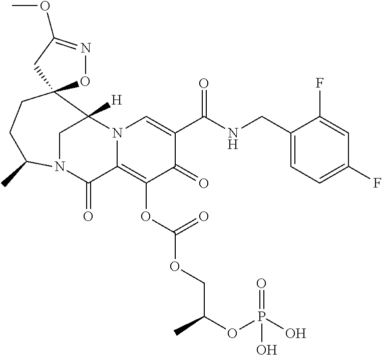 [SMILES: COC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1] |
| 39 | (3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl ((S)-2- (phosphonooxy)propyl) carbonate | 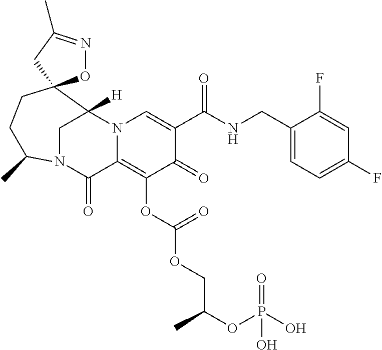 [SMILES: CC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1] |
| 40 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl 4- (phosphonooxy)butanoate | 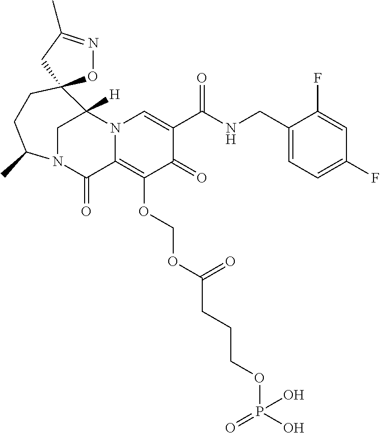 [SMILES: CC1=NO[C@@]2(CC[C@H](C)N3C[C@H]2n2cc(C(=O)NCc4ccc(F)cc4F)c(=O)c(OCOC(=O)CCCOP(=O)(O)O)c2C3=O)C1] |
| 41 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl 3,3- dimethyl-4-(phosphonooxy)butanoate | 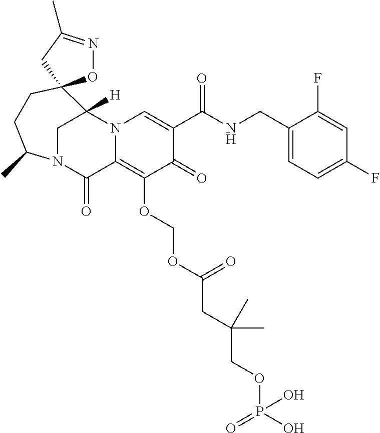 [SMILES: CC1=NO[C@@]2(CC[C@H](C)N3C[C@H]2n2cc(C(=O)NCc4ccc(F)cc4F)c(=O)c(OCOC(=O)CC(C)(C)COP(=O)(O)O)c2C3=O)C1] |
| 42 | N-(4-((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methoxy)-2,2- dimethyl-4-oxobutyl)-N- (((phosphonooxy)methoxy)carbonyl)glycine | 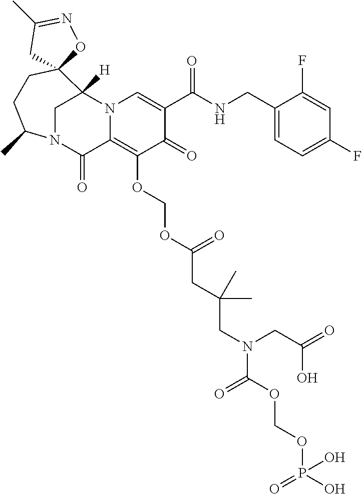 [SMILES: CC1=NO[C@@]2(CC[C@H](C)N3C[C@H]2n2cc(C(=O)NCc4ccc(F)cc4F)c(=O)c(OCOC(=O)CC(C)(C)CN(CC(=O)O)C(=O)OCOP(=O)(O)O)c2C3=O)C1] |
| 43 | N-(4-((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methoxy)-3,3- dimethyl-4-oxobutyl)-N- (((phosphonooxy)methoxy)carbonyl)glycine | 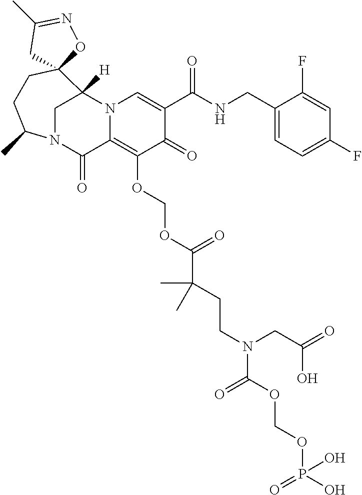 [SMILES: CC1=NO[C@@]2(CC[C@H](C)N3C[C@H]2n2cc(C(=O)NCc4ccc(F)cc4F)c(=O)c(OCOC(=O)C(C)(C)CCN(CC(=O)O)C(=O)OCOP(=O)(O)O)c2C3=O)C1] |
| 44 | N-(2-(1-(((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′- yl)oxy)methoxy)carbonyl)cyclopropyl)ethyl)-N- (((phosphonooxy)methoxy)carbonyl)glycine | 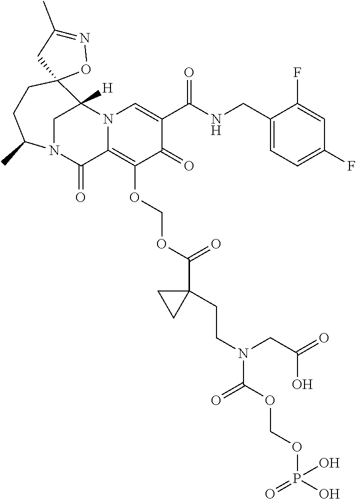 [SMILES: CC1=NO[C@@]2(CC[C@H](C)N3C[C@H]2n2cc(C(=O)NCc4ccc(F)cc4F)c(=O)c(OCOC(=O)C4(CCN(CC(=O)O)C(=O)OCOP(=O)(O)O)CC4)c2C3=O)C1] |
| 45 | N-(((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methoxy)carbonyl)- N-methylglycine | 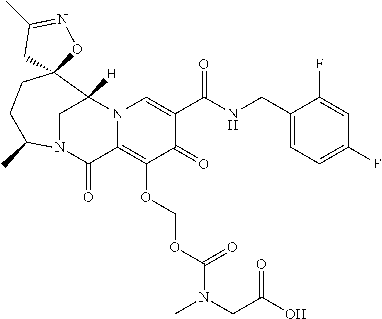 [SMILES: CC1=NO[C@@]2(CC[C@H](C)N3C[C@H]2n2cc(C(=O)NCc4ccc(F)cc4F)c(=O)c(OCOC(=O)N(C)CC(=O)O)c2C3=O)C1] |
| 46 | N-(((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methoxy)carbonyl)- N-(2-(phosphonooxy)ethyl)glycine | 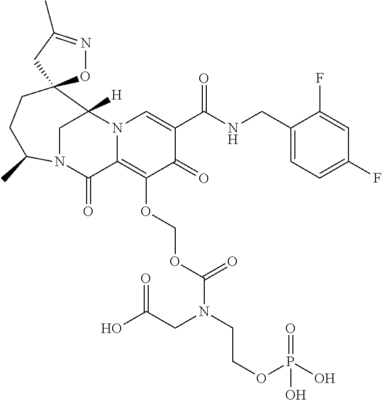 [SMILES: CC1=NO[C@@]2(CC[C@H](C)N3C[C@H]2n2cc(C(=O)NCc4ccc(F)cc4F)c(=O)c(OCOC(=O)N(CCOP(=O)(O)O)CC(=O)O)c2C3=O)C1] |
| 47 | (3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl dimethylcarbamate | 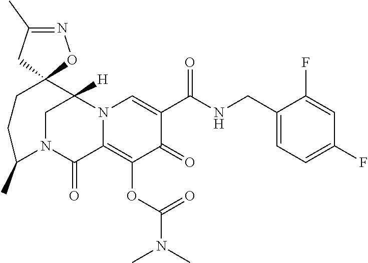 [SMILES: CC1=NO[C@@]2(CC[C@H](C)N3C[C@H]2n2cc(C(=O)NCc4ccc(F)cc4F)c(=O)c(OC(=O)N(C)C)c2C3=O)C1] |
| 48 | N-((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)carbonyl)-N-(2- (phosphonooxy)ethyl)glycine | 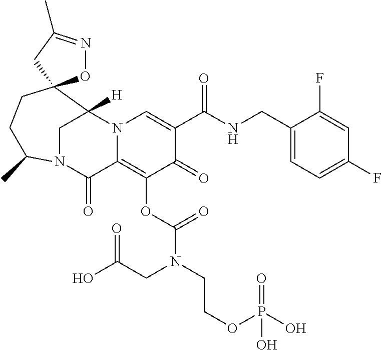 [SMILES: CC1=NO[C@@]2(CC[C@H](C)N3C[C@H]2n2cc(C(=O)NCc4ccc(F)cc4F)c(=O)c(OC(=O)N(CCOP(=O)(O)O)CC(=O)O)c2C3=O)C1] |
| 49 | (3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl ((phosphonooxy)methyl) ethane-1,2-diylbis(methylcarbamate) | 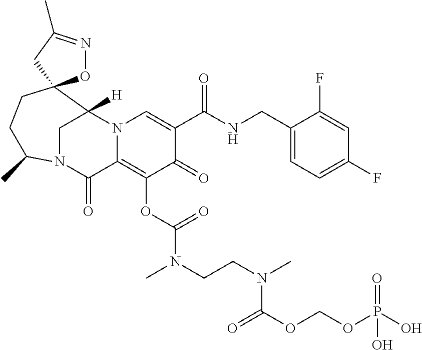 [SMILES: CC1=NO[C@@]2(CC[C@H](C)N3C[C@H]2n2cc(C(=O)NCc4ccc(F)cc4F)c(=O)c(OC(=O)N(C)CCN(C)C(=O)OCOP(=O)(O)O)c2C3=O)C1] |
| 50 | N-(2-(((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′- yl)oxy)carbonyl)(methyl)amino)ethyl)-N- (((phosphonooxy)methoxy)carbonyl)glycine | 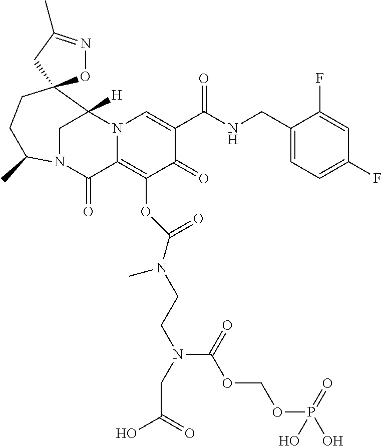 [SMILES: CC1=NO[C@@]2(CC[C@H](C)N3C[C@H]2n2cc(C(=O)NCc4ccc(F)cc4F)c(=O)c(OC(=O)N(C)CCN(CC(=O)O)C(=O)OCOP(=O)(O)O)c2C3=O)C1] |
| 51 | N-((((3′,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)carbonyl)-N-(2- (methyl(((phosphonooxy)methoxy)carbonyl) amino)ethyl)glycine | 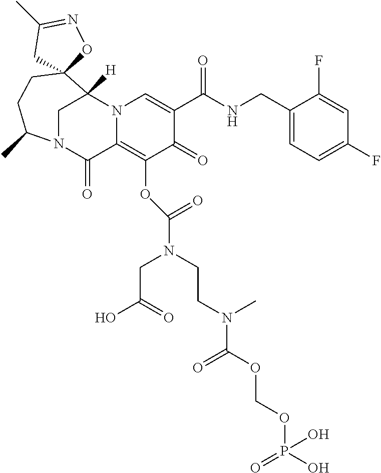 [SMILES: CC1=NO[C@@]2(CC[C@H](C)N3C[C@H]2n2cc(C(=O)NCc4ccc(F)cc4F)c(=O)c(OC(=O)N(CCN(C)C(=O)OCOP(=O)(O)O)CC(=O)O)c2C3=O)C1] |
| 52 | N-(2-((carboxymethyl)((((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′- yl)oxy)carbonyl)amino)ethyl)-N- (((phosphonooxy)methoxy)carbonyl)glycine | 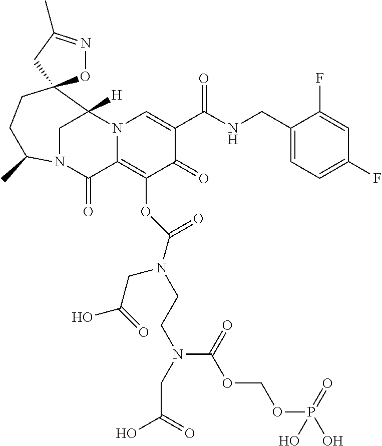 [SMILES: CC1=NO[C@@]2(CC[C@H](C)N3C[C@H]2n2cc(C(=O)NCc4ccc(F)cc4F)c(=O)c(OC(=O)N(CCN(CC(=O)O)C(=O)OCOP(=O)(O)O)CC(=O)O)c2C3=O)C1] |
| 53 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((R)-2-(phosphonooxy)propyl) carbonate | 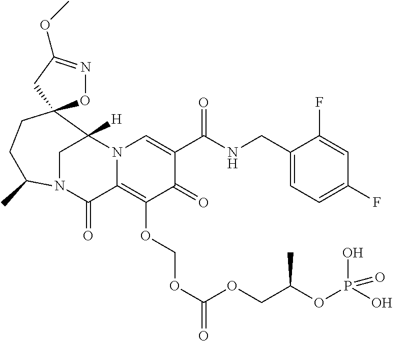 [SMILES: COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1] |
| 54 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((R)-1-(phosphonooxy)propan-2- yl) carbonate | 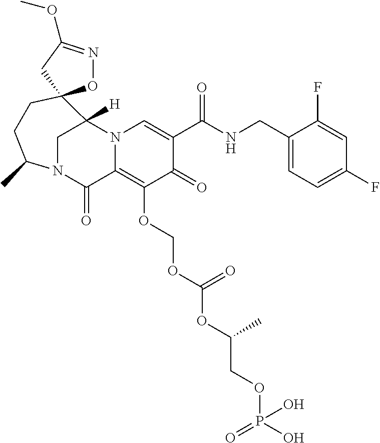 [SMILES: COC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1] |
| 55 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl (2-methyl-3- (phosphonooxy)butan-2-yl) carbonate | 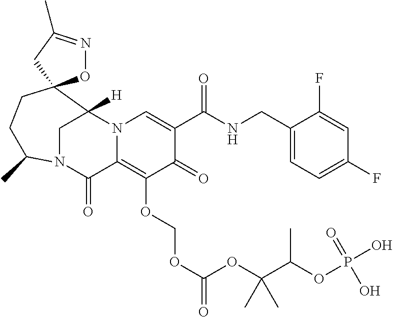 [SMILES: CC1=NO[C@@]2(CC[C@H](C)N3C[C@H]2n2cc(C(=O)NCc4ccc(F)cc4F)c(=O)c(OCOC(=O)OC(C)(C)C(C)OP(=O)(O)O)c2C3=O)C1] |
| 56 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl (3-methyl-3- (phosphonooxy)butan-2-yl) carbonate | 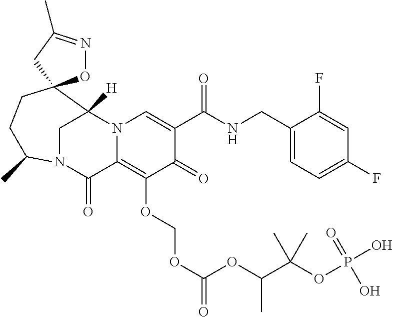 [SMILES: CC1=NO[C@@]2(CC[C@H](C)N3C[C@H]2n2cc(C(=O)NCc4ccc(F)cc4F)c(=O)c(OCOC(=O)OC(C)C(C)(C)OP(=O)(O)O)c2C3=O)C1] |
| 57 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2S,3R)-3- (phosphonooxy)butan-2-yl) carbonate | 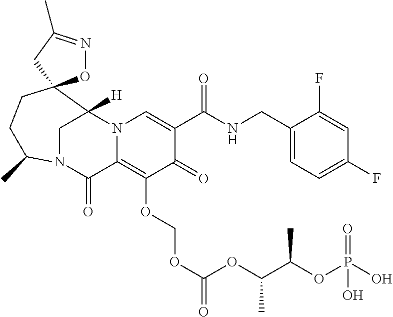 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1] |
| 58 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2R,3S)-3- (phosphonooxy)butan-2-yl) carbonate | 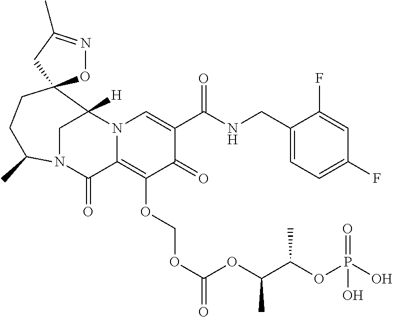 [SMILES: CC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1] |
| 59 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((2S,3R)-3- (phosphonooxy)butan-2-yl) carbonate | 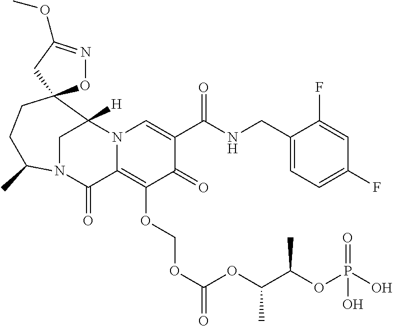 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1] |
| 60 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((2R,3S)-3- (phosphonooxy)butan-2-yl) carbonate | 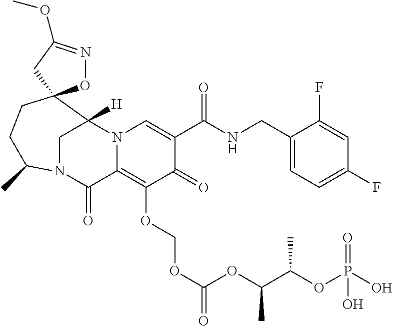 [SMILES: COC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1] |
| 61 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((2R,3R)-3- (phosphonooxy)butan-2-yl) carbonate | 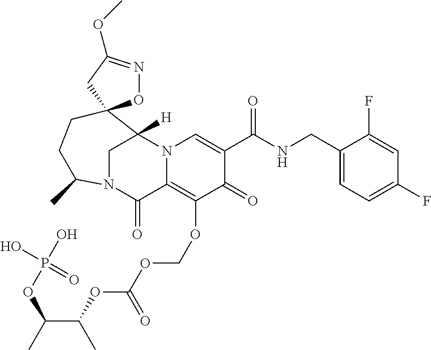 [SMILES: COC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1] |
| 62 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl (2-methyl-1- (phosphonooxy)propan-2-yl) carbonate | 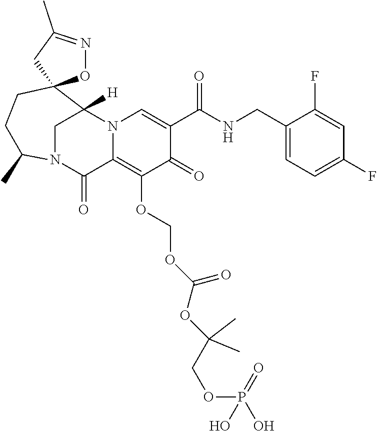 [SMILES: CC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1] |
| 63 | (((3′S,5S,7′R)-10′-((2,4- difluorobenzyl)carbamoyl)-3-methoxy-3′- methyl-1′,11′-dioxo-1′,4′,5′,11′-tetrahydro-tetrahydro- 3′H,4H,7′H-spiro[isoxazole-5,6′- [2,7]methanopyrido[1,2-a][1,4]diazonin]-12′- yl)oxy)methyl (2-methyl-1- (phosphonooxy)propan-2-yl) carbonate | 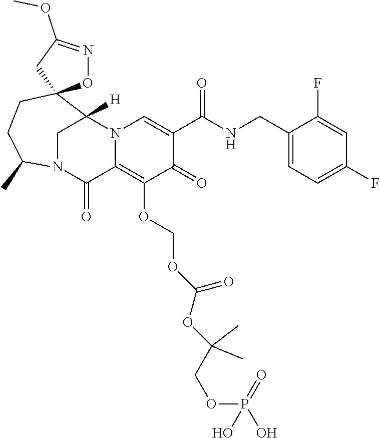 [SMILES: COC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1] |
| 64 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((R)-2- (phosphonooxy)butyl) carbonate | 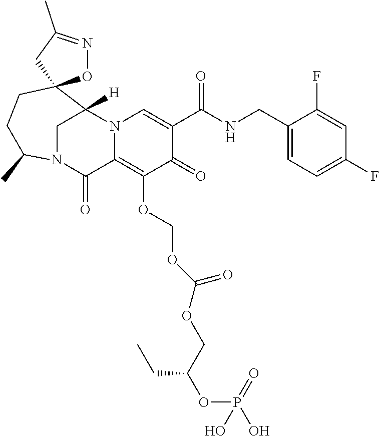 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O] |
| 65 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((R)-2-(phosphonooxy)butyl) carbonate | 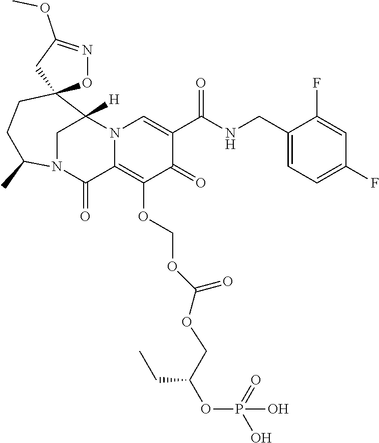 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O] |
| 66 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((S)-2- (phosphonooxy)butyl) carbonate | 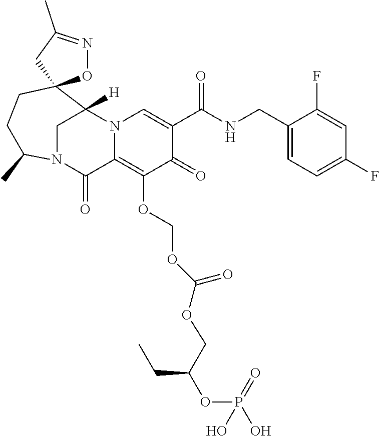 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O] |
| 67 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((S)-2-(phosphonooxy)butyl) carbonate | 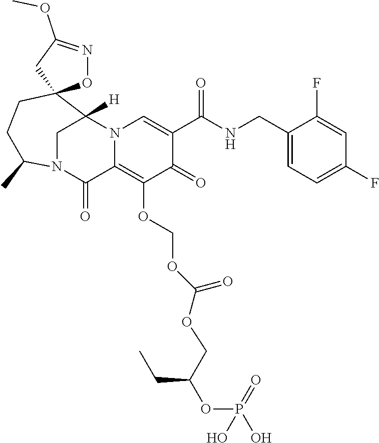 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O] |
| 68 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((S)-2-methyl-3- (phosphonooxy)propyl) carbonate | 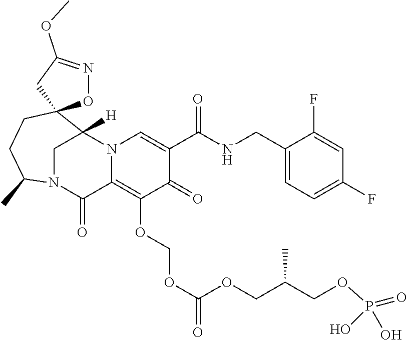 [SMILES: COC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1] |
| 69 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((S)-2- methyl-3-(phosphonooxy)propyl) carbonate | 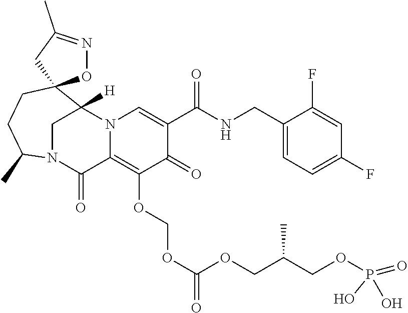 [SMILES: CC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1] |
| 70 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl) ((R)-2- methyl-3-(phosphonooxy)propyl) carbonate | 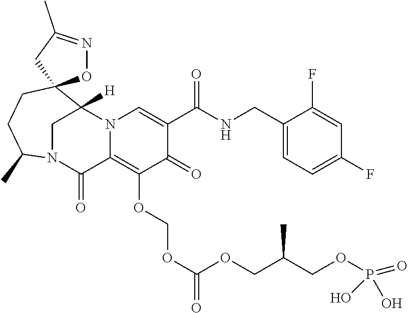 [SMILES: CC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1] |
| 71 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((R)-2-methyl-3- (phosphonooxy)propyl) carbonate | 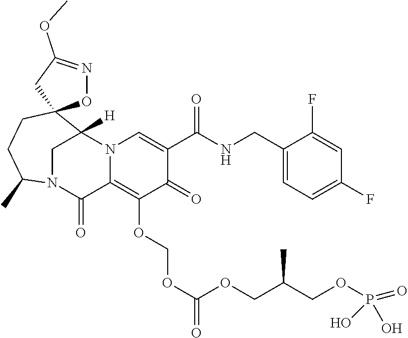 [SMILES: COC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1] |
| 72 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2S,3S)-3- (phosphonooxy)pentan-2-yl) carbonate | 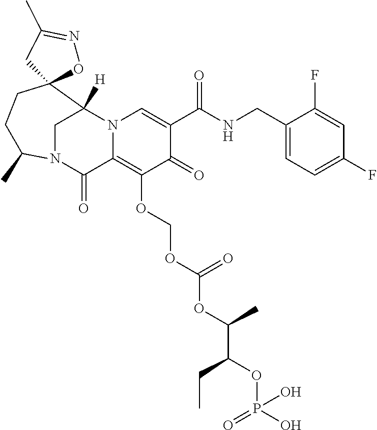 [SMILES: CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2] |
| 73 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2S,3R)-3- (phosphonooxy)pentan-2-yl) carbonate | 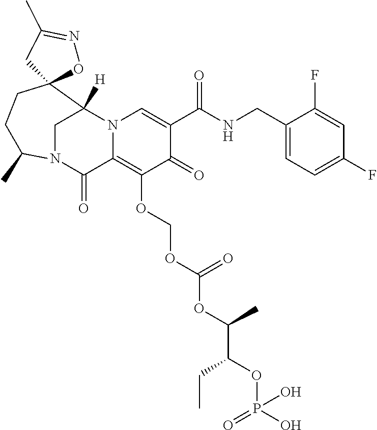 [SMILES: CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2] |
| 74 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((2S,3S)-3- (phosphonooxy)pentan-2-yl) carbonate | 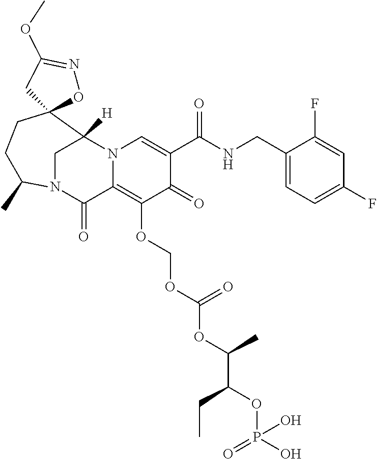 [SMILES: CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2] |
| 75 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((2S,3R)-3- (phosphonooxy)pentan-2-yl) carbonate | 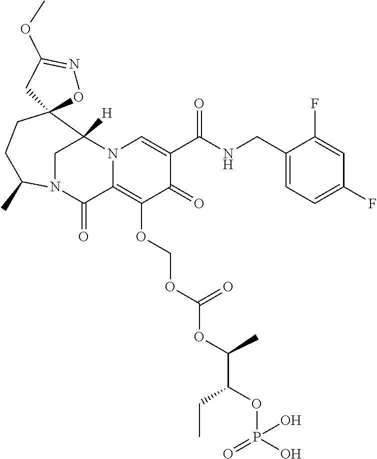 [SMILES: CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2] |
| 76 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2R,3R)-3- (phosphonooxy)pentan-2-yl) carbonate | 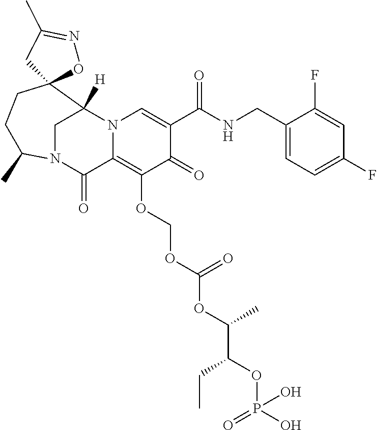 [SMILES: CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2] |
| 77 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((2R,3S)-3- (phosphonooxy)pentan-2-yl) carbonate | 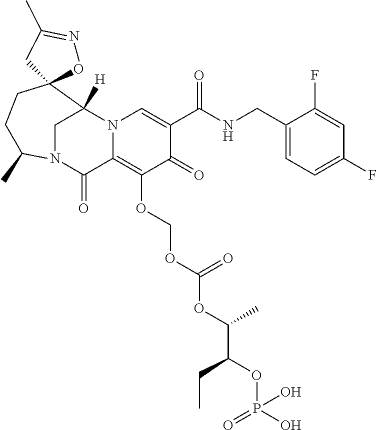 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2] |
| 78 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((2R,3R)-3- (phosphonooxy)pentan-2-yl) carbonate | 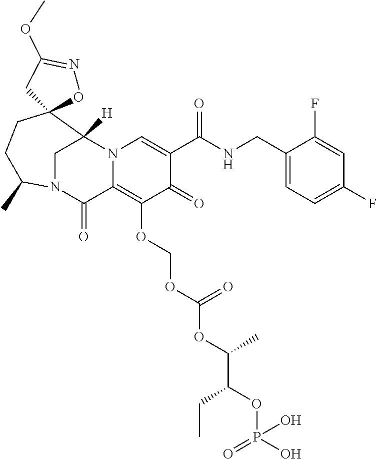 [SMILES: CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2] |
| 79 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((2R,3S)-3- (phosphonooxy)pentan-2-yl) carbonate | 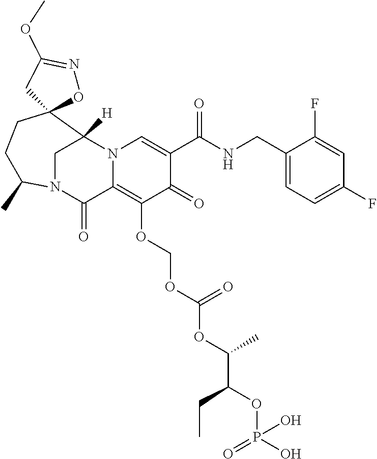 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2] |
| 80 | (((3′S,5S,7′R)-10′-((2,4- difluorobenzyl)carbamoyl)-3,3′-dimethyl-1′,11′- dioxo-1′,4′,5′,11′-tetrahydro-3′H,4H,7′H- spiro[isoxazole-5,6′-[2,7]methanopyrido[1,2- a][1,4]diazonin]-12′-yl)oxy)methyl ((R)-3- methyl-2-(phosphonooxy)butyl) carbonate | 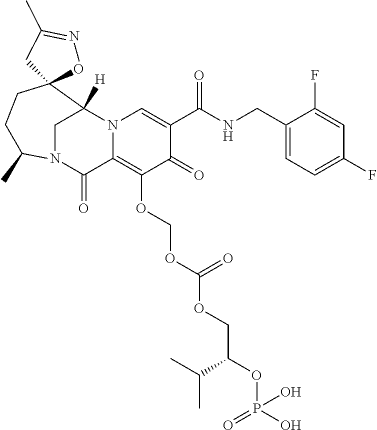 [SMILES: CC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1] |
| 81 | (((3′S,5S,7′R)-10′-((2,4- difluorobenzyl)carbamoyl)-3-methoxy-3′- methy-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((R)-3-methyl-2- (phosphonooxy)butyl) carbonate | 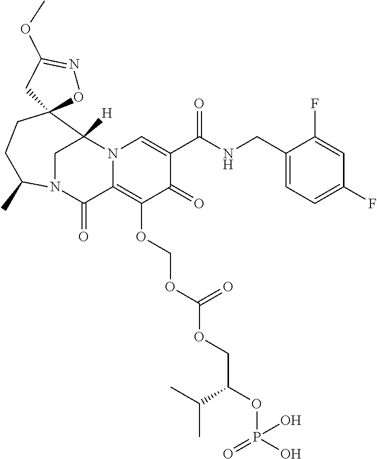 [SMILES: COC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1] |
| 82 | (((3′S,5S,7′R)-10′-((2,4- difluorobenzyl)carbamoyl)-3-methoxy-3′- methyl-1′,11′-dioxo-1′,4′,5′,11′-tetrahydro- 3′H,4H,7′H-spiro[isoxazole-5,6′- [2,7]methanopyrido[1,2-a][1,4]diazonin]-12′- yl)oxy)methyl ((S)-3-methyl-2- (phosphonooxy)butyl) carbonate | 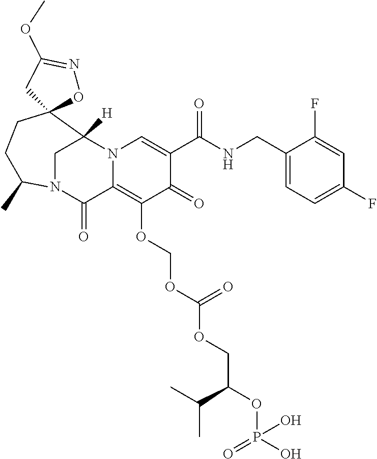 [SMILES: COC1=NO[C@@]2(CC[C@H](C)N3C[C@H]2n2cc(C(=O)NCc4ccc(F)cc4F)c(=O)c(OCOC(=O)OC[C@@H](OP(=O)(O)O)C(C)C)c2C3=O)C1] |

In some embodiments, the compounds disclosed herein have therapeutic activity. In some embodiments, the compounds disclosed herein are prodrugs, which upon administration to the human body can be converted to compounds having therapeutic activity. In some embodiments, the compounds disclosed herein are prodrugs of certain compounds disclosed in U.S. application Ser. No. 18/296,285; International Publication No. WO 2023/196875; and U.S. Pat. Nos. 12,054,496 and 12,024,528 the disclosures of which are incorporated herein by reference in their entireties. For example, the compounds of Formula I or Formula I′ disclosed herein can be converted to compounds of Formula II:

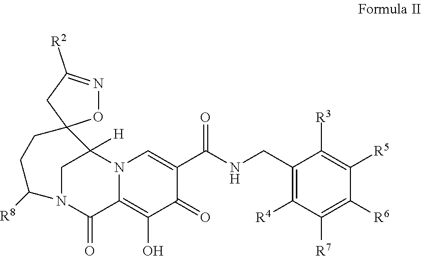

or pharmaceutically acceptable salts thereof, wherein R², R³, R⁴, R⁵, R⁶, R⁷, and R⁸ are as defined according to any other embodiment described herein.

In some embodiments, the compound of Formula II is a compound of Formula II′:

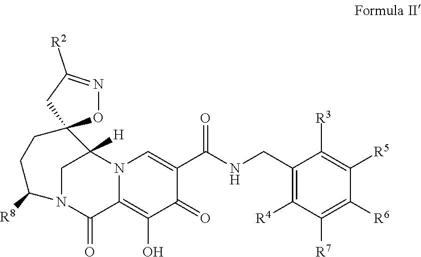

or pharmaceutically acceptable salts thereof, wherein R², R³, R⁴, R⁵, R⁶, R⁷, and R⁸ are as defined according to any other embodiment described herein.

In some embodiments, the compound of Formula II or Formula II′ is

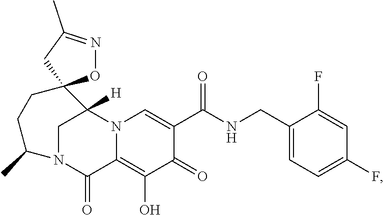 [SMILES: CC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof.

In some embodiments, the compound of Formula II or Formula II′ is

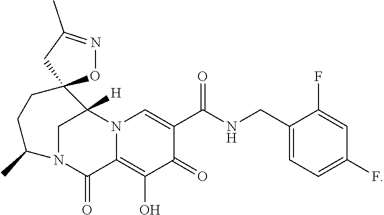 [SMILES: CC1=NO[C@@]2(CC[C@H](C)N3C[C@H]2n2cc(C(=O)NCc4ccc(F)cc4F)c(=O)c(O)c2C3=O)C1]

In some embodiments, the compound of Formula II or Formula II′ is

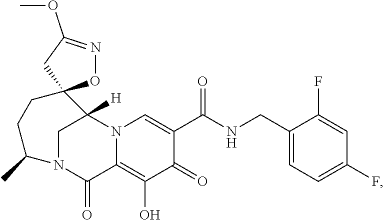 [SMILES: COC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof.

In some embodiments, the compound of Formula II or Formula II′ is

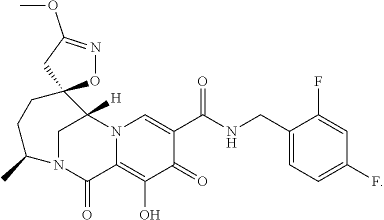 [SMILES: COC1=NO[C@@]2(CC[C@H](C)N3C[C@H]2n2cc(C(=O)NCc4ccc(F)cc4F)c(=O)c(O)c2C3=O)C1]

The present invention includes a method of converting a compound of Formula I, or a pharmaceutically acceptable salt thereof, or a compound of Formula I′, or a pharmaceutically acceptable salt thereof, to Formula II, or a pharmaceutically acceptable salt thereof, or Formula II′, or a pharmaceutically acceptable salt thereof. A compound of Formula I, or a pharmaceutically acceptable salt thereof, or a compound of Formula I′, or a pharmaceutically acceptable salt thereof, may be converted to a compound of Formula II, or a pharmaceutically acceptable salt thereof, or Formula II′, or a pharmaceutically acceptable salt thereof, by (1) contacting the compound of Formula I, or a pharmaceutically acceptable salt thereof, or Formula I′, or a pharmaceutically acceptable salt thereof, with cell-containing aqueous media capable of converting —OR¹ to —OH (e.g., either enzymatically or chemically through acid or base hydrolysis); or (2) administering the compound to a subject whereby the compound of Formula I, or pharmaceutically acceptable salt thereof, or Formula I′, or pharmaceutically acceptable salt thereof, is converted to a compound of Formula II, or a pharmaceutically acceptable salt thereof, or Formula II′, or a pharmaceutically acceptable salt thereof, through biologic pathways (e.g., enzymes) or through contact with biologic fluids and/or tissues conducive to converting —OR¹ to —OH (e.g., through acid or base hydrolysis).

The present invention further provides the treatment of HIV infection in a subject in need thereof, by contacting the subject with a compound of Formula II, or a pharmaceutically acceptable salt thereof, or Formula II′, or a pharmaceutically acceptable salt thereof, whereby the compound of Formula II, or a pharmaceutically acceptable salt thereof, or Formula II′, or a pharmaceutically acceptable salt thereof, is generated within the subject upon administration to the subject of a compound of Formula I, or a pharmaceutically acceptable salt thereof, or Formula I′, or a pharmaceutically acceptable salt thereof. In some embodiments, the compound of Formula II or Formula II′ that is generated within the subject is Intermediate B or Intermediate C, as described below in the Example section. In some embodiments, the treatment of HIV infection in a subject in need thereof uses a combination disclosed herein comprising a compound of Formula II, or a pharmaceutically acceptable salt thereof.

In some embodiments, the methods of therapeutic treatment comprise administering a compound of Formula I as a free acid or in the form of a pharmaceutically acceptable salt. The compound of Formula I, or its pharmaceutically acceptable salts, or Formula I′, or its pharmaceutically acceptable salts, can be present within a tablet in solvated or unsolvated form, and references to “a compound Formula I” include both of these forms. In particular, references to a compound of Formula I, or a pharmaceutically acceptable salt thereof, or Formula I′, or a pharmaceutically acceptable salt thereof, include pharmaceutically acceptable salts of a compound of Formula I or Formula I′ and pharmaceutically acceptable hydrates of a compound of Formula I or Formula I′. Additionally, the term “a compound of Formula I” or “a compound of Formula I′” is meant to refer to either or both a single molecule of a compound of Formula I (or a pharmaceutically acceptable salt thereof), or a preparation of a compound of Formula I (or a pharmaceutically acceptable salt thereof), or Formula I′ (or a pharmaceutically acceptable salt thereof), or a preparation of a compound of Formula I′ (or a pharmaceutically acceptable salt thereof), respectively, where the preparation may be characterized by bulk properties such as purity, particle size, and other characteristics.

As used herein, and in the absence of a specific reference to a particular pharmaceutically acceptable salt and/or solvate of a compound of Formula I, any dosages, whether expressed in e.g. milligrams or as a % by weight, should be taken as referring to the amount of a compound of Formula I free acid, i.e. the amount of:

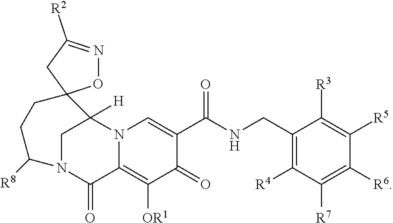

The pharmaceutical compositions described herein utilize a compound of Formula I, wherein the compound of Formula I is in the form of a free acid and/or a pharmaceutically acceptable salt thereof. In some embodiments, the pharmaceutical composition comprises the compound of Formula I. In some embodiments, the pharmaceutical composition comprises a pharmaceutically acceptable salt of the compound of Formula I.

As used herein, and in the absence of a specific reference to a particular pharmaceutically acceptable salt and/or solvate of a compound of Formula I′, any dosages, whether expressed in e.g. milligrams or as a % by weight, should be taken as referring to the amount of a compound of Formula I′ free acid, i.e. the amount of:

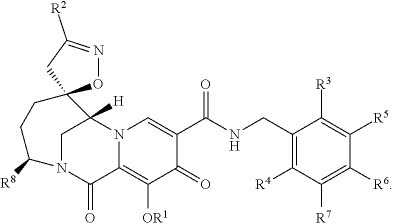

The pharmaceutical compositions described herein utilize a compound of Formula I′, wherein the compound of Formula I′ is in the form of a free acid and/or a pharmaceutically acceptable salt thereof. In some embodiments, the pharmaceutical composition comprises the compound of Formula I′. In some embodiments, the pharmaceutical composition comprises a pharmaceutically acceptable salt of the compound of Formula I′.

### IV. Pharmaceutical Compositions

Compounds provided herein are usually administered in the form of pharmaceutical compositions. Thus, provided herein are also pharmaceutical compositions that comprise one or more of the compounds provided herein or pharmaceutically acceptable salts, isomer, or a mixture thereof and one or more pharmaceutically acceptable vehicles selected from carriers, adjuvants and excipients. The compounds provided herein may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In one aspect, provided herein are pharmaceutical compositions comprising one or more compound provided herein (e.g., Compound 1 or Compound 1′ and/or a compound of Formula I, Formula I′, Formula II, or Formula II′), or a pharmaceutically acceptable salt thereof, and a pharmaceutically acceptable excipient or carrier. In some embodiments, the pharmaceutical compositions comprise a therapeutically effective amount of a compound provided herein, or a pharmaceutically acceptable salt thereof, and a pharmaceutically acceptable excipient or carrier.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds provided herein. Administration may be via, for example, capsule or enteric coated tablets. In making the pharmaceutical compositions that include at least one compound provided herein or pharmaceutically acceptable salts, isomer, or a mixture thereof, the active ingredient (such as a compound provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose, Vitamin E-Tocopherol polyethylene glycol succinate (Vitamin E-TPGS), and polyethoxylated castor oil (also known as Cremophor EL or Kolliphor EL), or any combinations thereof. The pharmaceutical compositions can additionally include lubricating agents such as talc, magnesium stearate, and mineral oil; wetting agents; emulsifying and suspending agents; preserving agents such as methyl and propylhydroxy-benzoates; sweetening agents; and flavoring agents; or any combinations thereof.

The pharmaceutical compositions that include at least one compound described herein or pharmaceutically acceptable salts, isomer, or a mixture thereof can be formulated so as to provide quick, sustained or delayed release of the active ingredient (such as a compound provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds provided herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or pharmaceutically acceptable salts, isomer, or a mixture thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, (i) Compound 1, and pharmaceutically acceptable salts thereof, and Compound 1′, and pharmaceutically acceptable salts thereof, and (ii) the compounds of Formula I, and pharmaceutically acceptable salts thereof, Formula I′, and pharmaceutically acceptable salts thereof, Formula II, and pharmaceutically acceptable salts thereof, and Formula II′, and pharmaceutically acceptable salts thereof, described herein are provided in the form of pharmaceutical compositions.

Compound 1, or a pharmaceutically acceptable salt thereof, or Compound 1′, or a pharmaceutically acceptable salt thereof, described herein may be present in the same pharmaceutical composition as the compounds of Formula I, or a pharmaceutically acceptable salt thereof, Formula I′, or a pharmaceutically acceptable salt thereof, Formula II, or a pharmaceutically acceptable salt thereof, or Formula II′, or a pharmaceutically acceptable salt thereof, described herein. Compound 1, or a pharmaceutically acceptable salt thereof, or Compound 1′, or a pharmaceutically acceptable salt thereof, described herein may be present in a different pharmaceutical composition as the compounds of Formula I, or a pharmaceutically acceptable salt thereof, Formula I′, or a pharmaceutically acceptable salt thereof, Formula II, or a pharmaceutically acceptable salt thereof, or Formula II′, or a pharmaceutically acceptable salt thereof, described herein.

Each pharmaceutical composition may comprise one or more pharmaceutically acceptable vehicles selected from carriers, adjuvants and excipients.

### V. Methods of Treatment

In one embodiment, methods of therapeutic treatment of an HIV (e.g., HIV-1 and/or HIV-2) infection in a subject comprising administering to the human a therapeutically effective amount of (a) Compound 1, or a pharmaceutically acceptable salt thereof, or Compound 1′, or a pharmaceutically acceptable salt thereof, and (b) a compound of Formula I, or a pharmaceutically acceptable salt thereof, or a compound of Formula I′, or a pharmaceutically acceptable salt thereof, are provided.

In some embodiments, the subject is a human.

In some embodiments, the subject is virologically suppressed.

In some embodiments, the subject is treatment experienced.

In some embodiments, the subject is treatment naive.

In some embodiments, (i) Compound 1, or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, are administered separately.

In some embodiments, (i) Compound 1, or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, are co-administered.

In some embodiments, (i) Compound 1, or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, are administered as a co-formulation.

In some embodiments, (i) Compound 1, or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, are administered once monthly, twice monthly, three times monthly, or four times monthly.

In some embodiments, (i) Compound 1, or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, are administered once monthly.

In some embodiments, Compound 1, or a pharmaceutically acceptable salt thereof, or Compound 1′, or a pharmaceutically acceptable salt thereof, is administered orally, intravenously, subcutaneously, or intramuscularly.

In some embodiments, the compound of Formula I, or a pharmaceutically acceptable salt thereof, or a compound of Formula I′, or a pharmaceutically acceptable salt thereof, is administered orally, intravenously, subcutaneously, or intramuscularly.

In some embodiments, both (i) Compound 1, or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, are administered orally, intravenously, subcutaneously, or intramuscularly.

In some embodiments, Compound 1, or a pharmaceutically acceptable salt thereof, or Compound 1′, or a pharmaceutically acceptable salt thereof, is administered orally.

In some embodiments, the compound of Formula I, or a pharmaceutically acceptable salt thereof, or a compound of Formula I′, or a pharmaceutically acceptable salt thereof, is administered orally.

In some embodiments, both (i) Compound 1, or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, are administered orally.

In some embodiments, both (i) Compound 1, or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, are administered orally once monthly.

In some embodiments, Compound 1 is administered as a free acid.

In some embodiments, Compound 1′ is administered as a free acid.

In some embodiments, the compound of Formula I is administered as a free acid.

In some embodiments, the compound of Formula I′ is administered as a free acid.

In some embodiments, the Compound 1 or Compound 1′ is administered as a free acid and the compound of Formula I or Formula I′ is administered as a free acid.

In another embodiment, a use of (i) Compound 1, or a pharmaceutically acceptable salt thereof, or Compound 1′, or a pharmaceutically acceptable salt thereof, and (ii) a compound of Formula I, or a pharmaceutically acceptable salt thereof, or a compound of Formula I′, or a pharmaceutically acceptable salt thereof, for therapeutic treatment of an HIV infection in a subject is provided.

In another embodiment, (i) Compound 1, or a pharmaceutically acceptable salt thereof, or Compound 1′, or a pharmaceutically acceptable salt thereof, and (ii) a compound of Formula I, or a pharmaceutically acceptable salt thereof, or a compound of Formula I′, or a pharmaceutically acceptable salt thereof, for use in a therapeutic treatment of an HIV infection in a subject is provided.

In another embodiment, (i) Compound 1, or a pharmaceutically acceptable salt thereof, a pharmaceutical composition of Compound 1, or pharmaceutically acceptable salt thereof, Compound 1′, or a pharmaceutically acceptable salt thereof, or a pharmaceutical composition of Compound 1′, or pharmaceutically acceptable salt thereof, and (ii) a compound of Formula I, or a pharmaceutically acceptable salt thereof, a pharmaceutical composition of Formula I, or pharmaceutically acceptable salt thereof, a compound of Formula I′, or a pharmaceutically acceptable salt thereof, or a pharmaceutical composition of Formula I′, or pharmaceutically acceptable salt thereof, for use in a therapeutic treatment of an HIV infection in a subject is provided.

In another embodiment, the use of (i) Compound 1, or a pharmaceutically acceptable salt thereof, or Compound 1′, or a pharmaceutically acceptable salt thereof, and (ii) a compound of Formula I, or a pharmaceutically acceptable salt thereof, or a compound of Formula I′, or a pharmaceutically acceptable salt thereof, as a research tool is disclosed.

In another embodiment, an article of manufacture comprising a composition effective to treat an HIV infection; and packaging material comprising a label which indicates that the composition can be used to treat infection by HIV is disclosed. Exemplary compositions comprise (i) Compound 1, or a pharmaceutically acceptable salt thereof, or Compound 1′, or a pharmaceutically acceptable salt thereof, and (ii) a compound of Formula I, or a pharmaceutically acceptable salt thereof or a compound of Formula I′, or a pharmaceutically acceptable salt thereof.

In still another embodiment, a method of inhibiting the replication of HIV is disclosed. The method comprises exposing the virus to an effective amount of (i) Compound 1,

or a pharmaceutically acceptable salt thereof, or Compound 1′, or a pharmaceutically acceptable salt thereof, and (ii) the compound of Formula I, or a pharmaceutically acceptable salt thereof, or a compound of Formula I′, or a pharmaceutically acceptable salt thereof, under conditions where replication of HIV is inhibited.

In another embodiment, the use of (i) Compound 1, or a pharmaceutically acceptable salt thereof, or Compound 1′, or a pharmaceutically acceptable salt thereof, and (ii) a compound of Formula I, or a pharmaceutically acceptable salt thereof, or a compound of Formula I′, or a pharmaceutically acceptable salt thereof, to inhibit the activity of the HIV capsid is disclosed.

In another embodiment, the use of (i) Compound 1, or a pharmaceutically acceptable salt thereof, or Compound 1′, or a pharmaceutically acceptable salt thereof, and (ii) a compound of Formula I, or a pharmaceutically acceptable salt thereof, or a compound of Formula I′, or a pharmaceutically acceptable salt thereof, to inhibit the replication of HIV is disclosed.

### VI. Administration

The compounds disclosed herein can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. In some embodiments, the administration is oral, intravenous, subcutaneous, or intramuscular. It will be appreciated that the preferred route may vary with, for example, the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered on a daily or intermittent schedule for the duration of the individual's life.

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The dosage may also be described as a total amount of a compound described herein administered per dose.

Dosage of Compound 1, or a pharmaceutically acceptable salt thereof, may be between about 1 mg and 4,000 mg, between about 2 to 3,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

In some embodiments, between about 500 mg and 1,500 mg of Compound 1, or a pharmaceutically acceptable salt thereof, may be administered to the subject.

In some embodiments, between about 500 mg and 1,500 mg of Compound 1, or a pharmaceutically acceptable salt thereof, may be administered to the subject orally. In some embodiments, between about 500 mg and 1,500 mg of Compound 1, or a pharmaceutically acceptable salt thereof, may be administered to the subject once monthly. In some embodiments, between about 500 mg and 1,500 mg of Compound 1, or a pharmaceutically acceptable salt thereof, may be administered to the subject orally once monthly.

In some embodiments, about 500 mg of Compound 1, or a pharmaceutically acceptable salt thereof, may be administered to the subject. In some embodiments, about 600 mg of Compound 1, or a pharmaceutically acceptable salt thereof, may be administered to the subject. In some embodiments, about 700 mg of Compound 1, or a pharmaceutically acceptable salt thereof, may be administered to the subject. In some embodiments, about 800 mg of Compound 1, or a pharmaceutically acceptable salt thereof, may be administered to the subject. In some embodiments, about 900 mg of Compound 1, or a pharmaceutically acceptable salt thereof, may be administered to the subject. In some embodiments, about 1,000 mg of Compound 1, or a pharmaceutically acceptable salt thereof, may be administered to the subject. In some embodiments, about 1,100 mg of Compound 1, or a pharmaceutically acceptable salt thereof, may be administered to the subject. In some embodiments, about 1,200 mg of Compound 1, or a pharmaceutically acceptable salt thereof, may be administered to the subject. In some embodiments, about 1,300 mg of Compound 1, or a pharmaceutically acceptable salt thereof, may be administered to the subject. In some embodiments, about 1,400 mg of Compound 1, or a pharmaceutically acceptable salt thereof, may be administered to the subject. In some embodiments, about 1,500 mg of Compound 1, or a pharmaceutically acceptable salt thereof, may be administered to the subject.

In some embodiments, about 1,000 mg of Compound 1, or a pharmaceutically acceptable salt thereof, may be administered to the subject orally. In some embodiments, between about 1,000 mg of Compound 1, or a pharmaceutically acceptable salt thereof, may be administered to the subject once monthly. In some embodiments, about 1,000 mg of Compound 1, or a pharmaceutically acceptable salt thereof, may be administered to the subject orally once monthly. In some embodiments, about 1,000 mg of Compound 1 may be administered to the subject orally once monthly.

Dosage of Compound 1′, or a pharmaceutically acceptable salt thereof, may be between about 1 mg and 4,000 mg, between about 2 to 3,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

In some embodiments, between about 500 mg and 1,500 mg of Compound 1′, or a pharmaceutically acceptable salt thereof, may be administered to the subject.

In some embodiments, between about 500 mg and 1,500 mg of Compound 1′, or a pharmaceutically acceptable salt thereof, may be administered to the subject orally. In some embodiments, between about 500 mg and 1,500 mg of Compound 1′, or a pharmaceutically acceptable salt thereof, may be administered to the subject once monthly. In some embodiments, between about 500 mg and 1,500 mg of Compound 1′, or a pharmaceutically acceptable salt thereof, may be administered to the subject orally once monthly.

In some embodiments, about 500 mg of Compound 1′, or a pharmaceutically acceptable salt thereof, may be administered to the subject. In some embodiments, about 600 mg of Compound 1′, or a pharmaceutically acceptable salt thereof, may be administered to the subject. In some embodiments, about 700 mg of Compound 1′, or a pharmaceutically acceptable salt thereof, may be administered to the subject. In some embodiments, about 800 mg of Compound 1′, or a pharmaceutically acceptable salt thereof, may be administered to the subject. In some embodiments, about 900 mg of Compound 1′, or a pharmaceutically acceptable salt thereof, may be administered to the subject. In some embodiments, about 1,000 mg of Compound 1′, or a pharmaceutically acceptable salt thereof, may be administered to the subject. In some embodiments, about 1,100 mg of Compound 1′, or a pharmaceutically acceptable salt thereof, may be administered to the subject. In some embodiments, about 1,200 mg of Compound 1′, or a pharmaceutically acceptable salt thereof, may be administered to the subject. In some embodiments, about 1,300 mg of Compound 1′, or a pharmaceutically acceptable salt thereof, may be administered to the subject. In some embodiments, about 1,400 mg of Compound 1′, or a pharmaceutically acceptable salt thereof, may be administered to the subject. In some embodiments, about 1,500 mg of Compound 1′, or a pharmaceutically acceptable salt thereof, may be administered to the subject.

In some embodiments, about 1,000 mg of Compound 1′, or a pharmaceutically acceptable salt thereof, may be administered to the subject orally. In some embodiments, between about 1,000 mg of Compound 1′, or a pharmaceutically acceptable salt thereof, may be administered to the subject once monthly. In some embodiments, about 1,000 mg of Compound 1′, or a pharmaceutically acceptable salt thereof, may be administered to the subject orally once monthly. In some embodiments, about 1,000 mg of Compound 1′ may be administered to the subject orally once monthly.

Dosage of a compound of Formula I, or a pharmaceutically acceptable salt thereof, may be between about 1 mg and 4,000 mg, between about 2 to 3,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

In some embodiments, about 1,000 mg of the compound of Formula I, or a pharmaceutically acceptable salt thereof, may be administered to the subject.

Dosage of a compound of Formula I′, or a pharmaceutically acceptable salt thereof, may be between about 1 mg and 4,000 mg, between about 2 to 3,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

In some embodiments, about 1,000 mg of the compound of Formula I′, or a pharmaceutically acceptable salt thereof, may be administered to the subject.

Dosage of a compound of Formula II, or a pharmaceutically acceptable salt or pharmaceutically acceptable tautomer thereof, may be between about 1 mg and 4,000 mg, between about 2 to 3,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

In some embodiments, about 1,000 mg of the compound of Formula II, or a pharmaceutically acceptable salt thereof, may be administered to the subject.

Dosage of a compound of Formula II′, or a pharmaceutically acceptable salt or pharmaceutically acceptable tautomer thereof, may be between about 1 mg and 4,000 mg, between about 2 to 3,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

In some embodiments, about 1,000 mg of the compound of Formula II′, or a pharmaceutically acceptable salt thereof, may be administered to the subject.

The dosage or dosing frequency of a compound of the present disclosure may be adjusted over the course of the treatment, based on the judgment of the administering physician.

The compounds of the present disclosure may be administered to an individual (e.g., a human) in a therapeutically effective amount. In some embodiments, the compound is administered once daily. In some embodiments, the compound is administered once every week. In some embodiments, the compound is administered once every month. In some embodiments, the compound is administered every two months. In some embodiments, the compound is administered every three months. In some embodiments, the compound is administered every four months. In some embodiments, the compound is administered every five months. In some embodiments, the compound is administered every six months. In some embodiments, the compound is administered every seven months. In some embodiments, the compound is administered every eight months. In some embodiments, the compound is administered every nine months. In some embodiments, the compound is administered every ten months. In some embodiments, the compound is administered every eleven months. In some embodiments, the compound is administered every year.

The compounds provided herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein include from about 0.3 mg to about 30 mg per dose, or from about 30 mg to about 300 mg per dose, or from about 0.3 g to about 30 mg per dose, or from about g to about 300 g per dose.

In some embodiments, the methods described herein comprise administering to the subject an initial daily dose of about 1 to 500 mg of a compound disclosed herein and increasing the dose by increments until clinical efficacy is achieved. Increments of about 5, 10, 25, 50, or 100 mg can be used to increase the dose. The dosage can be increased once a month.

When administered orally, the total daily dosage for a human subject may be between about 1 mg and 1,000 mg, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, or 1000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound disclosed herein is administered once daily in a method disclosed herein. In some embodiments, a compound disclosed herein is administered twice daily in a method disclosed herein. In some embodiments, a compound disclosed herein is administered once monthly in a method disclosed herein.

In some embodiments, a compound disclosed herein is administered once every 10 days. In some embodiments, a compound disclosed herein is administered once every 15 days. In some embodiments, a compound disclosed herein is administered once every 20 days. In some embodiments, a compound disclosed herein is administered once every 10-15 days. In some embodiments, a compound disclosed herein is administered once every 15-20 days. In some embodiments, a compound disclosed herein is administered once every 10-20 days. In some embodiments, a compound disclosed herein is administered once every month. In some embodiments, a compound disclosed herein is administered once every 2 months. In some embodiments, a compound disclosed herein is administered once every 3 months. In some embodiments, a compound disclosed herein is administered once every 4 months. In some embodiments, a compound disclosed herein is administered once every 5 months. In some embodiments, a compound disclosed herein is administered once every 6 months. In some embodiments, a compound disclosed herein is administered once every 8 months. In some embodiments, a compound disclosed herein is administered once every 10 months. In some embodiments, a compound disclosed herein is administered once every year.

The frequency of dosage of the compound of the present disclosure will be determined by the needs of the individual subject and can be, for example, once per day, once per week, once per two weeks, once per month, once per two months, once per three months, once per four months, once per six months, or less. The frequency of dosage of the compound of the present disclosure will be determined by the needs of the individual subject and can be once per month. Administration of the compound continues for as long as necessary to treat the HIV infection.

### VII. Kits and Articles of Manufacture

In one aspect, provided herein are kits that comprise (i) Compound 1, or a pharmaceutically acceptable salt, stereoisomer, or solvate thereof, or Compound 1′, or a pharmaceutically acceptable salt, stereoisomer, or solvate thereof, (ii) a compound of Formula I,

or a pharmaceutically acceptable salt, stereoisomer, prodrug, or solvate thereof, or a compound of Formula I′, or a pharmaceutically acceptable salt, stereoisomer, prodrug, or solvate thereof, and (iii) suitable packaging. In some embodiments, the kit further comprises instructions for use. In some embodiments, the kit comprises (i) Compound 1, or a pharmaceutically acceptable salt, stereoisomer, or solvate thereof, or Compound 1′, or a pharmaceutically acceptable salt, stereoisomer, or solvate thereof, (ii) a compound of Formula I, or a pharmaceutically acceptable salt, stereoisomer, prodrug, or solvate thereof, or a compound of Formula I′, or a pharmaceutically acceptable salt, stereoisomer, prodrug, or solvate thereof, and (iii) a label and/or instructions for use of the compounds in the treatment of the indications, including the diseases or conditions, described herein.

In one aspect, provided herein are articles of manufacture that comprise a compound described herein or pharmaceutically acceptable salts, isomer, or a mixture thereof in a suitable container. In some embodiments, the container may be a vial, jar, ampoule, preloaded syringe, or intravenous bag.

In one aspect, provided herein are kits that comprise (i) Compound 1, or a pharmaceutically acceptable salt, stereoisomer, or solvate thereof, or Compound 1′, or a pharmaceutically acceptable salt, stereoisomer, or solvate thereof, and (ii) a compound of Formula I, or a pharmaceutically acceptable salt, stereoisomer, prodrug, or solvate thereof, a compound of Formula I′, or a pharmaceutically acceptable salt, stereoisomer, prodrug, or solvate thereof, a compound of Formula II, or a pharmaceutically acceptable salt, stereoisomer, prodrug, or solvate thereof, or a compound of Formula II′, or a pharmaceutically acceptable salt, stereoisomer, prodrug, or solvate thereof. In some embodiments, the kit further comprises instructions for use. In some embodiments, the kit comprises (i) Compound 1, or a pharmaceutically acceptable salt, stereoisomer, or solvate thereof, or Compound 1′, or a pharmaceutically acceptable salt, stereoisomer, or solvate thereof, (ii) a compound of Formula I, or a pharmaceutically acceptable salt, stereoisomer, prodrug, or solvate thereof, a compound of Formula I′, or a pharmaceutically acceptable salt, stereoisomer, prodrug, or solvate thereof, a compound of Formula II, or a pharmaceutically acceptable salt, stereoisomer, prodrug, or solvate thereof, or a compound of Formula II′, or a pharmaceutically acceptable salt, stereoisomer, prodrug, or solvate thereof, and (iii) a label and/or instructions for use of the compounds in the treatment of the indications, including the diseases or conditions, described herein.

In some embodiments, the kit comprises (i) a pharmaceutical composition of Compound 1, or a pharmaceutically acceptable salt, stereoisomer, or solvate thereof, or a pharmaceutical composition of Compound 1′, or a pharmaceutically acceptable salt, stereoisomer, or solvate thereof, and (ii) a pharmaceutical composition of a compound of Formula I, or a pharmaceutically acceptable salt, stereoisomer, prodrug, or solvate thereof, a pharmaceutical composition of a compound of Formula I′, or a pharmaceutically acceptable salt, stereoisomer, prodrug, or solvate thereof, a pharmaceutical composition of a compound of Formula II, or a pharmaceutically acceptable salt, stereoisomer, prodrug, or solvate thereof, a pharmaceutical composition of a compound of Formula II′, or a pharmaceutically acceptable salt, stereoisomer, prodrug, or solvate thereof, and (iii) instructions for use.

### VII. Examples

### Intermediate A: (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (A)

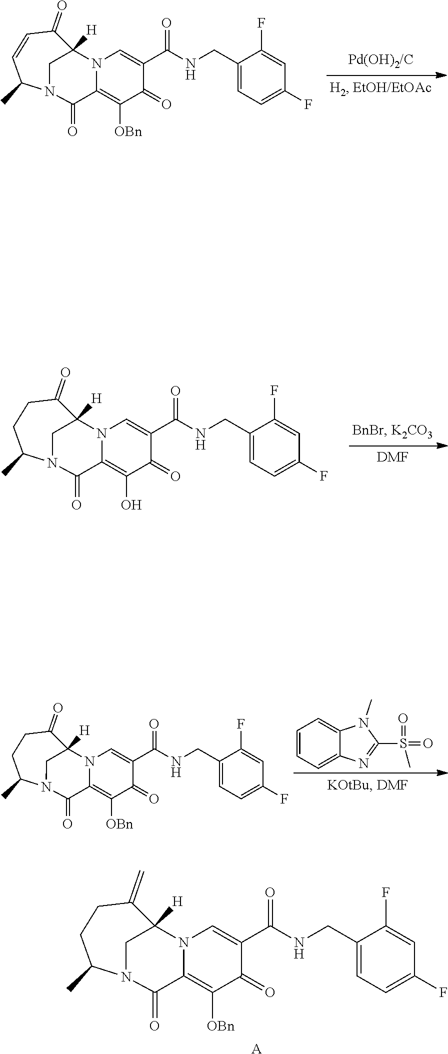

### Step 1: Synthesis of (3S,7R)-N-(2,4-difluorobenzyl)-12-hydroxy-3-methyl-1,6,11-trioxo-1,4,5,6,7,11-hexahydro-3H-2,7-methanopyrido[1,2-a][1,4]diazonine-10-carboxamide

To a solution of (3S,7R)-12-(benzyloxy)-N-(2,4-difluorobenzyl)-3-methyl-1,6,11-trioxo-1,6,7,11-tetrahydro-3H-2,7-methanopyrido[1,2-a][1,4]diazonine-10-carboxamide (17 g, 32.7 mmol) in EtOAc (200 mL) at room temperature was added EtOH (400 mL) followed by 20% Pd(OH)₂/C (50 wt % water, 7.6 g). The resulting mixture was degassed and flushed with nitrogen three times and then degassed and flushed with hydrogen three times before it was hydrogenated under hydrogen balloon for 4 hours. The reaction was then degassed and flushed with nitrogen, diluted with DCM, filtered through Celite®, concentrated and used directly in next step. MS (m/z) 432.124 [M+H]⁺.

### Step 2: Synthesis of (3S,7R)-12-(benzyloxy)-N-(2,4-difluorobenzyl)-3-methyl-1,6,11-trioxo-1,4,5,6,7,11-hexahydro-3H-2,7-methanopyrido[1,2-a][1,4]diazonine-O-carboxamide

(3S,7R)-N-(2,4-difluorobenzyl)-12-hydroxy-3-methyl-1,6,11-trioxo-1,4,5,6,7,11-hexahydro-3H-2,7-methanopyrido[1,2-a][1,4]diazonine-10-carboxamide (33.1 g, 76.7 mmol) from Step 1 was dissolved in DMF (300 mL) at room temperature and K₂CO₃ (16.0 g, 115.0 mmol) and benzyl bromide (13.1 g, 76.7 mmol) were added. The resulting mixture was then heated to 50° C. for 4.5 hours and then cooled to room temperature. The mixture was filtered through a pad of Celite® and the filter cake was rinsed with DMF (100 mL). Combined filtrate was carried directly into the next step. MS (m/z) 554.086 [M+H+MeOH]⁺.

### Step 3: Synthesis of(3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide

The solution of (3S,7R)-12-(benzyloxy)-N-(2,4-difluorobenzyl)-3-methyl-1,6,11-trioxo-1,4,5,6,7,11-hexahydro-3H-2,7-methanopyrido[1,2-a][1,4]diazonine-10-carboxamide (39.7 g, 76.1 mmol) in DMF (350 mL) was immersed into a room temperature water bath. 1-methyl-2-(methylsulfonyl)-1H-benzo[d]imidazole (20.81 g, 99.0 mmol) was added in one portion followed by potassium tert-butoxide (21.36 g, 190 mmol) in 5 portions. The reaction was removed from the water bath and stirred at room temperature for 1.5 hours. The reaction was then quenched slowly with 0.5N HCl in water (180 mL) and extracted with EtOAc three times. The combined organic layer was washed with water and brine, dried over sodium sulfate, filtered and concentrated. The residue was purified by normal phase silica gel chromatography, eluting with 0-100% EtOAc/hexane, to afford intermediate A. MS (m/z) 520.060 [M+H]⁺.

### Intermediate B: Preparation of (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (B)

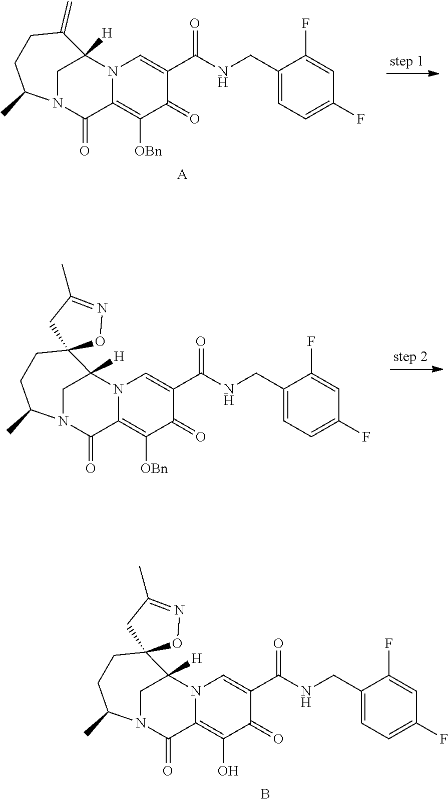

### Step 1: Preparation of (3′S,5S,7′R)-12′-(benzyloxy)-N-(2,4-difluorobenzyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

Into the solution of acetaldehyde oxime (666 mg, 11.3 mmol) in DMF (50 ml) was added N-chlorosuccinimide (1.51 g, 11.3 mmol) at room temperature, then heated to 60° C. for 1 h. After cooling to room temperature, (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (Intermediate A) (1.58 g, 3.04 mmol) and triethylamine (1.539 g, 15.2 mmol) were added at room temperature. After stirring at room temperature overnight, the reaction was quenched by adding sat. NaHCO₃ solution. The mixture was extracted with EtOAc, the organic phase was separated and dried over MgSO₄, filtered, concentrated down and purified by silica gel chromatography column (eluting with 0-100% EtOAc/hexane). MS (m/z) 577.135 [M+H]+

### Step 2: Preparation of (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (B)

To a solution of (3′S,5S,7′R)-12′-(benzyloxy)-N-(2,4-difluorobenzyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (64.7 mg, 0.112 mmol) in toluene (2 mL) was added TFA (0.5 mL). The reaction mixture was stirred at room temperature overnight. The reaction mixture was concentrated down, and the residue was purified by reverse phase HPLC, eluting with 10-90% acetonitrile in water to give intermediate B. MS (m/z) 487.12 [M+H]⁺. ¹H NMR (400 MHz, Chloroform-d) δ 10.53 (s, 1H), 8.43 (s, 1H), 7.37 (td, J=8.6, 6.3 Hz, 1H), 6.92-6.78 (m, 2H), 4.82-4.70 (m, 1H), 4.67 (t, J=4.8 Hz, 2H), 4.18 (d, J=2.2 Hz, 1H), 3.86 (dd, J=14.9, 1.9 Hz, 1H), 3.72 (dd, J=14.9, 2.7 Hz, 1H), 2.94 (d, J=17.8 Hz, 1H), 2.53 (d, J=17.7 Hz, 1H), 2.06 (s, 3H), 2.04-1.88 (m, 3H), 1.56 (dd, J=14.3, 11.2 Hz, 1H), 1.32 (d, J=6.6 Hz, 3H).

### Intermediate C: Synthesis of (3′S,5S,7′R)-N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

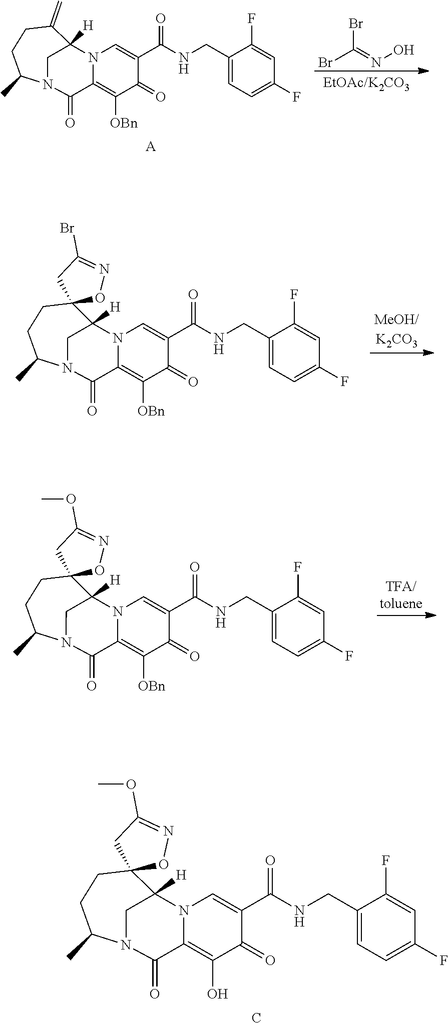

### Step 1: Synthesis of (3′S,5S,7′R)-12′-(benzyloxy)-3-bromo-N-(2,4-difluorobenzyl)-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

To a solution of (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (Intermediate A) (18.1 g, 34.8 mmol) in EtOAc (350 mL) at 0° C. was added dibromomethanone oxime (21.199 g, 105 mmol) followed by potassium carbonate (28.846 g, 209 mmol). The resulting mixture was stirred overnight before it was diluted with water (300 mL). The layers were separated. The aqueous layer was extracted with EtOAc (200 mL) and the combined organic phase was washed with brine, dried over sodium sulfate, filtered and concentrated. The residue was purified by normal phase silica gel chromatography, eluting with 0-80% EtOAc/Hexane to afford the title compound. MS (m/z) 640.904 [M+H]⁺. ¹H NMR (400 MHz, DMSO) δ 10.41 (t, J=6.0 Hz, 1H), 8.89 (s, 1H), 7.54-7.21 (m, 7H), 7.13-7.05 (m, 1H), 5.30 (d, J=10.5 Hz, 1H), 5.04 (d, J=10.5 Hz, 1H), 4.78 (s, 1H), 4.72-4.54 (m, 3H), 3.75-3.62 (m, 2H), 3.39 (d, J=17.6 Hz, 1H), 3.07 (d, J=17.7 Hz, 1H), 1.92-1.73 (m, 3H), 1.41 (t, J=12.9 Hz, 1H), 1.15 (d, J=6.7 Hz, 3H).

### Step 2: Synthesis of (3′S,5S,7′R)-12′-(benzyloxy)-N-(2,4-difluorobenzyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

To a mixture of (3′S,5S,7′R)-12′-(benzyloxy)-3-bromo-N-(2,4-difluorobenzyl)-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (23.2 g, 36.2 mmol) in MeOH (450 mL) and DMF (100 mL) at 55° C. was added potassium carbonate (12.496 g, 90.4 mmol). The reaction was stirred for 3 hrs and then cooled to room temperature. To this stirred mixture was added water (1100 mL) and stirring continued for 30 minutes. The mixture was filtered and the filter cake was rinsed with water, collected, and dried to give the title compound. MS (m/z) 593.207 [M+H]⁺. ¹H NMR (400 MHz, DMSO) δ 10.41 (t, J=6.0 Hz, 1H), 8.73 (s, 1H), 7.53-7.48 (m, 2H), 7.47-7.30 (m, 4H), 7.25 (ddd, J=10.5, 9.3, 2.6 Hz, 1H), 7.08 (tdd, J=8.5, 2.6, 1.0 Hz, 1H), 5.30 (d, J=10.5 Hz, 1H), 5.03 (d, J=10.5 Hz, 1H), 4.73-4.63 (m, 2H), 4.63-4.49 (m, 2H), 3.82 (s, 3H), 3.65 (d, J=2.0 Hz, 2H), 3.08 (d, J=16.8 Hz, 1H), 2.77 (d, J=16.9 Hz, 1H), 1.96-1.71 (m, 3H), 1.38 (dd, J=15.5, 10.2 Hz, 1H), 1.15 (d, J=6.6 Hz, 3H).

### Step 3: Synthesis of (3′S,5S,7′R)-N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (C)

To a solution of (3′S,5S,7′R)-12′-(benzyloxy)-N-(2,4-difluorobenzyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (19 g, 32.1 mmol) in toluene (64 mL) at room temperature was added TFA (32 mL) and stirred for 16 hrs. The solvents were removed by rotatory evaporator. The resulting residue was treated with EtOAc (60 mL) and concentrated, repeating three times. The residue was stirred with a mixture of toluene (42 mL)/MeOH (42 mL)/EtOAc (42 mL) at 70° C. for 1 hr and room temperature for 2 hours. The mixture was then filtered and the filter cake was rinsed with EtOAc, collected and dried to give title compound. MS (m/z) 503.263 [M+H]⁺. ¹H NMR (400 MHz, DMSO) δ 10.99 (s, 1H), 10.34 (t, J=5.9 Hz, 1H), 8.64 (s, 1H), 7.42 (td, J=8.7, 6.6 Hz, 1H), 7.25 (ddd, J=10.5, 9.3, 2.6 Hz, 1H), 7.08 (tdd, J=8.6, 2.6, 1.0 Hz, 1H), 4.74 (s, 1H), 4.63-4.47 (m, 3H), 3.81 (s, 3H), 3.80-3.67 (m, 2H), 2.93 (d, J=16.9 Hz, 1H), 2.70 (d, J=16.9 Hz, 1H), 1.95-1.76 (m, 3H), 1.41-1.31 (m, 1H), 1.19 (d, J=6.8 Hz, 3H).

### Intermediate D: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate

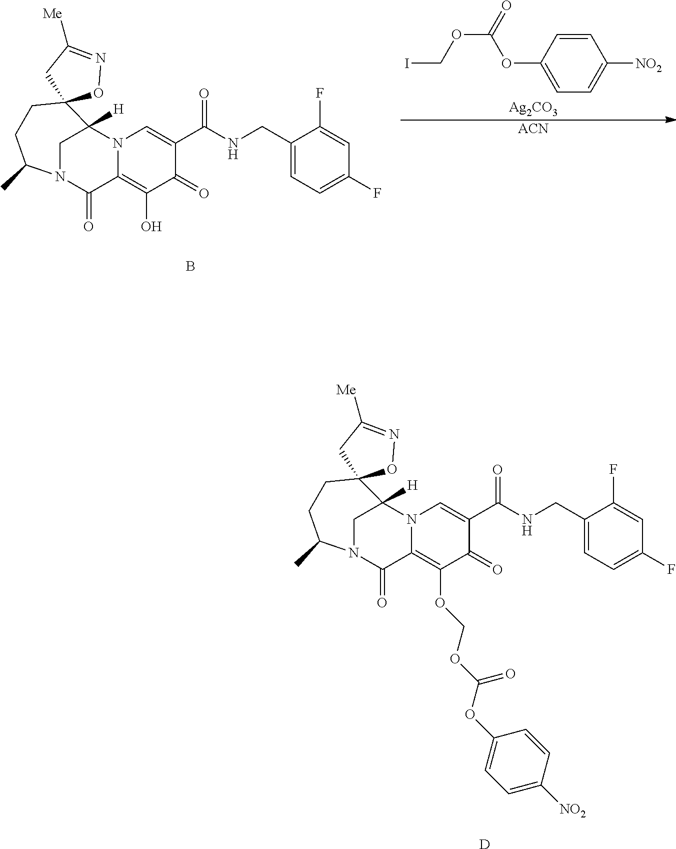

(3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 3.06 g, 6.29 mol) and iodomethyl (4-nitrophenyl) carbonate (4.23 g, 13.1 mmol), prepared according to WO2010011812, were mixed with acetonitrile (73 mL). Ag₂CO₃ (5.29 g, 19.2 mmol) was added in one portion. The slurry was stirred at room temperature overnight and filtered through celite. The filtrate was collected and concentrated to dryness. The residue was purified on silica gel column with 0-20% MeOH in DCM to afford the crude material upon concentration. The crude product was then dissolved in EtOAc (200 mL) and was treated with water (200 mL) with agitation. Organic phase was separated, dried over Na₂SO₄ and filtered. The filtrate was concentrated to afford Intermediate D. Calculated for C₃₂H₂₉F₂N₅O₁₀: 681.19, Found MS (ESI+): 682.01 [M+H]+.

### Intermediate E: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate

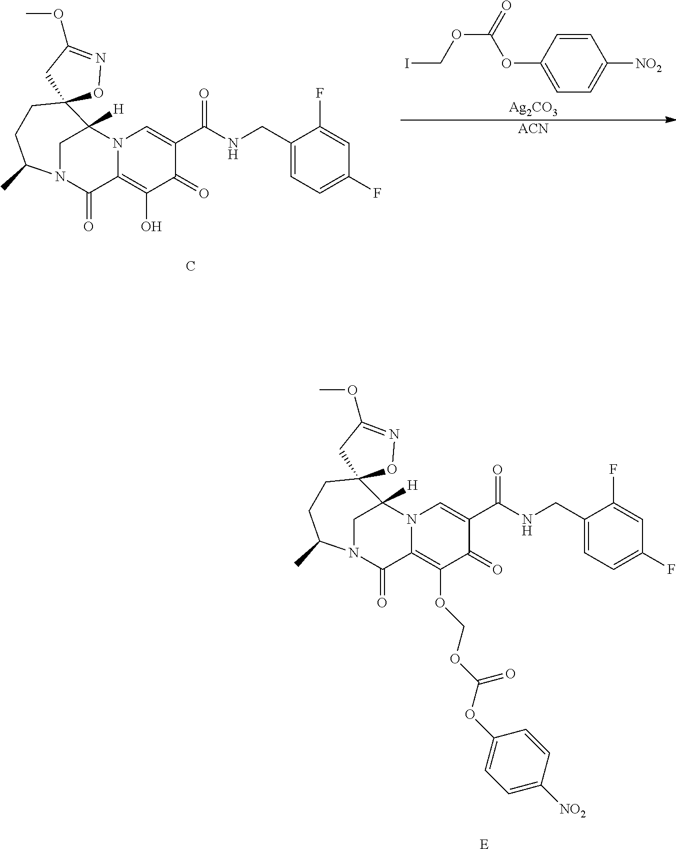

The title compound was prepared following a similar method for Intermediate D, except using Intermediate C instead of Intermediate B. LCMS-ESI+ (m/z): calcd H+ for C32H29F2N5O11, Theoretical: 697.18, Found: 697.845.

### Example 1: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl dihydrogen phosphate (1)

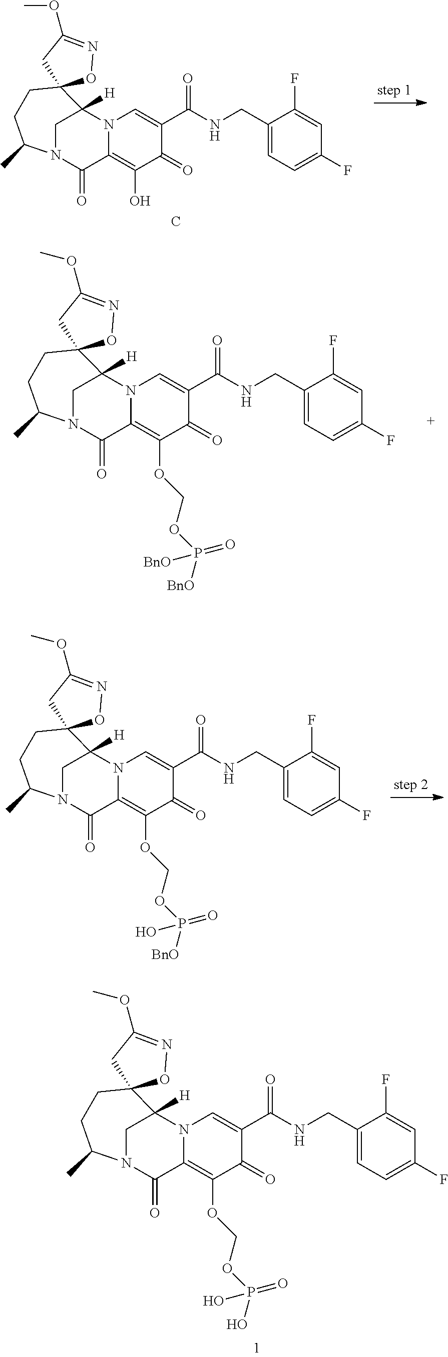

### Step 1: Synthesis of dibenzyl ((((3′S,5S,7′R)-10′-((2,4-difluorobenzyl)carbamoyl)-3-methoxy-3′-methyl-1′,11′-dioxo-1′,4′,5′11′-tetrahydro-3′H,4H,7′H-spiro[isoxazole-5,6′-[2,7]methanopyrido[1,2-a][1,4]diazonin]-12′-yl)oxy)methyl) phosphate and benzyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) hydrogen phosphate

To a solution of (3′S,5S,7′R)-N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C, 30 mg, 0.0597 mmol) in DMF (0.5 mL) at room temperature was added tetrabutylammonium iodide (14.7 mg, 0.0398 mmol) and potassium carbonate (11.0 mg, 0.0796 mmol). The resulting mixture was heated at 63° C. Dibenzyl chloromethyl phosphate (19.5 mg, 0.0597 mmol) was added to the hot mixture and the newly formed mixture was stirred for 30 minutes. The reaction was then cooled to room temperature and filtered. The mother liquor was purified by reverse phase preparative chromatography, fractions with desired mass were pooled and lyophilized. LCMS-ESI+ (m/z): calcd H+ for C₃₉H₃₉F₂N4010P, Theoretical: 792.24, Found: 792.697. LCMS-ESI+ (m/z): calcd H+ for C₃₂H₃₃F₂N4010P, Theoretical: 702.19, Found: 702.722.

### Step 2: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl dihydrogen phosphate (1)

The mixture of dibenzyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) phosphate and benzyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) hydrogen phosphate was dissolved in THF (10 mL) at room temperature. Platinum oxide (6.0 mg) was added and the resulting mixture was degassed and flushed with nitrogen three times. The reaction mixture was then degassed and flushed with hydrogen three times before it was hydrogenated under hydrogen balloon for 45 minutes. The reaction was then degassed and flushed with nitrogen, filtered, and concentrated. The residue was redissolved in DMF, filtered and purified by reverse phase preparative chromatography (5-100% MeCN/H₂O w/0.1% TFA). ¹H NMR (400 MHz, Acetonitrile-d3) δ 9.85 (t, J=6.0 Hz, 1H), 8.68 (s, 1H), 7.50-7.38 (m, 1H), 7.03-6.92 (m, 2H), 5.70-5.57 (m, 2H), 4.86-4.72 (m, 1H), 4.61 (d, J=5.5 Hz, 2H), 4.49 (s, 1H), 3.88 (s, 3H), 3.80-3.69 (m, 2H), 2.98 (d, J=17.3 Hz, 1H), 2.70 (d, J=17.3 Hz, 1H), 2.07-1.81 (m, 3H), 1.60-1.48 (m, 1H), 1.23 (d, J=6.7 Hz, 3H). LCMS-ESI+ (m/z): calcd H+ for C25H27F2N4O10P, Theoretical: 612.14, Found: 612.662.

### Example 2: Synthesis of N-(4-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-3,3-dimethyl-4-oxobutyl)-N-(((phosphonooxy)methoxy)carbonyl)glycine (2)

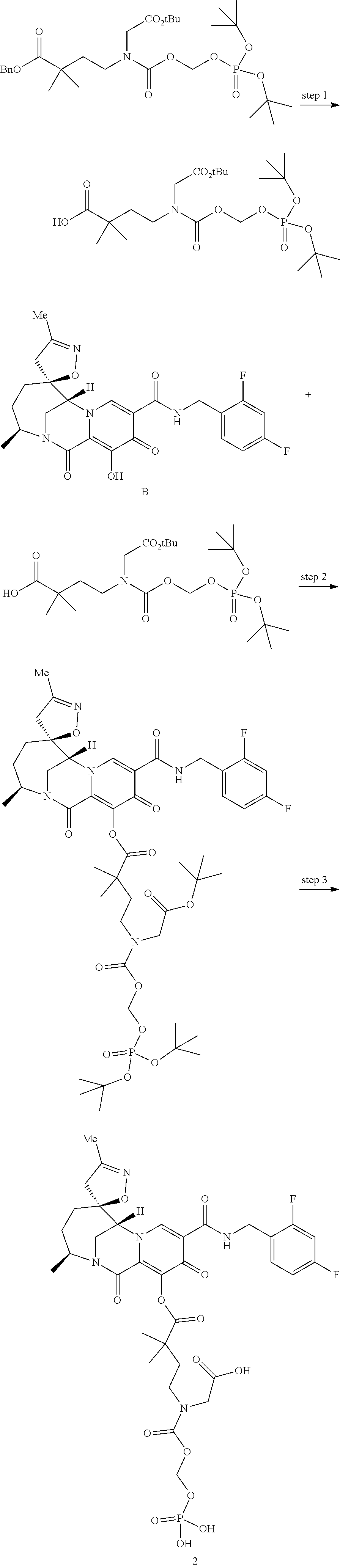

### Step 1: Synthesis of 4-(2-(tert-butoxy)-2-oxoethyl)((((di-tert-butoxyphosphoryl)oxy)methoxy)carbonyl)amino)-2,2-dimethylbutanoic acid

The solution of benzyl 4-((2-(tert-butoxy)-2-oxoethyl)((((di-tert-butoxyphosphoryl)oxy)methoxy)carbonyl)amino)-2,2-dimethylbutanoate (prepared according to WO2023102239) (200 mg, 0.332 mmol) in MeOH (20.0 mL) at room temperature was treated with platinum oxide (20.0 mg, 0.088 mmol). The resulting mixture was degassed and flushed with nitrogen three times and then degassed and flushed with hydrogen three times before it was hydrogenated under hydrogen balloon for 4 hours. The reaction was then degassed and flushed with nitrogen, diluted with DCM, filtered and concentrated and used directly in next step. LCMS-ESI+ (m/z): calcd H+ for C22H42NO10P, Theoretical: 511.25, Found: 511.512.

### Step 2: Synthesis of (3′S,5S,7′R)-10′-((2,4-difluorobenzyl)carbamoyl)-3,3′-dimethyl-1′,11′-dioxo-1′4′,5′,11′-tetrahydro-3′H,4H,7′H-spiro[isoxazole-5,6′-[2,7]methanopyrido[1,2-a][1,4]diazonin]-12′-yl 4-((2-(tert-butoxy)-2-oxoethyl)((((di-tert-butoxyphosphoryl)oxy)methoxy)carbonyl)amino)-2,2-dimethylbutanoate

To a mixture of (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 60 mg, 0.123 mmol) and 4-((2-(tert-butoxy)-2-oxoethyl)((((di-tert-butoxyphosphoryl)oxy)methoxy)carbonyl)amino)-2,2-dimethylbutanoic acid (75.7 mg, 0.148 mmol) in DMF (0.5 mL) at room temperature was added 1-ethyl-3-(3-dimethylaminopropyl) carbodiimide hydrochloride (30.6 mg, 0.16 mmol), DMAP (19.6 mg, 0.16 mmol), and triethylamine (25.0 mg, 0.247 mmol). After stirring for 16 hours, the reaction was diluted with EtOAc, washed with 0.5N aq HCl, water, brine, dried over sodium sulfate, filtered, concentrated and purified by normal phase chromatography. MS (m/z) 979.529 [M]+.

### Step 3: Synthesis of N-(4-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-3,3-dimethyl-4-oxobutyl)-N-(((phosphonooxy)methoxy)carbonyl)glycine (2)

(3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-((2-(tert-butoxy)-2-oxoethyl)((((di-tert-butoxyphosphoryl)oxy)methoxy)carbonyl)amino)-2,2-dimethylbutanoate (60 mg) was dissolved in DCM (3.0 mL) at room temperature and treated with TFA (1.0 mL) slowly. After stirred for 30 minutes, the reaction was then concentrated, dissolved in DMF, filtered and purified by reverse phase preparative HPLC (5-100% MeCN/H₂O w/0.1% TFA) to afford the title compound. ¹H NMR (400 MHz, Acetone-d6) δ 10.23 (s, 1H), 8.68 (d, J=7.1 Hz, 1H), 7.48 (td, J=8.6, 6.2 Hz, 1H), 7.09-6.89 (m, 2H), 5.72-5.53 (m, 2H), 4.86-4.72 (m, 1H), 4.72-4.55 (m, 3H), 4.36-4.13 (m, 2H), 4.07-3.77 (m, 2H), 3.74-3.44 (m, 2H), 3.29-3.16 (m, 1H), 2.85-2.69 (m, 1H), 2.01 (s, 5H), 1.97-1.83 (m, 3H), 1.67-1.43 (m, 1H), 1.36 (s, 6H), 1.26 (d, J=6.6 Hz, 3H). LCMS-ESI+ (m/z): calcd H+ for C34H40F2N5O14P, Theoretical: 811.23, Found: 811.769.

### Example 3: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl(2-(phosphonooxy)ethyl) carbonate (3)

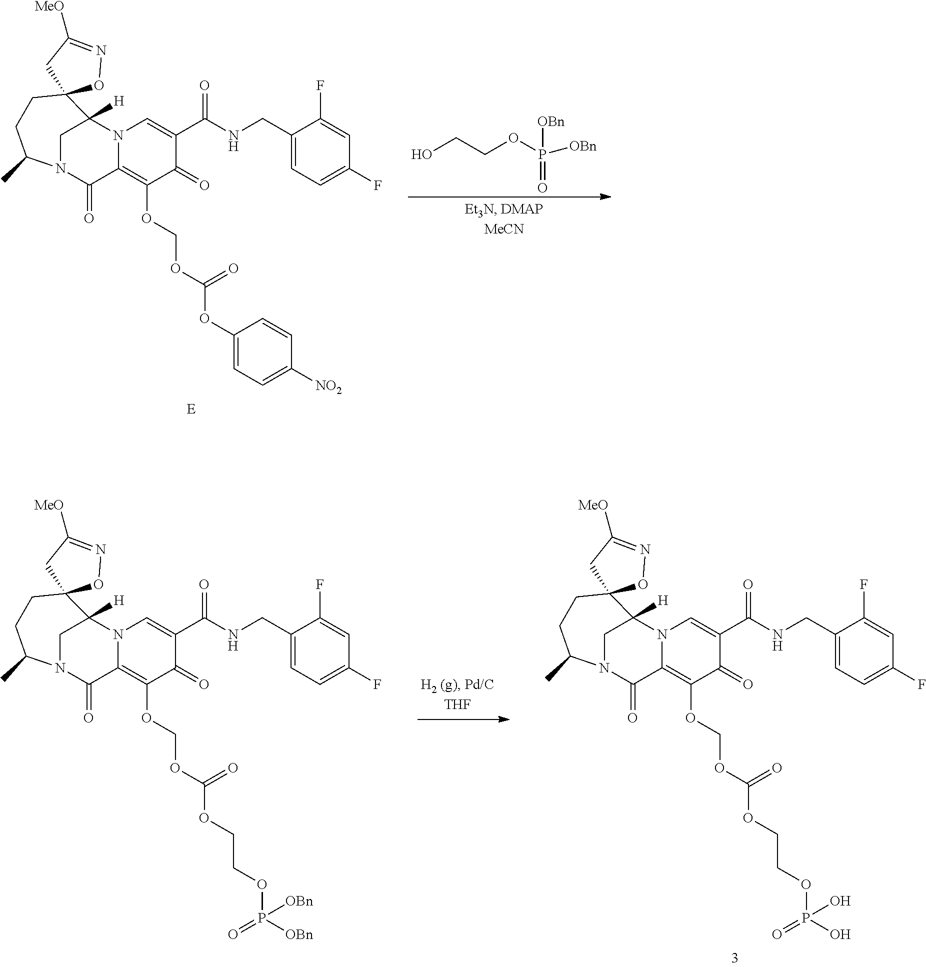

### Step 1: Preparation of 2-((bis(benzyloxy)phosphoryl)oxy)ethyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To a solution of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate E) (11.9 g, 17.1 mmol) in MeCN (250 mL) was added Et₃N (7.13 mL, 51.2 mmol) and DMAP (0.208 g, 1.71 mmol), followed by dibenzyl(2-hydroxyethyl) phosphate (16.5 g, 51.2 mmol), prepared according to WO2010039474. The reaction mixture was left to stir at room temperature for 16 h and concentrated. The residue was purified by silica gel column chromatography (0-100% EtOAc/hexane then 0-20% MeOH/DCM, and again using 50-100% EtOAc/hexane) to afford the title compound. MS (m/z) 880.82 [M+H]+.

### Step 2: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl(2-(phosphonooxy)ethyl) carbonate (3)

To a solution of 2-((bis(benzyloxy)phosphoryl)oxy)ethyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (6.90 g, 7.83 mmol) in THF (500 mL) was added 10% Pd/C (0.834 g, 0.783 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered through Celite and concentrated. The residue was dissolved in 1:1 MeCN/water and lyophilized to afford the title compound. MS (m/z) 700.90 [M+H]+. 1H NMR (400 MHz, DMSO-d6) δ 10.26 (t, J=6.0 Hz, 1H), 8.74 (s, 1H), 7.41 (td, J=8.7, 6.7 Hz, 1H), 7.24 (td, J=9.9, 2.6 Hz, 1H), 7.07 (td, J=8.5, 2.5 Hz, 1H), 5.80 (d, J=6.3 Hz, 1H), 5.64 (d, J=6.4 Hz, 1H), 4.71 (s, 1H), 4.62 (dq, J=14.5, 6.9 Hz, 1H), 4.55 (t, J=5.7 Hz, 2H), 4.23 (q, J=4.3 Hz, 2H), 4.00 (dt, J=7.1, 4.7 Hz, 2H), 3.81 (s, 3H), 3.75-3.59 (m, 2H), 3.05 (d, J=16.8 Hz, 1H), 2.75 (d, J=16.9 Hz, 1H), 1.98-1.65 (m, 3H), 1.37-1.29 (m, 1H), 1.15 (d, J=6.7 Hz, 3H).

### Example 4: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl(2-(phosphonooxy)ethyl) carbonate (4)

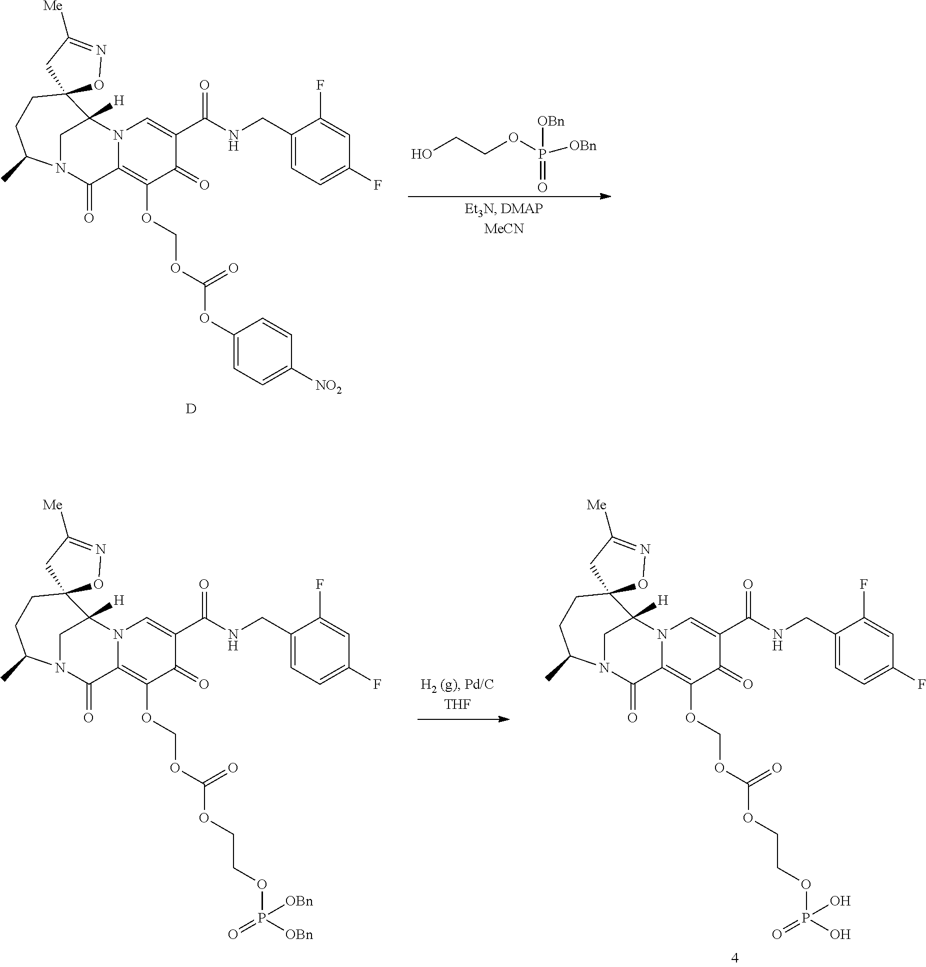

### Step 1: Preparation of 2-((bis(benzyloxy)phosphoryl)oxy)ethyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D) (2.50 g, 3.67 mmol) in MeCN (95 mL) was added Et₃N (2.56 mL, 18.3 mmol), DMAP (0.045 g, 0.367 mmol), and dibenzyl(2-hydroxyethyl) phosphate (2.36 g, 7.34 mmol), prepared according to WO2010039474. The reaction mixture was left to stir at room temperature for 16 h then diluted with EtOAc and quenched with aqueous NaHCO₃ solution. The phases were separated, and the aqueous phase was extracted with EtOAc. The combined organic phase was washed with brine, dried over Na₂SO₄, filtered, and concentrated. The residue was purified by silica gel column chromatography (20-100% EtOAc/hexane) and reverse phase prep HPLC (5-100% MeCN/H₂O) to afford the title compound. MS (m/z) 864.87 [M+H]+.

### Step 2: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl(2-(phosphonooxy)ethyl) carbonate (4)

To a solution of 2-((bis(benzyloxy)phosphoryl)oxy)ethyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.336 g, 0.389 mmol) in THF (42 mL) was added 5% Pd/C (0.083 g, 0.039 mmol). The flask was evacuated and backfilled with hydrogen gas (2×), then sparged with hydrogen for 2 min. The reaction mixture was left to stir under a hydrogen balloon atmosphere for 2 h. The reaction mixture was filtered through Celite and concentrated. The residue was dissolved in 1:1 MeCN/water and lyophilized to afford the title compound. MS (m/z) 684.82 [M+H]⁺. 1H NMR (400 MHz, DMSO-d6) δ 10.27 (t, J=6.0 Hz, 1H), 8.70 (s, 1H), 7.42 (td, J=8.7, 6.6 Hz, 1H), 7.24 (ddd, J=10.5, 9.3, 2.6 Hz, 1H), 7.07 (ddd, J=10.3, 8.2, 2.6 Hz, 1H), 5.81 (d, J=6.4 Hz, 1H), 5.65 (d, J=6.4 Hz, 1H), 4.64 (d, J=11.0 Hz, 2H), 4.59-4.46 (m, 2H), 4.22 (q, J=4.3 Hz, 2H), 3.99 (dd, J=7.2, 4.5 Hz, 2H), 3.76-3.61 (m, 2H), 3.00 (d, J=17.5 Hz, 1H), 2.63 (d, J=17.6 Hz, 1H), 1.94 (s, 3H), 1.89-1.67 (m, 3H), 1.35-1.22 (m, 1H), 1.15 (d, J=6.7 Hz, 3H).

### Example 5: Preparation of 1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl(2-(phosphonooxy)ethyl) carbonate (5)

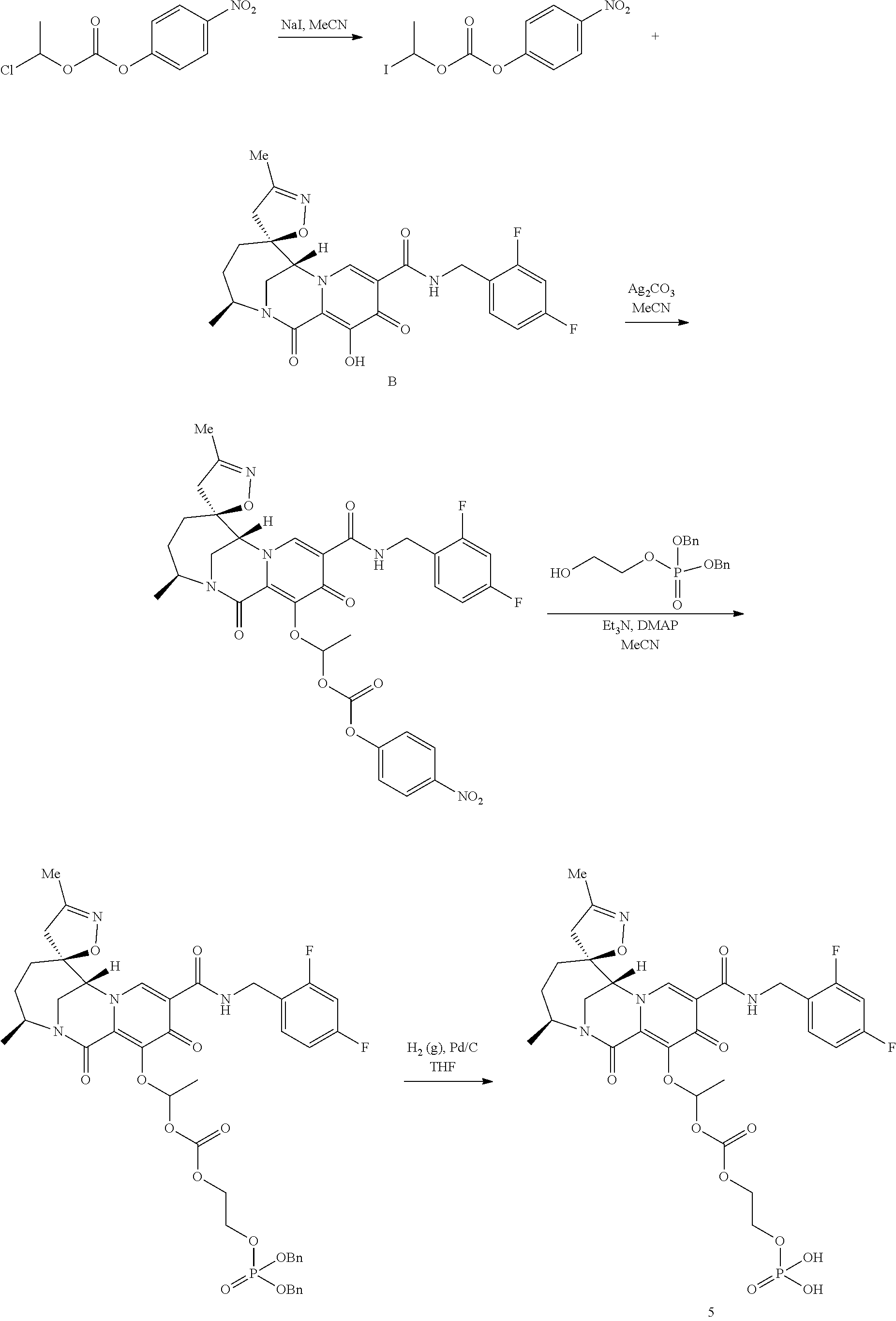

### Step 1: Preparation of 1-iodoethyl (4-nitrophenyl) carbonate

To a solution of 1-chloroethyl (4-nitrophenyl) carbonate (2.00 g, 8.14 mmol) in MeCN (16 mL) was added sodium iodide (8.54 g, 57.0 mmol) and heated to 50° C. After 16 h, the mixture was concentrated and suspended in EtOAc. The organic phase was washed with brine, Na₂S₂O₃, NaHCO₃ (2×), and brine. The organic phase was dried over Na₂SO₄, filtered, and concentrated to yield a residue, which was purified by silica gel column chromatography (0-100% EtOAc/hexane) to afford the title compound. MS (m/z) 336.02 [M]+.

### Step 2: Preparation of 1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl (4-nitrophenyl) carbonate

To a solution of 1-iodoethyl (4-nitrophenyl) carbonate (0.831 g, 2.47 mmol) in MeCN (10 mL) was added (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.400 g, 0.822 mmol) and silver carbonate (0.680 g, 2.47 mmol). The mixture was stirred at 50° C. for 16 h and concentrated. The residue was purified by reverse phase prep HPLC (10-100% MeCN/H₂O containing 0.1% TFA) to afford the title compound. MS (m/z) 695.39 [M]+.

### Step 3: Preparation of 2-((bis(benzyloxy)phosphoryl)oxy)ethyl (1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl) carbonate

To a solution of 1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl (4-nitrophenyl) carbonate (0.171 g, 0.246 mmol) in MeCN (10 mL) was added sequentially dibenzyl(2-hydroxyethyl) phosphate (0.238 g, 0.737 mmol), prepared according to WO2010039474, Et₃N (0.10 mL, 0.737 mmol), and DMAP (0.003 g, 0.025 mmol). The mixture was stirred at room temperature for 16 h and concentrated. The residue was redissolved in MeCN and purified by reverse phase prep HPLC (10-100% MeCN/water w/0.1% TFA) to afford the title compound. MS (m/z) 878.71 [M+H]+.

### Step 4: Preparation of 1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl(2-(phosphonooxy)ethyl) carbonate (5)

To a solution of 2-((bis(benzyloxy)phosphoryl)oxy)ethyl (1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl) carbonate (0.070 g, 0.080 mmol) in THF (5 mL) was added 10% Pd/C (0.008 g, 0.008 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered through Celite and concentrated. The residue was dissolved in 1:1 MeCN/water and lyophilized to afford the title compound. MS (m/z) 698.80 [M+H]⁺. 1H NMR (400 MHz, DMSO-d6) δ 10.28 (t, J=6.0 Hz, 1H), 8.70 (s, 1H), 7.42 (td, J=8.6, 6.5 Hz, 1H), 7.29-7.18 (m, 1H), 7.07 (td, J=8.6, 2.7 Hz, 1H), 6.50 (q, J=5.2 Hz, 1H), 4.71-4.60 (m, 2H), 4.60-4.48 (m, 2H), 4.11 (dt, J=10.6, 5.1 Hz, 1H), 4.07-4.01 (m, 1H), 3.94 (dd, J=7.1, 4.6 Hz, 2H), 3.81 (dt, J=7.4, 5.4 Hz, 1H), 3.53 (d, J=10.8 Hz, 1H), 2.97 (d, J=17.5 Hz, 1H), 2.58 (d, J=17.3 Hz, 1H), 1.95 (d, J=3.4 Hz, 3H), 1.87-1.73 (m, 3H), 1.60 (d, J=5.3 Hz, 3H), 1.19 (d, J=7.2 Hz, 1H), 1.14 (d, J=6.6 Hz, 3H).

### Example 6: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl(2-(phosphonooxy)ethyl) carbonate (6)

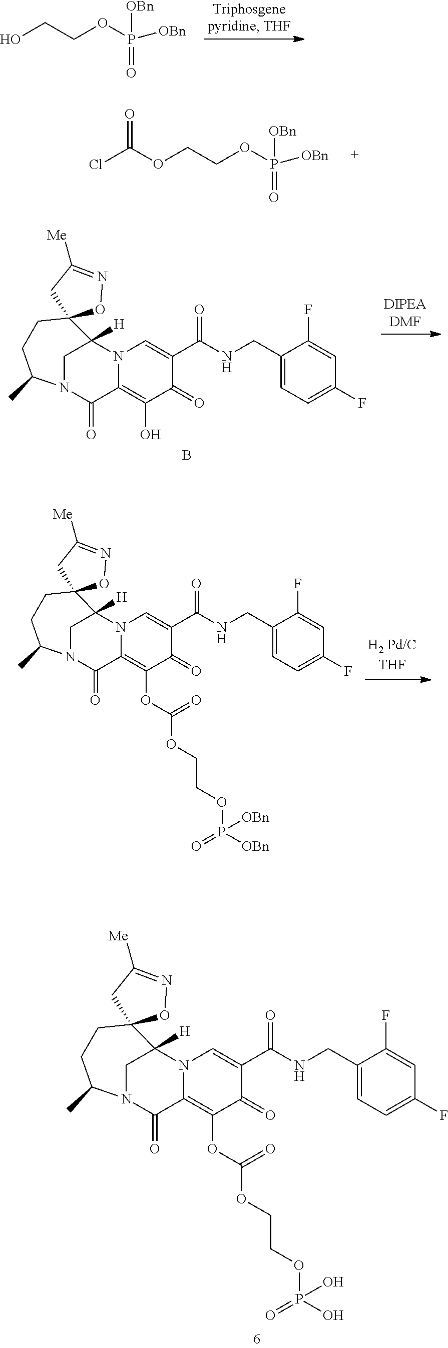

### Step 1: Preparation of 2-((bis(benzyloxy)phosphoryl)oxy)ethyl carbonochloridate

To a solution of triphosgene (0.034 g, 0.116 mmol) in THF (1.5 mL) was added pyridine (0.024 mL, 0.303 mmol) and the suspension was cooled to 0° C. To this mixture was added a solution of dibenzyl(2-hydroxyethyl) phosphate (0.075 g, 0.233 mmol), prepared according to WO2010039474, in THF (1.5 mL). The mixture was allowed to warm to room temperature and stir for 4 h. The reaction mixture was filtered through Celite, rinsing with DCM. The organic filtrate was washed with 1 N HCl, dried over Na₂SO₄, filtered, and concentrated to afford the title compound. MS (m/z) 366.80 [M+H2O—Cl]+.

### Step 2: Preparation of 2-((bis(benzyloxy)phosphoryl)oxy)ethyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate

To a mixture of (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.075 g, 0.154 mmol) and DIPEA (0.081 mL, 0.463 mmol) in DMF (1 mL) was added 2-((bis(benzyloxy)phosphoryl)oxy)ethyl carbonochloridate (0.089 g, 0.231 mmol) at 0° C. The reaction mixture was warmed to room temperature and stirred for 16 h. Additional portions of 2-((bis(benzyloxy)phosphoryl)oxy)ethyl carbonochloridate (0.089 g, 0.231 mmol) and DIPEA (0.081 mL, 0.463 mmol) were added and stirred for 16 h. The reaction mixture was diluted with EtOAc, washed with 5% LiCl (aq), water, and brine. The organic phase was dried over Na₂SO₄, filtered, and concentrated to afford a residue, which was purified by column chromatography (0-100% EtOAc/hexane) and reverse phase prep HPLC (5-100% MeCN/water) to afford the title compound. MS (m/z) 834.89 [M+H]+.

### Step 3: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl(2-(phosphonooxy)ethyl) carbonate (6)

To a solution of 2-((bis(benzyloxy)phosphoryl)oxy)ethyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate (0.014 g, 0.017 mmol) in THF (2 mL) was added 5% Pd/C (0.004 g, 0.002 mmol). The vial was evacuated and backfilled with hydrogen gas (2×), then sparged with hydrogen gas for 2 min. The reaction mixture was left to stir under a hydrogen balloon atmosphere for 2 h. The reaction mixture was filtered through Celite, rinsing with THE, and concentrated. The residue was dissolved in 1:1 MeCN/water and lyophilized to afford the title compound. MS (m/z) 654.99 [M+H]⁺. 1H NMR (400 MHz, DMSO-d6) δ 10.12 (t, J=6.0 Hz, 1H), 8.85 (s, 1H), 7.47-7.37 (m, 1H), 7.25 (td, J=10.0, 2.6 Hz, 1H), 7.14-7.02 (m, 1H), 4.72 (s, 1H), 4.56 (dq, J=15.3, 9.0, 7.7 Hz, 3H), 4.39-4.26 (m, 2H), 4.05 (d, J=6.2 Hz, 2H), 3.83 (d, J=15.8 Hz, 1H), 3.71 (d, J=15.2 Hz, 1H), 3.03 (d, J=17.4 Hz, 1H), 2.67 (d, J=17.7 Hz, 1H), 1.95 (s, 3H), 1.78 (d, J=19.4 Hz, 3H), 1.30-1.24 (m, 1H), 1.17 (d, J=6.6 Hz, 3H).

### Example 7: Preparation of N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)ethyl)-N-(((phosphonooxy)methoxy)carbonyl)glycine (7)

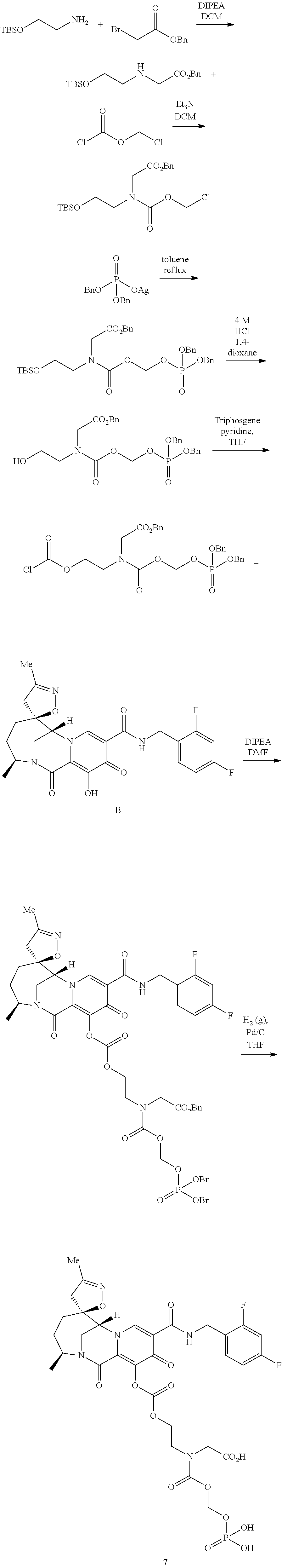

### Step 1: Preparation of benzyl(2-((tert-butyldimethylsilyl)oxy)ethyl)glycinate

To a stirred solution of 2-((tert-butyldimethylsilyl)oxy) ethan-1-amine (100 g, 571.4 mmol) in DCM (3.0 L) at 0° C. under argon was added benzyl 2-bromoacetate (64.85 g, 285.7 mmol) followed by DIPEA (81.0 g, 628.1 mmol). The reaction mixture was stirred for 16 h at room temperature. The reaction mixture was diluted with DCM and washed with water. The organic layer was dried over anhydrous Na₂SO₄, filtered, and concentrated under reduced pressure. The residue was purified by silica gel column chromatography (10-15% EtOAc in pet ether) to afford the title compound. MS (m/z) 324.25 [M+H]+.

### Step 2: Preparation of benzyl N-(2-((tert-butyldimethylsilyl)oxy)ethyl)-N-((chloromethoxy)carbonyl)glycinate

To a stirred solution of benzyl(2-((tert-butyldimethylsilyl)oxy)ethyl)glycinate (80 g, 247.6 mmol) in DCM (2.4 L) at 0° C. under argon was added chloromethyl chloroformate (41.5 g, 321.9 mmol) followed by Et₃N (62.5 g, 619 mmol). The mixture was stirred for 16 h at room temperature. The reaction mixture was diluted with DCM and washed with water. The organic layer was dried over anhydrous Na₂SO₄, filtered, and concentrated under reduced pressure. The crude residue was purified by silica gel column chromatography (20-30% EtOAc in pet ether) to afford the title compound. MS (m/z) 416.56 [M+H]+.

### Step 3: Preparation of benzyl N-((((bis(benzyloxy)phosphoryl)oxy)methoxy)carbonyl)-N-(2-((tert-butyldimethylsilyl)oxy)ethyl)glycinate

To a stirred solution of benzyl N-(2-((tert-butyldimethylsilyl)oxy)ethyl)-N-((chloromethoxy)carbonyl)glycinate (20 g, 48.08 mmol) in toluene (200 mL) at room temperature under argon was added silver dibenzylphosphate (37 g, 96.08 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (20-30% EtOAc in pet ether) to afford the title compound. MS (m/z) 658.21 [M+H]⁺.

### Step 4: Preparation of benzyl N-((((bis(benzyloxy)phosphoryl)oxy)methoxy)carbonyl)-N-(2-hydroxyethyl)glycinate

To a stirred solution of benzyl N-((((bis(benzyloxy)phosphoryl)oxy)methoxy)carbonyl)-N-(2-((tert-butyldimethylsilyl)oxy)ethyl)glycinate (13 g, 19.76 mmol) in 1,4-dioxane (390 mL) at 0° C. under argon was added 4 M HCl in 1,4-dioxane (9.8 mL, 39.2 mmol) under argon. The mixture was stirred for 4 h at room temperature and concentrated under reduced pressure. The crude residue was purified by silica gel column chromatography (50% EtOAc in pet-ether). The pure fractions were collected and concentrated under reduced pressure. The material was triturated with n-pentane, filtered, and dried under vacuum to obtain the title compound. MS (m/z) 544.25 [M+H]+.

### Step 5: Preparation of benzyl N-((((bis(benzyloxy)phosphoryl)oxy)methoxy)carbonyl)-N-(2-((chlorocarbonyl)oxy)ethyl)glycinate

To a solution of triphosgene (0.409 g, 1.38 mmol) in THF (12 mL) was added pyridine (0.289 mL, 3.59 mmol) and the suspension was cooled to 0° C. To this was added a solution of benzyl N-((((bis(benzyloxy)phosphoryl)oxy)methoxy)carbonyl)-N-(2-hydroxyethyl)glycinate (1.50 g, 2.76 mmol) in THF (12 mL). The suspension was allowed to warm to room temperature and stir for 4 h. The reaction mixture was filtered through Celite and rinsed with DCM. The organic filtrate was washed with 1 N HCl, dried over Na₂SO₄, filtered, and concentrated. MS (m/z) 605.70 [M+H]+.

### Step 6: Preparation of benzyl N-((((bis(benzyloxy)phosphoryl)oxy)methoxy)carbonyl)-N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)ethyl)glycinate

To a suspension of (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.895 g, 1.84 mmol) and DIPEA (0.96 mL, 5.52 mmol) in DMF (8 mL) was added benzyl N-((((bis(benzyloxy)phosphoryl)oxy)methoxy)carbonyl)-N-(2-((chlorocarbonyl)oxy)ethyl)glycinate (1.67 g, 2.76 mmol) at 0° C. The reaction mixture was warmed to room temperature and stirred for 16 h. The reaction mixture was diluted with EtOAc and washed with 5% LiCl (aq), water, and brine. The organic phase was dried over Na₂SO₄, filtered, and concentrated to afford a residue, which was purified by silica gel column chromatography (0-100% EtOAc/hexane). The residue was then purified by reverse phase prep HPLC (5-100% MeCN/water) to afford the title compound. MS (m/z) 1055.96 [M+H]+.

### Step 7: Preparation of N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)ethyl)-N-(((phosphonooxy)methoxy)carbonyl)glycine (7)

To a solution of benzyl N-((((bis(benzyloxy)phosphoryl)oxy)methoxy)carbonyl)-N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)ethyl)glycinate (1.32 g, 1.25 mmol) in THF (100 mL) was added 10% Pd/C (0.133 g, 0.125 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered, rinsed with THE, and concentrated to afford the title compound. MS (m/z) 785.88 [M+H]+. 1H NMR (400 MHz, DMSO-d6) δ 10.12 (t, J=6.0 Hz, 1H), 8.85 (s, 1H), 7.42 (td, J=8.7, 6.5 Hz, 1H), 7.25 (ddd, J=10.4, 9.3, 2.6 Hz, 1H), 7.08 (td, J=8.7, 2.7 Hz, 1H), 5.54-5.38 (m, 2H), 4.72 (s, 1H), 4.56 (qt, J=14.9, 6.7 Hz, 3H), 4.32 (dt, J=14.7, 5.8 Hz, 2H), 4.04 (d, J=25.5 Hz, 2H), 3.82 (d, J=16.5 Hz, 1H), 3.71 (d, J=14.8 Hz, 1H), 3.67-3.51 (m, 2H), 3.03 (d, J=17.5 Hz, 1H), 2.66 (d, J=17.5 Hz, 1H), 1.95 (s, 3H), 1.87-1.72 (m, 3H), 1.25 (dd, J=15.8, 9.3 Hz, 1H), 1.17 (d, J=6.7 Hz, 3H).

### Example 8: Preparation of N-(2-((((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)ethyl)-N-(((phosphonooxy)methoxy)carbonyl)glycine (8)

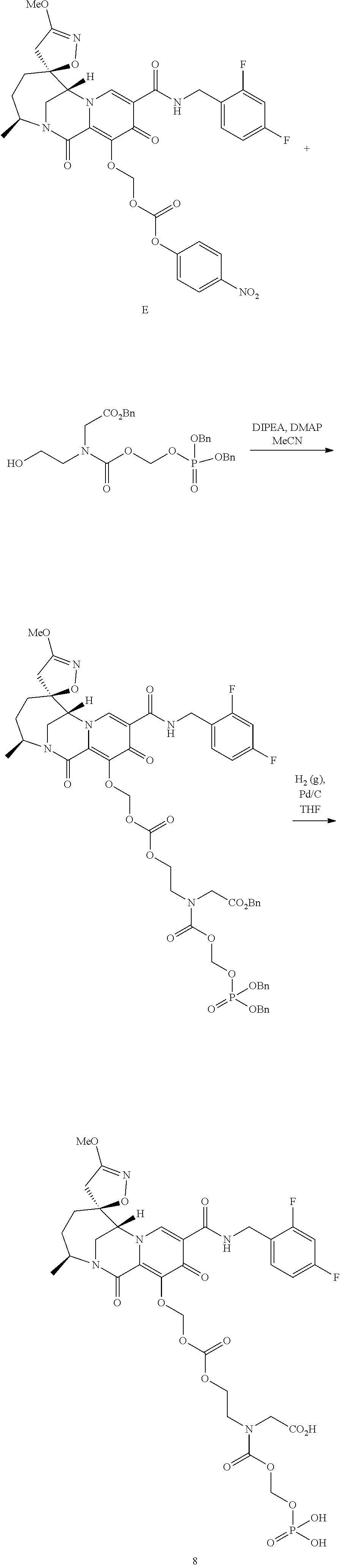

### Step 1: Preparation of benzyl N-((((bis(benzyloxy)phosphoryl)oxy)methoxy)carbonyl)-N-(2-((((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)ethyl)glycinate

To a solution of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate E, 0.100 g, 0.143 mmol) in MeCN (2 mL) was added Et₃N (0.060 mL, 0.430 mmol) and DMAP (0.002 g, 0.014 mmol), followed by benzyl N-((((bis(benzyloxy)phosphoryl)oxy)methoxy)carbonyl)-N-(2-hydroxyethyl)glycinate (0.234 g, 0.430 mmol), prepared according to Step 4 of Example 7. The reaction mixture was left to stir at room temperature for 16 h and was concentrated. The residue was purified by silica gel column chromatography (0-100% EtOAc/hexane, then 0-20% MeOH/DCM) to afford the title compound. MS (m/z) 1101.92 [M+H]+.

### Step 2: Preparation of N-(2-((((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)ethyl)-N-(((phosphonooxy)methoxy)carbonyl)glycine (8)

To a solution of benzyl N-((((bis(benzyloxy)phosphoryl)oxy)methoxy)carbonyl)-N-(2-((((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)ethyl)glycinate (0.063 g, 0.057 mmol) in THF (5 mL) was added 5% Pd/C (0.012 g, 0.006 mmol). The vial was evacuated and backfilled with hydrogen gas (2×), then sparged with hydrogen for 2 min. The reaction mixture was left to stir under a hydrogen balloon atmosphere for 2 h. The reaction mixture was filtered and concentrated to yield a crude residue, which was dissolved in 1:1 MeCN/water and purified by reverse phase prep HPLC (10-100% MeCN/water w/0.1% TFA) to afford the title compound after lyophilization. MS (m/z) 831.82 [M+H]+. 1H NMR (400 MHz, DMSO-d6) δ 10.26 (t, J=6.0 Hz, 1H), 8.75 (s, 1H), 7.42 (q, J=8.3 Hz, 1H), 7.24 (td, J=9.9, 2.7 Hz, 1H), 7.07 (td, J=8.5, 2.5 Hz, 1H), 5.82 (d, J=6.5 Hz, 1H), 5.64 (dd, J=6.5, 2.7 Hz, 1H), 5.44 (dd, J=14.8, 13.0 Hz, 2H), 4.71 (s, 1H), 4.58 (dt, J=20.7, 7.7 Hz, 3H), 4.20 (t, J=6.5 Hz, 2H), 4.04 (s, 1H), 3.98 (s, 1H), 3.82 (s, 3H), 3.68 (d, J=6.5 Hz, 2H), 3.54 (t, J=5.2 Hz, 2H), 3.05 (d, J=16.8 Hz, 1H), 2.75 (d, J=16.9 Hz, 1H), 1.98-1.69 (m, 3H), 1.32 (s, 1H), 1.15 (d, J=6.6 Hz, 3H).

### Example 9: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((phosphonooxy)methyl) ethane-1,2-diylbis(methylcarbamate) (9)

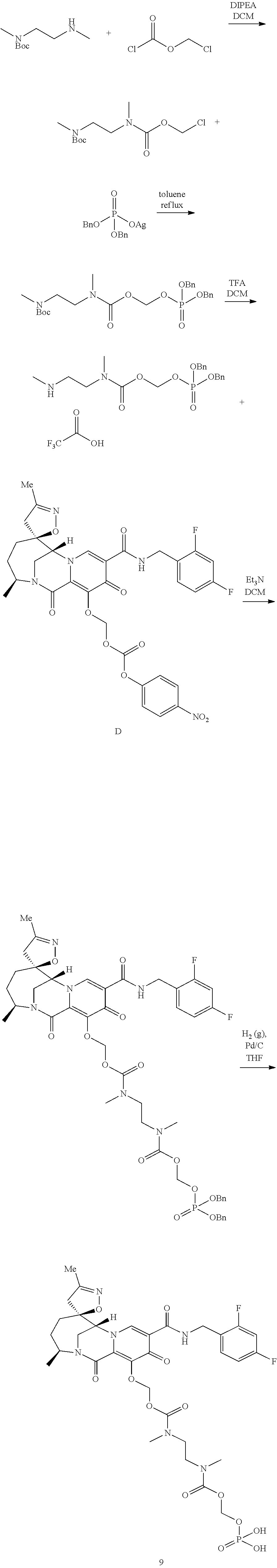

### Step 1: Preparation of tert-butyl (chloromethyl) ethane-1,2-diylbis(methylcarbamate)

To a solution of tert-butyl methyl(2-(methylamino)ethyl) carbamate (0.500 g, 2.66 mmol) in DCM (3 mL) was added DIPEA (0.694 mL, 3.98 mmol) and chloromethyl chloroformate (0.283 mL, 3.19 mmol) at 0° C. The reaction mixture was stirred at 0° C. for 2 h and concentrated. The residue was purified by silica gel column chromatography (10-100% EtOAc/hexane) to afford the title compound. MS (m/z) 180.99 [M-Boc+H]+.

### Step 2: Preparation of ((bis(benzyloxy)phosphoryl)oxy)methyl tert-butyl ethane-1,2-diylbis(methylcarbamate)

To a solution of tert-butyl (chloromethyl) ethane-1,2-diylbis(methylcarbamate) (0.100 g, 0.356 mmol) in toluene (12 mL) was added silver dibenzylphosphate (0.412 g, 1.07 mmol) and the reaction mixture was heated to reflux for 2 h. The reaction mixture was cooled to rt and concentrated. The residue was purified by silica gel column chromatography (10-100% EtOAc/hexane) to afford the title compound. MS (m/z) 545.03 [M+Na]+.

### Step 3: Preparation of ((bis(benzyloxy)phosphoryl)oxy)methyl methyl(2-(methylamino)ethyl) carbamate; trifluoroacetic acid adduct

To a solution of ((bis(benzyloxy)phosphoryl)oxy)methyl tert-butyl ethane-1,2-diylbis(methylcarbamate) (0.186 g, 0.356 mmol) in DCM (2.5 mL) at 0° C. was added a solution of TFA (0.872 mL, 11.4 mmol) in DCM (1 mL). The reaction mixture was stirred at 0° C. for 1 h. The reaction mixture was concentrated to dryness, redissolved in 1 mL of DCM, and concentrated again. The residue was dried under high vacuum to afford the title compound as a 2,2,2-trifluoroacetic acid adduct. MS (m/z) 423.03 [M+H]+.

### Step 4: Preparation of ((bis(benzyloxy)phosphoryl)oxy)methyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) ethane-1,2-diylbis(methylcarbamate)

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D) (0.130 g, 0.191 mmol) and ((bis(benzyloxy)phosphoryl)oxy)methyl methyl(2-(methylamino)ethyl) carbamate trifluoroacetic acid adduct (0.153 g, 0.286 mmol) in DCM (8 mL) at room temperature was added Et₃N (0.798 mL, 5.72 mmol). The reaction mixture was concentrated and purified by reverse phase prep HPLC (5-100% MeCN/water w/0.1% TFA) and lyophilized to afford the title compound. MS (m/z) 964.76 [M+H]+.

### Step 5: Preparation of (((3′S,5S,7′R)-10′-((2,4-difluorobenzyl)carbamoyl)-3,3′-dimethyl-1′,11′-dioxo-1′,4′,5′11′-tetrahydro-3′H,4H,7′H-spiro[isoxazole-5,6′-[2,7]methanopyrido[1,2-a][1,4]diazonin]-12′-yl)oxy)methyl ((phosphonooxy)methyl) ethane-1,2-diylbis(methylcarbamate) (9)

To a solution of ((bis(benzyloxy)phosphoryl)oxy)methyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) ethane-1,2-diylbis(methylcarbamate) (0.084 g, 0.087 mmol) in THF (8 mL) was added 5% Pd/C (0.018 g, 0.009 mmol). The vial was evacuated and backfilled with hydrogen gas (2×), then sparged with hydrogen for 2 min. The reaction mixture was left to stir under a hydrogen balloon atmosphere for 2 h. The reaction mixture was filtered through Celite, rinsed with THE, and concentrated. The residue was dissolved in 1:1 MeCN/water and lyophilized to afford the title compound. MS (m/z) 784.80 [M+H]+. 1H NMR (400 MHz, DMSO-d6) δ 10.32 (t, J=6.0 Hz, 1H), 8.67 (d, J=1.8 Hz, 1H), 7.43 (td, J=8.6, 6.6 Hz, 1H), 7.25 (ddd, J=10.5, 9.3, 2.6 Hz, 1H), 7.08 (td, J=8.6, 2.6 Hz, 1H), 5.76-5.56 (m, 2H), 5.48-5.33 (m, 2H), 4.64 (d, J=10.9 Hz, 2H), 4.59-4.50 (m, 2H), 3.69 (d, J=13.9 Hz, 1H), 3.65 (s, 1H), 3.33 (s, 4H), 3.03-2.93 (m, 1H), 2.88-2.74 (m, 6H), 2.63 (dd, J=17.9, 4.8 Hz, 1H), 1.95 (s, 3H), 1.77 (dd, J=16.9, 9.8 Hz, 3H), 1.37-1.20 (m, 1H), 1.15 (d, J=6.6 Hz, 3H).

### Example 10: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3-(phosphonooxy)propyl) carbonate (10)

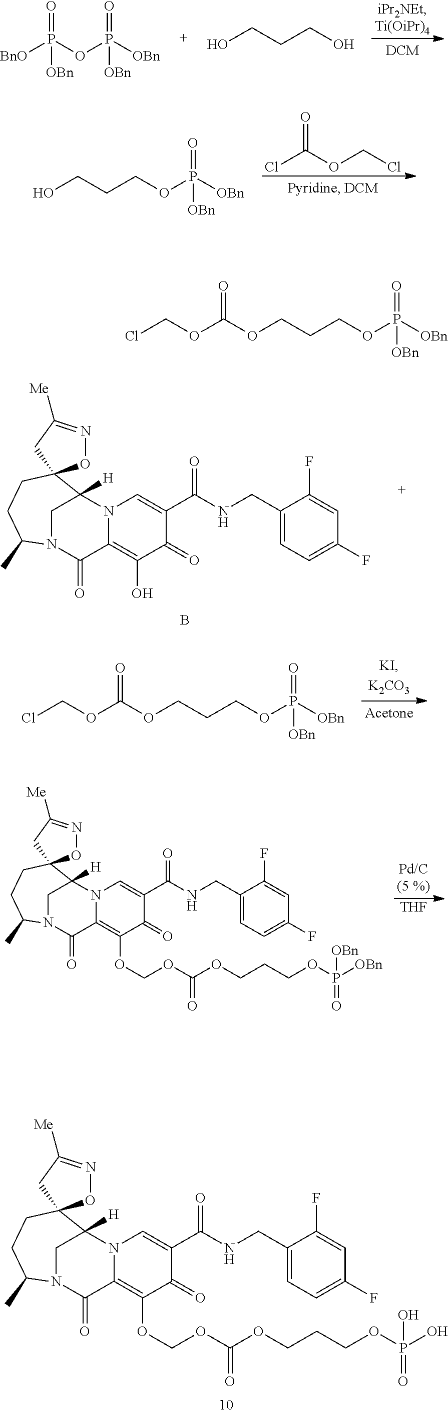

### Step 1: Preparation of dibenzyl (3-hydroxypropyl) phosphate

To the solution of propane-1,3-diol (0.22 g, 2.89 mmol) was added N,N-diisopropylethylamine (0.79 ml, 4.4 mmol) followed by tetrabenzylpyrophosphate (0.95 g, 1.76 mmol) and titanium (IV) isopropoxide (0.085 g, 0.3 mmol) sequentially. The resulting mixture was stirred at room temperature for 4 hrs. Then the reaction mixture was filtered through a pad of silica gel/magnesium sulfate mixture (20:1), rinsing the solids with ethyl acetate/hexane. The filtrate was dried over sodium sulfate, filtered, concentrated under reduced pressure. The residue was purified by normal phase chromatography on a silica gel to afford the title compound. MS (m/z) 337.2 [M+H]+.

### Step 2: Preparation of 3-((bis(benzyloxy)phosphoryl)oxy)propyl (chloromethyl) carbonate

Dibenzyl 3-hydroxypropyl phosphate (0.5 g, 1.5 mmol) was added to a stirred mixture of chloromethyl chloroformate (0.145 ml, 1.64 mmol) in CH2Cl2 (10 mL) then followed by the addition of pyridine (0.14 mL, 1.87 mmol) dropwise at room temperature. The reaction was stirred for overnight before it was diluted with DCM and washed sequentially with HCl (0.5M) and water. The organic layer was dried over magnesium sulfate, filtered, and evaporated to obtain the title compound. MS (m/z) 428.9 [M+H]+.

### Step 3: Preparation of 3-((bis(benzyloxy)phosphoryl)oxy)propyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To the solution of (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.08 g, 0.164 mmol) in acetone (20 mL) was added KI (0.036 mg, 0.21 mmol) and K₂CO₃ (0.046 g, 0.329 mmol) followed by 3-((bis(benzyloxy)phosphoryl)oxy)propyl (chloromethyl) carbonate (0.085 g, 0.197 mmol). The resulting mixture was left to stir at room temperature for overnight and then was concentrated. The residue was purified by silica gel column chromatography (0-100% EtOAc/Hexane) to afford the title compound. MS (m/z) 878.9 [M+H]+.

### Step 4: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3-(phosphonooxy)propyl) carbonate (10)

To the solution of 3-((bis(benzyloxy)phosphoryl)oxy)propyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.08 g, 0.009 mmol) in THF (20 ml) was added Pd/C (5 wt %) (0.020 g, 0.0009 mmol). The resulting mixture was purged with hydrogen three times before it was stirred under hydrogen for 1 h. The reaction was then filtered through Celite® and concentrated. The resulting residue was purified by reverse phase preparative HPLC to afford the title compound. MS (m/z) 698.88 [M+H]+. 1H NMR (400 MHz, DMSO-d6) δ 10.28 (t, J=6.0 Hz, 1H), 8.71 (s, 1H), 7.43 (td, J=8.7, 6.6 Hz, 1H), 7.25 (ddd, J=10.6, 9.3, 2.6 Hz, 1H), 7.13-7.03 (m, 1H), 5.83 (d, J=6.5 Hz, 1H), 5.64 (d, J=6.5 Hz, 1H), 4.71-4.47 (m, 5H), 4.14 (ddt, J=10.9, 6.3, 4.4 Hz, 2H), 3.88 (dt, J=7.5, 6.3 Hz, 2H), 3.68 (t, J=2.3 Hz, 2H), 3.01 (d, J=17.5 Hz, 1H), 2.63 (d, J=17.6 Hz, 1H), 1.97-1.86 (m, 4H), 1.91-1.75 (m, 2H), 1.79-1.69 (m, 1H), 1.35-1.23 (m, 1H), 1.16 (d, J=6.7 Hz, 3H).

### Example 11: Preparation (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-(phosphonooxy)propyl) carbonate (11)

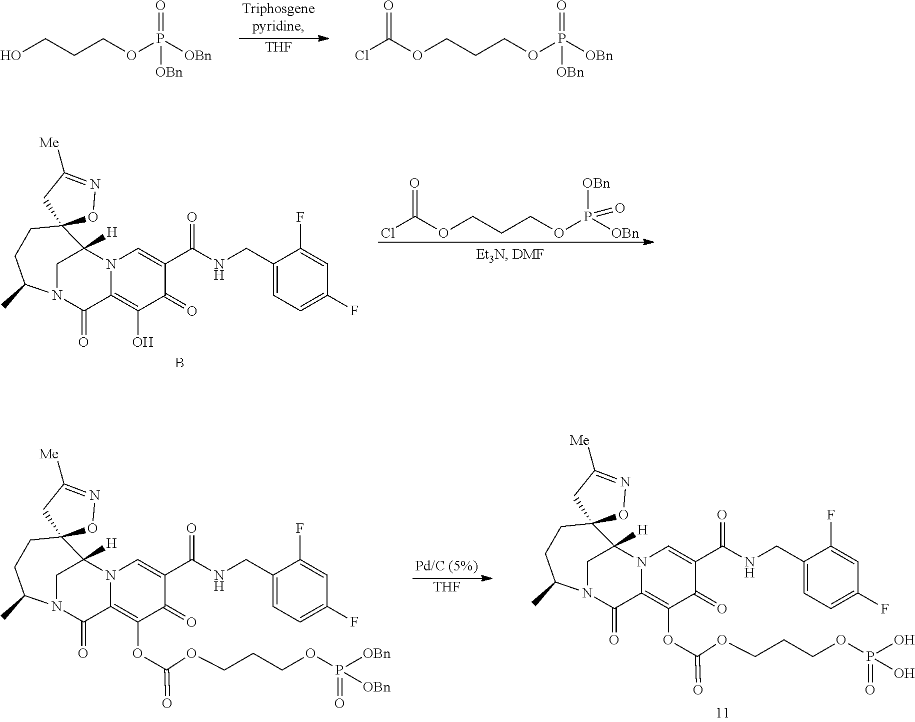

### Step 1: Preparation 3-((bis(benzyloxy)phosphoryl)oxy)propyl carbonochloridate

To a solution of triphosgene (0.088 g, 0.297 mmol) in THF (1 mL) was added pyridine (90.062 mL, 0.773 mmol) and the resulting suspension was cooled to 0° C. To this mixture was added a solution of dibenzyl (3-hydroxypropyl) phosphate (0.2 g, 0.595 mmol) in THF (1 mL). The suspension was allowed to warm to room temperature and stir for 4 hours. The mixture was filtered through Celite and rinsed with DCM. The filtrate was washed with 1 N HCl, dried over Na₂SO₄, filtered, concentrated, and used directly in next step.

### Step 2: Preparation 3-((bis(benzyloxy)phosphoryl)oxy)propyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate

To a suspension of (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.1 g, 0.21 mmol) and DIPEA (0.11 mL, 0.62 mmol) in DMF (1 mL) was added 3-((bis(benzyloxy)phosphoryl)oxy)propyl carbonochloridate (0.123 g, 0.31 mmol) at 0° C. The reaction mixture was then warmed to rt and stirred overnight. The reaction mixture was diluted with EtOAc, washed with 5% LiCl (aq), water, and brine. The organic phase was dried over MgSO₄, filtered, and concentrated to afford a residue, which was purified by silica gel column chromatography (0-100% EtOAc/hexane) to afford the title compound. MS (m/z) 849.40 [M+H]+.

### Step 3: Preparation (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-(phosphonooxy)propyl) carbonate (11)

The title compound was prepared following the same method as Example 10 step 4, except using 3-((bis(benzyloxy)phosphoryl)oxy)propyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate instead of 3-((bis(benzyloxy)phosphoryl)oxy)propyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate. MS (m/z) 668.88 [M+H]+. 1H NMR (400 MHz, Chloroform-d) δ 10.27 (s, 1H), 8.59 (s, 1H), 7.42-7.32 (m, 1H), 6.94-6.75 (m, 2H), 4.74 (d, J=8.5 Hz, 1H), 4.61 (d, J=5.7 Hz, 2H), 4.43 (s, 2H), 4.22 (d, J=34.6 Hz, 3H), 3.81 (s, 2H), 3.09 (d, J=17.8 Hz, 1H), 2.63 (d, J=17.9 Hz, 1H), 2.08 (m, 5H), 2.01-1.81 (m, 3H), 1.57 (t, J=10.3 Hz, 1H), 1.25 (d, J=6.5 Hz, 3H).

### Example 12: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2,2-dimethyl-3-(phosphonooxy)propyl) carbonate (12)

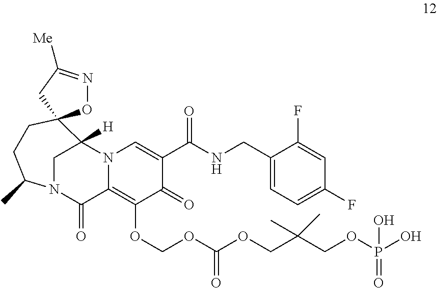 [SMILES: CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

The title compound was made following the same method as Example 10, except in step 1, propane-1,3-diol was replaced by 2,2-dimethylpropane-1,3-diol. MS (m/z) 726.91 [M+H]⁺. 1H NMR (400 MHz, Chloroform-d) δ 10.28 (s, 1H), 8.43 (s, 1H), 7.38 (q, J=7.9 Hz, 1H), 6.82 (q, J=9.3 Hz, 2H), 5.93 (d, J=6.6 Hz, 1H), 5.79 (d, J=6.7 Hz, 1H), 4.85 (d, J=8.9 Hz, 1H), 4.62 (q, J=14.2, 12.8 Hz, 2H), 4.22 (s, 1H), 3.98 (d, J=10.4 Hz, 1H), 3.90 (d, J=10.3 Hz, 1H), 3.77 (m, 4H), 3.03 (d, J=17.8 Hz, 1H), 2.60 (d, J=17.8 Hz, 1H), 2.08 (s, 3H), 1.94 (s, 3H), 1.52 (m, 1H), 1.30-1.23 (m, 3H), 0.97 (d, J=7.3 Hz, 6H).

### Example 13: Preparation (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2,2-dimethyl-3-(phosphonooxy)propyl) carbonate (13)

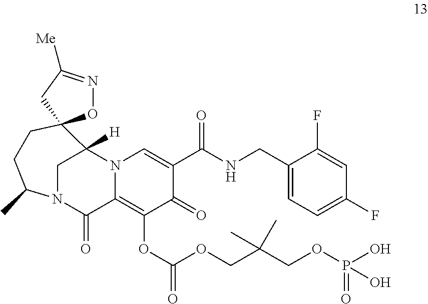 [SMILES: CC1=NO[C@@]2(CC[C@H](C)N3C[C@H]2n2cc(C(=O)NCc4ccc(F)cc4F)c(=O)c(OC(=O)OCC(C)(C)COP(=O)(O)O)c2C3=O)C1]

The title compound was made following the same method as Example 11, except in step 1, propane-1,3-diol was replaced by 2,2-dimethylpropane-1,3-diol. MS (m/z) 696.9 [M+H]⁺. 1H NMR (400 MHz, Chloroform-d) & 10.23 (t, J=6.0 Hz, 1H), 8.56 (s, 1H), 7.35 (td, J=8.5, 6.3 Hz, 1H), 6.90-6.76 (m, 2H), 4.79-4.68 (m, 1H), 4.62 (d, J=5.9 Hz, 2H), 4.25 (d, J=2.2 Hz, 1H), 4.12 (s, 2H), 3.88-3.76 (m, 4H), 3.08 (d, J=17.9 Hz, 1H), 2.62 (d, J=17.9 Hz, 1H), 2.07 (s, 3H), 1.99-1.85 (m, 3H), 1.59-1.50 (m, 1H), 1.25 (d, J=6.6 Hz, 3H), 1.00 (d, J=4.5 Hz, 6H).

### Example 14: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl(2-(dimethylamino)ethyl) carbonate (14)

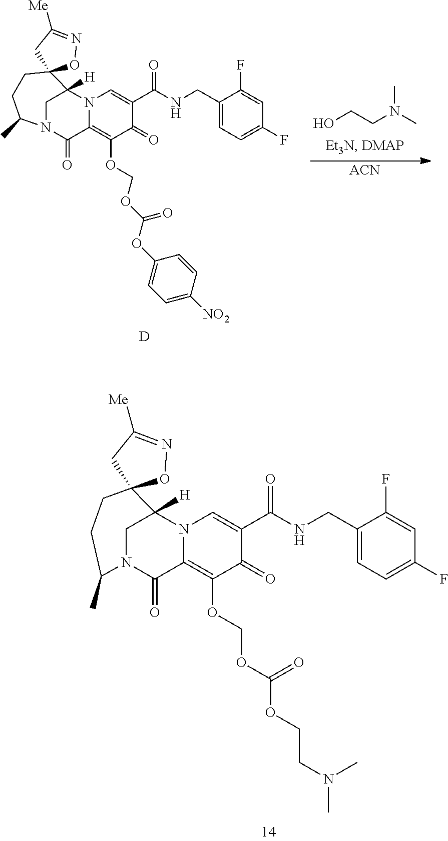

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D, 32 mg, 0.0467 mmol) and 2-(dimethylamino) ethanol (21 mg, 0.233 mmol) were mixed with acetonitrile (1 ml) at room temperature. Then Et₃N (23.6 mg, 0.233 mmol) and DMAP (0.854 mg, 0.007 mmol) were added. The resulting reaction mixture was then stirred at rt for 17 h. Reaction mixture was purified with reverse phase prep-HPLC with 0-100% acetonitrile in water with 0.1% TFA to give the title compound as TFA salt after lyophilization. Calculated for C₃₀H₃₅F₂N₅O₈: 631.25, Found MS (ESI+): 632.41 [M+H]+; ¹H NMR (400 MHz, Acetonitrile-d₃) δ 10.23 (s, 1H), 8.52 (s, 1H), 7.58-7.30 (m, 1H), 6.98 (ddt, J=13.5, 8.5, 3.1 Hz, 2H), 5.97 (d, J=6.5 Hz, 1H), 5.76 (d, J=6.6 Hz, 1H), 4.86-4.68 (m, 1H), 4.61 (d, J=5.9 Hz, 2H), 4.51 (qt, J=13.3, 5.0 Hz, 2H), 4.31 (s, 1H), 3.70 (d, J=2.3 Hz, 2H), 3.43 (t, J=5.0 Hz, 2H), 3.06 (d, J=17.9 Hz, 1H), 2.85 (s, 6H), 2.61 (d, J=17.8 Hz, 1H), 1.99 (s, 3H), 1.94-1.78 (m, 3H), 1.43 (dd, J=15.4, 10.8 Hz, 1H), 1.21 (d, J=6.7 Hz, 3H).

### Example 15: Preparation of 2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-N,N,Ntrimethylethan-1-aminium trifluoroacetic acid (15)

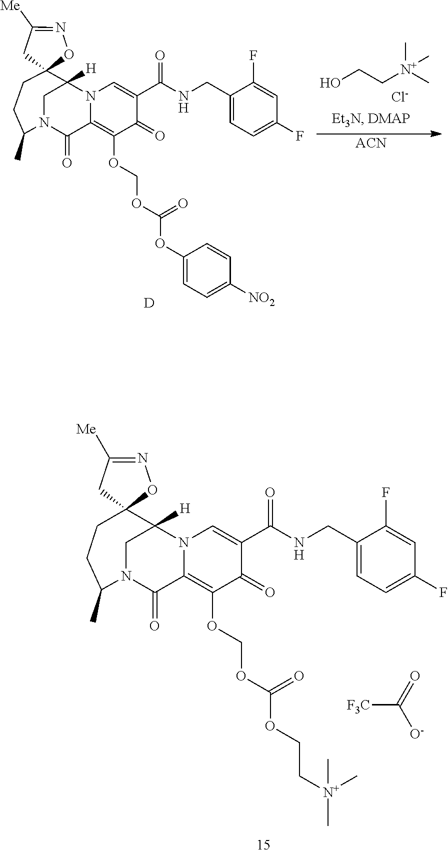

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D, 33 mg, 0.0484 mmol) and 2-hydroxy-N,N,N-trimethylethan-1-aminium chloride (33.8 mg, 0.242 mmol) were mixed with acetonitrile (1 ml) at rt. Then Et₃N (24.5 mg, 0.242 mmol) and DMAP (0.854 mg, 0.007 mmol) were added. The resulting reaction mixture was then stirred at rt for 17 h. Reaction mixture was purified with reverse phase prep-HPLC with 0-100% acetonitrile in water with 0.1% TFA to afford the title compound as TFA salt after lyophilization. Calculated for C₃₁H₃₈F₂N₅O₈⁺: 646. 27, Found MS (ESI+): 646.14 [M]⁺. 1H NMR (400 MHz, CD3CN) δ 10.25 (t, J=5.8 Hz, 1H), 8.54 (s, 1H), 7.44 (td, J=8.8, 6.4 Hz, 1H), 7.04-6.93 (m, 2H), 6.03 (d, J=6.6 Hz, 1H), 5.79 (d, J=6.6 Hz, 1H), 4.72 (dt, J=9.9, 6.9 Hz, 1H), 4.61 (d, J=6.0 Hz, 2H), 4.67-4.48 (m, 2H), 4.32 (s, 1H), 3.70 (t, J=2.0 Hz, 2H), 3.67 (t, J=4.7 Hz, 2H), 3.14 (s, 9H), 3.07-3.03 (dd, J=17.8, 1.1 Hz, 1H), 2.61 (dd, J=17.8, 1.1 Hz, 1H), 1.99 (d, J=1.1 Hz, 3H), 1.94-1.79 (m, 3H), 1.45-1.34 (m, 1H), 1.22 (d, J=6.7 Hz, 3H).

### Example 16: Preparation of 2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)-N,N,N-trimethylethan-1-aminium trifluoroacetic acid (16)

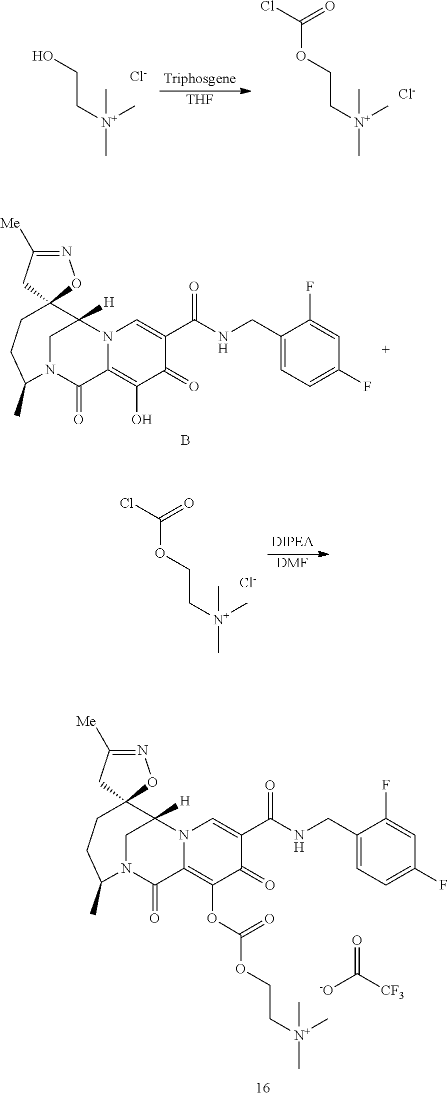

### Step 1: Synthesis of 2-((chlorocarbonyl)oxy)-N,N,N-trimethylethan-1-aminium chloride

2-hydroxy-N,N,N-trimethylethan-1-aminium chloride (1 g, 7.16 mmol) was mixed with THF (50 mL) with stirring at room temperature to afford a slurry. Triphosgene (2.34 g, 7.88 mmol) was added in 4 portions at 0° C. The reaction mixture was then warmed up to rt and stirred for 24 hrs. The reaction mixture was filtered and the solid was collected. Hexane (3×30 mL) was used to wash the solid. The solid product 2-((chlorocarbonyl)oxy)-N,N,N-trimethylethan-1-aminium chloride was put under high vacuum for 3 h and used directly for next step. MS (m/z) 166.10 [M]⁺.

### Step 2: Synthesis of 2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)-N,N,N-trimethylethan-1-aminium trifluoroacetic acid salt (16)

(3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 103 mg, 0.212 mmol) was dissolved in DMF (3 mL) at rt. DIPEA (137 mg, 1.06 mmol) and 2-((chlorocarbonyl)oxy)-N,N,N-trimethylethan-1-aminium chloride (66 mg, 0.327 mmol) were added sequentially at room temperature. The reaction mixture was stirred for 17 h. Purification directly on reverse phase preparative HPLC with ACN/water (containing 0.1% TFA) gave title compound (16). Calculated for C₃₀H₃₆F₂N₅O₇⁺: 616.26, Found MS (M+): 616.37; 1H NMR (400 MHz, CD3CN) δ 10.11 (t, J=5.9 Hz, 1H), 8.63 (s, 1H), 7.44 (td, J=8.8, 6.5 Hz, 1H), 7.04-6.93 (m, 2H), 4.76-4.66 (m, 3H), 4.61 (d, J=6.0 Hz, 2H), 4.40 (d, J=2.5 Hz, 1H), 3.75 (d, J=2.3 Hz, 2H), 3.74-3.70 (m, 2H), 3.17 (s, 9H), 3.06 (d, J=17.8 Hz, 1H), 2.62 (d, J=17.8 Hz, 1H), 1.99 (s, 3H), 1.95-1.82 (m, 3H), 1.38 (ddt, J=13.2, 10.0, 5.0 Hz, 1H), 1.22 (d, J=6.7 Hz, 3H).

### Example 17: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)propyl) carbonate (17)

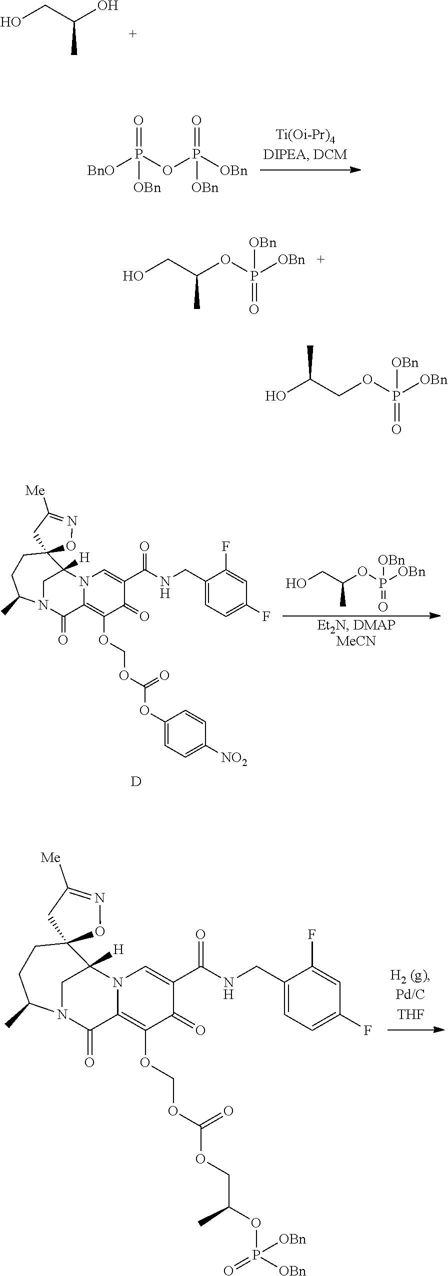

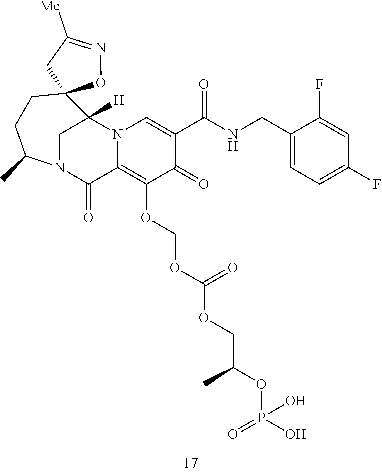 [SMILES: CC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1]

### Step 1: Preparation of(S)-dibenzyl (1-hydroxypropan-2-yl) phosphate and (S)-dibenzyl(2-hydroxypropyl) phosphate

To a mixture of(S)-propane-1,2-diol (0.44 mL, 4.16 mmol) in DCM (2.7 mL) was added DIPEA (1.66 mL, 9.25 mmol) followed by tetrabenzylpyrophosphate (2.00 g, 3.71 mmol) and Ti(i-PrO)₄ (0.19 mL, 0.631 mmol). The mixture was stirred at room temperature for 16 h. MgSO₄ and silica gel were added to reaction, stirred for 10 min, and filtered, rinsing with 4:3 EtOAc/hexane. The filtrate was concentrated and purified by reverse phase prep HPLC (10-100% MeCN/water) to afford the title compounds as an inseparable mixture of regioisomers (rr=1.1:1) by NMR. MS (m/z) 336.99 [M+H]+.

### Step 2: Preparation of(S)-2-((bis(benzyloxy)phosphoryl)oxy)propyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D) (0.250 g, 0.367 mmol) in MeCN (5 mL) was added Et₃N (0.153 mL, 1.10 mmol) and DMAP (0.004 g, 0.037 mmol), followed by the mixture of(S)-dibenzyl (1-hydroxypropan-2-yl) phosphate and (S)-dibenzyl(2-hydroxypropyl) phosphate (0.370 g, 1.10 mmol) prepared in Step 1. The reaction mixture was left to stir at room temperature for 16 h and concentrated. The residue was purified by silica gel column chromatography (0-100% EtOAc/hexane, then 0-20% MeOH/DCM) to afford the title compound. MS (m/z) 878.88 [M+H]⁺.

### Step 3: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)propyl) carbonate (17)

To a solution of(S)-2-((bis(benzyloxy)phosphoryl)oxy)propyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.068 g, 0.077 mmol) in THF (5 mL) was added 10% Pd/C (0.008 g, 0.008 mmol). The vial was evacuated and backfilled with hydrogen gas (2×), then sparged with hydrogen for 2 min. The reaction mixture was left to stir under a hydrogen balloon atmosphere for 2 h. The reaction mixture was filtered and concentrated. The crude residue was dissolved in 1:1 MeCN/water and purified by reverse phase prep HPLC (10-100% MeCN w/0.1% TFA) to afford the title compound after lyophilization. MS (m/z) 698.91 [M+H]⁺. 1H NMR (400 MHz, DMSO-d6) δ 10.28 (t, J=5.9 Hz, 1H), 8.71 (s, 1H), 7.42 (td, J=8.6, 6.7 Hz, 1H), 7.29-7.20 (m, 1H), 7.08 (td, J=8.5, 2.7 Hz, 1H), 5.84 (d, J=6.4 Hz, 1H), 5.64 (d, J=6.4 Hz, 1H), 4.63 (d, J=3.2 Hz, 2H), 4.61-4.48 (m, 2H), 4.42 (dq, J=11.9, 5.5 Hz, 1H), 4.13 (dd, J=11.2, 5.2 Hz, 1H), 4.04 (dd, J=11.2, 4.7 Hz, 1H), 3.78-3.58 (m, 2H), 3.00 (d, J=17.5 Hz, 1H), 2.64 (d, J=17.6 Hz, 1H), 1.95 (s, 3H), 1.88-1.66 (m, 3H), 1.36-1.24 (m, 1H), 1.22 (d, J=6.3 Hz, 3H), 1.16 (d, J=6.7 Hz, 3H).

### Example 18: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-(phosphonooxy)propan-2-yl) carbonate (18)

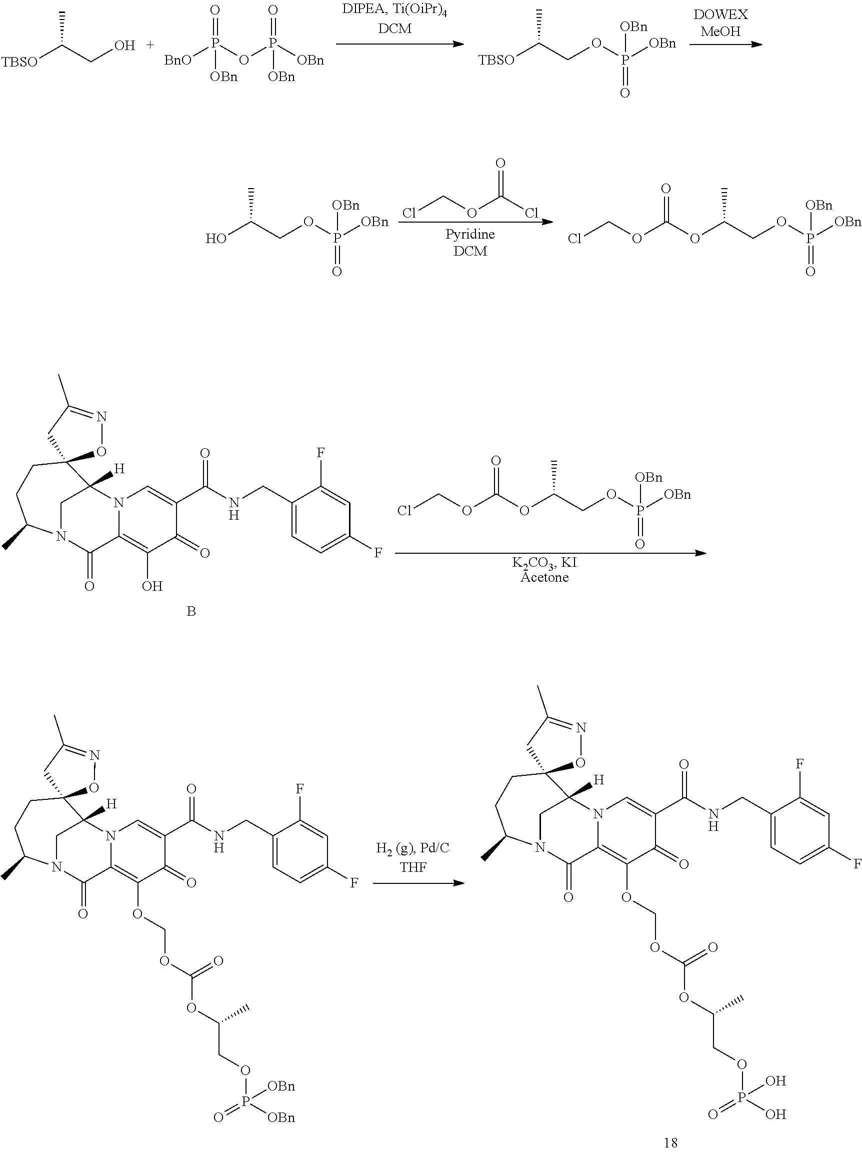

### Step 1: Preparation of (R)-dibenzyl(2-((tert-butyldimethylsilyl)oxy)propyl) phosphate

To a mixture of (R)-2-((tert-butyldimethylsilyl)oxy)propan-1-ol (0.396 g, 2.08 mmol) in DCM (1.35 mL) was added DIPEA (0.83 mL, 4.62 mmol) followed by tetrabenzylpyrophosphate (1.00 g, 1.86 mmol) and Ti(i-PrO)₄ (0.094 mL, 0.316 mmol). The mixture was stirred at room temperature for 16 h. MgSO₄ and silica gel were added to reaction, stirred for 10 min, and filtered, rinsing with 4:3 EtOAc/hexanes. The filtrate was concentrated and purified by reverse phase prep HPLC (10-100% MeCN/water) to afford the title compound. MS (m/z) 451.66 [M+H]+.

### Step 2: Preparation of (R)-dibenzyl(2-hydroxypropyl) phosphate

To a solution of (R)-dibenzyl(2-((tert-butyldimethylsilyl)oxy)propyl) phosphate (0.344 g, 0.763 mmol) in MeOH (3.4 mL) was added Dowex resin 50 W×8 (hydrogen form) (3.40 g) at rt. After 6 h, the mixture was filtered, concentrated, and purified by silica gel column chromatography (0-100% EtOAc/hexanes) to afford the title compound. MS (m/z) 336.74 [M+H]+.

### Step 3: Preparation of (R)-1-((bis(benzyloxy)phosphoryl)oxy)propan-2-yl (chloromethyl) carbonate

To a solution of (R)-dibenzyl(2-hydroxypropyl) phosphate (0.316 g, 0.940 mmol) in DCM (22 mL) at 0° C. was added chloromethyl chloroformate (0.184 mL, 2.07 mmol) followed by pyridine (0.190 mL, 2.35 mmol). The reaction mixture was allowed to warm to rt and stir 1 h. The reaction mixture was quenched with water and 1 N HCl. The phases were separated and aqueous phase was extracted with DCM (3×). The combined organic phase was washed with brine, dried over Na₂SO₄, filtered, and concentrated. The residue was purified by column chromatography (0-100% EtOAc/hexanes) to afford the title compound. MS (m/z) 428.78 [M+H]+.

### Step 4: Preparation of (R)-1-((bis(benzyloxy)phosphoryl)oxy)propan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To a solution of (R)-1-((bis(benzyloxy)phosphoryl)oxy)propan-2-yl (chloromethyl) carbonate (0.15 g, 0.35 mmol) in acetone (3.4 mL) was added Intermediate B (0.142 g, 0.292 mmol), KI (0.063 g, 0.379 mmol), and K₂CO₃ (0.081 g, 0.584 mmol). The reaction mixture was left to stir overnight at rt. The reaction mixture was partitioned between EtOAc and water and the phases separated. The aqueous phase was extracted with EtOAc and the combined organic phase was washed with brine, dried over Na₂SO₄, filtered, and concentrated. The residue was suspended in MeCN and purified by reverse phase prep HPLC (10-100% MeCN/water). The pooled fractions were concentrated to remove MeCN and extracted with EtOAc (3×). The combined organic phase was dried over Na₂SO₄, filtered, and concentrated. The residue was purified by silica gel column chromatography (0-100% EtOAc/hexanes) to afford the title compound. MS (m/z) 878.77 [M+H]+.

### Step 5: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-(phosphonooxy)propan-2-yl) carbonate (18)

To a solution of (R)-1-((bis(benzyloxy)phosphoryl)oxy)propan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.035 g, 0.040 mmol) in THF (4 mL) was added 10% Pd/C (0.004 g, 0.004 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered through Celite and concentrated. The residue was dissolved in 1:1 MeCN/water and lyophilized to afford the title compound. MS (m/z) 698.96 [M+H]⁺. 1H NMR (400 MHz, DMSO-d6) δ 10.28 (t, J=5.9 Hz, 1H), 8.71 (s, 1H), 7.43 (td, J=8.6, 6.5 Hz, 1H), 7.25 (ddd, J=10.5, 9.3, 2.6 Hz, 1H), 7.15-6.99 (m, 1H), 5.86 (d, J=6.4 Hz, 1H), 5.59 (d, J=6.4 Hz, 1H), 4.89-4.73 (m, 1H), 4.73-4.60 (m, 2H), 4.60-4.45 (m, 2H), 3.87 (dt, J=6.4, 4.6 Hz, 2H), 3.69 (dd, J=4.7, 2.3 Hz, 2H), 3.00 (d, J=17.6 Hz, 1H), 2.63 (d, J=17.6 Hz, 1H), 1.95 (s, 3H), 1.88-1.67 (m, 3H), 1.37-1.22 (m, 2H), 1.20 (d, J=6.4 Hz, 3H), 1.16 (d, J=6.7 Hz, 3H).

### Example 19: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-1-(phosphonooxy)propan-2-yl) carbonate (19)

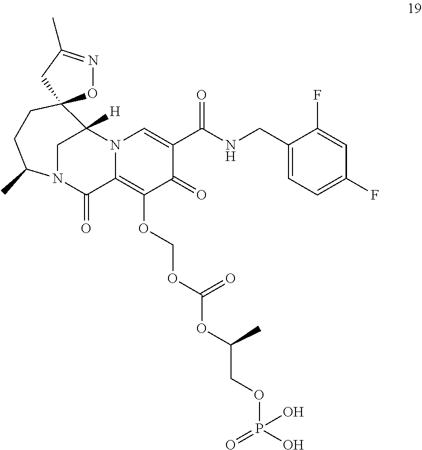 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

The title compound was prepared in a manner similar to Example 18, except using (S)-2-((tert-butyldimethylsilyl)oxy)propan-1-ol instead of (R)-2-((tert-butyldimethylsilyl)oxy)propan-1-ol in Step 1. MS (m/z) 698.83 [M+H]⁺. 1H NMR (400 MHz, DMSO-d6) δ 10.27 (t, J=5.9 Hz, 1H), 8.70 (s, 1H), 7.42 (td, J=8.6, 6.5 Hz, 1H), 7.29-7.20 (m, 1H), 7.07 (td, J=8.6, 2.6 Hz, 1H), 5.88-5.73 (m, 1H), 5.72-5.55 (m, 1H), 4.79 (dq, J=11.7, 6.2, 5.6 Hz, 1H), 4.61 (d, J=12.2 Hz, 2H), 4.59-4.47 (m, 2H), 3.85 (t, J=5.9 Hz, 2H), 3.68 (d, J=2.3 Hz, 2H), 3.00 (d, J=17.5 Hz, 1H), 2.64 (d, J=17.7 Hz, 1H), 1.94 (s, 3H), 1.85-1.70 (m, 3H), 1.33-1.22 (m, 1H), 1.20 (d, J=6.4 Hz, 3H), 1.15 (d, J=6.7 Hz, 3H).

### Example 20: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-(phosphonooxy)pentanoate (20)

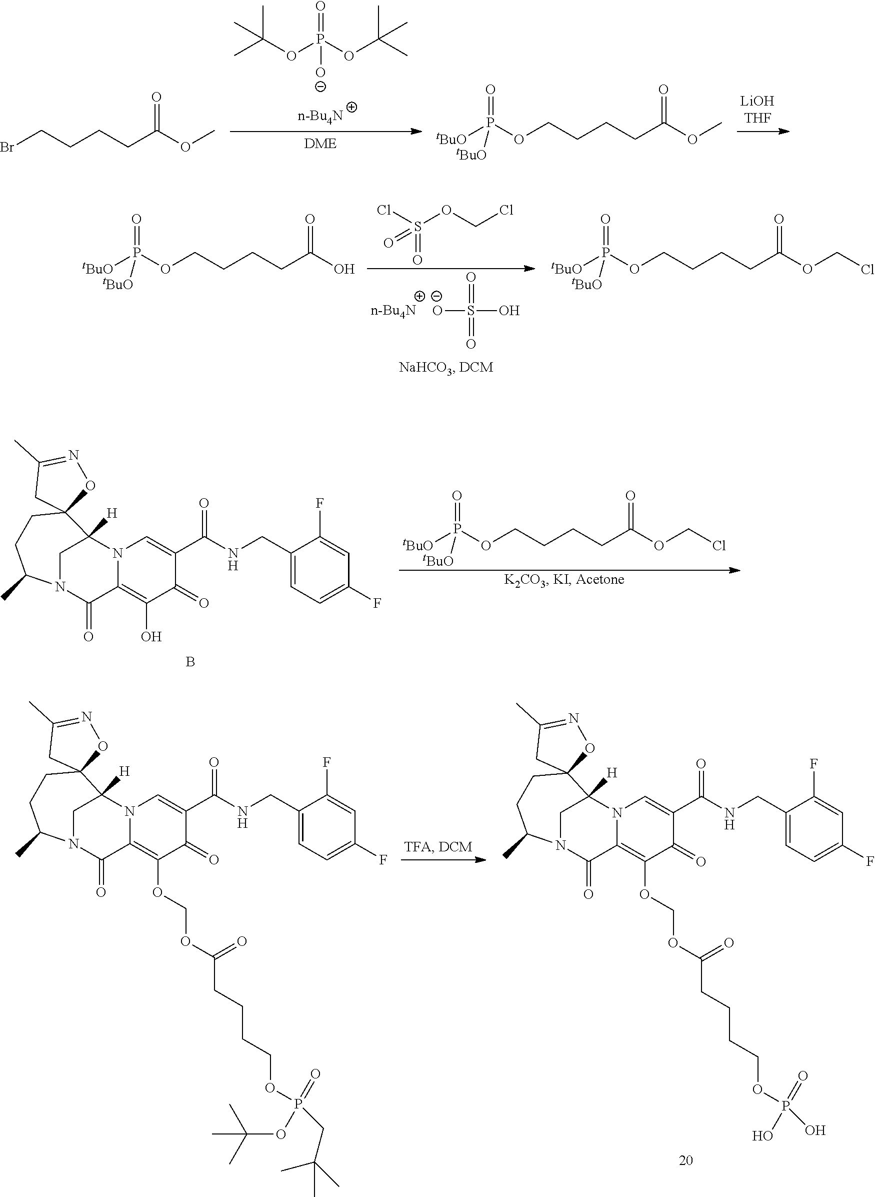

### Step 1: Preparation methyl 5-((di-tert-butoxyphosphoryl)oxy)pentanoate

A mixture of methyl 5-bromopentanoate (1 g, 5.13 mmol) and tetra-n-butylammonium di-tert-butylphosphate (2.78 g, 6.15 mmol) in dimethoxyethane (10 mL) was stirred at 80° C. for 3 hours. The reaction was cooled to ambient temperature, quenched with brine, and extracted with ethyl acetate (2×). The combined organic phase was dried over anhydrous MgSO₄, filtered, and concentrated under reduced pressure. The residue was purified by silica gel column chromatography to provide the title compound. MS (m/z) 324.7 [M+H]+.

### Step 2: Preparation 5-((di-tert-butoxyphosphoryl)oxy)pentanoic acid

A solution of 5-((di-tert-butoxyphosphoryl)oxy)pentanoate (1.32 g, 4.07 mmol) in tetrahydrofuran (15 mL) and methanol (10 mL) was treated with a solution of lithium hydroxide monohydrate (2.5 N) (3.58 ml, 8.95 mmol). The mixture was stirred for 3 hours, and then most of the solvent was evaporated under reduced pressure. The residue was diluted with 2 mL of water and acidified with 5% citric acid until pH 5. The resulting emulsion was extracted with ethyl acetate (2×). The combined organic phase was washed with brine, dried over anhydrous MgSO₄, filtered, and concentrated under reduced pressure to provide the title compound. MS (m/z) 311.1 [M+H]+.

### Step 3: Preparation chloromethyl 5-((di-tert-butoxyphosphoryl)oxy)pentanoate

A biphasic mixture of 5-di-tert-butoxyphosphoryloxypentanoic acid (1.8 g, 5.8 mmol), tetrabutylammonium hydrogen sulfate (0.197 g, 0.58 mmol), and sodium bicarbonate (2.92 g, 34.8 mmol) in water (9 mL) and DCM (18 mL) was cooled to 0° C. While stirring, chloromethyl chlorosulfate (1.9 g, 11.6 mmol) was added dropwise. The reaction mixture was allowed to stir 4 h, slowly warming to rt. Water was added and the aqueous phase was extracted with DCM (3×). The combined organic phase was washed with sodium bicarbonate and brine, dried over Na₂SO₄, filtered, and concentrated. The crude product was purified by column chromatography (0-100% EtOAc/hexanes) to afford title compound. MS (m/z) 358.8 [M+H]+.

### Step 4: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-((di-tert-butoxyphosphoryl)oxy)pentanoate

To a mixture of (3′S,5S,7′R)-N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.1 g, 0.26 mmol) and chloromethyl 5-di-tert-butoxyphosphoryloxypentanoate (0.147 g, 0.41 mmol) in acetone (1 mL) was added K₂CO₃ (0.0568 g, 0.411 mmol) and KI (0.044 g, 0.267 mmol). The resulting mixture was stirred at room temperature overnight. The mixture was diluted with EtOAc and was washed with H2O and brine. The organic phase was dried with MgSO₄ and the solvent was removed in vacuo. The residue was purified by silica gel column chromatography to provide the title compound. MS (m/z) 808.85 [M+H]+.

### Step 5: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-(phosphonooxy)pentanoate (20)

To a mixture of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-((di-tert-butoxyphosphoryl)oxy)pentanoate (0.14 g, 0.173 mmol) in DCM (10 mL) was added trifluoroacetic acid (1 mL) at 0° C. The mixture was stirred at room temperature for 3 h and the solvent was removed in vacuo. The resulting residue was purified by prep HPLC to provide the title compound. MS (m/z) 696.9 [M+H]+. 1H NMR (400 MHz, DMSO-d6) δ 10.30 (t, J=6.0 Hz, 1H), 8.69 (s, 1H), 7.43 (td, J=8.7, 6.6 Hz, 1H), 7.25 (ddd, J=10.4, 9.3, 2.6 Hz, 1H), 7.08 (td, J=8.5, 2.6 Hz, 1H), 5.81 (d, J=6.3 Hz, 1H), 5.59 (d, J=6.2 Hz, 1H), 4.71-4.48 (m, 4H), 3.79 (q, J=6.3 Hz, 2H), 3.68 (d, J=2.2 Hz, 2H), 3.00 (d, J=17.5 Hz, 1H), 2.64 (d, J=17.6 Hz, 1H), 2.37-2.28 (m, 2H), 1.95 (s, 3H), 1.79 (dtd, J=19.1, 14.6, 8.7 Hz, 3H), 1.56 (m, 4H), 1.35-1.22 (m, 1H), 1.16 (d, J=6.6 Hz, 3H).

### Example 21: Preparation of N-(2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)ethyl)-N-(((phosphonooxy)methoxy)carbonyl)glycine (21)

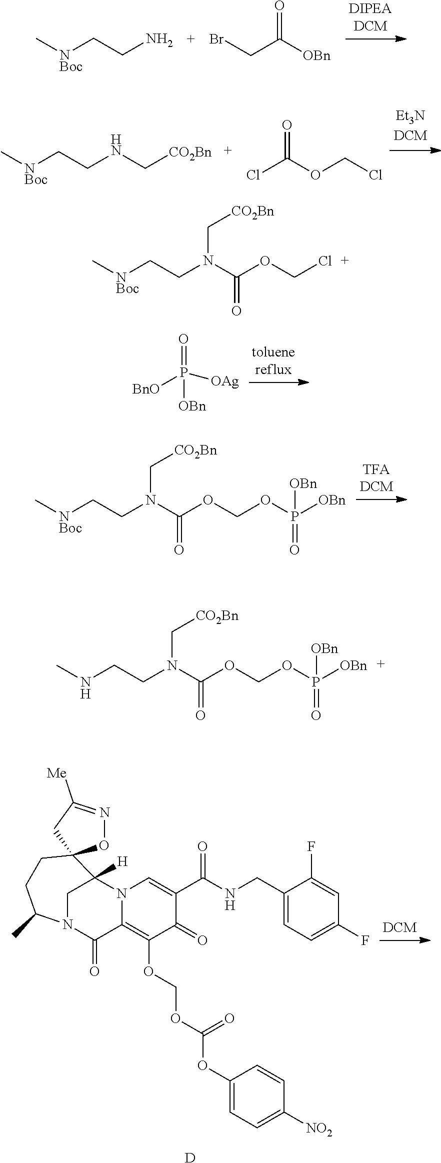

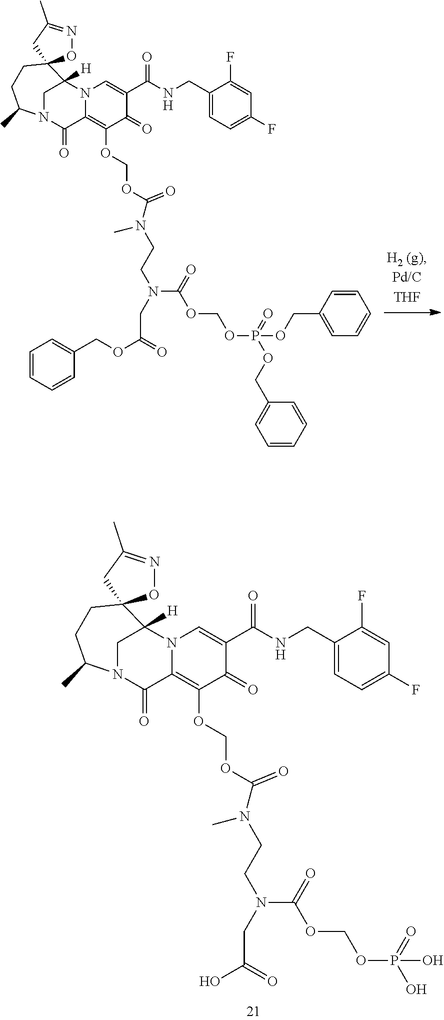 [SMILES: CC1=NO[C@@]2(CC[C@H](C)N3C[C@H]2n2cc(C(=O)NCc4ccc(F)cc4F)c(=O)c(OCOC(=O)N(C)CCN(CC(=O)OCc4ccccc4)C(=O)OCOP(=O)(OCc4ccccc4)OCc4ccccc4)c2C3=O)C1 ; CC1=NO[C@@]2(CC[C@H](C)N3C[C@H]2n2cc(C(=O)NCc4ccc(F)cc4F)c(=O)c(OCOC(=O)N(C)CCN(CC(=O)O)C(=O)OCOP(=O)(O)O)c2C3=O)C1]

### Step 1: Preparation of benzyl(2-((tert-butoxycarbonyl)amino)ethyl)glycinate

To a stirred solution of tert-butyl(2-aminoethyl) carbamate (3.0 g, 17.2 mmol) in DCM (90 mL) at 0° C. under argon was added benzyl 2-bromoacetate (1.97 g, 8.6 mmol) followed by DIPEA (2.45 g, 18.9 mmol). The reaction mixture was stirred for 16 h at room temperature. The reaction mixture was diluted with DCM and washed with water. The organic layer was dried over anhydrous Na₂SO₄, filtered, and concentrated under reduced pressure. The residue was purified by silica gel column chromatography (20-50% EtOAc/Hexanes) to afford the title compound. MS (m/z) 323.2 [M+H]+.

### Step 2: Preparation of benzyl N-(2-((tert-butoxycarbonyl)amino)ethyl)-N-((chloromethoxy)carbonyl)glycinate

To a stirred solution of benzyl(2-((tert-butoxycarbonyl)amino)ethyl)glycinate (2.8 g, 8.68 mmol) in DCM (45 mL) at 0° C. under argon was added chloromethyl chloroformate (1.46 g, 11.3 mmol) followed by Et₃N (2.2 g, 21.7 mmol). The mixture was stirred for 16 h at room temperature. The reaction mixture was diluted with DCM and washed with water. The organic layer was dried over anhydrous Na₂SO₄, filtered, and concentrated under reduced pressure. The crude residue was purified by silica gel column chromatography (20-50% EtOAc/Hexanes) to afford the title compound. MS (m/z) 415.6 [M+H]+.

### Step 3: Preparation of benzyl N-((((bis(benzyloxy)phosphoryl)oxy)methoxy)carbonyl)-N-(2-((tert-butoxycarbonyl)amino)ethyl)glycinate

To a stirred solution of benzyl N-(2-((tert-butoxycarbonyl)amino)ethyl)-N-((chloromethoxy)carbonyl)glycinate (3.25 g, 7.83 mmol) in toluene (20 mL) at room temperature under argon was added silver dibenzylphosphate (3.92 g, 10.2 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (30-70% EtOAc/Hexanes) to afford the title compound. MS (m/z) 657.6 [M+H]+.

### Step 4: Preparation of benzyl N-(2-aminoethyl)-N-((((bis(benzyloxy)phosphoryl)oxy)methoxy)carbonyl)glycinate

To a stirred solution of benzyl N-((((bis(benzyloxy)phosphoryl)oxy)methoxy)carbonyl)-N-(2-((tert-butoxycarbonyl)amino)ethyl)glycinate (100 mg, 0.15 mmol) in DCM (1 mL) at 0° C. under argon was added 2,2,2-trifluoroacetic acid (0.375 mL) in 0.5 mL of DCM under argon. The mixture was stirred for 40 minutes at room temperature and concentrated under reduced pressure. The crude residue was washed with NaHCO₃(aq) twice. The organic layer was dried with Na₂SO₄ and filtered. The mixture was concentrated and dried under vacuum to obtain the title compound. MS (m/z) 557.6 [M+H]+.

### Step 5: benzyl N-((((bis(benzyloxy)phosphoryl)oxy)methoxy)carbonyl)-N-(2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)ethyl)glycinate

To a solution of benzyl N-(2-aminoethyl)-N-((((bis(benzyloxy)phosphoryl)oxy)methoxy)carbonyl)glycinate (200 mg, 0.36 mmol) in DCM (2 mL) was added (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D, 100 mg, 0.15 mmol) at 0° C. The reaction mixture was warmed to room temperature and stirred for 4 h. The reaction mixture was concentrated to afford a residue, which was purified by reverse phase prep HPLC (5-100% MeCN/water) to afford the title compound. MS (m/z) 1099.2 [M+H]+.

### Step 6: Preparation of N-(2-((((((3′S,5S,7′R)-10′-((2,4-difluorobenzyl)carbamoyl)-3,3′-dimethyl-1′,11′-dioxo-1′4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)ethyl)-N-(((phosphonooxy)methoxy)carbonyl)glycine (21)

To a solution of benzyl N-((((bis(benzyloxy)phosphoryl)oxy)methoxy)carbonyl)-N-(2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)ethyl)glycinate (70 mg, 0.064 mmol) in THF (5 mL) was added 10% Pd/C (6.8 mg, 0.064 mmol). The flask was evacuated and backfilled with hydrogen gas (2×), then sparged with hydrogen for 2 min. The reaction mixture was left to stir under a hydrogen balloon atmosphere for 6 h. The reaction mixture was filtered, rinsed with THE, and concentrated to afford a residue, which was purified by reverse phase prep HPLC (5-100% MeCN/water) to afford the title compound. MS (m/z) 829.1 [M+H]⁺. 1H NMR (400 MHz, Methanol-d4) δ 8.56 (s, 1H), 7.46 (td, J=8.5, 6.3 Hz, 1H), 7.10-6.80 (m, 2H), 5.93-5.80 (m, 1H), 5.78-5.48 (m, 3H), 4.85-4.75 (m, 2H), 4.73-4.55 (m, 2H), 4.48 (d, J=2.2 Hz, 1H), 4.12 (s, 1H), 4.06 (d, J=4.5 Hz, 1H), 3.92-3.70 (m, 2H), 3.62-3.43 (m, 3H), 3.17 (d, J=17.9 Hz, 1H), 3.05-2.87 (m, 3H), 2.71 (d, J=18.1 Hz, 1H), 2.06 (s, 3H), 2.00-1.86 (m, 3H), 1.61-1.46 (m, 1H), 1.26 (dd, J=6.7, 1.3 Hz, 3H).

### Example 22: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-(phosphonooxy)propyl) carbonate (22)

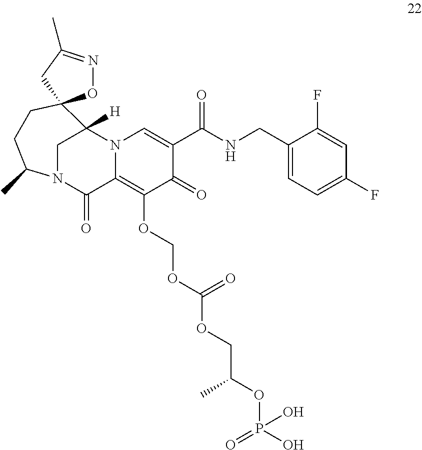 [SMILES: CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1]

The title compound was prepared in a manner similar to Example 18, except using (R)-1-((tert-butyldimethylsilyl)oxy)propan-2-ol instead of (R)-2-((tert-butyldimethylsilyl)oxy)propan-1-ol in Step 1. MS (m/z) 697.2 [M−H]⁻. ¹H NMR (400 MHz, Chloroform-d) & 10.36 (t, J=6.1 Hz, 1H), 8.49 (s, 1H), 7.36 (td, J=8.5, 6.3 Hz, 1H), 6.90-6.77 (m, 2H), 5.90 (d, J=6.5 Hz, 1H), 5.75 (d, J=6.4 Hz, 1H), 4.83 (q, J=7.9, 7.4 Hz, 1H), 4.62 (d, J=5.9 Hz, 2H), 4.22 (s, 1H), 4.23-4.06 (m, 2H), 3.85-3.77 (m, 1H), 3.73 (dd, J=15.1, 2.7 Hz, 1H), 3.08 (d, J=17.9 Hz, 1H), 2.62 (d, J=17.9 Hz, 1H), 2.08 (s, 3H), 1.94 (d, J=7.0 Hz, 4H), 1.63-1.52 (m, 1H), 1.34-1.22 (m, 6H).

### Example 23: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)propyl) carbonate (23)

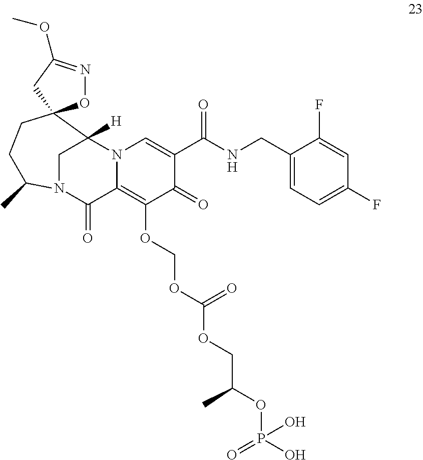 [SMILES: COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1]

The title compound was prepared in a manner similar to Example 18, except using (S)-1-((tert-butyldimethylsilyl)oxy)propan-2-ol instead of (R)-2-((tert-butyldimethylsilyl)oxy)propan-1-ol in Step 1 and Intermediate C instead of Intermediate B in Step 4. MS (m/z) 714.96 [M+H]⁺. 1H NMR (400 MHz, DMSO-d6) δ 10.27 (t, J=6.0 Hz, 1H), 8.75 (s, 1H), 7.42 (td, J=8.6, 6.6 Hz, 1H), 7.25 (td, J=9.9, 2.5 Hz, 1H), 7.08 (td, J=8.6, 2.6 Hz, 1H), 5.82 (d, J=6.4 Hz, 1H), 5.64 (d, J=6.4 Hz, 1H), 4.75-4.48 (m, 4H), 4.41 (dq, J=11.8, 5.7 Hz, 1H), 4.13 (dd, J=11.1, 5.2 Hz, 1H), 4.04 (dd, J=11.1, 4.7 Hz, 1H), 3.82 (s, 3H), 3.75-3.59 (m, 2H), 3.05 (d, J=16.8 Hz, 1H), 2.76 (d, J=16.9 Hz, 1H), 1.97-1.68 (m, 3H), 1.35-1.29 (m, 1H), 1.21 (d, J=6.4 Hz, 3H), 1.16 (d, J=6.7 Hz, 3H).

### Example 24: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-1-(phosphonooxy)propan-2-yl) carbonate (24)

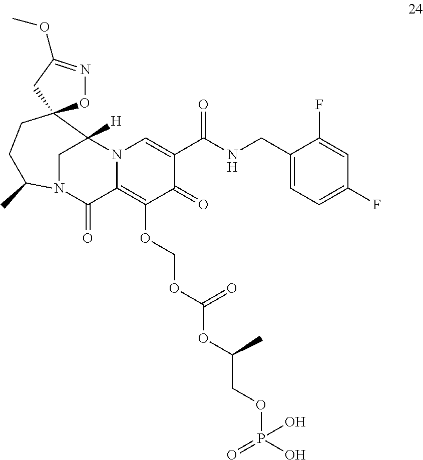 [SMILES: COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

The title compound was prepared in a manner similar to Example 18, except using (S)-2-((tert-butyldimethylsilyl)oxy)propan-1-ol instead of (R)-2-((tert-butyldimethylsilyl)oxy)propan-1-ol in Step 1 and Intermediate C instead of Intermediate B in Step 4. MS (m/z) 714.95 [M+H]⁺. 1H NMR (400 MHz, DMSO-d6) δ 10.27 (t, J=5.9 Hz, 1H), 8.75 (s, 1H), 7.42 (td, J=8.7, 6.7 Hz, 1H), 7.25 (ddd, J=10.5, 9.3, 2.6 Hz, 1H), 7.13-7.00 (m, 1H), 5.89-5.73 (m, 1H), 5.73-5.53 (m, 1H), 4.88-4.75 (m, 1H), 4.71 (d, J=2.7 Hz, 1H), 4.69-4.58 (m, 1H), 4.54 (q, J=9.1, 7.7 Hz, 2H), 3.87 (s, 2H), 3.82 (s, 3H), 3.75-3.60 (m, 2H), 3.05 (d, J=16.8 Hz, 1H), 2.77 (d, J=16.9 Hz, 1H), 1.91 (dd, J=15.3, 5.8 Hz, 1H), 1.88-1.69 (m, 2H), 1.40-1.26 (m, 1H), 1.21 (d, J=6.5 Hz, 3H), 1.15 (d, J=6.6 Hz, 3H).

### Example 25: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-(phosphonooxy)butan-2-yl) carbonate (25)

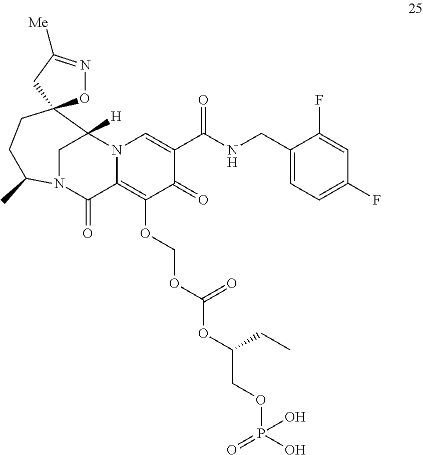 [SMILES: CC[C@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 10, except using (2R)-butane-1,2-diol instead of propane-1,3-diol in Step 1. MS (m/z) 713.8 [M+H]+. 1H NMR (400 MHz, DMSO) § 10.40-10.15 (m, 1H), 8.78-8.62 (m, 1H), 7.52-7.33 (m, 1H), 7.33-7.17 (m, 1H), 7.15-6.94 (m, 1H), 5.90 (d, J=6.5 Hz, 1H), 5.61 (d, J=6.5 Hz, 1H), 4.72-4.47 (m, 5H), 4.32-4.07 (m, 1H), 3.99-3.81 (m, 1H), 3.81-3.58 (m, 2H), 3.07-2.92 (m, 1H), 2.72-2.58 (m, 1H), 1.95 (s, 3H), 1.89-1.69 (m, 3H), 1.69-1.50 (m, 2H), 1.35-1.20 (m, 1H), 1.20-1.09 (m, 3H), 0.94-0.80 (m, 3H).

### Example 26: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-1-(phosphonooxy)butan-2-yl) carbonate (26)

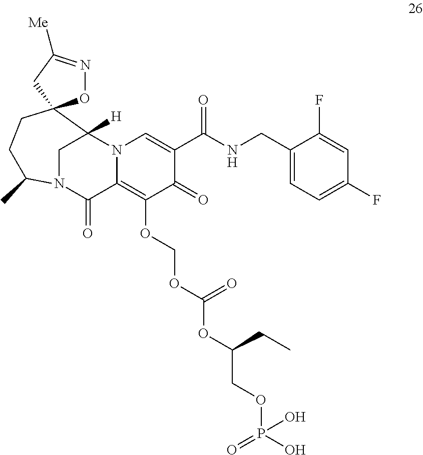 [SMILES: CC[C@@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 10, except using (2S)-butane-1,2-diol instead of propane-1,3-diol in Step 1. MS (m/z) 713.4 [M+H]⁺. 1H NMR (400 MHz, DMSO) δ 10.34-10.15 (m, 1H), 8.71 (s, 1H), 7.50-7.36 (m, 1H), 7.32-7.17 (m, 1H), 7.17-6.92 (m, 1H), 5.78 (d, J=6.5 Hz, 1H), 5.73 (d, J=6.5 Hz, 1H), 4.75-4.45 (m, 5H), 4.31-4.06 (m, 1H), 3.99-3.82 (m, 1H), 3.78-3.54 (m, 2H), 3.01 (d, J=17.6 Hz, 1H), 2.64 (d, J=17.7 Hz, 1H), 1.95 (s, 3H), 1.90-1.70 (m, 3H), 1.70-1.49 (m, 2H), 1.41-1.22 (m, 1H), 1.22-1.08 (m, 3H), 0.96-0.78 (m, 3H).

### Example 27: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3R)-3-(phosphonooxy)butan-2-yl) carbonate (27)

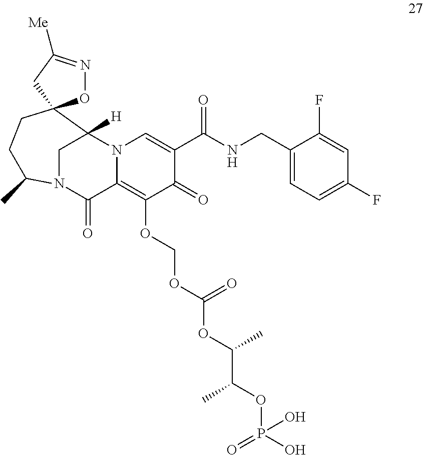 [SMILES: CC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1]

The title compound was prepared in a manner similar to Example 28, except using (2R,3R)-butane-2,3-diol instead of (2S,3S)-butane-2,3-diol in Step 1. MS (m/z) 713.1 [M+H]+. ¹H NMR (400 MHz, DMSO) δ 10.28 (t, J=6.0 Hz, 1H), 8.72 (s, 1H), 7.50-7.34 (m, 1H), 7.34-7.16 (m, 1H), 7.16-7.01 (m, 1H), 5.84 (d, J=6.6 Hz, 1H), 5.65 (d, J=6.5 Hz, 1H), 4.80-4.45 (m, 5H), 4.42-4.25 (m, 1H), 3.78-3.62 (m, 2H), 3.15-2.91 (m, 1H), 2.73-2.56 (m, 1H), 1.95 (s, 3H), 1.87-1.66 (m, 3H), 1.39-1.23 (m, 1H), 1.23-1.09 (m, 9H).

### Example 28: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)butan-2-yl) carbonate (28)

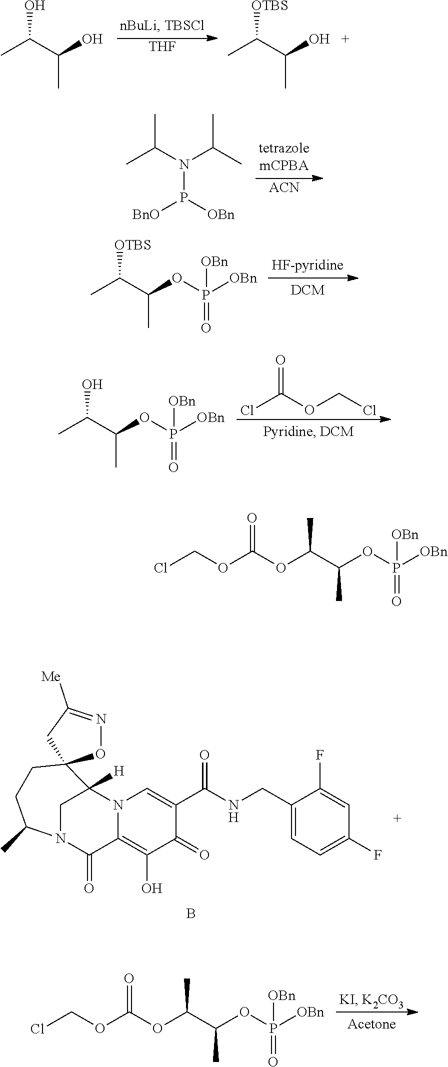

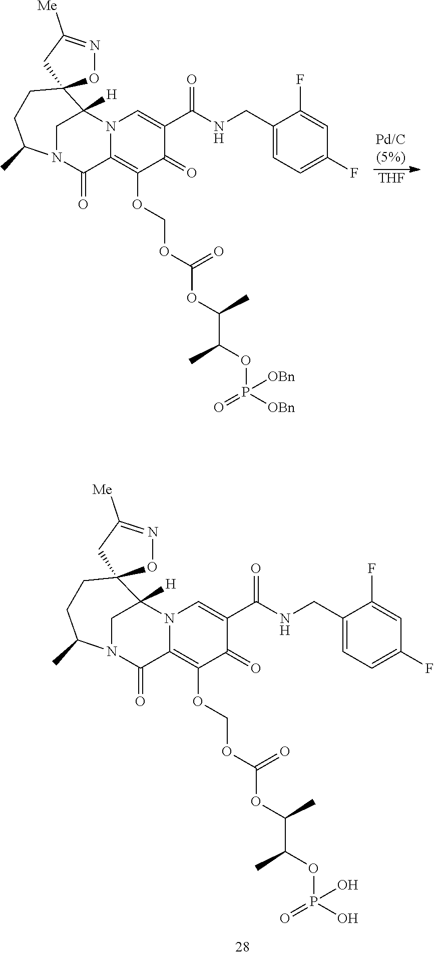 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(OCc4ccccc4)OCc4ccccc4)c2C3=O)C1 ; "THF" ; CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

### Step 1: Preparation of (2S,3S)-3-((tert-butyldimethylsilyl)oxy)butan-2-ol

To a stirred solution of (2S,3S)-butane-2,3-diol (2.0 g, 22.2 mmol) in anhydrous THF (40 mL) at −78° C., was added n-butyllithium (1.7 M, 9.7 mL, 24.4 mmol) and stirred at −78° C. for 30 min, followed by TBDMS-Cl (3.68 g, 24.4 mmol). After being stirred at room temperature for 24 h, the reaction mixture was cooled to 0° C., quenched with water (10 mL) slowly, washed with ammonium chloride solution (10 mL) and extracted with ethyl acetate (20 mL). The organic layer was washed with water (2×10 mL), and saturated NaCl solution (2×10 mL), dried over anhydrous Na₂SO₄, filtered and concentrated under reduced pressure and purified by silica gel chromatography (0-40% EtOAc/Hexanes) to afford the title compound. ¹H NMR (400 MHz, CDCl₃) δ 3.57 (p, J=6.1 Hz, 1H), 3.49 (p, J=6.2 Hz, 1H), 1.15 (m, 6H), 0.93 (s, 9H), 0.12 (s, 6H).

### Step 2: Preparation of dibenzyl ((2S,3S)-3-((tert-butyldimethylsilyl)oxy)butan-2-yl) phosphate

A solution of (2S,3S)-3-((tert-butyldimethylsilyl)oxy)butan-2-ol (2.5 g, 12.2 mmol) and dibenzyl diisopropylphosphoramidite (6.47 g, 18.3 mmol), and 1H-tetrazole (1.29 g, 18.3 mmol) in DCM (80 mL) was stirred at room temperature. After 1 h, the reaction mixture was cooled to 0° C. to which mCPBA (5.48 g, 24.5 mmol) was added portion wise. After being stirred for 10 min at 0° C., the reaction mixture was washed thoroughly with saturated sodium bicarbonate (3×30 mL) and saturated sodium thiosulfate (3×30 mL), and extracted with DCM (2×50 mL). The organic layer was washed with water and saturated NaCl (25 mL) solution, dried over anhydrous Na₂SO₄, filtered and concentrated under reduced pressure. The residue was purified using silica gel column chromatography (0-60% EtOAc/Hexanes) to provide the title compound. MS (m/z) 464.9 [M+H]+

### Step 3: Preparation of dibenzyl ((2S,3S)-3-hydroxybutan-2-yl) phosphate

To a stirred solution of dibenzyl ((2S,3S)-3-((tert-butyldimethylsilyl)oxy)butan-2-yl) phosphate (5.0 g, 10.8 mmol) in anhydrous DCM (50 mL) at 0° C., was added dropwise 70% HF-pyridine (70%, 1.67 mL). The reaction mixture was warmed to room temperature and stirred for 2 h. The reaction mixture was cooled to 0° C., partitioned between sodium bicarbonate solution (20 mL) and DCM, and extracted with DCM (2×50 mL). The organic layer was washed with H₂O (2×20 mL) and saturated NaCl solution (20 mL), dried over anhydrous Na₂SO₄, filtered and concentrated under reduced pressure. The residue was purified by silica gel column chromatography (0-80% EtOAc/Hexanes) to afford the title compound. MS (m/z) 350.9 [M+H]+

### Step 4: Preparation of (2S,3S)-3-(bis(benzyloxy)phosphoryl)oxy)butan-2-yl (chloromethyl) carbonate

Dibenzyl ((2S,3S)-3-hydroxybutan-2-yl) phosphate (1.77 g, 5.1 mmol) was added to a stirred mixture of chloromethyl chloroformate (0.9 mL, 10.1 mmol) in DCM (25 mL) then followed by the addition of pyridine (0.72 mL, 8.8 mmol) dropwise at room temperature. The reaction was stirred for 30 minutes before it was diluted with DCM (10 mL) and washed sequentially with 0.5 M HCl (5 mL) and water (10 mL). The organic layer was dried over magnesium sulfate, filtered, and evaporated to obtain the title compound. MS (m/z) 444.2 [M+H]+.

### Step 5: Preparation of (2S,3S)-3-((bis(benzyloxy)phosphoryl)oxy)butan-2-yl ((((3′S,5S,7′R)-10′-((2,4-difluorobenzyl)carbamoyl)-3,3′-dimethyl-1′,11′-dioxo-1′,4,5′,11′-tetrahydro-3′H,4H,7′H-spiro[isoxazole-5,6′-[2,7]methanopyrido[1,2-a][1,4]diazonin]-12′-yl)oxy)methyl) carbonate

To the solution of Intermediate B (1.1 g, 2.3 mmol) in acetone (15 mL) was added KI (938 mg, 5.7 mmol) and K₂CO₃ (625 mg, 4.5 mmol) followed by (2S,3S)-3-((bis(benzyloxy)phosphoryl)oxy)butan-2-yl (chloromethyl) carbonate (2.0 g, 4.5 mmol). The resulting mixture was left to stir at room temperature for 48 h and then was concentrated. The residue was dissolved in DCM (30 mL), washed with water (2×20 mL), separated, dried and concentrated under reduced pressure. The residue was purified by silica gel column chromatography (0-100% EtOAc/Hexanes) to afford the title compound. MS (m/z) 893.3 [M+H]+.

### Step 6: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)butan-2-yl) carbonate (28)

To a solution (2S,3S)-3-((bis(benzyloxy)phosphoryl)oxy)butan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (1.0 g, 1.12 mmol) in THF (20 mL) was added 5 wt % Pd/C (119 mg, 0.11 mmol). The resulting mixture was purged with hydrogen three times before it was stirred under hydrogen for 1 h. The reaction was then filtered through Celite® and concentrated. The resulting residue was purified by reverse phase preparative HPLC (10-100% acetonitrile 0.1% TFA/water 0.1% TFA to afford the title compound. MS (m/z) 713.7 [M+H]⁺. 1H NMR (400 MHz, DMSO) δ 10.28 (t, J=6.0 Hz, 1H), 8.71 (s, 1H), 7.52-7.34 (m, 1H), 7.34-7.19 (m, 1H), 7.19-7.02 (m, 1H), 5.84 (d, J=6.5 Hz, 1H), 5.66 (d, J=6.6 Hz, 1H), 4.75-4.47 (m, 5H), 4.41-4.15 (m, 1H), 3.79-3.59 (m, 2H), 3.00 (d, J=17.5 Hz, 1H), 2.74-2.58 (m, 1H), 1.95 (s, 3H), 1.89-1.67 (m, 3H), 1.41-1.22 (m, 1H), 1.22-1.05 (m, 9H).

### Example 29: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)butan-2-yl) carbonate (29)

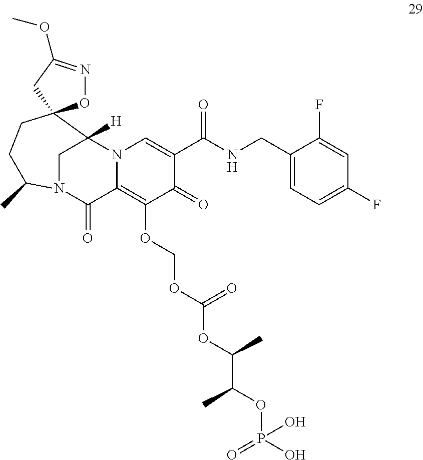 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

The title compound was prepared following a similar method as Example 28, except using Intermediate C instead of Intermediate B in Step 5. MS (m/z) 728.80 [M+H]⁺. 1H NMR (400 MHz, Methanol-d4) δ 8.76 (s, 1H), 7.44 (td, J=8.5, 6.3 Hz, 1H), 7.08-6.81 (m, 2H), 5.90 (d, J=6.7 Hz, 1H), 5.71 (d, J=6.6 Hz, 1H), 4.79 (qd, J=7.1, 6.6, 2.8 Hz, 2H), 4.71 (d, J=2.5 Hz, 1H), 4.60 (s, 2H), 4.51-4.34 (m, 1H), 3.85 (s, 3H), 3.83-3.74 (m, 2H), 3.19 (d, J=17.0 Hz, 1H), 2.76 (d, J=17.1 Hz, 1H), 2.04 (dd, J=13.8, 4.4 Hz, 1H), 1.94 (td, J=10.4, 9.5, 3.7 Hz, 2H), 1.63-1.39 (m, 1H), 1.38-1.18 (m, 9H).

### Example 30: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,4S)-4-(phosphonooxy)pentan-2-yl) carbonate (30)

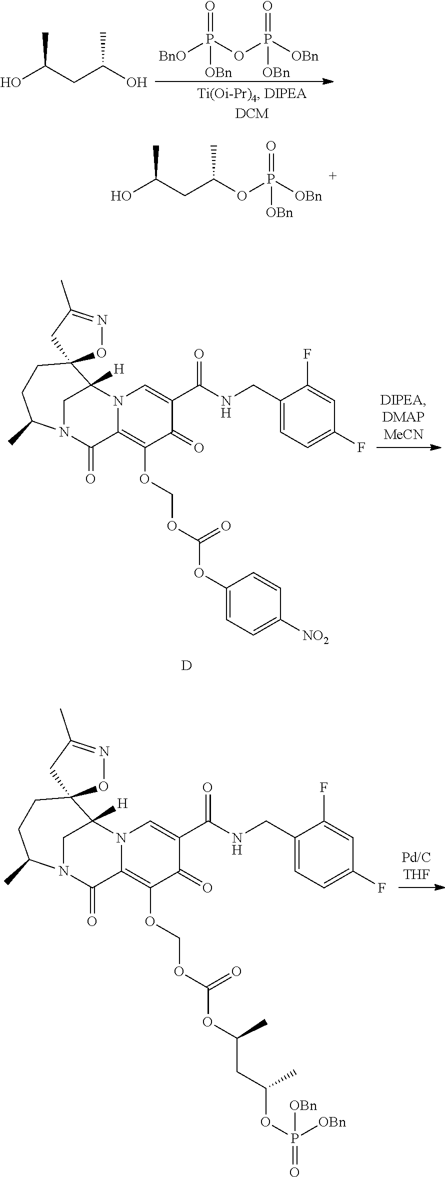

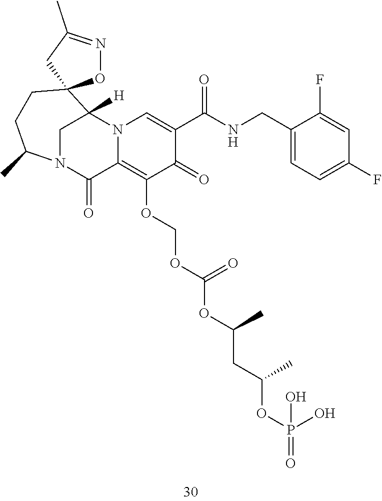 [SMILES: CC1=NO[C@@]2(CC[C@H](C)N3C[C@H]2n2cc(C(=O)NCc4ccc(F)cc4F)c(=O)c(OCOC(=O)O[C@@H](C)C[C@H](C)OP(=O)(O)O)c2C3=O)C1]

### Step 1: Preparation of dibenzyl ((2S,4S)-4-hydroxypentan-2-yl) phosphate

To a solution of (2S,4S)-pentane-2,4-diol (0.7 g, 6.72 mmol) in DCM (7 mL) was added N,N-diisopropylethylamine (1.76 mL, 10.1 mmol). To the resulting mixture was added dibenzyl dibenzyloxyphosphoryl phosphate (2.17 g, 4.03 mmol) and titanium (IV) isopropoxide (0.19 g, 0.67 mmol). The mixture was stirred at room temperature for 4 h then filtered through a column containing of silica gel and magnesium sulfate (20:1) and washed with ethyl acetate/hexanes (75%, 50 mL). The solution was dried over sodium sulfate, filtered, and concentrated under reduced pressure. The residue was purified by column chromatography on a silica gel to afford the title compound. MS (m/z) 364.9 [M+H]+.

### Step 2: Preparation of (2S,4S)-4-((bis(benzyloxy)phosphoryl)oxy)pentan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To the solution of Intermediate D (0.2 g, 0.29 mmol) in acetonitrile (2 mL) was added dibenzyl ((2S,4S)-4-hydroxypentan-2-yl) phosphate (0.21 g, 0.59 mmol), 4-dimethylaminopyridine (7.17 mg, 0.059 mmol) and N,N-diisopropylethylamine (0.076 g, 0.59 mmol). The resulting mixture was stirred at room temperature overnight. EtOAc was added to dilute and the mixture was washed with 0.5 N HCl, H₂O and brine. The organic phase was dried with MgSO₄, filtered, and concentrated. The resulting crude material was purified by silica gel column chromatography to obtain the title compound. MS (m/z) 906.7 [M+H]+.

### Step 3: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,4S)-4-(phosphonooxy)pentan-2-yl) carbonate (30)

To the solution of (2S,4S)-4-((bis(benzyloxy)phosphoryl)oxy)pentan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (45.8 mg, 0.0505 mmol)) in THF (10 mL) was added 5% wt Pd/C (5.38 mg, 0.0051 mmol). The flask was purged with hydrogen three times and left to stir under hydrogen for 1 h. The reaction mixture was filtered through Celite and the filtrate was concentrated. The resulting residue was purified by prep HPLC (5-100% MeCN/water w/0.1% TFA) to afford the title compound. MS (m/z) 726.8 [M+H]⁺. 1H NMR (400 MHz, Chloroform-d) § 10.19 (s, 1H), 8.67 (s, 1H), 7.39 (q, J=8.3 Hz, 1H), 6.95-6.66 (m, 2H), 5.97 (s, 1H), 5.53 (d, J=6.6 Hz, 1H), 5.12-4.75 (m, 2H), 4.63 (qd, J=15.0, 6.0 Hz, 4H), 4.34 (s, 1H), 3.96-3.56 (m, 2H), 2.64 (d, J=17.7 Hz, 1H), 2.08 (d, J=12.9 Hz, 3H), 2.02-1.85 (m, 3H), 1.79 (d, J=8.4 Hz, 2H), 1.67-1.45 (m, 1H), 1.30 (dd, J=24.7, 6.4 Hz, 9H).

### Example 31: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,4R)-4-(phosphonooxy)pentan-2-yl) carbonate (31)

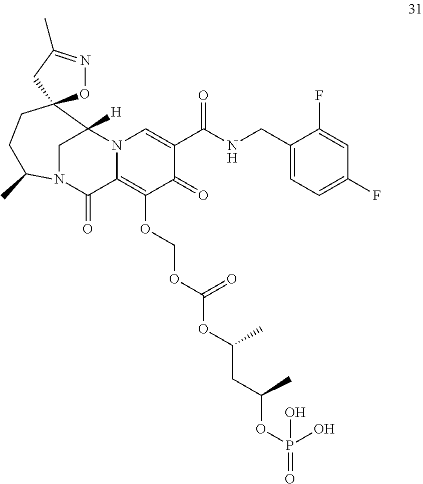 [SMILES: CC1=NO[C@@]2(CC[C@H](C)N3C[C@H]2n2cc(C(=O)NCc4ccc(F)cc4F)c(=O)c(OCOC(=O)O[C@H](C)C[C@@H](C)OP(=O)(O)O)c2C3=O)C1]

The title compound was prepared following a similar method as Example 30, except using (2R,4R)-pentane-2,4-diol instead of (2S,4S)-pentane-2,4-diol in Step 1. MS (m/z) 726.8 [M+H]+. 1H NMR (400 MHz, DMSO-d6) δ 10.29 (t, J=6.0 Hz, 1H), 8.71 (s, 1H), 7.43 (td, J=8.7, 6.6 Hz, 1H), 7.25 (ddd, J=10.5, 9.3, 2.6 Hz, 1H), 7.13-7.03 (m, 1H), 5.84 (d, J=6.4 Hz, 1H), 5.54 (d, J=6.4 Hz, 1H), 4.78-4.47 (m, 5H), 4.31-4.21 (m, 1H), 3.79-3.62 (m, 1H), 3.00 (d, J=17.6 Hz, 1H), 2.63 (d, J=17.5 Hz, 1H), 1.95 (s, 3H), 1.89-1.66 (m, 6H), 1.36-1.24 (m, 1H), 1.29-1.13 (m, 9H).

### Example 32: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2,2-dimethyl-3-(phosphonooxy)propyl) carbonate (32)

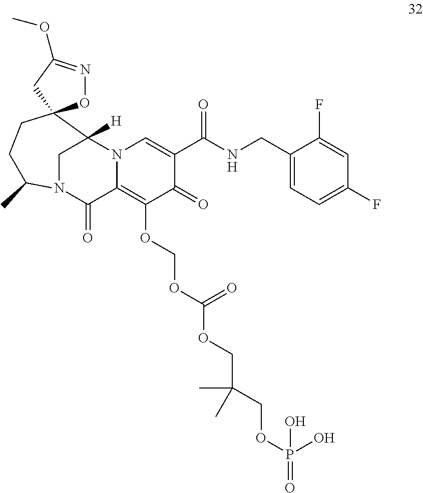 [SMILES: COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

The title compound was prepared following a similar method as Example 10, except using 2,2-dimethylpropane-1,3-diol instead of propane-1,3-diol in Step 1 and Intermediate C instead of Intermediate B in Step 3. MS (m/z) 742.9 [M+H]+. 1H NMR (400 MHz, DMSO-d6) δ10.28 (t, J=6.0 Hz, 1H), 8.75 (s, 1H), 7.42 (q, J=8.2 Hz, 1H), 7.25 (td, J=9.9, 2.5 Hz, 1H), 7.08 (td, J=8.5, 2.5 Hz, 1H), 5.79 (d, J=6.4 Hz, 1H), 5.67 (d, J=6.4 Hz, 1H), 4.71 (s, 1H), 4.63 (dt, J=10.4, 6.9 Hz, 1H), 4.61-4.47 (m, 2H), 3.88 (m, 2H), 3.82 (m, 2H), 3.75-3.61 (m, 2H), 3.59 (d, J=5.2 Hz, 2H), 3.05 (d, J=16.8 Hz, 1H), 2.75 (d, J=16.9 Hz, 1H), 1.91 (dd, J=15.4, 5.9 Hz, 1H), 1.88-1.69 (m, 2H), 1.35 (d, J=12.1 Hz, 2H), 1.15 (d, J=6.7 Hz, 3H), 0.88 (s, 6H).

### Example 33: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1-((phosphonooxy)methyl)cyclopropyl)methyl) carbonate (33)

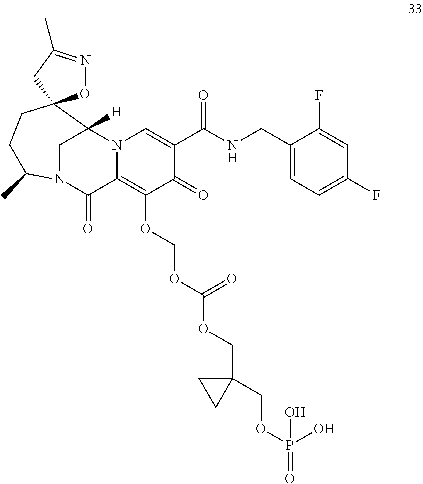 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)CC4)c2C3=O)C1]

The title compound was prepared in a manner similar to Example 17, except using cyclopropane-1,1-diyldimethanol instead of(S)-propane-1,2-diol in Step 1. MS (m/z) 725.0 [M+H]⁺. 1H NMR (400 MHz, Methanol-d4) δ 8.58 (s, 1H), 7.46 (td, J=8.5, 6.3 Hz, 1H), 7.07-6.90 (m, 2H), 5.91 (d, J=6.5 Hz, 1H), 5.72 (d, J=6.6 Hz, 1H), 4.86-4.76 (m, 1H), 4.74-4.59 (m, 2H), 4.48 (d, J=2.5 Hz, 1H), 4.13 (s, 2H), 3.99-3.73 (m, 4H), 3.16 (d, J=17.8 Hz, 1H), 2.78-2.66 (m, 1H), 2.06 (s, 3H), 2.03-1.87 (m, 3H), 1.63-1.47 (m, 1H), 1.26 (d, J=6.7 Hz, 3H), 0.68 (d, J=2.6 Hz, 4H).

### Example 34: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-(phosphonooxy)butyl) carbonate (34)

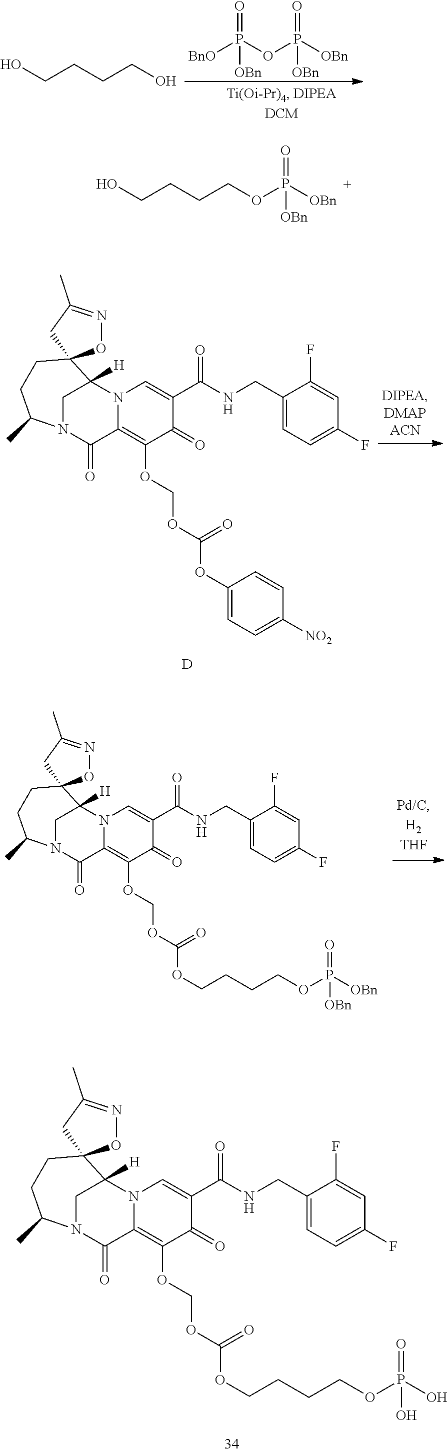

The title compound was prepared following a similar method as Example 30, except using butane-1,4-diol instead of (2S,4S)-pentane-2,4-diol. MS (m/z) 712.93 [M+H]+. 1H NMR (400 MHz, Chloroform-d) & 10.51 (t, J=6.0 Hz, 1H), 8.43 (s, 1H), 7.42-7.30 (m, 1H), 6.96-6.78 (m, 2H), 5.90 (dd, J=49.9, 6.5 Hz, 2H), 4.84 (q, J=8.2, 7.2 Hz, 1H), 4.71-4.52 (m, 2H), 4.29-4.11 (m, 3H), 4.00 (d, J=6.6 Hz, 2H), 3.85-3.59 (m, 2H), 3.08 (d, J=17.9 Hz, 1H), 2.62 (d, J=17.9 Hz, 1H), 2.08 (s, 3H), 2.05-1.85 (m, 3H), 1.72 (m, 4H), 1.58 (dd, J=15.8, 9.6 Hz, 1H), 1.37-1.13 (m, 3H).

### Example 35: Preparation of (phosphonooxy)methyl ((2S)-1-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)propan-2-yl)(methyl)carbamate (35)

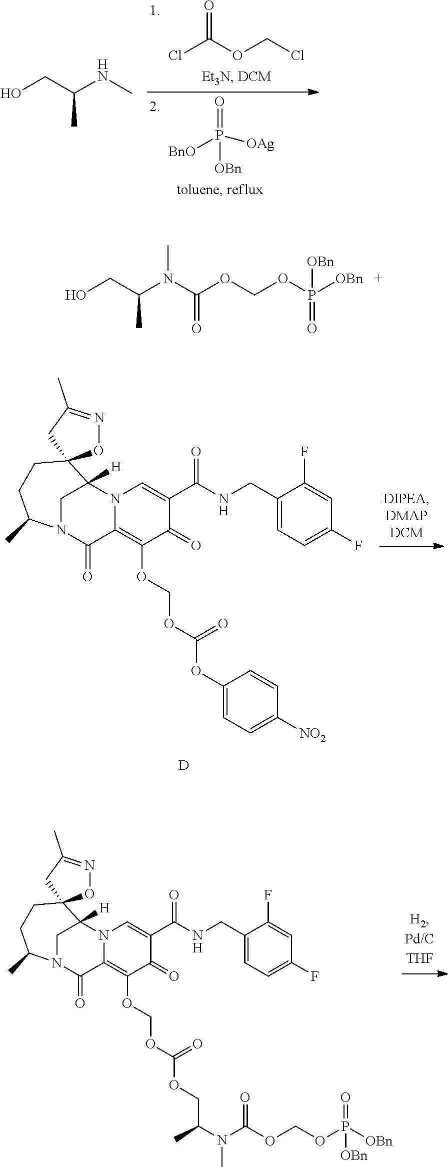

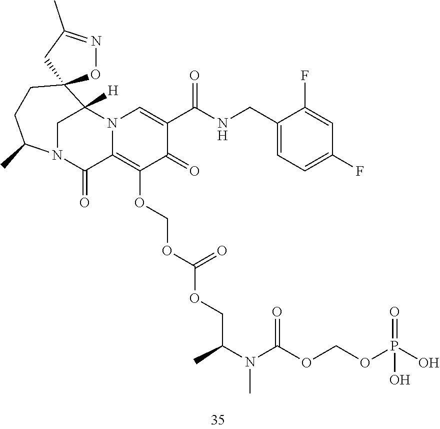 [SMILES: CC1=NO[C@@]2(CC[C@H](C)N3C[C@H]2n2cc(C(=O)NCc4ccc(F)cc4F)c(=O)c(OCOC(=O)OC[C@H](C)N(C)C(=O)OCOP(=O)(O)O)c2C3=O)C1]

### Step 1: Preparation of ((bis(benzyloxy)phosphoryl)oxy)methyl(S)-(1-hydroxypropan-2-yl)(methyl)carbamate

To a stirred solution of (2S)-2-(methylamino) propan-1-ol (1.2 g, 13.5 mmol) in DCM (80 mL) at 0° C. under argon was added chloromethyl chloroformate (2.08 g, 16.2 mmol) followed by triethylamine (3.5 g, 33.7 mmol). The mixture was stirred for 16 h at room temperature. The reaction mixture was diluted with DCM and washed with water. The organic layer was dried over anhydrous Na₂SO₄, filtered, and concentrated under reduced pressure. To the crude residue in toluene (60 mL) at room temperature under argon was added silver dibenzylphosphate (5.6 g, 15 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (30-70% EtOAc/Hexanes) to afford the title compound. MS (m/z) 424.3 [M+H]+.

### Steps 2-3: Preparation of (phosphonooxy)methyl (2S)-1-(((((3′S,5S,7′R)-10′-(2,4-difluorobenzyl)carbamoyl)-3,3′-dimethyl-1′, 11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)propan-2-yl)(methyl)carbamate (35)

The title compound was prepared in a manner similar to Example 4, except using ((bis(benzyloxy)phosphoryl)oxy)methyl(S)-(1-hydroxypropan-2-yl)(methyl)carbamate, N,N-diisopropylethylamine, and dichloromethane instead of dibenzyl(2-hydroxyethyl) phosphate, triethylamine, and acetonitrile in Step 1. MS (m/z) 786.1 [M+H]⁺. 1H NMR (400 MHz, Methanol-d4) δ 8.58 (s, 1H), 7.45 (td, J=8.5, 6.3 Hz, 1H), 7.04-6.88 (m, 2H), 5.92 (t, J=6.6 Hz, 1H), 5.73-5.50 (m, 3H), 4.86-4.75 (m, 1H), 4.73-4.56 (m, 2H), 4.56-4.41 (m, 2H), 4.25-4.05 (m, 2H), 3.89-3.71 (m, 2H), 3.17 (d, J=18.0 Hz, 1H), 2.86 (d, J=9.8 Hz, 3H), 2.72 (dd, J=18.0, 4.5 Hz, 1H), 2.06 (q, J=2.1, 1.5 Hz, 3H), 1.94 (q, J=11.7, 11.1 Hz, 3H), 1.55 (dd, J=10.0, 7.5 Hz, 1H), 1.26 (d, J=6.7 Hz, 3H), 1.20 (dd, J=9.4, 7.0 Hz, 3H).

### Example 36: Preparation of (phosphonooxy)methyl (1-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylpropan-2-yl)(methyl)carbamate (36)

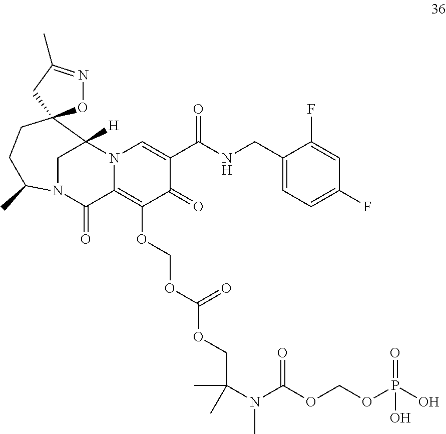 [SMILES: CC1=NO[C@@]2(CC[C@H](C)N3C[C@H]2n2cc(C(=O)NCc4ccc(F)cc4F)c(=O)c(OCOC(=O)OCC(C)(C)N(C)C(=O)OCOP(=O)(O)O)c2C3=O)C1]

The title compound was prepared in a manner similar to Example 35, except using 2-methyl-2-(methylamino) propan-1-ol instead of (2S)-2-(methylamino) propan-1-ol in Step 1. MS (m/z) 800.0 [M+H]⁺. 1H NMR (400 MHz, Methanol-d4) δ 8.58 (s, 1H), 7.54-7.37 (m, 1H), 7.05-6.93 (m, 2H), 5.89 (d, J=6.7 Hz, 1H), 5.74 (d, J=6.7 Hz, 1H), 5.57 (d, J=13.6 Hz, 2H), 4.85-4.75 (m, 1H), 4.71-4.59 (m, 2H), 4.47 (s, 1H), 4.41 (s, 2H), 3.79 (qd, J=15.2, 2.3 Hz, 2H), 3.16 (d, J=17.8 Hz, 1H), 3.01 (s, 3H), 2.76-2.66 (m, 1H), 2.06 (s, 3H), 1.95 (q, J=11.9, 10.4 Hz, 3H), 1.55 (dd, J=10.3, 7.8 Hz, 1H), 1.43 (s, 6H), 1.27 (d, J=6.7 Hz, 3H).

### Example 37: Preparation of N-(3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2,2-dimethylpropyl)-N-(((phosphonooxy)methoxy)carbonyl)glycine (37)

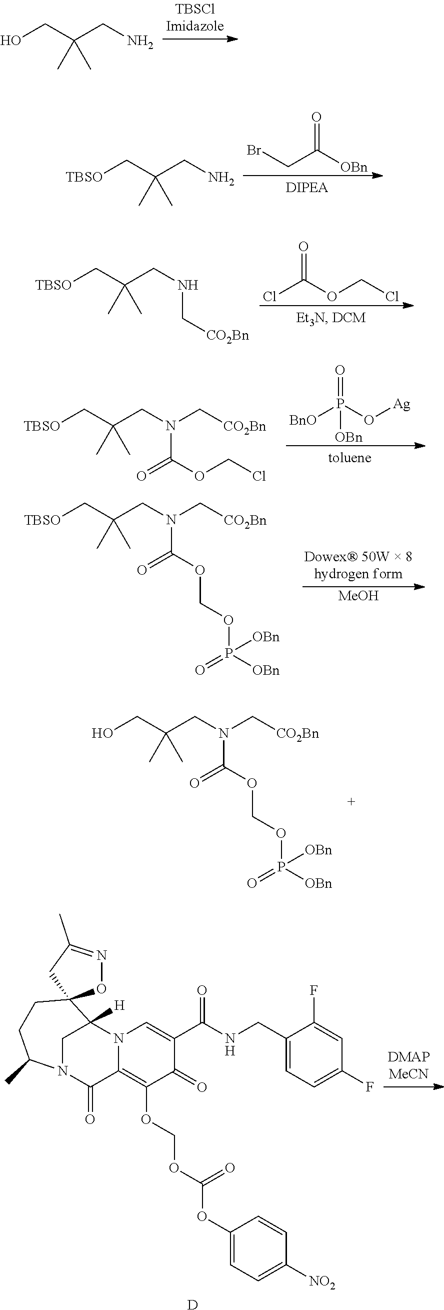

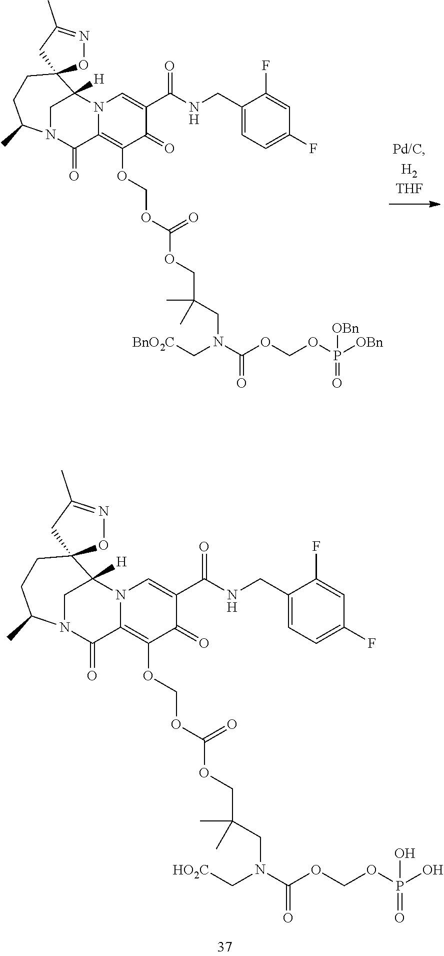

### Step 1: Preparation of 3-((tert-butyldimethylsilyl)oxy)-2,2-dimethylpropan-1-amine

To solution of 3-amino-2,2-dimethyl-propan-1-ol (4.5 g, 43.6 mmol) in DCM (40 mL) was added imidazole (5.94 g, 87.2 mmol), then followed by the addition of tert-butylchlorodimethylsilane (6.57 g, 43.6 mmol) dropwise at 0° C. The resulting solution was warmed up to room temperature and stirred of overnight. The solution was washed with H₂O, brine and dried with MgSO₄. The solvent was removed under vacuo to afford the title compound. MS (m/z) 217.9 [M+H]+.

### Step 2: Preparation of benzyl (3-((tert-butyldimethylsilyl)oxy)-2,2-dimethylpropyl)glycinate

To a stirred solution of 3-((tert-butyldimethylsilyl)oxy)-2,2-dimethylpropan-1-amine (4.7 g, 22 mmol) in CH2Cl₂ (40 mL) at 0° C. was added DIPEA (8.4 g, 65 mmol) followed by the addition of benzyl 2-bromoacetate (2.5 g, 11 mmol) in CH2Cl₂ (10 mL) dropwise. The reaction was stirred at 0° C. for 2 h. The solution was diluted with DCM and washed with H₂O and brine, then dried with MgSO₄. The solvent was removed under vacuo and the crude material was purified by silica gel column chromatography (0-10% DCM/MeOH) to obtain the title compound. MS (m/z) 366.23 [M+H]+.

### Step 3: Preparation of benzyl N-(3-((tert-butyldimethylsilyl)oxy)-2,2-dimethylpropyl)-N-((chloromethoxy)carbonyl)glycinate

To a stirred solution of benzyl (3-((tert-butyldimethylsilyl)oxy)-2,2-dimethylpropyl)glycinate (3 g, 8.21 mol) in DCM (30 mL) at −5° C. was added triethylamine (1.73 mL, 12.3 mmol) followed by the addition of chloromethyl carbonochloridate (0.98 g, 7.63 mmol) slowly. The mixture was stirred at −5° C. for 1 h. Saturated aqueous NaHCO₃ (30 mL) was added at 0° C. and the mixture was stirred for 10 minutes. EtOAc (100 mL) was added and the organic phase was washed with H₂O and brine, and dried over MgSO₄. The solvent was removed under vacuum to afford the title compound, which was used without further purification.

### Step 4: Preparation of benzyl N-((((bis(benzyloxy)phosphoryl)oxy)methoxy)carbonyl)-N-(3-((tert-butyldimethylsilyl)oxy)-2,2-dimethylpropyl)glycinate

To a stirred solution of benzyl N-(3-((tert-butyldimethylsilyl)oxy)-2,2-dimethylpropyl)-N-((chloromethoxy)carbonyl)glycinate (3.5 g, 7.64 mmol) in toluene (35 mL) was added silver dibenzyl phosphate (5.9 g, 15.3 mmol). The resulting mixture was stirred at reflux for overnight. The mixture was filtered through celite and washed with toluene. The solvent was removed under vacuum and the resulting crude residue was purified by silica gel column chromatography (0-100% EtOAc/hexane) to obtain the title compound. MS (m/z) 700.4 [M+H]+.

### Step 5: Preparation of benzyl N-((((bis(benzyloxy)phosphoryl)oxy)methoxy)carbonyl)-N-(3-hydroxy-2,2-dimethylpropyl)glycinate

To a stirred solution of benzyl N-((((bis(benzyloxy)phosphoryl)oxy)methoxy)carbonyl)-N-(3-((tert-butyldimethylsilyl)oxy)-2,2-dimethylpropyl)glycinate (0.49 g, 0.7 mmol) in MeOH (15 mL) was added Dowex® 50W×8 hydrogen form (0.5 g). The resulting mixture was stirred at room temperature for overnight. The mixture was filtered through Celite and the solvent was removed under vacuum. The resulting crude material was purified by silica gel column chromatography (0-100% EtOAc/hexane) to obtain the title compound. MS (m/z) 585.9 [M+H]+.

### Step 6: Preparation of benzyl N-((((bis(benzyloxy)phosphoryl)oxy)methoxy)carbonyl)-N-(3-((((((3′S,5S,7′R)-10′-((2,4-difluorobenzyl)carbamoyl)-3,3′-dimethyl-1′,11′-dioxo-1′,4,5′11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2,2-dimethylpropyl)glycinate

To a stirred solution of Intermediate D (0.28 g, 0.41 mmol) in acetonitrile (5 mL) was added benzyl N-((((bis(benzyloxy)phosphoryl)oxy)methoxy)carbonyl)-N-(3-hydroxy-2,2-dimethylpropyl)glycinate (0.29 g, 0.49 mmol) and 4-dimethylaminopyridine (0.05 g, 0.41 mmol). The resulting mixture was stirred at room temperature for overnight. The mixture was diluted with EtOAc and washed with H₂O, brine and dried by MgSO₄. The solvent was removed under vacuum and the resulting crude material was purified by silica gel column chromatography (0-100% EtOAc/hexane) to obtain the title compound. MS (m/z) 1128.9 [M+H]+.

### Step 7: Preparation of N-(3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2,2-dimethylpropyl)-N-(((phosphonooxy)methoxy)carbonyl)glycine (37)

A solution of benzyl N-((((bis(benzyloxy)phosphoryl)oxy)methoxy)carbonyl)-N-(3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2,2-dimethylpropyl)glycinate (0.26 g, 0.23 mmol) in THF (25 mL) was added 10% Pd/C (0.0245 g, 0.023 mmol). The mixture was purged with H₂ and stirred at room temperature for 1 hour. The mixture was filtered through Celite and washed with THF. The solvent was removed under vacuum and the residue was purified by prep-HPLC (5-100% MeCN/water containing 0.1% TFA) to obtain the title compound. MS (m/z) 857.96 [M+H]+. 1H NMR (400 MHz, Chloroform-d) δ 10.78-10.45 (m, 1H), 8.58 (d, J=4.2 Hz, 1H), 7.35 (td, J=8.4, 6.3 Hz, 1H), 6.90-6.79 (m, 2H), 6.00-5.69 (m, 2H), 5.53 (dd, J=37.4, 10.3 Hz, 2H), 4.83 (d, J=7.9 Hz, 1H), 4.62 (d, J=5.6 Hz, 2H), 4.30 (s, 1H), 3.93 (ddd, J=30.2, 25.1, 8.8 Hz, 4H), 3.74 (d, J=14.5 Hz, 2H), 3.31 (q, J=13.5, 11.7 Hz, 1H), 3.26-3.01 (m, 2H), 2.61 (d, J=18.0 Hz, 1H), 2.05 (s, 3H), 1.90 (d, J=18.2 Hz, 3H), 1.53 (s, 1H), 1.24 (d, J=6.4 Hz, 3H), 0.96 (dd, J=6.3, 3.2 Hz, 6H).

### Example 38: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((S)-2-(phosphonooxy)propyl) carbonate (38)

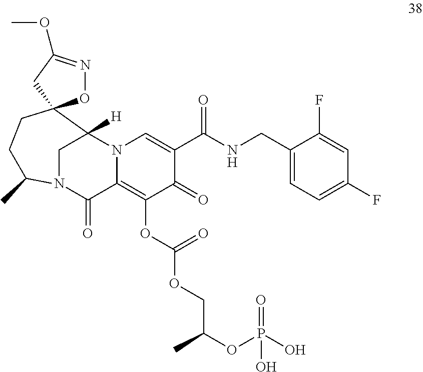 [SMILES: COC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1]

The title compound was prepared in a manner similar to Example 6 except using(S)-dibenzyl (1-hydroxypropan-2-yl) phosphate, prepared according to WO2019136112, instead of dibenzyl(2-hydroxyethyl) phosphate in Step 1 and Intermediate C instead of Intermediate B in Step 2. MS (m/z) 685.04 [M+H]⁺. 1H NMR (400 MHz, DMSO-d6) δ 10.12 (t, J=6.0 Hz, 1H), 8.90 (s, 1H), 7.42 (td, J=8.7, 6.6 Hz, 1H), 7.24 (ddd, J=10.4, 9.3, 2.6 Hz, 1H), 7.14-7.02 (m, 1H), 4.80 (s, 1H), 4.66-4.41 (m, 4H), 4.25 (dd, J=11.2, 4.4 Hz, 1H), 4.17 (dd, J=11.2, 4.4 Hz, 1H), 3.88-3.76 (m, 4H), 3.70 (dd, J=15.2, 1.9 Hz, 1H), 3.09 (d, J=16.7 Hz, 1H), 2.79 (d, J=16.9 Hz, 1H), 1.98-1.74 (m, 3H), 1.33-1.22 (m, 4H), 1.17 (d, J=6.7 Hz, 3H).

### Example 39: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((S)-2-(phosphonooxy)propyl) carbonate (39)

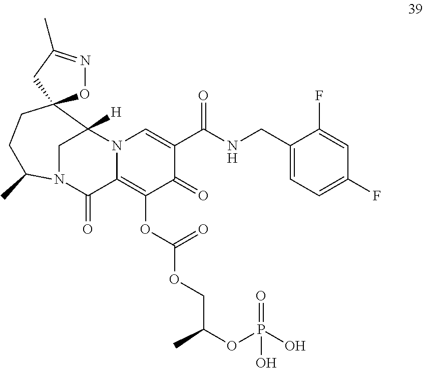 [SMILES: CC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1]

The title compound was prepared in a manner similar to Example 11, except using (S)-dibenzyl (1-hydroxypropan-2-yl) phosphate, prepared according to WO2019136112, instead of dibenzyl (3-hydroxypropyl) phosphate. MS (m/z) 669.1 [M+H]⁺. 1H NMR (400 MHz, DMSO-d6) δ 10.12 (t, J=6.0 Hz, 1H), 8.86 (s, 1H), 7.42 (td, J=8.7, 6.6 Hz, 1H), 7.25 (ddd, J=10.5, 9.3, 2.6 Hz, 1H), 7.08 (tdd, J=8.5, 2.6, 1.0 Hz, 1H), 4.72 (d, J=4.7 Hz, 1H), 4.63-4.52 (m, 3H), 4.48 (dtd, J=8.7, 6.8, 4.8 Hz, 1H), 4.26 (dd, J=11.1, 4.5 Hz, 1H), 4.18 (dd, J=11.1, 4.4 Hz, 1H), 3.83 (dd, J=15.2, 2.7 Hz, 1H), 3.71 (dt, J=13.9, 2.8 Hz, 1H), 3.03 (d, J=17.5 Hz, 1H), 2.67 (d, J=17.5 Hz, 1H), 1.95 (s, 3H), 1.84-1.73 (m, 3H), 1.28 (d, J=6.4 Hz, 3H), 1.26-1.21 (m, 1H), 1.17 (d, J=6.6 Hz, 3H).

### Example 40: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-(phosphonooxy)butanoate (40)

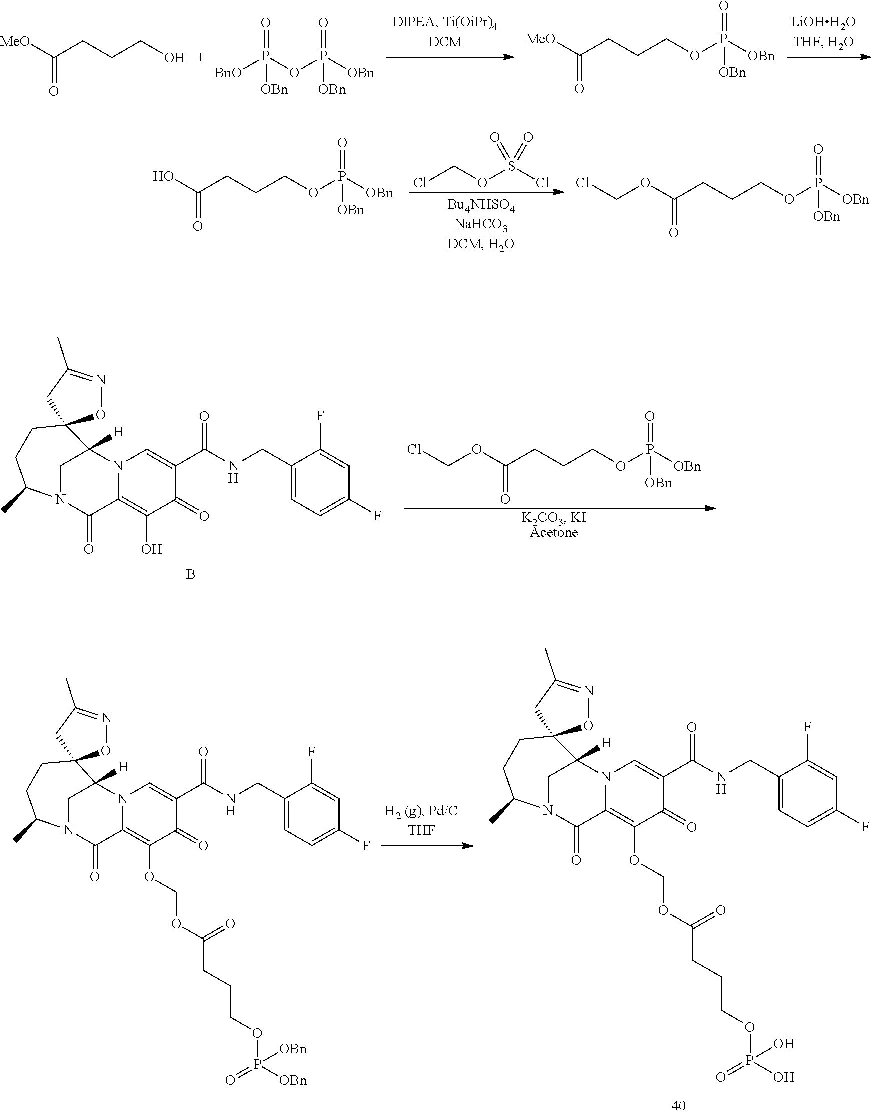

### Step 1: Preparation of methyl 4-((bis(benzyloxy)phosphoryl)oxy)butanoate

To a mixture of methyl 4-hydroxybutanoate (0.491 g, 4.16 mmol) in DCM (4 mL) was added DIPEA (1.66 mL, 9.25 mmol) followed by tetrabenzylpyrophosphate (2.00 g, 3.71 mmol) and Ti(i-PrO)₄ (0.187 mL, 0.631 mmol). The mixture was stirred at rt overnight. MgSO₄ and silica gel (4:1) were added to reaction, stirred for 10 min, then filtered and washed with 4:3 EtOAc/hexanes. The filtrate was concentrated and purified by reverse phase prep HPLC (10-100% MeCN/water) and lyophilized to afford the title compound. MS (m/z) 378.73 [M+H]+.

### Step 2: Preparation of 4-((bis(benzyloxy)phosphoryl)oxy)butanoic acid

To a solution of methyl 4-((bis(benzyloxy)phosphoryl)oxy)butanoate (0.224 g, 0.592 mmol) in THF (2.7 mL) and water (1.8 mL) at 0° C. was added LiOH—H₂O (0.0497 g, 1.18 mmol). The reaction mixture was stirred at 0° C. for 3 h and poured into 1 M HCl at 0° C. EtOAc was added and the phases were separated. The aqueous phase was extracted with EtOAc (2×) and the combined organic phase was washed with brine. The organic phase was dried over Na₂SO₄, filtered, and concentrated. The crude residue was dissolved in DCM/MeOH and purified by silica gel column chromatography (0-20% MeOH/DCM) to afford the title compound. MS (m/z) 364.83 [M+H]+.

### Step 3: Preparation of chloromethyl 4-((bis(benzyloxy)phosphoryl)oxy)butanoate

To a solution of 4-((bis(benzyloxy)phosphoryl)oxy)butanoic acid (0.154 g, 0.423 mmol) in DCM (1.5 mL) at 0° C. was added a suspension of NaHCO₃ (0.178 g, 2.11 mmol) in water (0.6 mL). Tetrabutylammonium hydrogen sulfate (0.0144 g, 0.042 mmol) was added to the biphasic mixture followed by chloromethyl chlorosulfate (0.107 mL, 1.06 mmol). The mixture was allowed to warm to rt and stir for 3 h. The mixture was partitioned between saturated aqueous NaHCO₃ and DCM. The phases were separated, and the aqueous phase was extracted with DCM (2×). The combined organic phase was washed with brine, dried over Na₂SO₄, filtered, and concentrated to afford the title compound. MS (m/z) 412.82 [M+H]+.

### Step 4: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((bis(benzyloxy)phosphoryl)oxy)butanoate

To a solution of chloromethyl 4-((bis(benzyloxy)phosphoryl)oxy)butanoate (0.127 g, 0.308 mmol) in acetone (3 mL) was added Intermediate B (0.125 g, 0.257 mmol), potassium iodide (0.056 g, 0.334 mmol), and potassium carbonate (0.071 g, 0.514 mmol). The reaction mixture was left to stir overnight at rt. The mixture was partitioned between EtOAc and water and the phases were separated. The organic phase was washed with brine, dried over Na₂SO₄, filtered, and concentrated to afford the title compound. MS (m/z) 862.85 [M+H]+.

### Step 5: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-(phosphonooxy)butanoate (40)

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((bis(benzyloxy)phosphoryl)oxy)butanoate (0.143 g, 0.166 mmol) in THF (17 mL) was added 10% Pd/C (0.018 g, 0.017 mmol). The flask was evacuated and backfilled with H₂ (g) (2×) then sparged with H₂ (g) for 2 min. The reaction mixture was left to stir under an atmosphere of hydrogen for 4 h then filtered, rinsing with THE. The filtrate was concentrated and the residue was purified by reverse phase prep HPLC (10-100% MeCN/water w/0.1% TFA) and lyophilized to afford the title compound. MS (m/z) 682.82 [M+H]⁺. 1H NMR (400 MHz, DMSO-d6) δ 10.29 (t, J=6.0 Hz, 1H), 8.69 (s, 1H), 7.42 (td, J=8.7, 6.6 Hz, 1H), 7.24 (ddd, J=10.5, 9.3, 2.6 Hz, 1H), 7.07 (tdd, J=8.6, 2.6, 1.0 Hz, 1H), 5.80 (d, J=6.2 Hz, 1H), 5.60 (d, J=6.3 Hz, 1H), 4.70-4.60 (m, 2H), 4.60-4.48 (m, 2H), 3.81 (q, J=6.7 Hz, 2H), 3.70-3.64 (m, 2H), 2.99 (d, J=17.6 Hz, 1H), 2.63 (d, J=17.6 Hz, 1H), 2.37 (t, J=7.4 Hz, 2H), 1.94 (s, 3H), 1.78 (ddt, J=17.2, 10.4, 6.1 Hz, 5H), 1.36-1.21 (m, 1H), 1.15 (d, J=6.7 Hz, 3H).

### Example 41: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3,3-dimethyl-4-(phosphonooxy)butanoate (41)

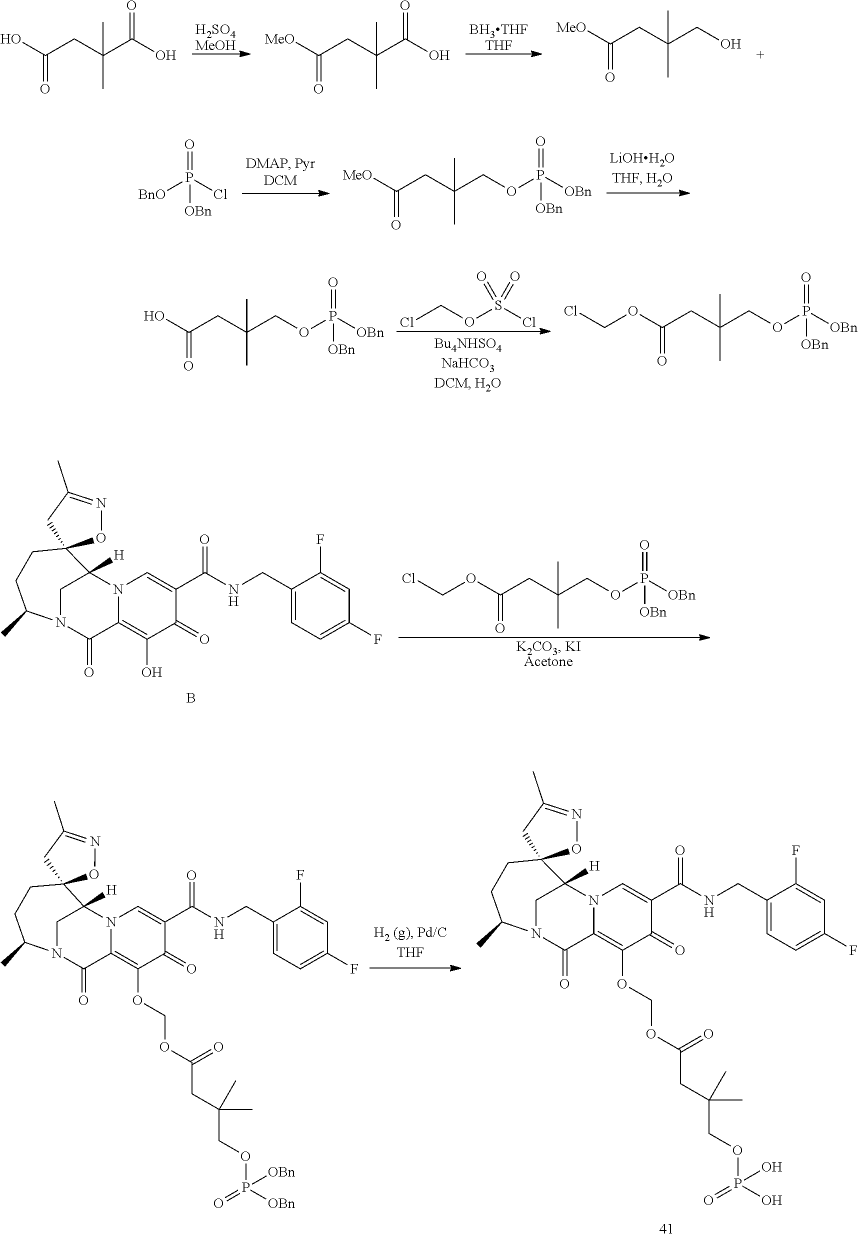

### Step 1: Preparation of 4-methoxy-2,2-dimethyl-4-oxobutanoic acid

To a solution of 2,2-dimethylsuccinic acid (10 g, 0.068 mol) in MeOH (50 mL) was added H₂SO₄ (conc, 0.6 g) at 0° C. It was stirred at r.t. overnight. After cooled to r.t., it was diluted with NaHCO₃ solution and washed with hexane. The aqueous phase was adjusted to pH=2 and extracted with EtOAc. The combined organic phase was dried and concentrated to give the title compound. ¹H NMR (400 MHz, CDCl₃) δ 3.68 (s, 3H), 2.62 (s, 2H), 1.31 (s, 6H).

### Step 2: Preparation of methyl 4-hydroxy-3,3-dimethylbutanoate

To a solution of 4-methoxy-2,2-dimethyl-4-oxobutanoic acid (10 g, 62.4 mmol) in THF (150 mL) was added BH₃·THF (1M in THF, 62 mL) at 0° C. It was stirred at r.t. overnight. The reaction mixture was quenched with MeOH (20 mL) at 0° C. and concentrated. It was purified by silica gel chromatography (PE:EtOAc=5:1 to 2:1) to afford the title compound. 1H NMR (400 MHz, CDCl₃) δ 3.68 (s, 3H), 3.40 (s, 2H), 2.32 (s, 2H), 0.99 (s, 6H).

### Step 3: Preparation of methyl 4-((bis(benzyloxy)phosphoryl)oxy)-3,3-dimethylbutanoate

Dibenzylphosphite (8.64 g, 32.96 mmol) was added to a solution of N-chlorosuccinimide (4.84 g, 36.24 mmol) in toluene (160 mL) at room temperature for 2 h. The mixture was filtered and the filtrate was evaporated under vacuum to give dibenzyl phosphorochloridate.

To a stirred solution of crude dibenzyl phosphorochloridate (5.6 g, 13.8 mmol) in DCM (50 mL) under argon with an ice bath were sequentially added methyl 4-hydroxy-3,3-dimethylbutanoate (1.35 g, 9.2 mmol), pyridine (1.45 g, 18.4 mmol) and DMAP (122 mg, 1 mmol). The reaction mixture was stirred at r.t. for 4 hours. H₂O was added and extracted with DCM (2×100 mL). The organic phase was dried and concentrated, then purified by silica gel column chromatography (PE:EtOAc=5:1) to give the title compound. MS (m/z) 407.1 [M+H]+.

### Step 4: Preparation of 4-((bis(benzyloxy)phosphoryl)oxy)-3,3-dimethylbutanoic acid

methyl 4-((bis(benzyloxy)phosphoryl)oxy)-3,3-dimethylbutanoate (12.7 g, 31.3 mmol) was added to a solution of LiOH·H₂O (10.5 g, 250 mmol) in H₂O/THF (1:1, 150 mL). It was stirred at 40° C. for 8 hours. After cooled to r.t., H₂O (300 mL) was added and it was washed with EtOAc (2×200 mL). The pH of the aqueous phase was adjusted to 6 with 1M HCl. It was extracted with EtOAc (2×200 mL). The combined organic phase was washed with H₂O, brine, dried (Na₂SO₄ anhydrous) and filtered then concentrated. It was purified by silica gel column chromatography to give the title compound. MS (m/z) 393.1 [M+H]+.

### Step 5: Preparation of chloromethyl 4-((bis(benzyloxy)phosphoryl)oxy)-3,3-dimethylbutanoate

To a stirred solution of 4-((bis(benzyloxy)phosphoryl)oxy)-3,3-dimethylbutanoic acid (2.2 g, 5.5 mmol) in DCM (30 mL) with an ice bath was added a suspension of NaHCO₃ (1.66 g, 26.8 mmol) in water (10 mL). The reaction mixture was stirred with an ice bath for 15 minutes. To the resulting white suspension were added Bu₄NHSO₄ (200 mg, 0.6 mmol) and chloromethyl chlorosulfate (1.76 g, 10.72 mmol) via syringe. The reaction mixture was warmed gradually to room temperature and stirred for 1-3 hours. The reaction mixture was purified by silica gel chromatography, eluting with PE: EtOAc (from 1/0 to 2/1) to afford the title compound. MS (m/z) 441.1 [M+H]+.

### Step 6: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((bis(benzyloxy)phosphoryl)oxy)-3,3-dimethylbutanoate

To a solution of chloromethyl 4-((bis(benzyloxy)phosphoryl)oxy)-3,3-dimethylbutanoate (0.136 g, 0.308 mmol) in acetone (3 mL) was added Intermediate B (0.125 g, 0.257 mmol), potassium iodide (0.056 g, 0.334 mmol), and potassium carbonate (0.071 g, 0.514 mmol). The reaction mixture was left to stir overnight at rt. The reaction mixture was partitioned between EtOAc and water and the phases separated. The organic phase was washed with brine, dried over Na₂SO₄, filtered, and concentrated to afford the title compound. MS (m/z) 890.82 [M+H]+.

### Step 7: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3,3-dimethyl-4-(phosphonooxy)butanoate (41)

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((bis(benzyloxy)phosphoryl)oxy)-3,3-dimethylbutanoate (0.196 g, 0.220 mmol) in THF (20 mL) was added 10% Pd/C (0.023 g, 0.022 mmol). The flask was evacuated and backfilled with H₂ (g) (2×) then sparged with H₂ (g) for 2 min. The reaction mixture was left to stir under an atmosphere of hydrogen for 4 h then filtered, rinsing with THF. The filtrate was concentrated and the residue was purified by reverse phase prep HPLC (10-100% MeCN/water w/0.1% TFA) and lyophilized to afford the title compound. MS (m/z) 710.79 [M+H]+. 1H NMR (400 MHz, DMSO-d6) δ 10.29 (t, J=6.0 Hz, 1H), 8.68 (s, 1H), 7.42 (td, J=8.7, 6.6 Hz, 1H), 7.24 (ddd, J=10.5, 9.3, 2.6 Hz, 1H), 7.13-7.01 (m, 1H), 5.78 (d, J=6.3 Hz, 1H), 5.59 (d, J=6.3 Hz, 1H), 4.68-4.58 (m, 2H), 4.54 (t, J=6.2 Hz, 2H), 3.67 (d, J=2.3 Hz, 2H), 3.61-3.54 (m, 2H), 2.99 (d, J=17.5 Hz, 1H), 2.63 (d, J=17.6 Hz, 1H), 2.25 (d, J=1.4 Hz, 2H), 1.94 (s, 3H), 1.88-1.68 (m, 3H), 1.30 (dd, J=15.5, 10.8 Hz, 1H), 1.15 (d, J=6.7 Hz, 3H), 0.92 (d, J=2.3 Hz, 6H).

### Example 42: Preparation of N-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2,2-dimethyl-4-oxobutyl)-N-(((phosphonooxy)methoxy)carbonyl)glycine (42)

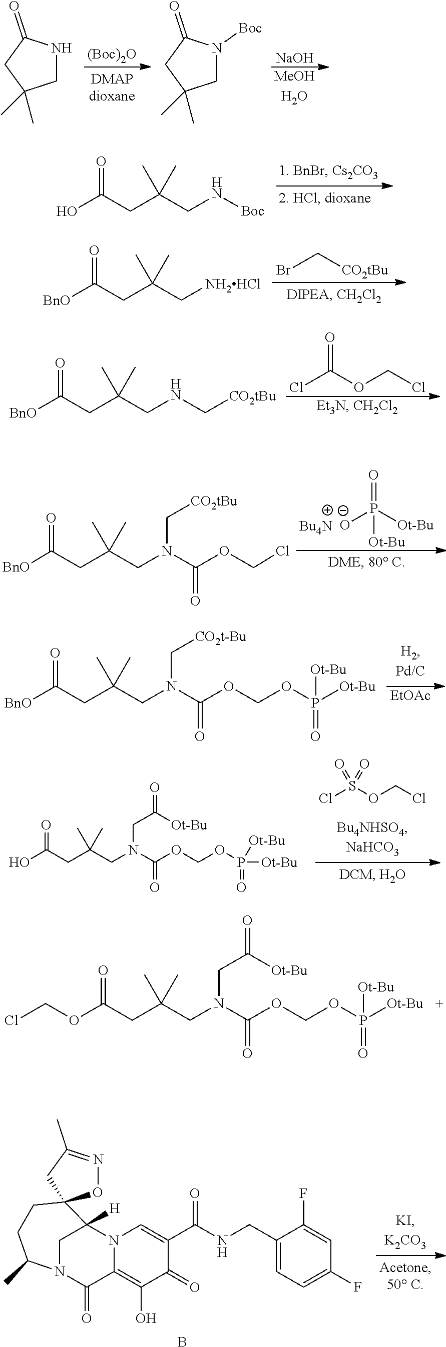

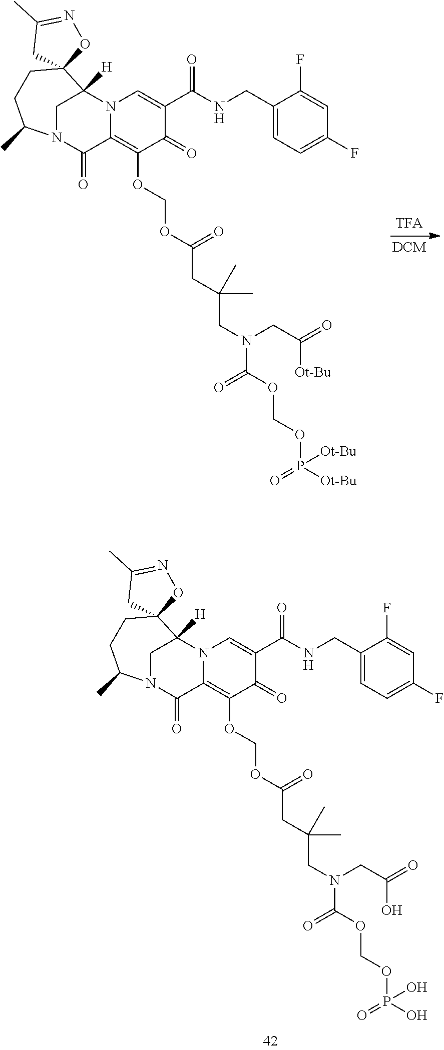 [SMILES: CC1=NO[C@@]2(CC[C@H](C)N3C[C@H]2n2cc(C(=O)NCc4ccc(F)cc4F)c(=O)c(OCOC(=O)CC(C)(C)CN(CC(=O)OC(C)(C)C)C(=O)OCOP(=O)(OC(C)(C)C)OC(C)(C)C)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)CN(CC(=O)O)C(=O)OCOP(=O)(O)O)c2C3=O)C1]

### Step 1: Preparation of tert-butyl 4,4-dimethyl-2-oxopyrrolidine-1-carboxylate

To a mixture of 4,4-dimethylpyrrolidin-2-one (67.8 g, 0.60 mol) and DMAP (87.9 g, 0.72 mol) in dioxane (1.5 L) was added (Boc) 20 (156.9 g, 0.72 mol) at rt. The mixture was stirred at 45° C. for 2 h. The reaction mixture was concentrated and the residue was dissolved in EtOAc (1.5 L), washed with 0.2N HCl (2×500 mL) and brine, dried over Na₂SO₄, concentrated in vacuum to give the title compound. MS (m/z) 158.2 [M-isobutene]⁺.

### Step 2: Preparation of 4-((tert-butoxycarbonyl)amino)-3,3-dimethylbutanoic acid

To a solution of tert-butyl 4,4-dimethyl-2-oxopyrrolidine-1-carboxylate (115 g, 540 mmol) in MeOH (1000 mL) was 2N NaOH (64.8 g, 1.62 mol) in H₂O (750 mL) at r.t. The mixture was stirred at r.t for 16 h. The reaction mixture was concentrated and the residue was adjusted to pH 3-4 with 2 N HCl, extracted with EtOAc (3×600 mL) and concentrated in vacuum to give the title compound. MS (m/z) 230.1 [M−H]+.

### Step 3: Preparation of benzyl 4-amino-3,3-dimethylbutanoate hydrochloride

To a solution of 4-((tert-butoxycarbonyl)amino)-3,3-dimethylbutanoic acid (118 g, 0.51 mol) in CH₃CN (1500 mL) was added Cs₂CO₃ (200 g, 0.61 mol) and BnBr (105 g, 0.61 mol) sequentially at r.t. The mixture was stirred at r.t for 16 h. The reaction mixture was filtered and concentrated, purified by chromatography on silica gel (0-10% EtOAc/PE) to give a residue. The residue was dissolved in dioxane (200 mL) and 4N HCl (200 mL, 797.5 mmol) was added at r.t. The mixture was stirred at r.t for 4 h and concentrated in vacuum to give the title compound as white solid. MS (m/z) 222.2 [M+H]⁺.

### Step 4: Preparation of benzyl 4-((2-(tert-butoxy)-2-oxoethyl)amino)-3,3-dimethylbutanoate

To a mixture of benzyl 4-amino-3,3-dimethylbutanoate hydrochloride (50 g, 199.2 mmol) and DIPEA (77.1 g, 597.6 mmol) in DCM (500 mL) was added t-butyl bromoacetate (42.7 g, 219.1 mmol) in DCM (50 mL) dropwise at 0° C. The mixture was stirred at 0° C. for 1.5 h. The reaction mixture was washed with 0.5 N HCl, water and brine to give the crude product in DCM. MS (m/z) 336.2 [M+H]⁺.

### Step 5: Preparation of benzyl 4-((2-(tert-butoxy)-2-oxoethyl)((chloromethoxy)carbonyl)amino)-3,3-dimethylbutanoate

To a stirred solution of benzyl 4-((2-(tert-butoxy)-2-oxoethyl)amino)-3,3-dimethylbutanoate (250 g, 0.75 mol) in DCM (7000 mL) at 0° C. was added Et₃N (188 g, 1.86 mol) and chloromethyl chloroformate (144 g, 1.12 mol). The reaction mixture was stirred at 0° C. for 2 h. The reaction was washed with sat. aq. solutions of NH₄Cl then brine. The organic layer was concentrated and purified by column chromatography (0-10% EtOAc/PE) to give the title product. MS (m/z) 426.2 [M−H]⁺.

### Step 6: Preparation of benzyl 4-((2-(tert-butoxy)-2-oxoethyl)((((di-tert-butoxyphosphoryl)oxy)methoxy)carbonyl)amino)-3,3-dimethylbutanoate

To a solution of benzyl 4-((2-(tert-butoxy)-2-oxoethyl) ((chloromethoxy)carbonyl)amino)-3,3-dimethylbutanoate (28 g, 65.4 mmol) in DME (200 mL) was added di-tert-butyl phosphate tetrabutylammonium salt (41.3 g, 91.6 mmol). The reaction mixture was stirred at 80° C. for 1 h. The mixture was cooled to room temperature, concentrated and dissolved in EtOAc. The organic layer was washed with water (3×), brine, dried over Na₂SO₄, filtered, and concentrated. The crude mixture was purified by column chromatography (0-50% EtOAc/PE) to afford the title compound. ¹H NMR (400 MHz, CDCl₃) δ: 7.34-7.33 (m, 5H), 5.60 (d, J=11.6 Hz, 1H), 5.56 (d, J=12.0 Hz, 1H), 5.10 (s, 2H), 3.96 (d, J=5.2 Hz, 2H), 3.32 (d, J=5.6 Hz, 2H), 2.33 (d, J=3.6 Hz, 2H), 1.48 (s, 18H), 1.45 (s, 9H), 1.03 (s, 6H).

### Step 7: Preparation of 4-((2-(tert-butoxy)-2-oxoethyl)((((di-tert-butoxyphosphoryl)oxy)methoxy)carbonyl)amino)-3,3-dimethylbutanoic acid

To a solution of benzyl 4-((2-(tert-butoxy)-2-oxoethyl)((((di-tert-butoxyphosphoryl)oxy)methoxy)carbonyl)amino)-3,3-dimethylbutanoate (4000 mg, 6.6 mmol) in EtOAc (25 mL) was added 10% Pd/C (1.4 g, 1.3 mmol). The flask was evacuated and backfilled with hydrogen gas (2×), then sparged with hydrogen for 2 min. The reaction mixture was left to stir under a hydrogen balloon atmosphere overnight. The reaction mixture was filtered, rinsed with EtOAc, and concentrated to afford the crude title compound. MS (m/z) 510.2 [M−H]+.

### Step 8: Preparation of chloromethyl 4-((2-(tert-butoxy)-2-oxoethyl)((((di-tert-butoxyphosphoryl)oxy)methoxy)carbonyl)amino)-3,3-dimethylbutanoate

A biphasic mixture of 4-((2-(tert-butoxy)-2-oxoethyl)((((di-tert-butoxyphosphoryl)oxy)methoxy)carbonyl)amino)-3,3-dimethylbutanoic acid (3 g, 5.86 mmol), tetrabutylammonium hydrogen sulfate (199 mg, 0.58 mmol), and sodium bicarbonate (3.94 g, 46.9 mmol) in water (15 mL) and DCM (15 mL) was cooled to 0° C. While stirring, chloromethyl chlorosulfate (1.94 g, 11.7 mmol) was added dropwise. The reaction mixture was allowed to stir at rt overnight. Brine was added and the aqueous phase was extracted with DCM (3×). The combined organic phase was concentrated. The crude product was purified by column chromatography (0-100% EtOAc/hexanes) to afford the title compound. MS (m/z) 581.9 [M+Na]⁺.

### Step 9: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((2-(tert-butoxy)-2-oxoethyl)((((di-tert-butoxyphosphoryl)oxy)methoxy)carbonyl)amino)-3,3-dimethylbutanoate

Intermediate B (107 mg, 0.22 mmol), K₂CO₃ (61 mg, 0.44 mmol) and KI (48 mg, 0.28 mmol) were mixed in acetone (3 mL) at room temperature. Then a solution of chloromethyl 4-((2-(tert-butoxy)-2-oxoethyl)((((di-tert-butoxyphosphoryl)oxy)methoxy)carbonyl)amino)-3,3-dimethylbutanoate (246 mg, 0.44 mmol) in acetone (1 mL) was added. The reaction mixture was heated at 50° C. for 3 h. The solvent was evaporated and the residue was dissolved in DCM and washed with saturated aqueous NH₄Cl (10 mL). The organic layer was separated and concentrated to dryness. The residue was purified by silica gel column chromatography (80% MeOH/DCM) to afford the title compound. MS (m/z) 1009.9 [M+H]⁺.

### Step 10: Preparation of N-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2,2-dimethyl-4-oxobutyl)-N-(((phosphonooxy)methoxy)carbonyl)glycine (42)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((2-(tert-butoxy)-2-oxoethyl)((((di-tert-butoxyphosphoryl)oxy)methoxy)carbonyl)amino)-3,3-dimethylbutanoate (250 mg, 0.21 mmol) was dissolved in DCM (3 mL) and TFA (0.3 mL) was added. The reaction mixture was stirred at rt for 5 h. The solvent was evaporated and the residue was purified with C-18 reverse phase column chromatography to afford the title compound. MS (m/z) 841.8 [M+H]⁺. 1H NMR (400 MHz, Methanol-d4) δ 8.57 (s, 1H), 7.46 (td, J=8.5, 6.4 Hz, 1H), 7.10-6.84 (m, 2H), 5.90 (dd, J=6.4, 2.2 Hz, 1H), 5.67 (dd, J=6.4, 1.1 Hz, 1H), 5.58 (dd, J=17.8, 13.2 Hz, 2H), 4.86-4.80 (m, 2H), 4.73-4.55 (m, 2H), 4.48 (d, J=2.4 Hz, 1H), 4.10 (d, J=24.4 Hz, 2H), 3.92-3.69 (m, 2H), 3.39 (dd, J=13.0, 3.8 Hz, 1H), 3.16 (d, J=17.9 Hz, 1H), 2.70 (d, J=18.0 Hz, 1H), 2.37 (d, J=8.9 Hz, 2H), 2.06 (s, 3H), 2.00-1.85 (m, 3H), 1.68-1.42 (m, 1H), 1.27 (dd, J=6.8, 1.5 Hz, 3H), 1.08-0.91 (m, 6H).

### Example 43: Preparation of N-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-3,3-dimethyl-4-oxobutyl)-N-(((phosphonooxy)methoxy)carbonyl)glycine (43)

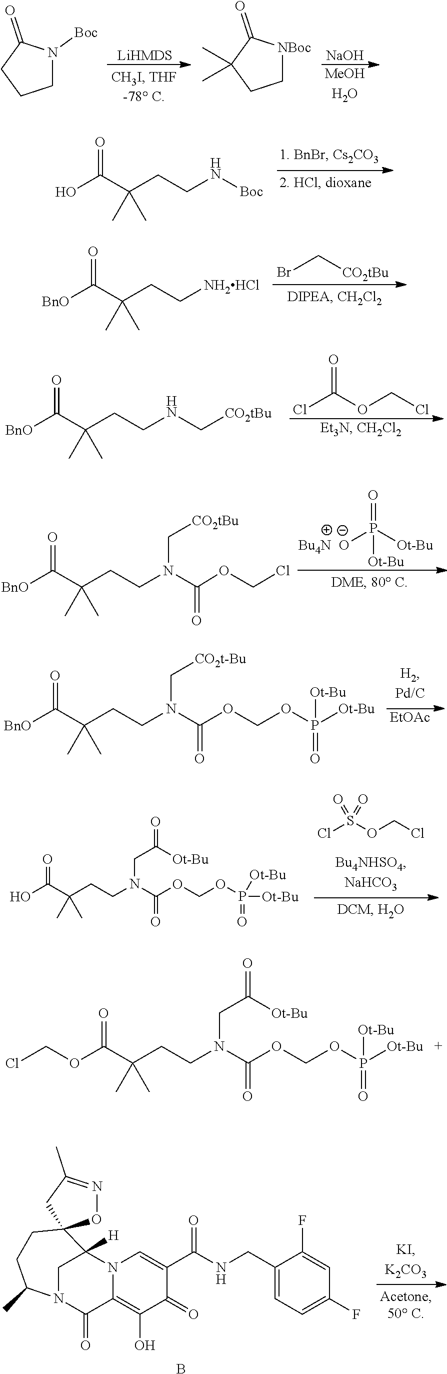

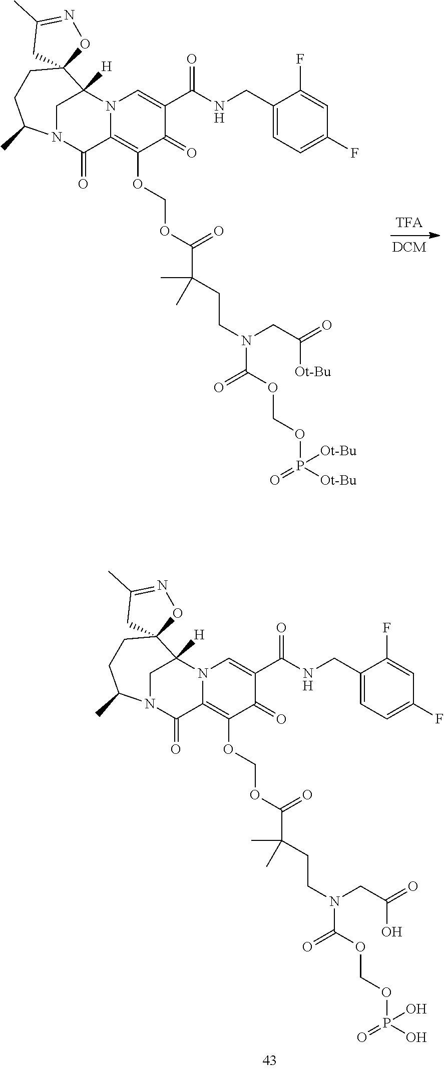 [SMILES: CC1=NO[C@@]2(CC[C@H](C)N3C[C@H]2n2cc(C(=O)NCc4ccc(F)cc4F)c(=O)c(OCOC(=O)C(C)(C)CCN(CC(=O)OC(C)(C)C)C(=O)OCOP(=O)(OC(C)(C)C)OC(C)(C)C)c2C3=O)C1 ; CC1=NO[C@@]2(CC[C@H](C)N3C[C@H]2n2cc(C(=O)NCc4ccc(F)cc4F)c(=O)c(OCOC(=O)C(C)(C)CCN(CC(=O)O)C(=O)OCOP(=O)(O)O)c2C3=O)C1]

### Step 1: Preparation of tert-butyl 3,3-dimethyl-2-oxopyrrolidine-1-carboxylate

To a mixture of tert-butyl 2-oxopyrrolidine-1-carboxylate (600 g, 3.24 mol) and CH3I (2303 g, 16.22 mol) in THF (3200 mL) was added 1M LiHMDS (8760 mL, 8.76 mol) at −78° C. The mixture was stirred at −78° C. for 1 h. The reaction mixture was quenched by saturated ammonium chloride solution, extracted with EtOAc (3×1000 mL), washed with brine, dried over Na₂SO₄ and concentrated. The residue was purified by column chromatography on silica gel (0-10% EtOAc/PE) to give the title compound. MS (m/z) 158.2 [M-isobutene]⁺.

### Step 2: Preparation of 4-((tert-butoxycarbonyl)amino)-2,2-dimethylbutanoic acid

To a solution of tert-butyl 3,3-dimethyl-2-oxopyrrolidine-1-carboxylate (370 g, 1.74 mol) in THF (1500 mL) and EtOH (1500 mL) was added NaOH (347 g, 5.0 mmol) in H₂O (700 mL) at r.t. The mixture was stirred at r.t for 16 h. The reaction mixture was adjusted pH to 3-4 with 2 N HCl, extracted with EtOAc (3×2000 mL), washed with brine, dried over Na₂SO₄ and concentrated in vacuum to give the title compound. MS (m/z) 230.1 [M−H]⁺.

### Step 3: Preparation of benzyl 4-amino-2,2-dimethylbutanoate hydrochloride

To a solution of 4-((tert-butoxycarbonyl)amino)-2,2-dimethylbutanoic acid (350 g, 1.52 mol) in MeCN (4900 mL) was added Cs₂CO₃ (593 g, 1.82 mol) and BnBr (311 g, 1.82 mol) sequentially at r.t. The mixture was stirred at r.t for 16 h. The reaction mixture was filtered, concentrated, and purified by chromatography on silica gel (0-10% EtOAc/PE) to afford a residue. To a solution of the purified residue in dioxane (1300 mL) was added 4N HCl/dioxane (1270 mL, 1.27 mol) at r.t. and stirred at r.t for 4 h. The reaction mixture was concentrated in vacuo to give the title compound. MS (m/z) 222.2 [M+H]⁺.

### Step 4: Preparation of benzyl 4-((2-(tert-butoxy)-2-oxoethyl)amino)-2,2-dimethylbutanoate

To a mixture of benzyl 4-amino-2,2-dimethylbutanoate hydrochloride (328 g, 1.27 mol) and DIPEA (494 g, 3.83 mol) in DCM (11 L) was added dropwise t-butyl bromoacetate (124 g, 0.32 mol) in DCM (500 mL) at 0° C. The mixture was stirred at 0° C. for 1 h and then warmed to room temp for 16 h. The reaction mixture was washed with water and brine. The organic layer was concentrated and purified by column chromatography (0-10% MeOH/CH2Cl2) to give the desired product. MS (m/z) 336.2 [M+H]⁺.

### Step 5: Preparation of benzyl 4-((2-(tert-butoxy)-2-oxoethyl)((chloromethoxy)carbonyl)amino)-2,2-dimethylbutanoate

To a stirred solution of benzyl 4-((2-(tert-butoxy)-2-oxoethyl)amino)-2,2-dimethylbutanoate (250 g, 0.75 mol) and triethylamine (188 g, 1.86 mol) in DCM (7000 mL) was added chloromethyl chloroformate (144 g, 1.12 mol) at 0° C. and stirred at 0° C. for 1 h. The reaction mixture was quenched by sat. aq. NH₄Cl, washed with brine, concentrated and purified by column chromatography (0-10% EtOAc/PE) to give the desired product. MS (m/z) 426.2 [M−H]⁺.

### Step 6: Preparation of benzyl 4-((2-(tert-butoxy)-2-oxoethyl)((((di-tert-butoxyphosphoryl)oxy)methoxy)carbonyl)amino)-2,2-dimethylbutanoate

To a solution of benzyl 4-((2-(tert-butoxy)-2-oxoethyl) ((chloromethoxy)carbonyl)amino)-2,2-dimethylbutanoate (21.2 g, 49.1 mmol) in DME (180 mL) was added di-tert-butyl phosphate tetrabutylammonium salt (31.5 g, 69.7 mmol). The reaction mixture was stirred at 80° C. for 1 h. The mixture was cooled to room temperature, concentrated and dissolved in EtOAc. The organic layer was washed with water (3×), brine, dried over Na₂SO₄, filtered, and concentrated. The crude mixture was purified by column chromatography (0-50% EtOAc/PE) to afford the title product. MS (m/z) 624.2 [M+Na]⁺. 1H NMR (400 MHz, CDCl3) δ: 7.36-7.31 (m, 5H), 5.58 (t, J=10.8 Hz, 2H), 5.10 (d, J=1.2 Hz, 2H), 3.79 (d, J=2.8 Hz, 2H), 3.31-3.26 (m, 2H), 1.85-1.80 (m, 2H), 1.48 (s, 9H), 1.47 (s, 9H), 1.45 (s, 9H), 1.23 (s, 3H), 1.22 (s, 3H).

### Step 7: Preparation of 4-((2-(tert-butoxy)-2-oxoethyl)((((di-tert-butoxyphosphoryl)oxy)methoxy)carbonyl)amino)-2,2-dimethylbutanoic acid

To a solution of benzyl 4-((2-(tert-butoxy)-2-oxoethyl)((((di-tert-butoxyphosphoryl)oxy)methoxy)carbonyl)amino)-2,2-dimethylbutanoate (1151 mg, 1.9 mmol) in EtOAc (25 mL) was added 10% Pd/C (407 mg, 0.38 mmol). The flask was evacuated and backfilled with hydrogen gas (2×), then sparged with hydrogen for 2 min. The reaction mixture was left to stir under a hydrogen balloon atmosphere overnight. The reaction mixture was filtered, rinsed with EtOAc, and concentrated to afford crude product. MS (m/z) 510.2 [M−H]+.

### Step 8: Preparation of chloromethyl 4-((2-(tert-butoxy)-2-oxoethyl)((((di-tert-butoxyphosphoryl)oxy)methoxy)carbonyl)amino)-2,2-dimethylbutanoate

A biphasic mixture of 4-((2-(tert-butoxy)-2-oxoethyl)((((di-tert-butoxyphosphoryl)oxy)methoxy)carbonyl)amino)-2,2-dimethylbutanoic acid (0.98 g, 1.92 mmol), tetrabutylammonium hydrogen sulfate (65 mg, 0.19 mmol), and sodium bicarbonate (1.29 g, 15.3 mmol) in water and DCM was cooled to 0° C. While stirring, chloromethyl chlorosulfate (0.63 g, 3.83 mmol) was added dropwise. The reaction mixture was allowed to stir at rt overnight. Brine was added and the aqueous phase was extracted with DCM (3×). The combined organic phase was concentrated. The crude product was purified by column chromatography (0-100% EtOAc/hexanes) to afford the title compound. MS (m/z) 581.9 [M+Na]+.

### Step 9: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((2-(tert-butoxy)-2-oxoethyl)((((di-tert-butoxyphosphoryl)oxy)methoxy)carbonyl)amino)-2,2-dimethylbutanoate

Intermediate B (115 mg, 0.24 mmol), K₂CO₃ (65 mg, 0.47 mmol) and KI (51 mg, 0.31 mmol) were mixed in acetone (3 mL) at room temperature. Then a solution of chloromethyl 4-((2-(tert-butoxy)-2-oxoethyl)((((di-tert-butoxyphosphoryl)oxy)methoxy)carbonyl)amino)-2,2-dimethylbutanoate (264 mg, 0.47 mmol) in acetone (1 mL) was added. The reaction mixture was heated at 50° C. for 3 h. The solvent was evaporated, dissolved in DCM, and washed with aqueous saturated NH₄Cl (10 mL). The organic layer was separated and concentrated to dryness. The residue was purified with silica gel column chromatography (80% MeOH/DCM) to afford the title compound. MS (m/z) 1009.8 [M+H]+.

### Step 10: Preparation of N-(4-((((3′S,5S,7′R)-10′-((2,4-difluorobenzyl)carbamoyl)-3,3′-dimethyl-1′,11′-dioxo-1′,4′,5′, 11′-tetrahydro-3′H,4H,7′H-spiro[isoxazole-5,6′-[2,7]methanopyrido[1,2-a][1,4]diazonin]-12′-yl)oxy)methoxy)-3,3-dimethyl-4-oxobutyl)-N-(((phosphonooxy)methoxy)carbonyl)glycine (43)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((2-(tert-butoxy)-2-oxoethyl)((((di-tert-butoxyphosphoryl)oxy)methoxy)carbonyl)amino)-2,2-dimethylbutanoate (155 mg, 0.15 mmol) was dissolved in DCM (3 mL) and TFA (0.3 mL) was added. The reaction mixture was stirred at rt for 5 h. The solvent was evaporated and the residue was purified by reverse phase C-18 column chromatography to afford the title compound. MS (m/z) 841.7 [M+H]⁺. 1H NMR (400 MHz, Methanol-d4) δ 8.57 (s, 1H), 7.59-7.27 (m, 1H), 7.07-6.86 (m, 2H), 5.86 (dd, J=11.1, 6.3 Hz, 1H), 5.70 (d, J=6.3 Hz, 1H), 5.65-5.43 (m, 2H), 4.81 (q, J=7.7 Hz, 1H), 4.72-4.54 (m, 2H), 4.52 (d, J=2.3 Hz, 1H), 4.12-4.04 (m, 1H), 4.02 (d, J=1.2 Hz, 1H), 3.89-3.68 (m, 2H), 3.39 (ddd, J=9.2, 5.7, 1.7 Hz, 1H), 3.30-3.25 (m, 1H), 3.24-3.05 (m, 1H), 2.70 (ddd, J=18.1, 2.9, 1.3 Hz, 1H), 2.05 (s, 3H), 1.99-1.71 (m, 5H), 1.64-1.46 (m, 1H), 1.26 (d, J=6.7 Hz, 3H), 1.22-1.12 (m, 6H).

### Example 44: Preparation of N-(2-(1-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)cyclopropyl)ethyl)-N-(((phosphonooxy)methoxy)carbonyl)glycine (44)

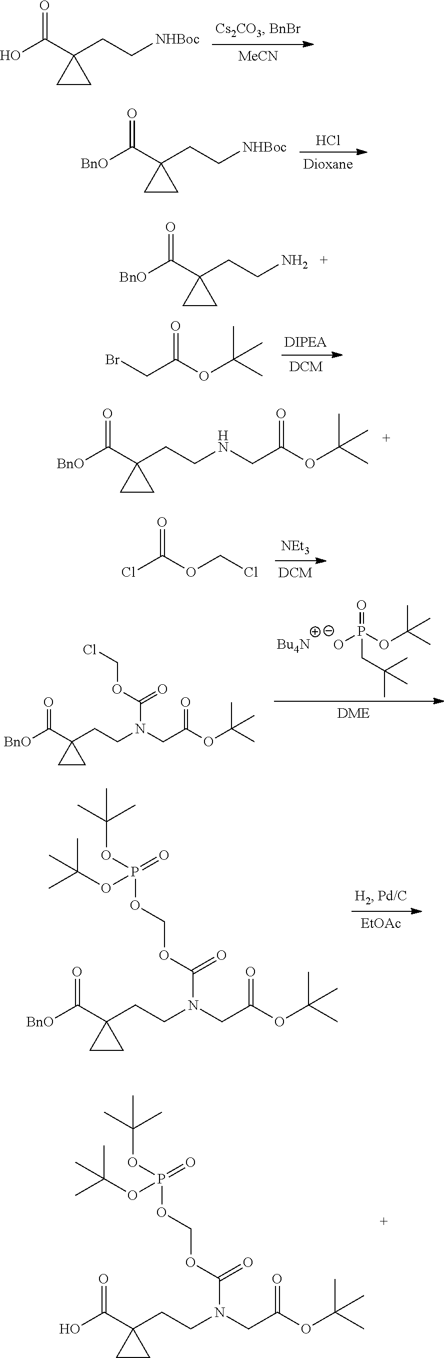

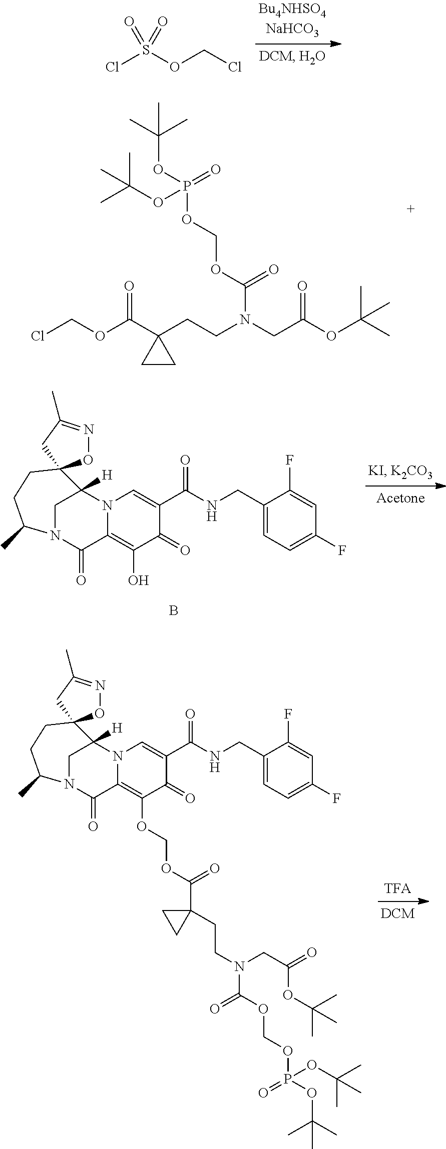

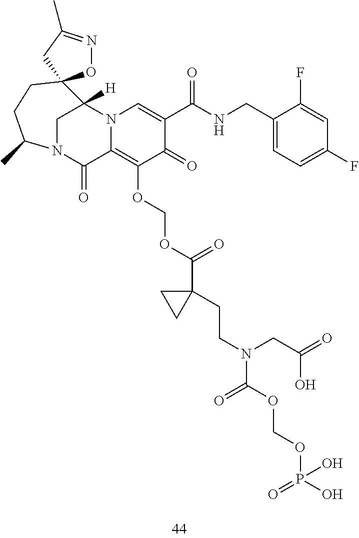 [SMILES: CC1=NO[C@@]2(CC[C@H](C)N3C[C@H]2n2cc(C(=O)NCc4ccc(F)cc4F)c(=O)c(OCOC(=O)C4(CCN(CC(=O)O)C(=O)OCOP(=O)(O)O)CC4)c2C3=O)C1]

### Step 1: Preparation of benzyl 1-(2-((tert-butoxycarbonyl)amino)ethyl)cyclopropane-1-carboxylate

1-(2-((tert-butoxycarbonyl)amino)ethyl)cyclopropane-1-carboxylic acid (25 g, 97 mmol) was dissolved in MeCN (200 mL), and Cs₂CO₃ and BnBr were added to the mixture. The mixture was stirred at RT overnight. The reaction mixture was then filtered and concentrated. The residue was purified by column chromatography to afford benzyl 1-(2-((tert-butoxycarbonyl)amino)ethyl)cyclopropane-1-carboxylate. MS (m/z) 264.0 [M-isobutene]⁺.

### Step 2: Preparation of benzyl 1-(2-aminoethyl)cyclopropane-1-carboxylate

Benzyl 1-(2-((tert-butoxycarbonyl)amino)ethyl)cyclopropane-1-carboxylate (5.1 g, 16 mmol) was treated with 4N HCl (64 mL) in dioxane (5 mL). After stirring for 1 h, the reaction mixture was concentrated to afford benzyl 1-(2-aminoethyl)cyclopropane-1-carboxylate. MS (m/z) 220.0 [M+H]⁺.

### Step 3: Preparation of benzyl 1-(2-((2-(tert-butoxy)-2-oxoethyl)amino)ethyl)cyclopropane-1-carboxylate

To a stirred suspension of benzyl 1-(2-aminoethyl)cyclopropane-1-carboxylate (4.1 g, 16 mmol) in CH2Cl₂ (100 mL) at 0° C. was added DIPEA (6.2 g, 48 mmol). t-Butyl bromoacetate (1.6 g, 8.0 mmol) in CH2Cl₂ (100 mL) was added dropwise over 30 min, then warmed to rt. The reaction mixture was then washed with 0.5 M aq. HCl solution and brine. The organic layer was dried over Na₂SO₄, filtered, and concentrated. The residue was purified by column chromatography to afford benzyl 1-(2-((2-(tert-butoxy)-2-oxoethyl)amino)ethyl)cyclopropane-1-carboxylate. MS (m/z) 334.10 [M+H]⁺.

### Step 4: Preparation of benzyl 1-(2-((2-(tert-butoxy)-2-oxoethyl) ((chloromethoxy)carbonyl)amino)ethyl)cyclopropane-1-carboxylate

To a solution of benzyl 1-(2-((2-(tert-butoxy)-2-oxoethyl)amino)ethyl)cyclopropane-1-carboxylate (1059 mg, 3.2 mmol) in DCM (15 mL) at 0° C. was added NEt₃ (0.48 g, 4.8 mmol), followed by addition of chloromethyl chloroformate (0.61 g, 4.8 mmol) dropwise. After stirring at 0° C. for 10 min, the reaction was washed with brine. The organic layer was dried over anhydrous MgSO₄, filtered, and concentrated. The reside was purified with flash column to afford benzyl 1-(2-(N-(2-(tert-butoxy)-2-oxoethyl)-2-chloroacetamido)ethyl)cyclopropane-1-carboxylate. MS (m/z) 369.90 [M-isobutene]⁺.

### Step 5: Preparation of benzyl 1-(2-((2-(tert-butoxy)-2-oxoethyl)((((di-tert-butoxyphosphoryl)oxy)methoxy)carbonyl)amino)ethyl)cyclopropane-1-carboxylate

To a solution of benzyl 1-(2-((2-(tert-butoxy)-2-oxoethyl) ((chloromethoxy)carbonyl)amino)ethyl)cyclopropane-1-carboxylate (840 mg, 2.0 mmol) in DCM (10 mL) was added tetrabutylammonium di-tert-butyl phosphate (1.2 g, 2.8 mmol) and the reaction was heated to 80° C. for 2 h. The reaction was then washed with brine. The organic layer was dried over anhydrous MgSO₄, filtered, and concentrated. The reside was purified by column chromatography to afford benzyl 1-(2-((2-(tert-butoxy)-2-oxoethyl)((((di-tert-butoxyphosphoryl)oxy)methoxy)carbonyl)amino)ethyl)cyclopropane-1-carboxylate. MS (m/z) 621.9 [M+Na]⁺.

### Step 6: Preparation of 1-(2-((2-(tert-butoxy)-2-oxoethyl)((((di-tert-butoxyphosphoryl)oxy)methoxy)carbonyl)amino)ethyl)cyclopropane-1-carboxylic acid

To a solution of benzyl 1-(2-((2-(tert-butoxy)-2-oxoethyl)((((di-tert-butoxyphosphoryl)oxy)methoxy)carbonyl)amino)ethyl)cyclopropane-1-carboxylate (648 mg, 1.1 mmol) in EtOAc (5 mL) was added 10% Pd/C (0.2 g, 0.22 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered, rinsed with EtOAc, and concentrated to afford 1-(2-((2-(tert-butoxy)-2-oxoethyl)((((di-tert-butoxyphosphoryl)oxy)methoxy)carbonyl)amino)ethyl)cyclopropane-1-carboxylic acid. MS (m/z) 531.9 [M+Na]⁺.

### Step 7: Preparation of chloromethyl 1-(2-((2-(tert-butoxy)-2-oxoethyl)((((di-tert-butoxyphosphoryl)oxy)methoxy)carbonyl)amino)ethyl)cyclopropane-1-carboxylate

A biphasic mixture of 1-(2-((2-(tert-butoxy)-2-oxoethyl)((((di-tert-butoxyphosphoryl)oxy)methoxy)carbonyl)amino)ethyl)cyclopropane-1-carboxylic acid (0.55 g, 1.35 mmol), tetrabutylammonium hydrogen sulfate (46 mg, 0.14 mmol) and sodium bicarbonate (0.91 g, 10.8 mmol) in water and DCM was cooled to 0° C. While stirring, chloromethyl chlorosulfate (0.44 g, 2.7 mmol) was added dropwise. The reaction mixture was allowed to stir at rt overnight. Brine was added and the aqueous phase was extracted with DCM (3×). The combined organic phase was concentrated. The crude product was purified by column chromatography (0-100% EtOAc/hexanes) to afford chloromethyl 1-(2-((2-(tert-butoxy)-2-oxoethyl) ((((di-tert-butoxyphosphoryl)oxy)methoxy)carbonyl)amino)ethyl)cyclopropane-1-carboxylate. MS (m/z) 580.1 [M+Na]⁺.

### Step 8: Preparation of (((3′S,5S,7′R)-10′-((2,4-difluorobenzyl)carbamoyl)-3,3′-dimethyl-1′,11′-dioxo-l′,4′,5′,11′-tetrahydro-3′H,4H,7′H-spiro[isoxazole-5,6′-[2,7]methanopyrido[1,2-a][1,4]diazonin]-12′-yl)oxy)methyl 1-(2-((2-(tert-butoxy)-2-oxoethyl)((((di-tert-butoxyphosphoryl)oxy)methoxy)carbonyl)amino)ethyl)cyclopropane-1-carboxylate

Intermediate B (250 mg, 0.51 mmol), K₂CO₃ (142 mg, 1 mmol) and KI (111 mg, 0.67 mmol) were mixed in acetone (5 mL) at room temperature. Then a solution of chloromethyl 1-(2-((2-(tert-butoxy)-2-oxoethyl)((((di-tert-butoxyphosphoryl)oxy)methoxy)carbonyl)amino)ethyl)cyclopropane-1-carboxylate (459 mg, 0.82 mmol) in acetone (1 mL) was added. The reaction mixture was heated at 50° C. for 3 h. The solvent was evaporated and then dissolved in DCM. Washed with aqueous saturated NH₄Cl (10 mL). The organic layer was separated and concentrated to dryness. The residue was purified by silica gel column chromatography (80% MeOH/DCM) to afford the title compound. MS (m/z) 1007.8 [M+H]⁺.

### Step 9: N-(2-(1-(((((3′S,5S,7′R)-10′-((2,4-difluorobenzyl)carbamoyl)-3,3′-dimethyl-1′,11′-dioxo-1′4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)cyclopropyl)ethyl)-N-(((phosphonooxy)methoxy)carbonyl)glycine (44)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 1-(2-((2-(tert-butoxy)-2-oxoethyl)((((di-tert-butoxyphosphoryl)oxy)methoxy)carbonyl)amino)ethyl)cyclopropane-1-carboxylate (51 mg, 0.05 mmol) was dissolved in DCM (2 mL) and TFA (0.3 mL) was added. The reaction mixture was stirred at rt for 5 h. The solvent was evaporated, the residue was purified reverse phase chromatography to afford the title compound. MS (m/z) 839.70 [M+H]⁺. 1H NMR (400 MHz, Methanol-d4) δ 8.58 (d, J=4.1 Hz, 1H), 7.45 (q, J=7.8 Hz, 1H), 7.11-6.85 (m, 2H), 5.86 (dd, J=12.8, 6.3 Hz, 1H), 5.68 (dd, J=6.4, 4.8 Hz, 1H), 5.53 (q, J=10.3, 6.1 Hz, 2H), 4.86-4.78 (m, 1H), 4.74-4.55 (m, 2H), 4.51 (d, J=2.4 Hz, 1H), 4.13-3.94 (m, 2H), 3.92-3.73 (m, 2H), 3.57-3.39 (m, 2H), 3.16 (d, J=17.9 Hz, 1H), 2.71 (dd, J=18.2, 3.3 Hz, 1H), 2.05 (s, 3H), 1.98-1.46 (m, 6H), 1.34-1.13 (m, 5H), 0.88 (dd, J=19.5, 3.0 Hz, 2H).

### Example 45: Preparation of N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-N-methylglycine (45)

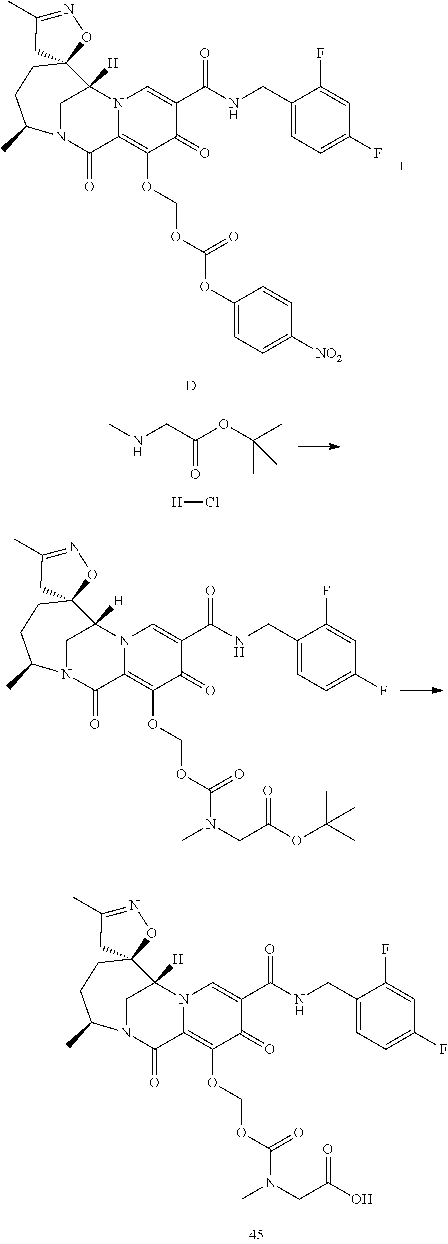

### Step 1: Preparation of tert-butyl N-(((((3′S,5S,7′R)-10′-((2,4-difluorobenzyl)carbamoyl)-3,3′-dimethyl-1′,11′-dioxo-1′4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-N-methylglycinate

To a mixture of Intermediate D (40.0 mg, 0.0587 mmol) in acetonitrile (1 mL) at room temperature was added tert-butyl methylglycinate hydrochloride HCl (21.3 mg, 0.117 mmol) and triethylamine (23.8 mg, 0.235 mmol). After being stirred for 30 minutes, the reaction was diluted with EtOAc, washed sequentially with water and brine, dried over sodium sulfate, filtered, and concentrated. The residue was purified by silica gel column chromatography (0-100% EtOAc/Hexane then 0-15% MeOH/EtOAc) to give title compound. MS (m/z) 687.86 [M]⁺.

### Step 2: Preparation of N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-N-methylglycine (45)

The residue from Step 1 was dissolved in DCM (2.0 mL) before it was cooled to 0° C. To this cold mixture was added TFA (0.4 mL). The mixture was stirred for 30 minutes and then removed from cooling bath and stirred at room temperature for 2 hours. The reaction was then concentrated, redissolved in DMF, filtered, and purified by reverse phase prep HPLC (10-100% MeCN/water containing 0.1% TFA) to give title compound. MS (m/z) 631.908 [M]⁺. 1H NMR (400 MHz, DMSO-d6) δ 10.32 (t, J=5.9 Hz, 1H), 8.66 (d, J=1.4 Hz, 1H), 7.43 (td, J=8.7, 6.6 Hz, 1H), 7.25 (ddd, J=10.5, 9.3, 2.6 Hz, 1H), 7.08 (td, J=8.6, 2.7 Hz, 1H), 5.72-5.59 (m, 2H), 4.71-4.50 (m, 4H), 3.94-3.85 (m, 2H), 3.62 (d, J=2.3 Hz, 2H), 3.00 (d, J=17.6 Hz, 1H), 2.87-2.79 (m, 3H), 2.63 (d, J=17.6 Hz, 1H), 1.95 (s, 3H), 1.86-1.70 (m, 3H), 1.39-1.27 (m, 1H), 1.16 (t, J=6.2 Hz, 3H).

### Example 46: Preparation of N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-N-(2-(phosphonooxy)ethyl)glycine (46)

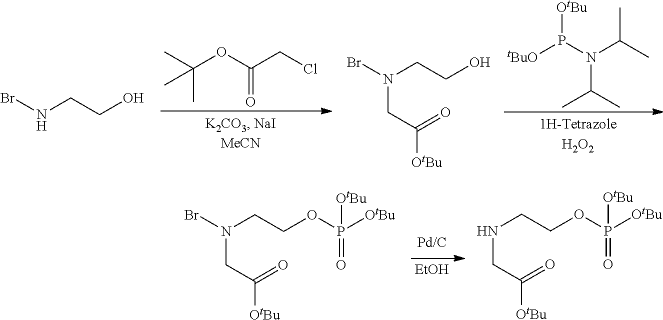

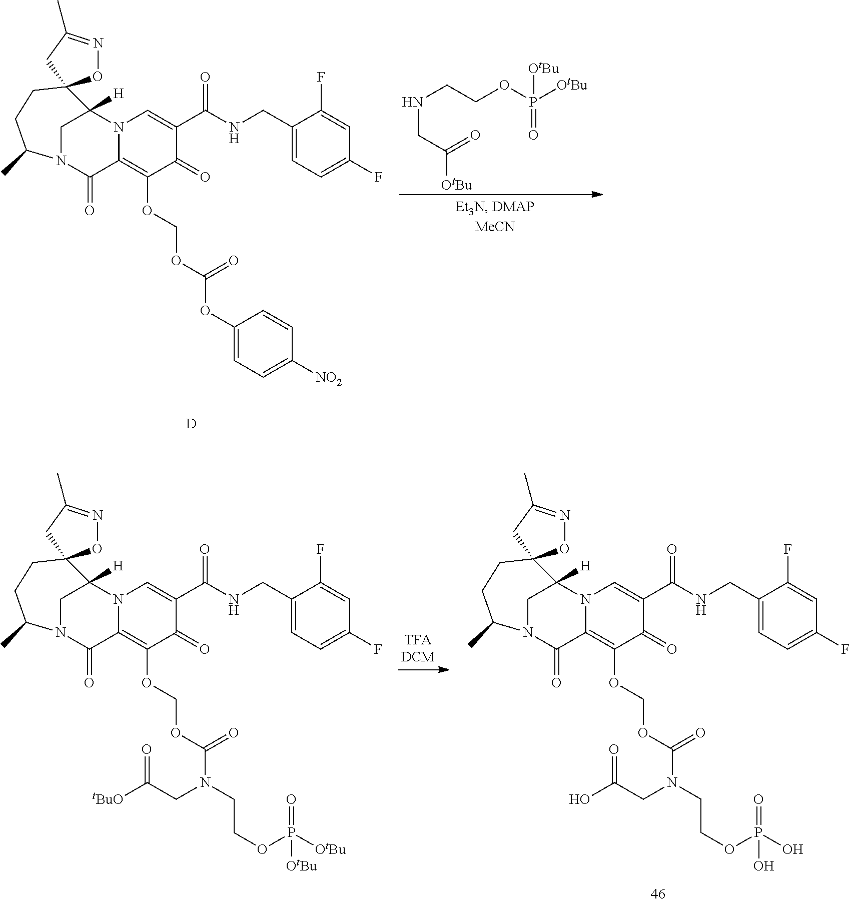

### Step 1: Preparation of tert-butyl N-benzyl-N-(2-hydroxyethyl)glycinate

To mixture of 2-(benzylamino) ethanol (2 g, 13.2 mmol) in acetonitrile (20 mL) was added tert-butyl 2-chloroacetate (2.2 g, 14.5 mmol), K₂CO₃ (2.19 g, 15.9 mmol) and NaI (0.40 g, 2.65 mmol). The resulting mixture was stirred at reflux for overnight. Ethyl acetate was added to dilute the mixture, and the organic phase was washed with H₂O and brine. The solvent was removed under reduced pressure to obtain the title compound, which was used without further purification. MS (m/z) 265.9 [M+H]⁺.

### Step 2: Preparation of tert-butyl N-benzyl-N-(2-((di-tert-butoxyphosphoryl)oxy)ethyl)glycinate

To a mixture of tert-butyl N-benzyl-N-(2-hydroxyethyl)glycinate (1 g, 3.77 mmol) in THF (10 mL) was added 1H-tetrazole (0.53 g, 7.54 mmol) and di-tert-butyl N,N-diisopropylphosphoramidite (1.57 g, 5.65 mmol). The resulting mixture was stirred at room temperature for 16 hours. The mixture was cooled to 0° C. and 30% H₂O₂ aqueous solution (4.70 g, 41.5 mmol) was added to the mixture. After 15 minutes, the cooling bath was removed and the mixture was stirred for an additional 6 h. Aqueous 10% Na₂SO₃ (50 mL) was added to the mixture with water bath cooling. After 25 minutes, EtOAc was added and the phases separated. The organic phase was washed with H₂O, brine and dried with MgSO₄. The solvent was removed under reduced pressure and the residue was purified by silica gel column chromatography (0-100% EtOAc/hexane) to obtain the title compound. MS (m/z) 458.4 [M+H]⁺.

### Step 3: Preparation of tert-butyl(2-((di-tert-butoxyphosphoryl)oxy)ethyl)glycinate

To a mixture of tert-butyl N-benzyl-N-(2-((di-tert-butoxyphosphoryl)oxy)ethyl)glycinate (1.04 g, 2.27 mmol) in EtOH (20 mL) was added 10% wt. Pd/C (0.242 g, 0.27 mmol). The resulting mixture was purged with H₂. After 1 h, the reaction mixture was filtered through celite and the filter cake was washed with EtOH. The filtrate was concentrated under reduced pressure to provide the title compound, which was used without further purification. MS (m/z) 368.4 [M+H]⁺.

### Step 4: Preparation of N-(2-((di-tert-butoxyphosphoryl)oxy)ethyl)-N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)glycine

To a mixture of Intermediate D (0.2 g, 0.29 mmol) in acetonitrile (5 mL) was added tert-butyl(2-((di-tert-butoxyphosphoryl)oxy)ethyl)glycinate (0.22 g, 0.587 mmol) followed by the addition of triethylamine (0.082 mL, 0.587 mmol) and DMAP (0.358 g, 0.029 mmol). The resulting mixture was stirred at room temperature overnight. The reaction mixture was diluted with EtOAc and washed with 1N HCl, H₂O and brine. The solvent was removed under reduced pressure and the residue was purified by silica gel column chromatography (0-100% EtOAc/hexane) to obtain the title compound. MS (m/z) 910.7 [M+H]⁺.

### Step 5: Preparation of N-(((((3′S,5S,7′R)-10′-((2,4-difluorobenzyl)carbamoyl)-3,3′-dimethyl-1′,11′-dioxo-1′,4′,5′, 11′-tetrahydro-3′H,4H,7′H-spiro[isoxazole-5,6′-[2,7]methanopyrido[1,2-a][1,4]diazonin]-12′-yl)oxy)methoxy)carbonyl)-N-(2-(phosphonooxy)ethyl)glycine (46)

To a mixture of N-(2-((di-tert-butoxyphosphoryl)oxy)ethyl)-N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)glycine (0.18 g, 0.198 mmol) in DCM (5 mL) was added TFA (0.5 mL) at 0° C. The mixture was warmed to room temperature and stirred for 2 h. The solvent was removed under vacuo and the resulting residue was purified by prep-HPLC to obtain the title compound. MS (m/z) 741.9 [M+H]⁺. 1H NMR (400 MHz, DMSO-d6) δ 10.31 (td, J=6.0, 3.5 Hz, 1H), 8.66 (d, J=5.8 Hz, 1H), 7.43 (td, J=8.7, 6.6 Hz, 1H), 7.25 (ddd, J=10.4, 9.3, 2.6 Hz, 1H), 7.08 (dddd, J=8.7, 7.6, 2.7, 1.3 Hz, 1H), 5.78-5.70 (m, 1H), 5.64 (d, J=6.1 Hz, 1H), 4.76-4.44 (m, 4H), 4.06-3.94 (m, 1H), 3.96-3.82 (m, 3H), 3.75-3.60 (m, 2H), 3.43 (dt, J=16.8, 6.4 Hz, 2H), 3.00 (d, J=17.5 Hz, 1H), 2.64 (dd, J=17.6, 4.3 Hz, 1H), 1.95 (s, 3H), 1.83-1.65 (m, 3H), 1.34 (dd, J=16.2, 8.3 Hz, 1H), 1.16 (dd, J=6.7, 2.7 Hz, 3H).

### Example 47: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl dimethylcarbamate (47)

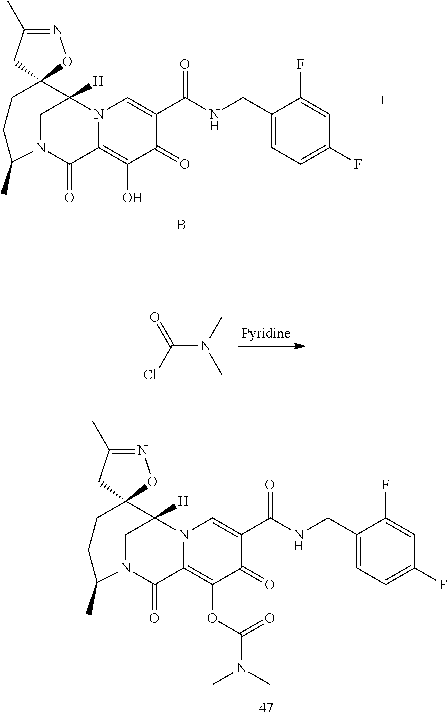

N,N-dimethyl carbamic chloride (221 mg, 2.06 mmol) was added to a suspension of Intermediate B (100 mg, 0.206 mmol) in pyridine (2 mL). The resulting suspension was heated at 50° C. for 2 h. The reaction mixture was then concentrated to dryness. The residue was placed under high vacuum overnight. The residue was dissolved in DMF and purified by reverse phase prep HPLC (10-100% MeCN/water) to afford the title compound. MS (m/z): calculated for C₂₇H₂₉F₂N₅O₆, 557.21; found, 557.99 [M+H]+. 1H NMR (400 MHz, DMSO) δ 10.22 (t, J=5.9 Hz, 1H), 8.76 (s, 1H), 7.42 (td, J=8.7, 6.6 Hz, 1H), 7.25 (ddd, J=10.5, 9.3, 2.6 Hz, 1H), 7.08 (tdd, J=8.6, 2.7, 1.0 Hz, 1H), 4.68 (s, 1H), 4.66-4.47 (m, 3H), 3.82-3.63 (m, 2H), 3.05-2.97 (m, 4H), 2.88 (s, 3H), 2.65 (d, J=17.5 Hz, 1H), 1.95 (s, 3H), 1.89-1.70 (m, 3H), 1.36-1.2 (m, 1H), 1.16 (d, J=6.7 Hz, 3H).

### Example 48: Preparation of N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-N-(2-(phosphonooxy)ethyl)glycine (48)

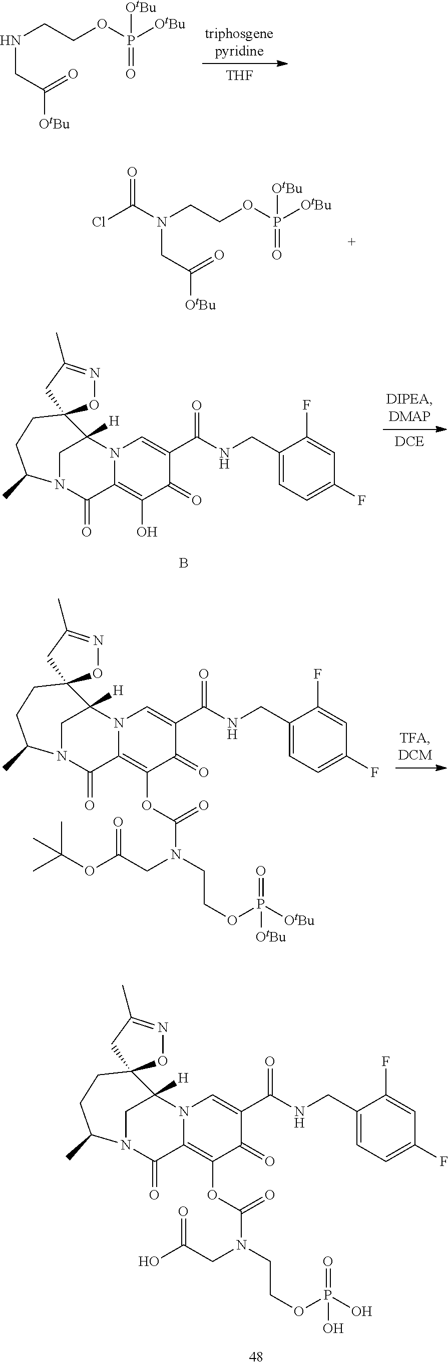

### Step 1: Preparation of tert-butyl N-(chlorocarbonyl)-N-(2-((di-tert-butoxyphosphoryl)oxy)ethyl)glycinate

To a solution of triphosgene (0.12 g, 0.41 mmol) in THF (6 mL) at 0° C. was added pyridine (0.09 ml, 1.22 mmol). To the resulting suspension was added a solution of tert-butyl 2-(2-ditert-butoxyphosphoryloxyethylamino) acetate (0.3 g, 0.817 mmol), prepared according to Example 46, dissolved in THF (3 mL). The suspension was allowed to warm to rt and stir for 3 h. The reaction mixture was filtered through Celite and rinsed with DCM/ethyl ether. The filtrate was concentrated and used directly in next step.

### Step 2: Preparation of tert-butyl N-(2-((di-tert-butoxyphosphoryl)oxy)ethyl)-N-((((3′S,5S,7′R)-10′-((2,4-difluorobenzyl)carbamoyl)-3,3′-dimethyl-1′,11′-dioxo-1′4′,5′,11′-tetrahydro-3′H,4H,7′H-spiro[isoxazole-5,6′-[2,7]methanopyrido[1,2-a][ 1,4]diazonin]-12′-yl)oxy)carbonyl)glycinate

To a suspension of Intermediate B (0.25 g, 0.514 mmol) in DCE (5 ml) was added tert-butyl N-(chlorocarbonyl)-N-(2-((di-tert-butoxyphosphoryl)oxy)ethyl)glycinate (0.33 g, 0.77 mmol), followed by the addition of DMAP (0.063 g, 0.514 mmol) and DIPEA (0.45 mL, 2.57 mmol) at 0° C. The reaction mixture was warmed to rt and stirred overnight. The reaction mixture was diluted with EtOAc, washed with HCl (IN), water, and brine. The organic phase was dried over MgSO₄, filtered, and concentrated to afford a residue, which was purified by column chromatography (0-100% EtOAc/hexanes) to afford the title compound. MS (m/z) 880.5 [M+H]⁺.

### Step 3: Preparation of N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-N-(2-(phosphonooxy)ethyl)glycine (48)

To mixture of tert-butyl N-(2-((di-tert-butoxyphosphoryl)oxy)ethyl)-N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)glycinate (0.094 g, 0.11 mmol) in DCM (3 mL) was added TFA (0.3 mL) at 0° C. The mixture was warmed to room temperature and stirred for 2 h. The solvent was removed under vacuo and the resulting residue was purified by prep-HPLC (5-95% MeCN/water containing 0.5% TFA) to obtain the title compound. MS (m/z) 711.96 [M+H]⁺. 1H NMR (400 MHz, DMSO-d6) δ 10.19 (q, J=6.0 Hz, 1H), 8.78 (d, J=30.7 Hz, 1H), 7.42 (td, J=8.6, 6.7 Hz, 1H), 7.30-7.21 (m, 1H), 7.10-6.98 (m, 1H), 4.91-4.35 (m, 5H), 4.28-3.88 (m, 4H), 3.77-3.35 (m, 3H), 3.00 (d, J=17.5 Hz, 1H), 2.74-2.59 (m, 1H), 1.95 (s, 3H), 1.86-1.71 (m, 3H), 1.33-1.02 (m, 4H).

### Example 49: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((phosphonooxy)methyl) ethane-1,2-diylbis(methylcarbamate) (49)

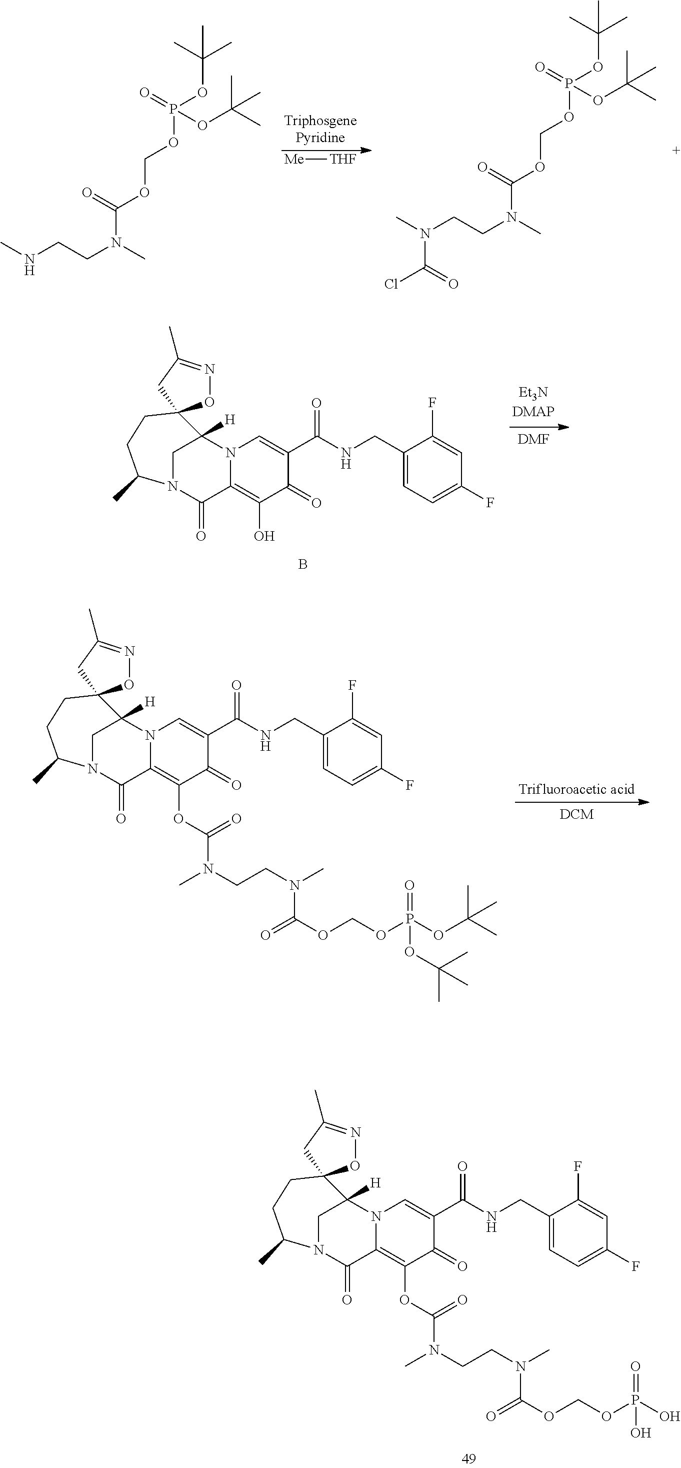

### Step 1: Preparation of ((di-tert-butoxyphosphoryl)oxy)methyl(2-((chlorocarbonyl)(methyl)amino)ethyl) (methyl)carbamate

((Di-tert-butoxyphosphoryl)oxy)methyl methyl(2-(methylamino)ethyl) carbamate (268 mg, 0.756 mmol), prepared according to WO2023102523, was dissolved in Me-THF (30 mL) at rt. Under argon balloon, the solution was cooled down to 0° C. Then pyridine (120 mg, 1.59 mmol) was added with stirring. After 10 min, triphosgene (202 mg, 0.681 mmol) was added in one portion as solid. After 3 h, the reaction mixture was diluted with EtOAc (10 mL) and was then treated with HCl (1N) (10 mL) and brine (20 mL). The organic phase was separated and dried over Na₂SO₄. The resulting solution was concentrated to afford the title compound. MS (m/z): calculated for C₁₅H₃₀ClN₂O₇P, 416.15; found, 416.90 [M+H]+.

### Step 2: Preparation of ((di-tert-butoxyphosphoryl)oxy)methyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) ethane-1,2-diylbis(methylcarbamate)

Intermediate B (72 mg, 0.148 mmol) and ((di-tert-butoxyphosphoryl)oxy)methyl(2-((chlorocarbonyl)(methyl)amino)ethyl) (methyl)carbamate (96 mg, 0.230 mmol) were dissolved in DMF (5 mL) at rt. Triethylamine (45 mg, 0.444 mmol) and DMAP (11 mg, 0.089 mmol) were added sequentially. The reaction mixture was stirred at rt for 2 hrs. The reaction mixture was then diluted with EtOAc (10 mL) and treated with saturated aqueous solution of NH₄Cl (10 mL). The organic phase was separated and the aqueous phase was extracted with EtOAc (1×10 mL). The combined organic phase was washed with water (20 mL) and brine (20 mL) and concentrated. The residue was purified by silica gel column chromatography (0-100% EtOAc/hexanes) to afford the title compound. MS (m/z): calculated for C₃₉H₅₃F₂N₆O₁₂P, 866.34; found, 866.70 [M+H]⁺.

### Step 3: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((phosphonooxy)methyl) ethane-1,2-diylbis(methylcarbamate) (49)

((Di-tert-butoxyphosphoryl)oxy)methyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) ethane-1,2-diylbis(methylcarbamate) (22 mg, 0.0254 mmol) was dissolved in DCM (7 mL) at rt. The solution was cooled down to 0° C. under argon atmosphere. Trifluoroacetic acid (1 g, 9.15 mmol) was added dropwise. The reaction was stirred at 0° C. for 70 min and concentrated under reduced pressure. The residue was purified by reversed phase prep HPLC (10-100% acetonitrile/water with 0.1% trifluoroacetic acid) to afford the title compound. MS (m/z): calculated for C₃₁H₃₇F₂N₆O₁₂P, 754.22; found, 754.92 [M+H]+. ¹H NMR (400 MHz, DMSO) δ 10.24-10.17 (m, 1H), 8.76 (d, J=36.3 Hz, 1H), 7.42 (q, J=8.4 Hz, 1H), 7.24 (ddd, J=10.4, 9.3, 2.6 Hz, 1H), 7.13-7.03 (m, 1H), 5.48 (t, J=11.8 Hz, 2H), 4.67-3.28 (m, 10H), 3.11-2.88 (m, 7H), 2.66 (d, J=17.4 Hz, 1H), 1.95 (s, 3H), 1.86-1.67 (m, 3H), 1.43-1.22 (m, 1H), 1.16 (d, J=6.7 Hz, 3H).

### Example 50: Preparation of N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)ethyl)-N-(((phosphonooxy)methoxy)carbonyl)glycine (50)

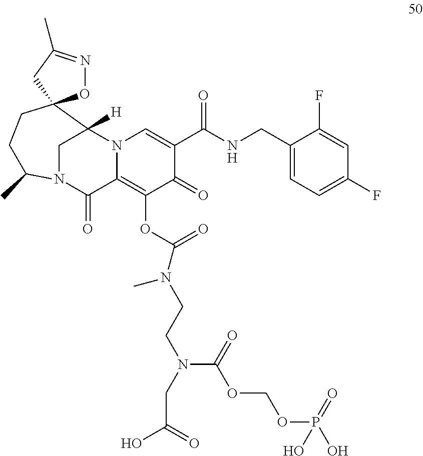 [SMILES: CC1=NO[C@@]2(CC[C@H](C)N3C[C@H]2n2cc(C(=O)NCc4ccc(F)cc4F)c(=O)c(OC(=O)N(C)CCN(CC(=O)O)C(=O)OCOP(=O)(O)O)c2C3=O)C1]

The title compound was prepared in a similar manner as Example 11, except using benzyl N-((((bis(benzyloxy)phosphoryl)oxy)methoxy)carbonyl)-N-(2-(methylamino)ethyl)glycinate, prepared according to Example 21, instead of dibenzyl (3-hydroxypropyl) phosphate in Step 1. MS (m/z) 799.1 [M+H]+. 1H NMR (400 MHz, Methanol-d4) δ 8.65 (d, J=3.0 Hz, 1H), 7.46 (q, J=7.9 Hz, 1H), 7.07-6.88 (m, 2H), 5.77-5.45 (m, 2H), 4.83-4.57 (m, 3H), 4.51 (s, 1H), 4.37-4.12 (m, 2H), 3.83 (s, 2H), 3.77-3.54 (m, 3H), 3.24-3.12 (m, 2H), 3.07 (s, 2H), 2.70 (d, J=17.9 Hz, 1H), 2.07 (s, 3H), 2.03-1.84 (m, 3H), 1.60 (s, 1H), 1.42 (s, 1H), 1.26 (d, J=6.8 Hz, 3H).

### Example 51: Preparation of N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-N-(2-(methyl(((phosphonooxy)methoxy)carbonyl)amino)ethyl)glycine (51)

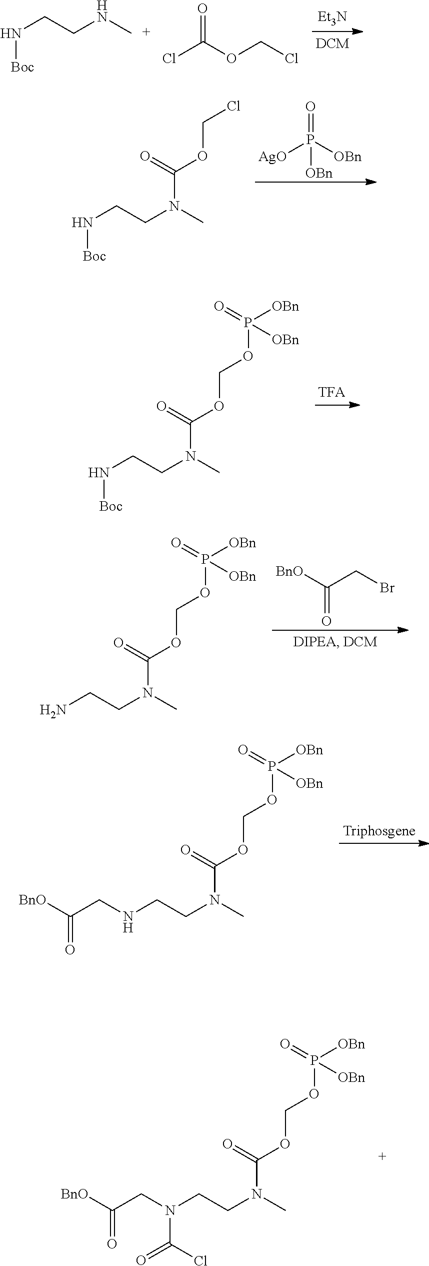

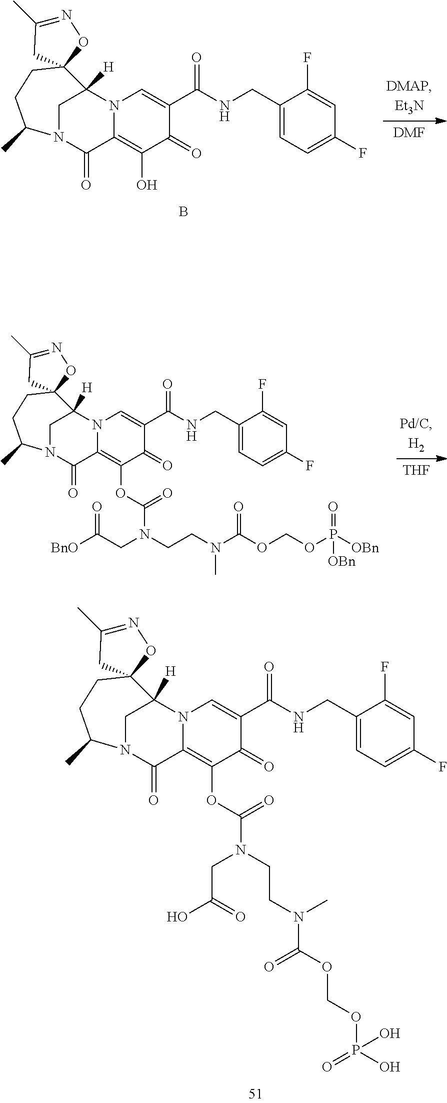

### Step 1: Synthesis of chloromethyl(2-((tert-butoxycarbonyl)amino)ethyl) (methyl)carbamate

To a solution of tert-butyl(2-(methylamino)ethyl) carbamate (1.02 g, 5.85 mmol) in DCM (60.0 mL) at 0° C. was added chloromethyl carbonochloridate (0.83 g, 6.44 mmol) followed by triethylamine (0.711 g, 7.02 mmol). The reaction was stirred at 0° C. for 30 minutes. The reaction was then diluted with DCM, washed sequentially with 1N HCl, water, brine, dried over sodium sulfate, filtered, and concentrated to give the title compound.

### Step 2: Synthesis of ((bis(benzyloxy)phosphoryl)oxy)methyl(2-((tert-butoxycarbonyl)amino)ethyl) (methyl)carbamate

The mixture of chloromethyl(2-((tert-butoxycarbonyl)amino)ethyl) (methyl)carbamate (1.56 g, 5.85 mmol) and ((bis(benzyloxy)phosphoryl)oxy) silver (2.93 g, 7.6 mmol) in toluene (14.0 mL) was heated at 110° C. for 16 hours. The reaction was cooled to room temperature, filtered through a pad of Celite, concentrated and purified by normal phase silica gel chromatography (0-70% EtOAc/Hexane) to give the title compound. MS (m/z) 508.740 [M+H]⁺

### Step 3: Synthesis of ((bis(benzyloxy)phosphoryl)oxy)methyl(2-aminoethyl) (methyl)carbamate

A solution of ((bis(benzyloxy)phosphoryl)oxy)methyl(2-((tert-butoxycarbonyl)amino)ethyl) (methyl)carbamate (4.09 g, 8.04 mmol) in DCM (80 mL) at 0° C. was treated with TFA (23.2 mL). After one hour, the reaction was quenched with saturated sodium bicarbonate until pH ~7. The mixture was concentrated to remove DCM and extracted with EtOAc (3×). The combined organic layer was washed with brine, dried over sodium sulfate, filtered and concentrated to give the title compound. MS (m/z) 409.038 [M+H]⁺

### Step 4: Synthesis of benzyl(2-(((((bis(benzyloxy)phosphoryl)oxy)methoxy)carbonyl)(methyl)amino)ethyl)glycinate

To a solution of ((bis(benzyloxy)phosphoryl)oxy)methyl(2-aminoethyl) (methyl)carbamate (118.3 mg, 0.29 mmol) in DCM (8.0 mL) at room temperature was added benzyl 2-bromoacetate (39.8 mg, 0.17 mmol) followed by DIPEA (22 mg, 0.17 mmol). The reaction was stirred for 1 hour before it was diluted with DCM, washed with saturated sodium bicarbonate, brine, dried over sodium sulfate, filtered and concentrated. The residue was purified by normal phase silica gel chromatography (20-100% EtOAc/Hexane then 0-15% MeOH/EtOAc) to afford the title compound. MS (m/z) 557.074 [M+H]⁺

### Step 5: Synthesis of benzyl N-(2-(((((bis(benzyloxy)phosphoryl)oxy)methoxy)carbonyl)(methyl)amino)ethyl)-N-(chlorocarbonyl)glycinate

To a solution of benzyl(2-(((((bis(benzyloxy)phosphoryl)oxy)methoxy)carbonyl)(methyl)amino)ethyl)glycinate (50 mg, 0.09 mmol) in DCM (2.0 mL) at 0° C. was added pyridine (9.23 mg, 0.117 mmol) followed by bis(trichloromethyl) carbonate (24.0 mg, 0.081 mmol). After stirring for 30 minutes, the reaction was diluted with DCM, washed sequentially with 1N HCl, water, brine, dried over sodium sulfate, filtered and concentrated to give the title compound. MS (m/z) 618.754 [M+H]⁺

### Step 6: Synthesis of benzyl N-(2-(((((bis(benzyloxy)phosphoryl)oxy)methoxy)carbonyl)(methyl)amino)ethyl)-N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)glycinate

To a mixture of Intermediate B (20 mg, 0.04 mmol) in DMF (1.0 mL) at room temperature was added benzyl N-(2-(((((bis(benzyloxy)phosphoryl)oxy)methoxy)carbonyl)(methyl)amino)ethyl)-N-(chlorocarbonyl)glycinate (76.3 mg, 0.12 mmol) followed by triethylamine (16.6 mg, 0.164 mmol) and DMAP (0.5 mg, 0.004 mmol). After stirring overnight, the reaction was diluted with EtOAc, washed with water and brine, dried over sodium sulfate, filtered, and concentrated. The residue was purified by normal phase silica gel chromatography (0-100% EtOAc/Hexane then 0-15% MeOH/EtOAc) to give title compound. MS (m/z) 1068.713 [M]⁺.

### Step 7: Synthesis of N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-N-(2-(methyl(((phosphonooxy)methoxy)carbonyl)amino)ethyl)glycine (51)

To a solution of benzyl N-(2-(((((bis(benzyloxy)phosphoryl)oxy)methoxy)carbonyl)(methyl)amino)ethyl)-N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)glycinate (36.0 mg, 0.034 mmol) in a mixture of MeOH (0.2 mL) and EtOAc (2.0 mL) at room temperature was added 5 wt % Pd/C (2.0 mg). The reaction was degassed and flushed with nitrogen then degassed and flushed with hydrogen three times before it was hydrogenated under hydrogen balloon overnight. The reaction was then degassed and flushed with nitrogen and filtered through a pad of Celite, rinsing the filter cake with EtOAc. The filtrate was concentrated and purified by reverse phase preparative HPLC (10-100% MeCN/water containing 0.1% TFA) to give the title compound. MS (m/z) 798.799 [M]⁺. ¹H NMR (400 MHz, Methanol-d4) δ 8.67-8.59 (m, 1H), 7.46 (q, J=8.2 Hz, 1H), 7.04-6.89 (m, 2H), 5.71-5.53 (m, 2H), 4.83-4.72 (m, 1H), 4.72-4.59 (m, 2H), 4.54-4.46 (m, 1H), 4.31-4.09 (m, 2H), 3.89-3.54 (m, 6H), 3.16 (dd, J=17.7, 4.6 Hz, 1H), 3.10-2.98 (m, 3H), 2.70 (d, J=17.9 Hz, 1H), 2.07 (s, 3H), 2.01-1.85 (m, 3H), 1.69-1.49 (m, 1H), 1.27 (dd, J=6.8, 2.2 Hz, 3H).

### Example 52: Preparation of N-(2-((carboxymethyl)((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)amino)ethyl)-N-(((phosphonooxy)methoxy)carbonyl)glycine (52)

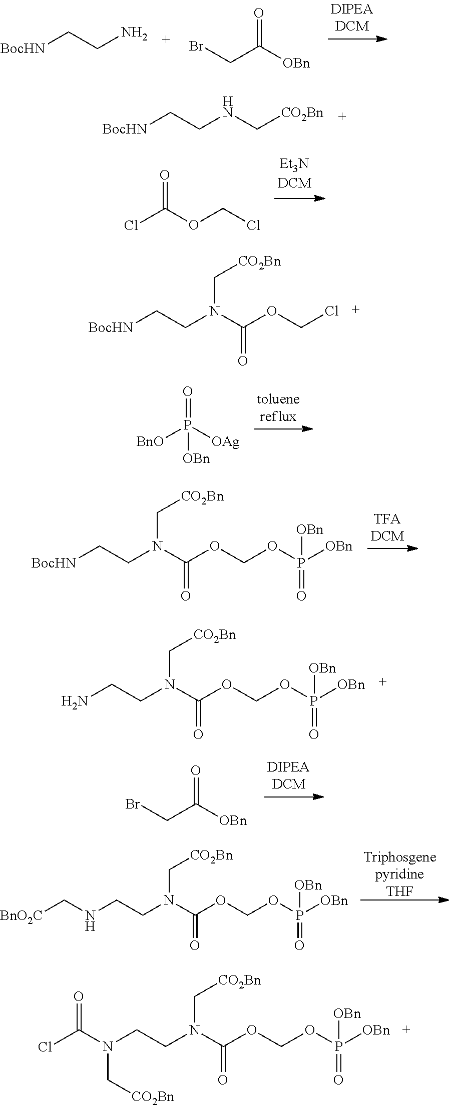

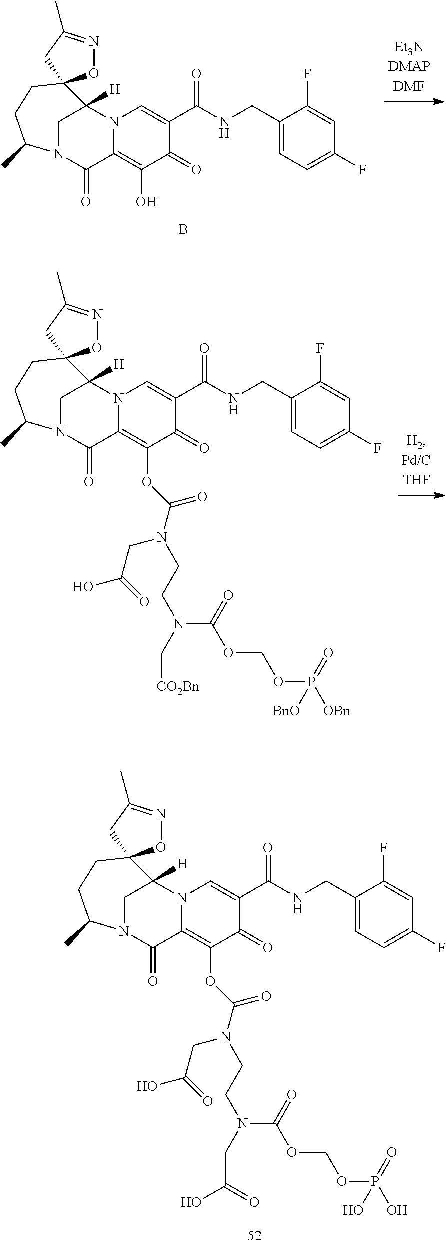

### Step 1: Preparation of benzyl(2-((tert-butoxycarbonyl)amino)ethyl)glycinate

To a stirred solution of tert-butyl N-(2-aminoethyl) carbamate (5.0 g, 31.2 mmol) in DCM (30 mL) at 0° C. under argon was added benzyl 2-bromoacetate (3.57 g, 15.6 mmol) followed by DIPEA (3.1 g, 24 mmol). The reaction mixture was stirred for 16 h at room temperature. The reaction mixture was diluted with DCM and washed with water. The organic layer was dried over anhydrous Na₂SO₄, filtered, and concentrated under reduced pressure. The residue was purified by silica gel column chromatography (20-50% EtOAc/Hexanes) to afford the title compound. MS (m/z) 309.1 [M+H]+.

### Step 2: Preparation of benzyl N-(2-((tert-butoxycarbonyl)amino)ethyl)-N-((chloromethoxy)carbonyl)glycinate

To a stirred solution of benzyl(2-((tert-butoxycarbonyl)amino)ethyl)glycinate (5.0 g, 16.2 mmol) in DCM (50 mL) at 0° C. under argon was added chloromethyl chloroformate (2.51 g, 19.5 mmol) followed by Et₃N (2.46 g, 24.3 mmol). The mixture was stirred for 16 h at room temperature. The reaction mixture was diluted with DCM and washed with water. The organic layer was dried over anhydrous Na₂SO₄, filtered, and concentrated under reduced pressure. The crude residue was purified by silica gel column chromatography (20-50% EtOAc/Hexanes) to afford the title compound. MS (m/z) 401.3 [M+H]⁺.

### Step 3: Preparation of benzyl N-((((bis(benzyloxy)phosphoryl)oxy)methoxy)carbonyl)-N-(2-(tert-butoxycarbonyl)amino)ethyl)glycinate

To a stirred solution of benzyl N-(2-((tert-butoxycarbonyl)amino)ethyl)-N-((chloromethoxy)carbonyl)glycinate (6.5 g, 16.2 mmol) in toluene (50 mL) at room temperature under argon was added silver dibenzylphosphate (8.12 g, 21.1 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (30-70% EtOAc/Hexanes) to afford the title compound. MS (m/z) 643.2 [M+H]⁺.

Steps 4-5: Preparation of benzyl(2-((2-(benzyloxy)-2-oxoethyl) ((((bis(benzyloxy)phosphoryl)oxy)methoxy)carbonyl)amino)ethyl)glycinate

To a stirred solution of benzyl N-((((bis(benzyloxy)phosphoryl)oxy)methoxy)carbonyl)-N-(2-((tert-butoxycarbonyl)amino)ethyl)glycinate (0.5 g, 0.78 mmol) in DCM (3 mL) at 0° C. under argon was added 2,2,2-trifluoroacetic acid (0.6 mL) in 0.5 mL of DCM under argon. The mixture was stirred for 40 minutes at room temperature and concentrated under reduced pressure. To the crude mixture in DCM (10 mL) at 0° C. under argon was added benzyl 2-bromoacetate (107.0 mg, 0.47 mmol) followed by triethylamine (394 mg, 3.89 mmol). The reaction mixture was stirred for 16 h at room temperature. The reaction mixture was diluted with DCM and washed with water. The organic layer was dried over anhydrous Na₂SO₄, filtered, and concentrated under reduced pressure to afford the title compound. MS (m/z) 691.3 [M+H]+.

### Steps 6-8: Preparation of N-(2-((carboxymethyl)((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)amino)ethyl)-N-(((phosphonooxy)methoxy)carbonyl)glycine (52)

The title compound was prepared in a manner similar to Steps 1-3 of Example 11, except using benzyl(2-((2-(benzyloxy)-2-oxoethyl) ((((bis(benzyloxy)phosphoryl)oxy)methoxy)carbonyl)amino)ethyl)glycinate instead of dibenzyl (3-hydroxypropyl) phosphate in Step 1 and including 0.2 equiv of DMAP in Step 2. MS (m/z) 842.9 [M+H]+. 1H NMR (400 MHz, Methanol-d4) δ 8.63 (d, J=5.4 Hz, 1H), 7.46 (q, J=8.0 Hz, 1H), 7.07-6.89 (m, 2H), 5.73-5.45 (m, 2H), 4.83-4.42 (m, 4H), 4.45-4.03 (m, 4H), 3.95-3.50 (m, 6H), 3.25-3.09 (m, 1H), 2.69 (d, J=17.9 Hz, 1H), 2.06 (s, 3H), 2.01-1.79 (m, 3H), 1.62 (d, J=13.6 Hz, 1H), 1.30-1.17 (m, 3H).

### Example 53: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-(phosphonooxy)propyl) carbonate (53)

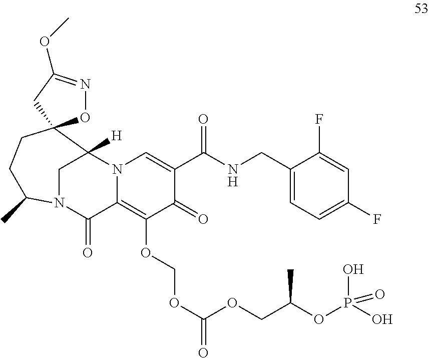 [SMILES: COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1]

The title compound was prepared in a manner similar to Example 18, except using (R)-1-((tert-butyldimethylsilyl)oxy)propan-2-ol instead of (R)-2-((tert-butyldimethylsilyl)oxy)propan-1-ol in Step 1, and Intermediate C is used instead of Intermediate B in Step 4. MS (m/z) 714.9 [M−H]+. 1H NMR (400 MHz, DMSO-d6) δ 10.27 (t, J=5.9 Hz, 1H), 8.75 (s, 1H), 7.42 (td, J=8.7, 6.6 Hz, 1H), 7.25 (ddd, J=10.5, 9.3, 2.6 Hz, 1H), 7.08 (ddd, J=10.5, 7.9, 2.5 Hz, 1H), 5.79 (d, J=6.4 Hz, 1H), 5.68 (d, J=6.4 Hz, 1H), 4.72 (m, 2H), 4.64 (dt, J=11.1, 7.4 Hz, 1H), 4.55 (t, J=5.9 Hz, 2H), 4.53-4.39 (m, 1H), 4.15 (dd, J=11.2, 5.2 Hz, 1H), 4.04 (dd, J=11.3, 4.6 Hz, 1H), 3.82 (s, 3H), 3.75-3.61 (m, 2H), 3.06 (d, J=16.9 Hz, 1H), 2.76 (dd, J=16.9, 5.2 Hz, 1H), 1.96-1.85 (m, 1H), 1.76 (dd, J=23.5, 12.0 Hz, 2H), 1.39-1.28 (m, 1H), 1.24-1.12 (m, 6H).

### Example 54: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-(phosphonooxy)propan-2-yl) carbonate (54)

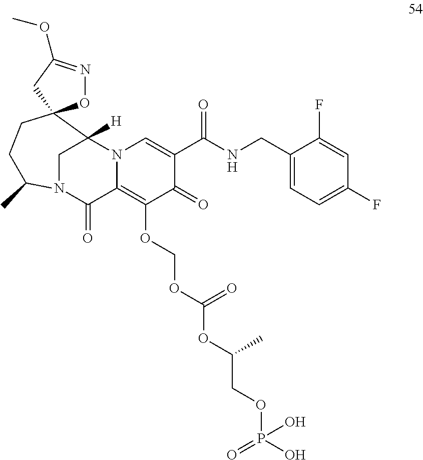 [SMILES: COC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1]

The title compound was prepared in a manner similar to Example 18, except using Intermediate C instead of Intermediate B in Step 4. MS (m/z) 715.00 [M+H]⁺. ¹H NMR (400 MHz, DMSO) δ 10.28 (t, J=6.0 Hz, 1H), 8.75 (s, 1H), 7.54-7.33 (m, 1H), 7.33-7.16 (m, 1H), 7.16-6.96 (m, 1H), 5.85 (d, J=6.4 Hz, 1H), 5.59 (d, J=6.4 Hz, 1H), 4.93-4.76 (m, 1H), 4.74-4.47 (m, 5H), 3.98-3.78 (m, 5H), 3.78-3.59 (m, 2H), 3.05 (d, J=16.8 Hz, 1H), 2.75 (d, J=16.9 Hz, 1H), 1.95-1.69 (m, 4H), 1.44-1.26 (m, 1H), 1.26-1.10 (m, 6H).

### Intermediates F and G: Preparation of 3-((bis(benzyloxy)phosphoryl)oxy)-2-methylbutan-2-yl (chloromethyl) carbonate (Intermediate F) and 3-((bis(benzyloxy)phosphoryl)oxy)-3-methylbutan-2-yl (chloromethyl) carbonate (Intermediate G)

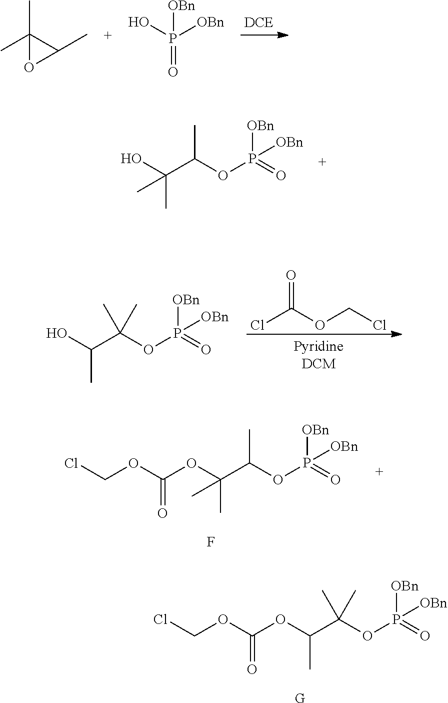

### Step 1: Synthesis of mixture of dibenzyl (3-hydroxy-3-methylbutan-2-yl) phosphate and dibenzyl (3-hydroxy-2-methylbutan-2-yl) phosphate

Into a solution of dibenzyl hydrogen phosphate (1.925 g, 6.92 mmol) in DCM (24 mL), 2,2,3-trimethyloxirane (0.596 g 6.92 mmol) was added at rt. After stirring for overnight, solvent was removed and the residue was purified by column chromatography (0-100% EtOAc/Hexanes) on silica gel to provide the mixture of title products. MS (m/z) 364.9 [M+H]⁺.

### Step 2: Synthesis of 3-((bis(benzyloxy)phosphoryl)oxy)-2-methylbutan-2-yl (chloromethyl) carbonate (Intermediate D) and 3-((bis(benzyloxy)phosphoryl)oxy)-3-methylbutan-2-yl (chloromethyl) carbonate (Intermediate E)

Into the mixture of dibenzyl (3-hydroxy-3-methylbutan-2-yl) phosphate in DCM (20 mL) at 0° C., was added, chloromethyl carbonochloridate (0.442 g, 3.43 mmol), and pyridine (326 mg, 4.12 mmol). After addition, the reaction was allowed to warm to rt for overnight. Then the reaction mixture was extracted with ethyl acetate and washed with brine. After drying with anhydrous MgSO₄, the solvent was removed and the residue was purified by silica gel column chromatography (0-100% EtOAc/Hexanes) to provide the two title products.

Intermediate F (Peak 1): 3-((bis(benzyloxy)phosphoryl)oxy)-2-methylbutan-2-yl (chloromethyl) carbonate: ¹H NMR (400 MHz, Chloroform-d) δ 7.37 (s, 10H), 5.64 (d, J=6.2 Hz, 1H), 5.55 (d, J=6.3 Hz, 1H), 5.13-5.06 (m, 3H), 5.05 (d, J=2.3 Hz, 1H), 4.85-4.75 (m, 1H), 1.51 (d, J=8.7 Hz, 6H), 1.33 (d, J=6.5 Hz, 3H).

Intermediate G (Peak 2): 3-((bis(benzyloxy)phosphoryl)oxy)-3-methylbutan-2-yl (chloromethyl) carbonate: ¹H NMR (400 MHz, Chloroform-d) δ 7.43-7.3 (m, 10H), 5.68 (d, J=0.8 Hz, 2H), 5.03 (dd, J=7.8, 3.9 Hz, 4H), 4.9 (qd, J=6.5, 1.2 Hz, 1H, 1.54 (d, J=4.2 Hz, 6H), 1.3 (d, J=6.5 Hz, 3H)

### Example 55: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl(2-methyl-3-(phosphonooxy)butan-2-yl) carbonate (55)

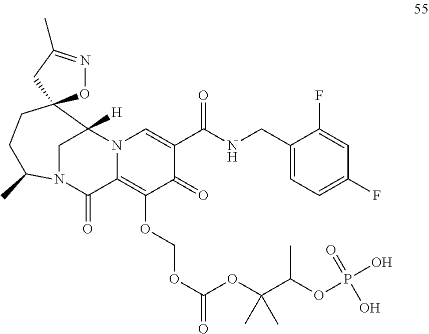 [SMILES: CC1=NO[C@@]2(CC[C@H](C)N3C[C@H]2n2cc(C(=O)NCc4ccc(F)cc4F)c(=O)c(OCOC(=O)OC(C)(C)C(C)OP(=O)(O)O)c2C3=O)C1]

The title compound was prepared in a manner similar to Example 18 except using 3-((bis(benzyloxy)phosphoryl)oxy)-2-methylbutan-2-yl (chloromethyl) carbonate (Intermediate F) instead of (R)-1-((bis(benzyloxy)phosphoryl)oxy)propan-2-yl (chloromethyl) carbonate in Step 4. MS (m/z) 727.97 [M+H]⁺. ¹H NMR (400 MHz, DMSO) δ 10.28 (t, J=6.1 Hz, 1H), 8.71 (s, 1H), 7.50-7.36 (m, 1H), 7.36-7.17 (m, 1H), 7.17-7.01 (m, 1H), 5.91-5.73 (m, 1H), 5.73-5.58 (m, 1H), 4.78-4.42 (m, 5H), 3.69 (s, 2H), 3.44-3.34 (m, 2H), 3.00 (d, J=17.5 Hz, 1H), 2.63 (d, J=17.6 Hz, 1H), 1.95 (s, 3H), 1.89-1.66 (m, 3H), 1.46-1.23 (m, 6H), 1.23-1.13 (m, 6H), 1.14-1.06 (m, 1H).

### Example 56: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3-methyl-3-(phosphonooxy)butan-2-yl) carbonate (56)

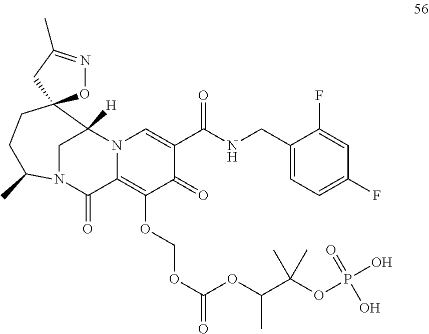 [SMILES: CC1=NO[C@@]2(CC[C@H](C)N3C[C@H]2n2cc(C(=O)NCc4ccc(F)cc4F)c(=O)c(OCOC(=O)OC(C)C(C)(C)OP(=O)(O)O)c2C3=O)C1]

The title compound was prepared in a manner similar to Example 18 except using 3-((bis(benzyloxy)phosphoryl)oxy)-3-methylbutan-2-yl (chloromethyl) carbonate (Intermediate G) instead of (R)-1-((bis(benzyloxy)phosphoryl)oxy)propan-2-yl (chloromethyl) carbonate in Step 4. MS (m/z) 727.619 [M+H]⁺. ¹H NMR (400 MHz, DMSO) δ 10.40-10.23 (m, 1H), 8.77-8.62 (m, 1H), 7.56-7.34 (m, 1H), 7.34-7.18 (m, 1H), 7.18-7.02 (m, 1H), 5.86-5.66 (m, 1H), 5.66-5.49 (m, 1H), 4.77-4.69 (m, 1H), 4.69-4.47 (m, 4H), 4.47-4.29 (m, 1H), 3.77-3.62 (m, 2H), 3.46-3.34 (m, 1H), 3.00 (d, J=17.3 Hz, 1H), 2.75-2.59 (m, 1H), 1.95 (s, 3H), 1.87-1.68 (m, 3H), 1.50-1.23 (m, 7H), 1.23-0.98 (m, 6H).

### Intermediates H and I: Preparation (2R,3S)-3-((bis(benzyloxy)phosphoryl)oxy)butan-2-yl (chloromethyl) carbonate (Intermediate H) and (2S,3R)-3-((bis(benzyloxy)phosphoryl)oxy)butan-2-yl (chloromethyl) carbonate (Intermediate I)

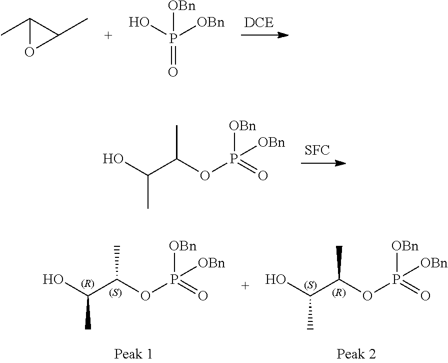

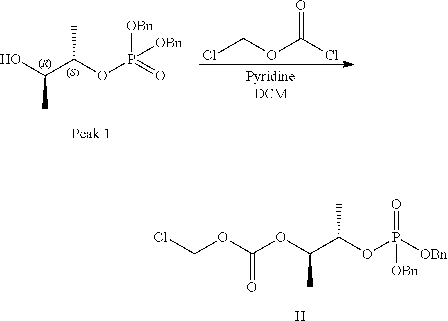

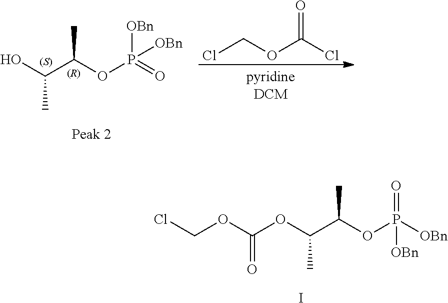

### Step 1: Synthesis of mixture of dibenzyl (3-hydroxybutan-2-yl) phosphate

Into a solution of dibenzyl hydrogen phosphate (2.42 g, 33.6 mmol) in DCM (24 mL), 2,3-dimethyloxirane (0.596 g 6.92 mmol) was added at rt. After stirring for overnight, solvent was removed and the residue was purified by column chromatography (0-100% EtOAc/Hexanes) on silica gel to provide the mixture of title products. MS (m/z) 350.8 [M+H]⁺.

### Step 2: Preparation of dibenzyl ((2S,3R)-3-hydroxybutan-2-yl) phosphate (Peak 1) and dibenzyl ((2R,3S)-3-hydroxybutan-2-yl) phosphate (Peak 2)

SFC separation of dibenzyl (3-hydroxybutan-2-yl) phosphate (Column: IG 4.6×100 mm 5mic, 3 mL/min, 20% EtOH).

(Peak 1): dibenzyl ((2S,3R)-3-hydroxybutan-2-yl) phosphate: MS (m/z): 350.8 [M+H]+.

(Peak 2): dibenzyl ((2R,3S)-3-hydroxybutan-2-yl) phosphate: MS (m/z): 350.9 [M+H]+.

### Step 3: Preparation of (2R,3S)-3-((bis(benzyloxy)phosphoryl)oxy)butan-2-yl (chloromethyl) carbonate (Intermediate H) and (2S,3R)-3-((bis(benzyloxy)phosphoryl)oxy)butan-2-yl (chloromethyl) carbonate (Intermediate I)

Into the mixture of dibenzyl ((2S,3R)-3-hydroxybutan-2-yl) phosphate in DCM (20 mL) at 0° C., was added, chloromethyl carbonochloridate (0.237 g, 1.84 mmol), and pyridine (165 mg, 2.09 mmol). After addition, the reaction was allowed to warm to rt for overnight. Then the reaction mixture was extracted with ethyl acetate and washed with brine. After drying with anhydrous MgSO₄, the solvent was removed and the residue was purified by silica gel column chromatography (0-100% EtOAc/Hexanes) to provide the two title products.

(2R,3S)-3-((bis(benzyloxy)phosphoryl)oxy)butan-2-yl (chloromethyl) carbonate (Intermediate H): MS (m/z): 442.7 [M+H]+.

(2S,3R)-3-((bis(benzyloxy)phosphoryl)oxy)butan-2-yl (chloromethyl) carbonate (Intermediate I): MS (m/z): 442.9 [M+H]+.

### Example 57: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3R)-3-(phosphonooxy)butan-2-yl) carbonate (57)

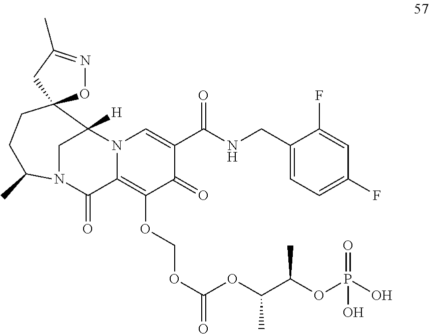 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1]

The title compound was prepared in a manner similar to Example 18 except using (2S,3R)-3-((bis(benzyloxy)phosphoryl)oxy)butan-2-yl (chloromethyl) carbonate (Intermediate I) instead of (R)-1-((bis(benzyloxy)phosphoryl)oxy)propan-2-yl (chloromethyl) carbonate in Step 4. MS (m/z) 712.98 [M+H]⁺. 1H NMR (400 MHz, DMSO) δ 10.28 (t, J=5.9 Hz, 1H), 8.70 (s, 1H), 7.53-7.36 (m, 1H), 7.36-7.15 (m, 1H), 7.15-6.99 (m, 1H), 5.85 (d, J=6.5 Hz, 1H), 5.60 (d, J=6.5 Hz, 1H), 4.78-4.39 (m, 5H), 4.28-4.09 (m, 1H), 3.79-3.58 (m, 2H), 3.00 (d, J=17.5 Hz, 1H), 2.63 (d, J=17.6 Hz, 1H), 1.94 (s, 3H), 1.89-1.67 (m, 3H), 1.44-1.21 (m, 3H), 1.23-0.98 (m, 9H).

### Example 58: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3S)-3-(phosphonooxy)butan-2-yl) carbonate (58)

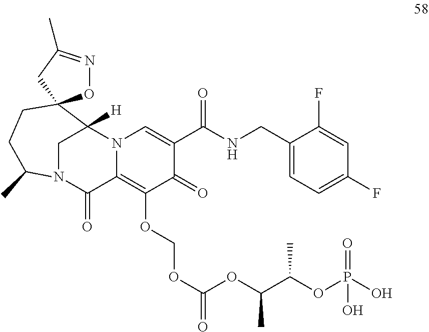 [SMILES: CC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1]

The title compound was prepared in a manner similar to Example 18 except using (2R,3S)-3-((bis(benzyloxy)phosphoryl)oxy)butan-2-yl (chloromethyl) carbonate (Intermediate H) instead of (R)-1-((bis(benzyloxy)phosphoryl)oxy)propan-2-yl (chloromethyl) carbonate in Step 4. MS (m/z) 712.9 [M+H]⁺. 1H NMR (400 MHz, DMSO-d6) δ 10.28 (t, J=5.9 Hz, 1H), 8.70 (s, 1H), 7.43 (td, J=8.6, 6.5 Hz, 1H), 7.35-7.18 (m, 1H), 7.08 (td, J=8.5, 2.6 Hz, 1H), 5.79-5.65 (m, 2H), 4.74-4.41 (m, 5H), 4.25-4.12 (m, 2H), 3.69 (s, 2H), 3.00 (d, J=17.5 Hz, 1H), 2.64 (d, J=17.5 Hz, 1H), 1.95 (s, 4H), 1.78 (p, J=6.7 Hz, 3H), 1.33-1.23 (m, 1H), 1.21-1.01 (m, 9H).

### Example 59: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3R)-3-(phosphonooxy)butan-2-yl) carbonate (59)

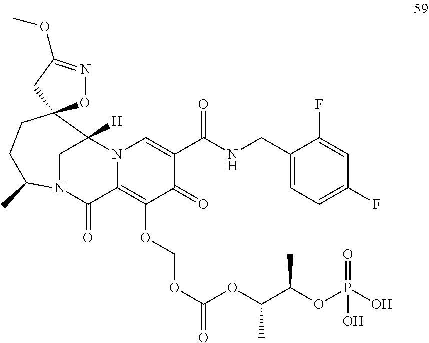 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1]

The title compound was prepared in a manner similar to Example 57 except using Intermediate C instead of Intermediate B in Step 4. MS (m/z) 728.98 [M+H]+. ¹H NMR (400 MHz, DMSO) δ 10.28 (t, J=5.9 Hz, 1H), 8.74 (s, 1H), 7.51-7.33 (m, 1H), 7.33-7.18 (m, 1H), 7.18-6.99 (m, 1H), 5.83 (d, J=6.4 Hz, 1H), 5.60 (d, J=6.4 Hz, 1H), 4.77-4.47 (m, 5H), 4.31-4.11 (m, 1H), 3.82 (s, 3H), 3.74-3.61 (m, 2H), 3.05 (d, J=16.9 Hz, 1H), 2.75 (d, J=16.9 Hz, 1H), 1.95-1.65 (m, 3H), 1.43-1.28 (m, 3H), 1.21-1.12 (m, 9H).

### Example 60: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3S)-3-(phosphonooxy)butan-2-yl) carbonate (60)

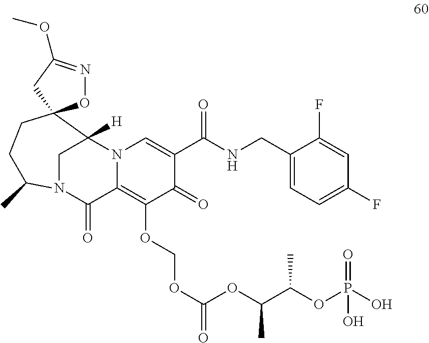 [SMILES: COC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1]

The title compound was prepared in a manner similar to Example 59 except using Intermediate C instead of Intermediate B in Step 4. MS (m/z) 728.8 [M+H]⁺. 1H NMR (400 MHz, DMSO) δ 10.27 (t, J=5.9 Hz, 1H), 8.74 (s, 1H), 7.52-7.32 (m, 1H), 7.32-7.16 (m, 1H), 7.16-7.00 (m, 1H), 5.83-5.66 (m, 2H), 4.74-4.46 (m, 5H), 4.32-4.14 (m, 1H), 3.82 (s, 3H), 3.73-3.61 (m, 2H), 3.05 (d, J=16.8 Hz, 1H), 2.85-2.71 (m, 1H), 1.95-1.69 (m, 3H), 1.40-1.31 (m, 3H), 1.20-1.10 (m, 9H).

### Example 61: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3R)-3-(phosphonooxy)butan-2-yl) carbonate (61)

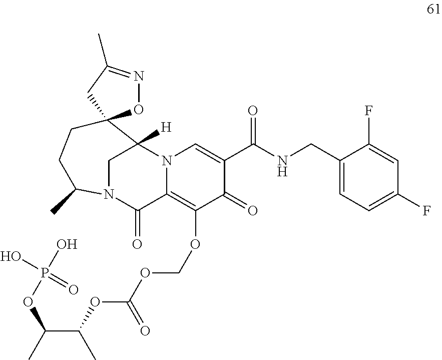 [SMILES: CC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1]

The title compound was prepared in a manner similar to Example 29, except using dibenzyl [(1R,2R)-2-hydroxy-1-methyl-propyl] phosphate instead of dibenzyl [(1S,2S)-2-hydroxy-1-methyl-propyl] phosphate. MS (m/z) 728.70 [M+H]⁺. 1H NMR (400 MHz, MeOD) δ 8.73 (d, J=2.2 Hz, 1H), 7.53-7.30 (m, 1H), 7.08-6.84 (m, 2H), 5.93 (d, J=6.6 Hz, 1H), 5.68 (d, J=6.6 Hz, 1H), 4.90-4.76 (m, 3H), 4.76-4.65 (m, 1H), 4.61 (s, 2H), 4.55-4.36 (m, 1H), 3.93-3.85 (m, 3H), 3.85-3.71 (m, 2H), 3.20 (d, J=17.0 Hz, 1H), 2.75 (d, J=17.0 Hz, 1H), 2.15-1.89 (m, 4H), 1.66-1.49 (m, 1H), 1.38-1.18 (m, 10H).

### Example 62: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl(2-methyl-1-(phosphonooxy)propan-2-yl) carbonate (62)

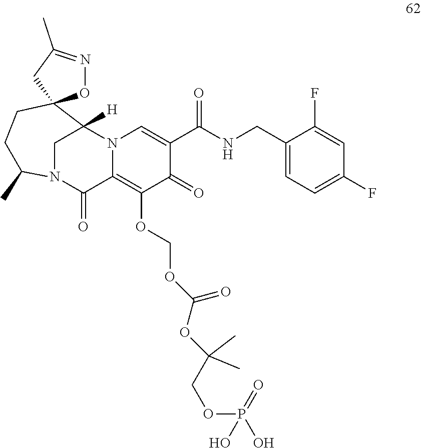 [SMILES: CC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1]

The title compound was made following the same method as Example 10, except using 2-methylpropane-1,2-diol instead of propane-1,3-diol in Step 1. MS (m/z) 713.3 [M+H]⁺. 1H NMR (400 MHz, Methanol-d4) δ 8.58 (s, 1H), 7.45 (td, J=8.5, 6.4 Hz, 1H), 7.05-6.86 (m, 2H), 5.84 (d, J=6.6 Hz, 1H), 5.65 (d, J=6.6 Hz, 1H), 4.92-4.77 (m, 1H), 4.73-4.55 (m, 2H), 4.49 (d, J=2.2 Hz, 1H), 4.06 (h, J=5.2 Hz, 2H), 3.90-3.70 (m, 2H), 3.16 (d, J=17.8 Hz, 1H), 2.77-2.62 (m, 1H), 2.06 (s, 3H), 2.01-1.85 (m, 3H), 1.56 (dt, J=10.2, 6.4 Hz, 1H), 1.48 (d, J=3.5 Hz, 6H), 1.27 (d, J=6.7 Hz, 3H).

### Example 63: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl(2-methyl-1-(phosphonooxy)propan-2-yl) carbonate (63)

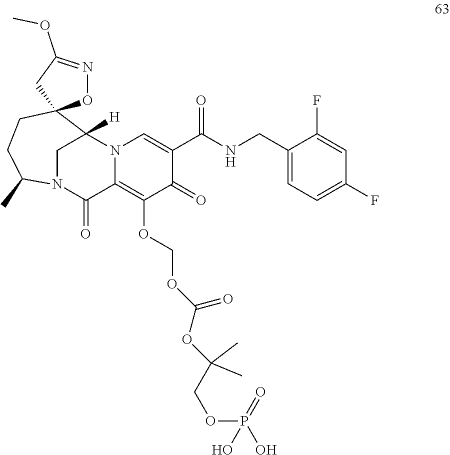 [SMILES: COC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1]

The title compound was made following the same method as Example 10, except using 2-methylpropane-1,2-diol instead of propane-1,3-diol in Step 1 and Intermediate C instead of Intermediate B in Step 3. MS (m/z) 729.2 [M+H]⁺. 1H NMR (400 MHz, Methanol-d4) δ 8.64 (s, 1H), 7.46 (td, J=8.5, 6.3 Hz, 1H), 7.05-6.91 (m, 2H), 5.83 (d, J=6.6 Hz, 1H), 5.65 (d, J=6.6 Hz, 1H), 4.92-4.77 (m, 1H), 4.63 (d, J=8.8 Hz, 3H), 4.18-3.98 (m, 2H), 3.90 (s, 3H), 3.86-3.73 (m, 2H), 3.18 (d, J=17.1 Hz, 1H), 2.77 (d, J=17.1 Hz, 1H), 2.13-1.86 (m, 3H), 1.65-1.52 (m, 1H), 1.48 (d, J=3.5 Hz, 6H), 1.27 (d, J=6.7 Hz, 3H).

### Intermediate J: Preparation of (2R)-1-[tert-butyl(dimethyl) silyl]oxybutan-2-ol (Intermediate J)

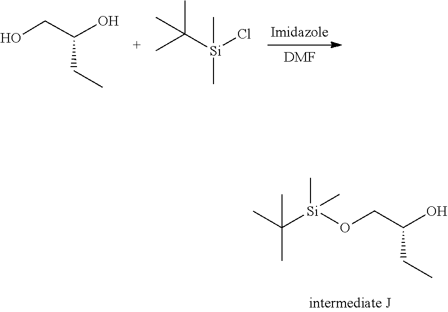

To a stirred solution of (2R)-butane-1,2-diol (1.0 g, 11.1 mmol), imidazole (755 mg, 11.1 mmol), and DMAP (136 mg, 1.11 mmol) in DMF (3 mL) at 70° C. under argon was added tert-butyl-chloro-dimethyl-silane (1.84 g, 12.2 mmol). The mixture was stirred for 3 hours. The reaction mixture was diluted with EtOAc (50 mL) and washed with saturated NH₄Cl and water. The organic layer was dried over anhydrous Na₂SO₄, filtered, and concentrated under reduced pressure and purified by silica gel column chromatography (20-70% EtOAc/Hexanes) to afford the title compound. MS (m/z) 205.1 [M+H]⁺.

### Example 64: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-(phosphonooxy)butyl) carbonate (64)

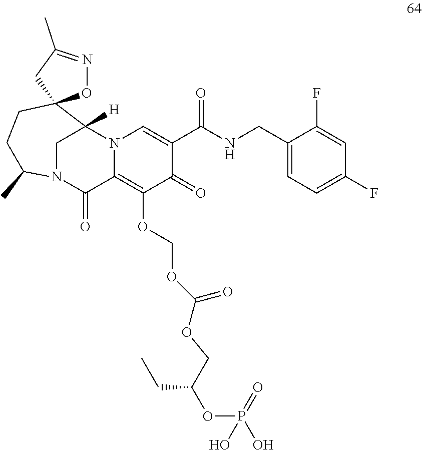 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O]

The title compound was made following the same method as Example 18, except using (2R)-1-[tert-butyl(dimethyl) silyl]oxybutan-2-ol instead of (R)-2-((tert-butyldimethylsilyl)oxy)propan-1-ol. MS (m/z) 713.1 [M+H]⁺. 1H NMR (400 MHz, DMSO-d6) § 10.28 (t, J=6.0 Hz, 1H), 8.72 (s, 1H), 7.42 (td, J=8.7, 6.6 Hz, 1H), 7.25 (ddd, J=10.4, 9.3, 2.6 Hz, 1H), 7.16-7.03 (m, 1H), 5.81 (d, J=6.4 Hz, 1H), 5.69 (d, J=6.4 Hz, 1H), 4.75-4.48 (m, 4H), 4.32-4.04 (m, 3H), 3.79-3.63 (m, 2H), 3.01 (d, J=17.6 Hz, 1H), 2.65 (d, J=17.6 Hz, 1H), 1.95 (s, 3H), 1.91-1.69 (m, 3H), 1.69-1.49 (m, 2H), 1.38-1.20 (m, 1H), 1.16 (d, J=6.6 Hz, 3H), 0.87 (t, J=7.4 Hz, 3H).

### Example 65: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-(phosphonooxy)butyl) carbonate (65)

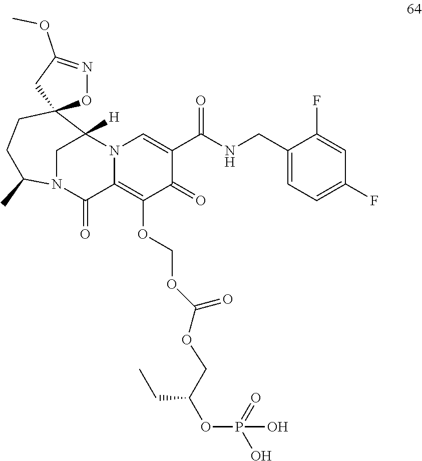 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O]

The title compound was made following the same method as Example 1, except using (2R)-1-[tert-butyl(dimethyl) silyl]oxybutan-2-ol instead of (R)-2-((tert-butyldimethylsilyl)oxy)propan-1-ol in Step 1 and Intermediate C instead of Intermediate B in Step 4. MS (m/z) 729.1 [M+H]⁺. 1H NMR (400 MHz, DMSO-d6) δ 10.27 (t, J=6.0 Hz, 1H), 8.76 (s, 1H), 7.42 (td, J=8.7, 6.6 Hz, 1H), 7.25 (ddd, J=10.5, 9.3, 2.6 Hz, 1H), 7.14-6.92 (m, 1H), 5.80 (d, J=6.4 Hz, 1H), 5.69 (d, J=6.4 Hz, 1H), 4.77-4.49 (m, 4H), 4.34-4.18 (m, 2H), 4.11 (dd, J=11.0, 4.4 Hz, 1H), 3.82 (s, 3H), 3.77-3.61 (m, 2H), 3.06 (d, J=16.8 Hz, 1H), 2.77 (d, J=16.9 Hz, 1H), 2.01-1.86 (m, 1H), 1.86-1.69 (m, 2H), 1.69-1.48 (m, 2H), 1.40-1.24 (m, 1H), 1.16 (d, J=6.7 Hz, 3H), 0.87 (t, J=7.4 Hz, 3H).

### Intermediate K: Preparation of (2S)-1-[tert-butyl(dimethyl) silyl]oxybutan-2-ol (Intermediate K)

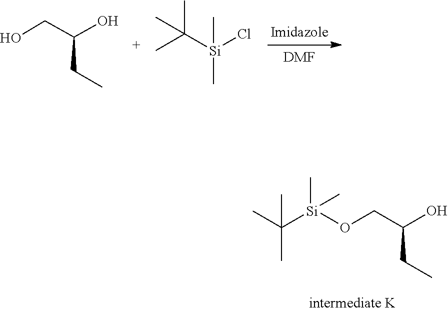

To a stirred solution of (2S)-butane-1,2-diol (1.0 g, 11.1 mmol), imidazole (755 mg, 11.1 mmol), and DMAP (136 mg, 1.11 mmol) in DMF (3 mL) at 70° C. under argon was added tert-butyl-chloro-dimethyl-silane (1.84 g, 12.2 mmol). The mixture was stirred for 3 hours. The reaction mixture was diluted with EtOAc (50 mL) and washed with saturated NH₄Cl and water. The organic layer was dried over anhydrous Na₂SO₄, filtered, concentrated under reduced pressure and purified by silica gel column chromatography (20-70% EtOAc/Hexanes) to afford the title compound. MS (m/z) 205.1 [M+H]⁺.

### Example 66: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)butyl) carbonate (66)

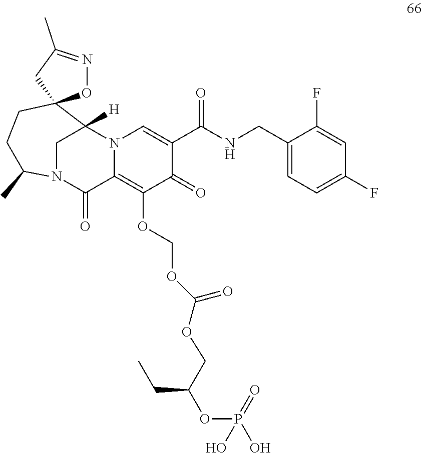 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O]

The title compound was made following the same method as Example 18, except using (2S)-1-[tert-butyl(dimethyl) silyl]oxybutan-2-ol instead of (R)-2-((tert-butyldimethylsilyl)oxy)propan-1-ol. MS (m/z) 713.1 [M+H]⁺. 1H NMR (400 MHz, Methanol-d4) δ 8.58 (s, 1H), 7.57-7.37 (m, 1H), 7.09-6.88 (m, 2H), 5.93 (d, J=6.5 Hz, 1H), 5.71 (d, J=6.5 Hz, 1H), 4.91-4.76 (m, 1H), 4.72-4.59 (m, 2H), 4.47 (s, 1H), 4.44-4.35 (m, 1H), 4.33-4.21 (m, 2H), 3.86-3.70 (m, 2H), 3.23-3.11 (m, 1H), 2.71 (d, J=17.9 Hz, 1H), 2.06 (s, 3H), 2.00-1.86 (m, 3H), 1.84-1.65 (m, J=6.8 Hz, 2H), 1.64-1.46 (m, 1H), 1.27 (d, J=6.7 Hz, 3H), 1.00 (t, J=7.5 Hz, 3H).

### Example 67: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)butyl) carbonate (67)

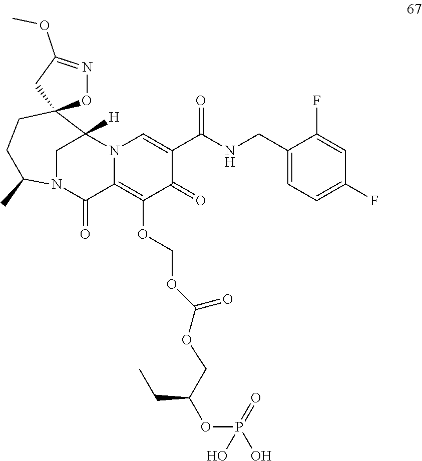 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O]

The title compound was made following the same method as Example 1, except using (2S)-1-[tert-butyl(dimethyl) silyl]oxybutan-2-ol instead of (R)-2-((tert-butyldimethylsilyl)oxy)propan-1-ol in Step 1 and Intermediate C instead of Intermediate B in Step 4. MS (m/z) 729.1 [M+H]⁺. 1H NMR (400 MHz, Methanol-d4) δ 8.66 (s, 1H), 7.45 (td, J=8.5, 6.4 Hz, 1H), 7.05-6.85 (m, 2H), 5.93 (d, J=6.5 Hz, 1H), 5.70 (d, J=6.6 Hz, 1H), 4.80 (q, J=8.2, 7.8 Hz, 1H), 4.63 (s, 3H), 4.44-4.35 (m, 1H), 4.35-4.17 (m, 2H), 3.90 (s, 3H), 3.80 (dd, J=4.0, 2.3 Hz, 2H), 3.19 (d, J=17.0 Hz, 1H), 2.77 (d, J=17.1 Hz, 1H), 2.11-1.87 (m, 3H), 1.84-1.64 (m, 2H), 1.56 (ddd, J=15.5, 9.2, 3.8 Hz, 1H), 1.26 (d, J=6.7 Hz, 3H), 1.00 (t, J=7.5 Hz, 3H).

### Intermediates L and M: Preparation of (R)-3-((bis(benzyloxy)phosphoryl)oxy)-2-methylpropyl (chloromethyl) carbonate (Intermediate L) and (S)-3-((bis(benzyloxy)phosphoryl)oxy)-2-methylpropyl (chloromethyl) carbonate (Intermediate M)

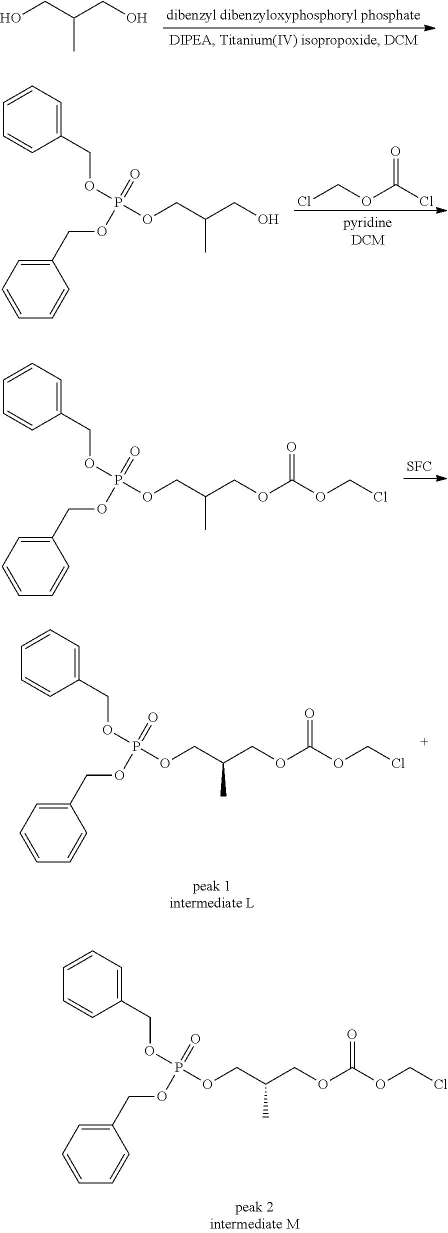

Step 1: Preparation of mixture of dibenzyl (3-hydroxy-2-methylpropyl) phosphate: Into a solution of dibenzyl dibenzyloxyphosphoryl phosphate (3 g, 5.57 mmol) and 2-methylpropane-1,3-diol (0.8 g, 8.91 mmol) in DCM (30 mL), DIPEA (1.8 g, 13.9 mmol) and titanium (IV) isopropoxide (269 mg, 0.95 mmol) were added at rt. After stirring for overnight, solvent was removed and the residue was purified by column chromatography (0-100% EtOAc/Hexanes) on silica gel to provide the mixture of title products. MS (m/z) 350.9 [M+H]⁺.

### Step 2: Preparation of mixture of 3-((bis(benzyloxy)phosphoryl)oxy)-2-methylpropyl (chloromethyl) carbonate

Into the mixture of dibenzyl (3-hydroxy-2-methyl-propyl) phosphate (1.4 g, 4 mmol) in DCM (50 mL) at 0° C. chloromethyl carbonochloridate (1.29 g, 0.89 mmol), and pyridine (948 mg, 12 mmol) were added. After addition, the reaction was allowed to warm to rt for overnight. Then the reaction mixture was extracted with ethyl acetate and washed with brine. After drying with anhydrous MgSO₄, the solvent was removed and the residue was submitted to SFC chiral separation.

### Step 3: Preparation of (R)-3-((bis(benzyloxy)phosphoryl)oxy)-2-methylpropyl (chloromethyl) carbonate (Intermediate L) and (S)-3-((bis(benzyloxy)phosphoryl)oxy)-2-methylpropyl (chloromethyl) carbonate (Intermediate M)

SFC separation of 3-((bis(benzyloxy)phosphoryl)oxy)-2-methylpropyl (chloromethyl) carbonate (Column: IG 4.6×100 mm 5mic, 3 mL/min, 20% EtOH).

(Peak 1): (R)-3-((bis(benzyloxy)phosphoryl)oxy)-2-methylpropyl (chloromethyl) carbonate (Intermediate L): MS (m/z): 442.9 [M+H]⁺.

(Peak 2): (S)-3-((bis(benzyloxy)phosphoryl)oxy)-2-methylpropyl (chloromethyl) carbonate (Intermediate M): MS (m/z): 442.9 [M+H]⁺.

### Example 68: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-methyl-3-(phosphonooxy)propyl) carbonate (68)

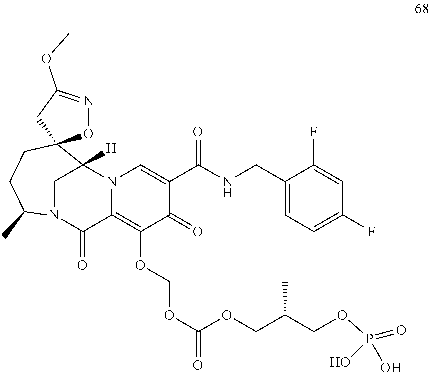 [SMILES: COC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1]

The title compound was prepared following a similar method as Example 57 except using Intermediate M instead of Intermediate B in Step 4. MS (m/z) 728.9 [M+H]⁺. 1H NMR (400 MHz, DMSO-d6) δ 10.27 (t, J=6.0 Hz, 1H), 8.74 (s, 1H), 7.42 (td, J=8.7, 6.7 Hz, 1H), 7.25 (ddd, J=12.9, 7.9, 2.7 Hz, 1H), 7.08 (td, J=8.6, 2.6 Hz, 1H), 5.80 (d, J=6.6 Hz, 1H), 5.65 (d, J=6.4 Hz, 1H), 4.71-4.52 (m, 4H), 4.05 (dd, J=10.4, 5.8 Hz, 1H), 3.98-3.87 (m, 1H), 3.82 (s, 3H), 3.71-3.59 (m, 4H), 3.07-2.90 (m, 1H), 2.76 (dd, J=22.1, 5.2 Hz, 1H), 2.09-1.74 (m, 4H), 1.36 (m, 1H), 1.15 (d, J=6.7 Hz, 3H), 0.88 (d, J=6.8 Hz, 3H).

### Example 69: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-methyl-3-(phosphonooxy)propyl) carbonate (69)

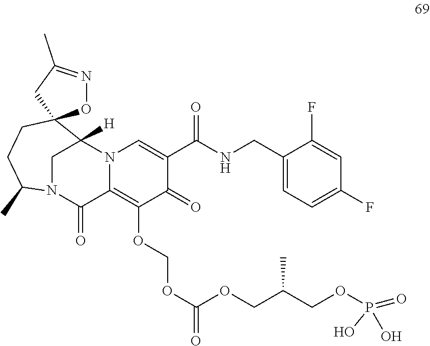 [SMILES: CC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1]

The title compound was prepared following a similar method as Example 18 except using (R)-3-((bis(benzyloxy)phosphoryl)oxy)-2-methylpropyl (chloromethyl) carbonate (Intermediate M) instead of (R)-1-((bis(benzyloxy)phosphoryl)oxy)propan-2-yl (chloromethyl) carbonate in Step 4. MS (m/z) 712.6 [M+H]⁺. ¹H NMR (400 MHz, DMSO) δ 10.28 (t, J=6.0 Hz, 1H), 8.71 (s, 1H), 7.42 (q, J=8.4 Hz, 1H), 7.36-7.18 (m, 1H), 7.10-7.03 (m, 1H), 5.83 (d, J=6.5 Hz, 1H), 5.65 (d, J=6.5 Hz, 1H), 4.66-4.53 (m, 4H), 4.05 (dd, J=10.6, 5.7 Hz, 1H), 3.94 (t, J=8.6 Hz, 1H), 3.71 (d, J=21.9 Hz, 4H), 2.96 (d, J=10.4 Hz, 1H), 2.69-2.60 (m, 1H), 2.05 (dd, J=12.4, 6.2 Hz, 1H), 1.95 (d, J=8.9 Hz, 3H), 1.82-1.73 (m, 3H), 1.29-1.13 (m, 5H), 0.89 (d, J=6.7 Hz, 3H).

### Example 70: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-methyl-3-(phosphonooxy)propyl) carbonate (70)

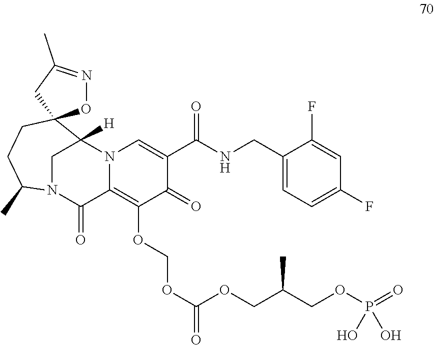 [SMILES: CC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1]

The title compound was prepared following a similar method as Example 18 except using(S)-3-((bis(benzyloxy)phosphoryl)oxy)-2-methylpropyl (chloromethyl) carbonate (Intermediate L) instead of (R)-1-((bis(benzyloxy)phosphoryl)oxy)propan-2-yl (chloromethyl) carbonate in Step 4. MS (m/z) 712.6 [M+H]⁺. 1H NMR (400 MHz, DMSO-d6) δ 10.28 (t, J=6.0 Hz, 1H), 8.71 (s, 1H), 7.43 (td, J=8.7, 6.6 Hz, 1H), 7.25 (ddd, J=10.5, 9.3, 2.6 Hz, 1H), 7.13-7.03 (m, 1H), 5.82 (d, J=6.5 Hz, 1H), 5.66 (d, J=6.5 Hz, 1H), 4.63 (s, 2H), 4.62-4.47 (m, 2H), 4.06 (dd, J=10.6, 6.0 Hz, 1H), 3.95 (dd, J=10.6, 6.3 Hz, 1H), 3.79-3.65 (m, 4H), 3.01 (d, J=17.6 Hz, 1H), 2.63 (d, J=17.6 Hz, 1H), 2.07 (dq, J=12.7, 6.3 Hz, 1H), 1.95 (s, 3H), 1.78 (ddd, J=21.2, 10.7, 5.6 Hz, 3H), 1.29 (dd, J=15.0, 11.6 Hz, 1H), 1.16 (d, J=6.7 Hz, 3H), 0.90 (d, J=6.9 Hz, 3H).

### Example 71: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-methyl-3-(phosphonooxy)propyl) carbonate (71)

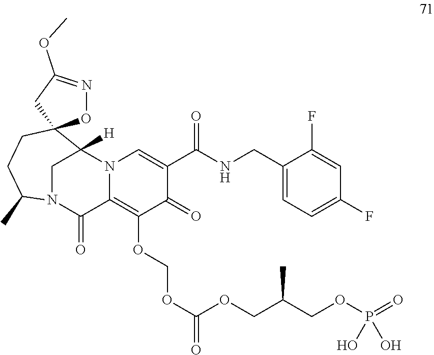 [SMILES: COC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1]

The title compound was prepared following a similar method as Example 57 except using Intermediate L instead of Intermediate B in Step 4. MS (m/z) 728.9 [M+H]⁺. 1H NMR (400 MHz, DMSO-d6) δ 10.27 (t, J=5.9 Hz, 1H), 8.75 (s, 1H), 7.42 (td, J=8.7, 6.6 Hz, 1H), 7.25 (ddd, J=10.5, 9.3, 2.6 Hz, 1H), 7.08 (td, J=8.6, 2.8 Hz, 1H), 5.80 (d, J=6.4 Hz, 1H), 5.66 (d, J=6.4 Hz, 1H), 4.71 (s, 1H), 4.66-4.58 (m, 1H), 4.61-4.47 (m, 2H), 4.07 (dd, J=10.6, 6.0 Hz, 1H), 3.95 (dd, J=10.6, 6.3 Hz, 1H), 3.82 (s, 3H), 3.79-3.64 (m, 4H), 3.05 (d, J=16.8 Hz, 1H), 2.76 (d, J=16.9 Hz, 1H), 2.08 (h, J=6.3 Hz, 1H), 1.91 (dq, J=13.5, 7.6, 5.9 Hz, 1H), 1.87-1.69 (m, 2H), 1.33 (dd, J=15.5, 10.8 Hz, 1H), 1.15 (d, J=6.7 Hz, 3H), 0.90 (d, J=6.8 Hz, 3H).

### Intermediates N and O: Preparation of (2S,3S)-3-((bis(benzyloxy)phosphoryl)oxy)pentan-2-yl (chloromethyl) carbonate (Intermediate N) and (2S,3R)-3-((bis(benzyloxy)phosphoryl)oxy)pentan-2-yl (chloromethyl) carbonate (Intermediate O)

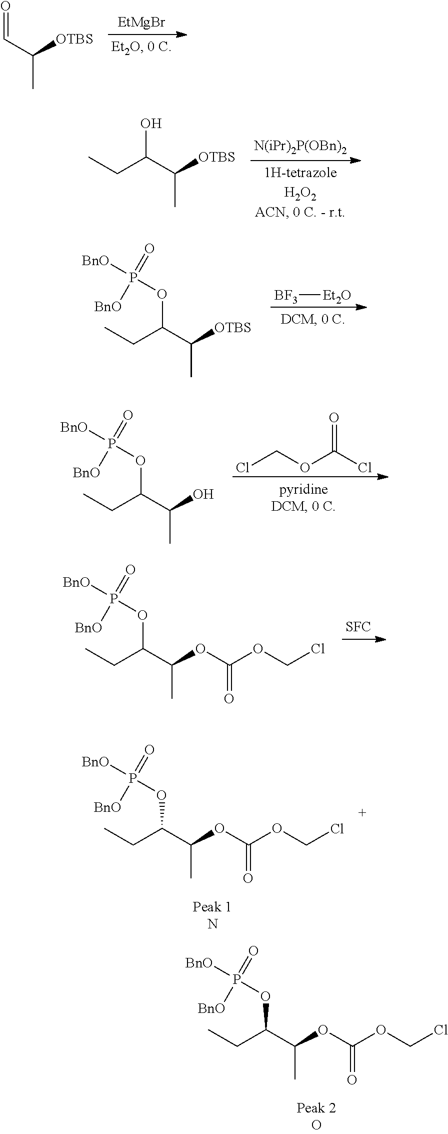

### Step 1: Synthesis of (2S)-2-((tert-butyldimethylsilyl)oxy)pentan-3-ol

To a solution of(S)-2-((tert-butyldimethylsilyl)oxy)propanal (2.0 g, 10.6 mmol) in diethyl ether (0.2 M) at 0° C. was added ethylmagnesium bromide (3.0 M in diethyl ether, 3.90 mL, 11.7 mmol) dropwise. The reaction was then warmed to room temperature and stirred at room temperature for 16 h. The reaction mixture was then quenched with saturated ammonium chloride, extracted with ethyl acetate, dried over magnesium sulfate, filtered, concentrated to afford the title compound as a mixture of diastereomers. The product was used directly in the next step without further purification.

### Step 2: Synthesis of dibenzyl ((2S)-2-((tert-butyldimethylsilyl)oxy)pentan-3-yl) phosphate

To a solution of (2S)-2-((tert-butyldimethylsilyl)oxy)pentan-3-ol (2.2 g, 10.1 mmol) in acetonitrile (0.1 M) at 0° C. was added 1H-tetrazole (0.85 g, 12.1 mmol) followed by N-dibenzyloxyphosphanyl-N-isopropyl-propan-2-amine (4.26 g, 12.1 mmol). The reaction mixture was stirred at 0° C. for 1 h. Then hydrogen peroxide (30% in water, 1.48 mL, 20.1 mmol) was added dropwise, and the reaction mixture was stirred for an addition 30 min. The reaction was then quenched with saturated sodium thiosulfate and saturated sodium bicarbonate, extracted with DCM, dried over magnesium sulfate, filtered, dried, concentrated and purified by normal phase silica gel chromatography (0-100% EtOAc in Hexanes) to afford the title compound. ¹H NMR (400 MHz, CDCl₃) δ 7.42-7.31 (m, 10H), 5.12-4.97 (m, 4H), 4.30-4.11 (m, 1H), 4.08-3.92 (m, 1H), 1.88-1.48 (m, 2H), 1.17-1.07 (m, 2H), 1.04-0.93 (m, 3H), 0.93-0.85 (m, 9H), 0.11-0.00 (m, 6H).

### Step 3: Synthesis of dibenzyl ((2S)-2-hydroxypentan-3-yl) phosphate

To a solution of dibenzyl ((2S)-2-((tert-butyldimethylsilyl)oxy)pentan-3-yl) phosphate (2.89 g, 5.74 mmol) in DCM (0.3 M) at 0° C. was added boron trifluoride diethyl etherate (1.81 mL, 6.88 mmol) dropwise. The reaction mixture was then stirred at 0° C. for 30 min. The reaction mixture was quenched with saturated sodium bicarbonate, extracted with DCM, dried, concentrated and purified by silica gel chromatography (0-100% EtOAc in Hexanes) to afford the title compound. MS (m/z): 365.0 [M+H]⁺.

### Step 4: Synthesis of (2S)-3-(bis(benzyloxy)phosphoryl)oxy)pentan-2-yl (chloromethyl) carbonate

To a solution of dibenzyl ((2S)-2-hydroxypentan-3-yl) phosphate (1.0 g, 2.74 mmol) in DCM at 0° C. (0.2 M) was added chloromethyl chloroformate (0.268 mL, 3.02 mmol) followed by pyridine (0.277 mL, 3.43 mmol) dropwise. The reaction mixture was stirred for 30 min, then quenched with water and 1 M HCl. The reaction was extracted with DCM, dried, filtered and concentrated to afford the title compound used directly in the next step without further purification. MS (m/s): 456.9 [M+H]⁺.

### Step 5: Synthesis of (2S,3S)-3-((bis(benzyloxy)phosphoryl)oxy)pentan-2-yl (chloromethyl) carbonate (Intermediate N) and (2S,3R)-3-((bis(benzyloxy)phosphoryl)oxy)pentan-2-yl (chloromethyl) carbonate (Intermediate O)

SFC separation of (2S)-3-((bis(benzyloxy)phosphoryl)oxy)pentan-2-yl (chloromethyl) carbonate (Column: AS-H 5 μm 20×250 mm, 50 mL/min, 15% EtOH).

Peak 1:(2S,3S)-3-((bis(benzyloxy)phosphoryl)oxy)pentan-2-yl (chloromethyl) carbonate (Intermediate N): MS (m/z): 456.8 [M+H]⁺.

Peak 2: (2S,3R)-3-((bis(benzyloxy)phosphoryl)oxy)pentan-2-yl (chloromethyl) carbonate (Intermediate O): MS (m/z): 456.9 [M+H]⁺.

### Example 72: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)pentan-2-yl) carbonate (72)

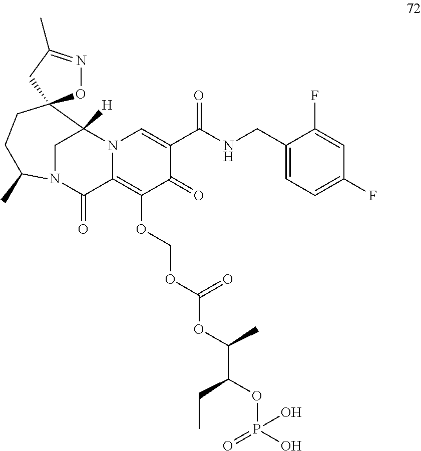 [SMILES: CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared following a similar method as Example 18 except using (2S,3S)-3-((bis(benzyloxy)phosphoryl)oxy)pentan-2-yl (chloromethyl) carbonate (Intermediate N) instead of (R)-1-((bis(benzyloxy)phosphoryl)oxy)propan-2-yl (chloromethyl) carbonate in Step 4. MS (m/z): 726.9 [M+H]⁺. 1H NMR (400 MHz, MeOD) δ 8.58 (s, 1H), 7.55-7.32 (m, 1H), 7.06-6.83 (m, 2H), 6.02-5.86 (m, 1H), 5.85-5.59 (m, 1H), 5.00-4.90 (m, 1H), 4.69-4.60 (m, 3H), 4.47 (s, 1H), 4.22 (s, 1H), 3.93-3.73 (m, 2H), 3.21-3.08 (m, 1H), 2.95 (d, J=53.0 Hz, 2H), 2.78-2.64 (m, 1H), 2.07 (s, 3H), 1.98-1.88 (m, 3H), 1.75-1.64 (m, 2H), 1.64-1.46 (m, 1H), 1.37-1.16 (m, 7H), 0.99 (t, J=7.4 Hz, 3H).

### Example 73: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3R)-3-(phosphonooxy)pentan-2-yl) carbonate (73)

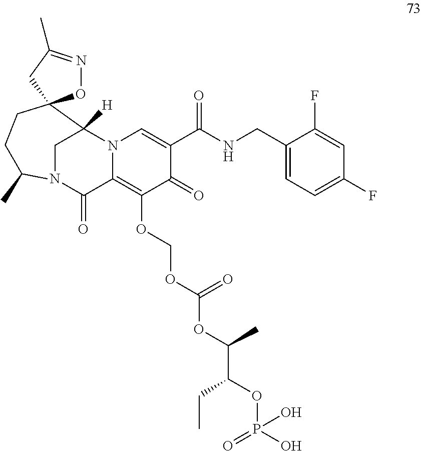 [SMILES: CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared following a similar method as Example 18 except using (2S,3S)-3-((bis(benzyloxy)phosphoryl)oxy)pentan-2-yl (chloromethyl) carbonate (Intermediate O) instead of (R)-1-((bis(benzyloxy)phosphoryl)oxy)propan-2-yl (chloromethyl) carbonate in Step 4. MS (m/z): 726.9 [M+H]⁺. 1H NMR (400 MHz, MeOD) δ 8.58 (s, 1H), 7.58-7.34 (m, 1H), 7.08-6.87 (m, 2H), 5.94 (d, J=6.5 Hz, 1H), 5.70 (d, J=6.5 Hz, 1H), 4.88-4.74 (m, 1H), 4.65 (s, 2H), 4.47 (s, 1H), 4.35-4.23 (m, 1H), 3.91-3.65 (m, 2H), 3.16 (d, J=17.9 Hz, 1H), 2.95 (d, J=53.0 Hz, 3H), 2.70 (d, J=17.9 Hz, 1H), 2.06 (s, 3H), 2.02-1.85 (m, 4H), 1.78-1.64 (m, 2H), 1.62-1.49 (m, 1H), 1.36-1.18 (m, 6H), 1.01 (t, J=7.4 Hz, 3H).

### Example 74: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)pentan-2-yl) carbonate (74)

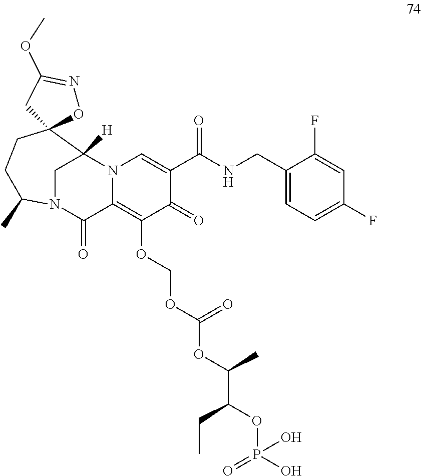 [SMILES: CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 72 except using Intermediate C instead of Intermediate B in Step 4. MS (m/z): 742.9 [M+H]⁺. 1H NMR (400 MHz, MeOD) δ 8.69-8.50 (m, 1H), 7.57-7.40 (m, 1H), 7.07-6.89 (m, 2H), 5.93 (d, J=6.6 Hz, 1H), 5.78-5.58 (m, 1H), 5.06-4.87 (m, 1H), 4.83 (t, J=5.6 Hz, 3H), 4.75-4.54 (m, 3H), 4.35-4.13 (m, 1H), 3.98-3.66 (m, 4H), 3.11-2.98 (m, 3H), 2.98-2.64 (m, 2H), 2.12-1.86 (m, 3H), 1.83-1.51 (m, 3H), 1.42 (s, 2H), 1.35-1.21 (m, 4H), 1.07-0.92 (m, 2H).

### Example 75: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3R)-3-(phosphonooxy)pentan-2-yl) carbonate (75)

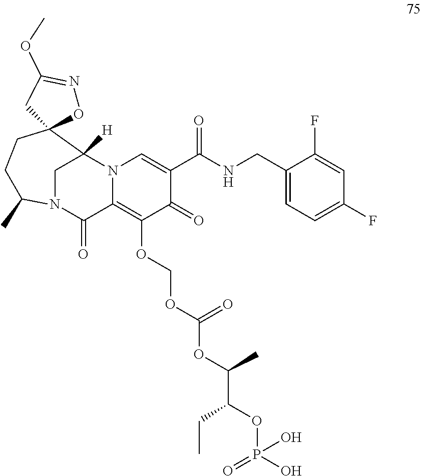 [SMILES: CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 73 except using Intermediate C instead of Intermediate B in Step 4. MS (m/z): 726.9 [M+H]⁺. 1H NMR (400 MHz, MeOD) δ 8.62 (s, 1H), 7.52-7.36 (m, 1H), 7.07-6.86 (m, 2H), 5.93 (d, J=6.5 Hz, 1H), 5.69 (d, J=6.5 Hz, 1H), 4.75-4.49 (m, 5H), 4.26 (s, 1H), 3.99-3.73 (m, 5H), 3.23-3.06 (m, 1H), 3.07-2.85 (m, 2H), 2.77 (d, J=17.1 Hz, 1H), 2.14-1.88 (m, 4H), 1.81-1.65 (m, 2H), 1.65-1.53 (m, 1H), 1.35-1.20 (m, 6H), 1.01 (t, J=7.4 Hz, 3H).

### Intermediates P and Q: Preparation of (2R,3R)-3-((bis(benzyloxy)phosphoryl)oxy)pentan-2-yl (chloromethyl) carbonate (Intermediate P) and (2R,3S)-3-((bis(benzyloxy)phosphoryl)oxy)pentan-2-yl (chloromethyl) carbonate (Intermediate Q)

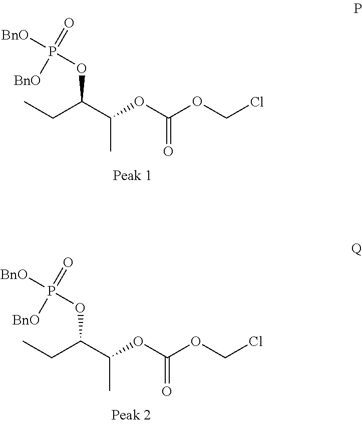 [SMILES: CC[C@@H](OP(=O)(OCc1ccccc1)OCc1ccccc1)[C@@H](C)OC(=O)OCCl ; CC[C@H](OP(=O)(OCc1ccccc1)OCc1ccccc1)[C@@H](C)OC(=O)OCCl]

The title compounds were prepared in a manner similar to Intermediate N and Intermediate O, except using (R)-2-((tert-butyldimethylsilyl)oxy)propanal instead of(S)-2-((tert-butyldimethylsilyl)oxy)propanal in step 1.

SFC separation of (2R)-3-((bis(benzyloxy)phosphoryl)oxy)pentan-2-yl (chloromethyl) carbonate (Column: AD-H 5 μm 21×250 mm, 60 mL/min, 5% EtOH—NH₃).

Peak 1:(2R,3R)-3-((bis(benzyloxy)phosphoryl)oxy)pentan-2-yl (chloromethyl) carbonate (Intermediate P): MS (m/z): 456.8 [M+H]⁺.

Peak 2: (2R,3S)-3-((bis(benzyloxy)phosphoryl)oxy)pentan-2-yl (chloromethyl) carbonate (Intermediate Q): MS (m/z): 456.8 [M+H]⁺.

### Example 76: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3R)-3-(phosphonooxy)pentan-2-yl) carbonate (76)

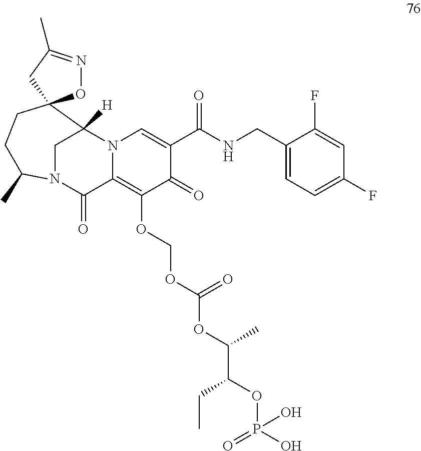 [SMILES: CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 18 except using (2R,3R)-3-((bis(benzyloxy)phosphoryl)oxy)pentan-2-yl (chloromethyl) carbonate (Intermediate P) instead of (R)-1-((bis(benzyloxy)phosphoryl)oxy)propan-2-yl (chloromethyl) carbonate in Step 4. MS (m/z): 726.9 [M+H]⁺. ¹H NMR (400 MHz, MeOD) δ 8.58 (s, 1H), 7.59-7.30 (m, 1H), 7.14-6.88 (m, 2H), 6.06-5.57 (m, 2H), 4.86-4.55 (m, 3H), 4.48 (s, 1H), 4.35-4.20 (m, 1H), 3.91-3.67 (m, 2H), 3.21-3.12 (m, 2H), 2.95 (d, J=53.0 Hz, 2H), 2.71 (d, J=17.9 Hz, 1H), 2.17-2.01 (m, 4H), 2.01-1.86 (m, 3H), 1.77-1.64 (m, 2H), 1.64-1.47 (m, 1H), 1.34-1.18 (m, 6H), 1.00 (t, J=7.4 Hz, 3H).

### Example 77: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3S)-3-(phosphonooxy)pentan-2-yl) carbonate (77)

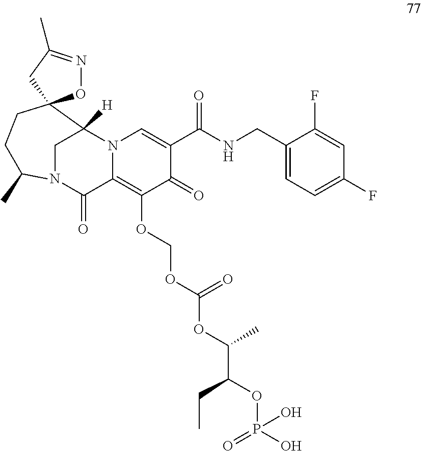 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 18 except using (2R,3S)-3-((bis(benzyloxy)phosphoryl)oxy)pentan-2-yl (chloromethyl) carbonate (Intermediate Q) instead of (R)-1-((bis(benzyloxy)phosphoryl)oxy)propan-2-yl (chloromethyl) carbonate in Step 4. MS (m/z): 726.9 [M+H]⁺. ¹H NMR (400 MHz, MeOD) δ 8.58 (s, 1H), 7.63-7.33 (m, 1H), 7.24-6.82 (m, 2H), 5.89 (d, J=6.6 Hz, 1H), 5.75 (d, J=6.7 Hz, 1H), 5.00-4.90 (m, 1H), 4.75-4.45 (m, 4H), 4.20 (s, 1H), 3.87-3.57 (m, 3H), 3.22-3.09 (m, 1H), 2.95 (d, J=52.9 Hz, 1H), 2.72 (d, J=17.9 Hz, 1H), 2.09-2.03 (m, 4H), 2.00-1.85 (m, 3H), 1.85-1.47 (m, 2H), 1.37-1.22 (m, 7H), 1.07-0.94 (m, 3H).

### Example 78: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3R)-3-(phosphonooxy)pentan-2-yl) carbonate (78)

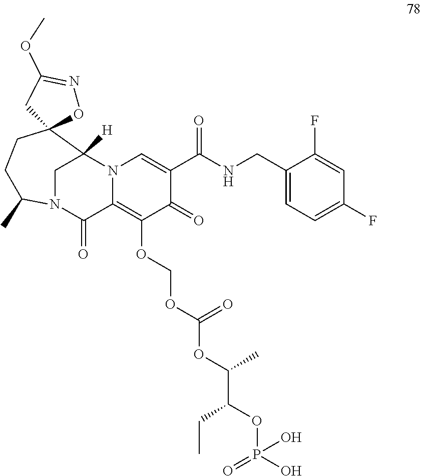 [SMILES: CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 76 except using Intermediate C instead of Intermediate B in Step 4. MS (m/z): 742.9 [M+H]⁺. 1H NMR (400 MHz, MeOD) δ 8.63 (s, 1H), 7.61-7.36 (m, 1H), 7.12-6.59 (m, 2H), 5.95-5.72 (m, 2H), 4.87-4.74 (m, 1H), 4.71-4.55 (m, 3H), 4.35-4.18 (m, 1H), 3.96-3.87 (m, 3H), 3.89-3.69 (m, 2H), 3.18 (d, J=17.1 Hz, 1H), 2.95 (d, J=53.0 Hz, 2H), 2.78 (d, J=17.1 Hz, 1H), 2.13-1.90 (m, 4H), 1.79-1.50 (m, 3H), 1.36-1.21 (m, 7H), 1.00 (t, J=7.4 Hz, 3H).

### Example 79: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3S)-3-(phosphonooxy)pentan-2-yl) carbonate (79)

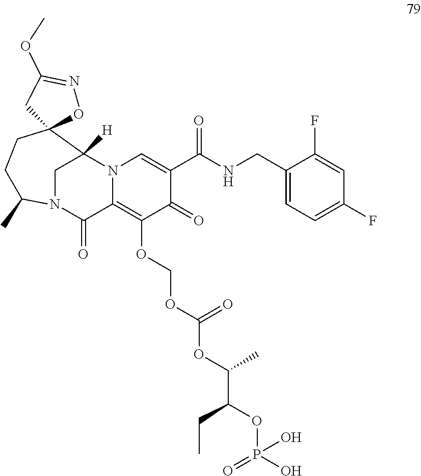 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 77 except using Intermediate C instead of Intermediate B in Step 4. MS (m/z): 742.9 [M+H]⁺. ¹HNMR (400 MHz, MeOD) δ 8.62 (s, 1H), 7.49-7.40 (m, 1H), 7.09-6.82 (m, 2H), 5.89 (d, J=6.6 Hz, 1H), 5.74 (d, J=6.7 Hz, 1H), 5.03-4.89 (m, 1H), 4.85-4.75 (m, 1H), 4.70-4.57 (m, 3H), 4.28-4.17 (m, 1H), 3.91 (s, 3H), 3.90-3.73 (m, 2H), 3.24-3.12 (m, 1H), 2.95 (d, J=53.0 Hz, 2H), 2.78 (d, J=17.1 Hz, 1H), 2.16-1.84 (m, 3H), 1.84-1.48 (m, 3H), 1.36-1.18 (m, 7H), 1.05-0.92 (m, 3H).

### Intermediates R and S: (R)-2-((bis(benzyloxy)phosphoryl)oxy)-3-methylbutyl (chloromethyl) carbonate (Intermediate R) and (S)-2-((bis(benzyloxy)phosphoryl)oxy)-3-methylbutyl (chloromethyl) carbonate (Intermediate S)

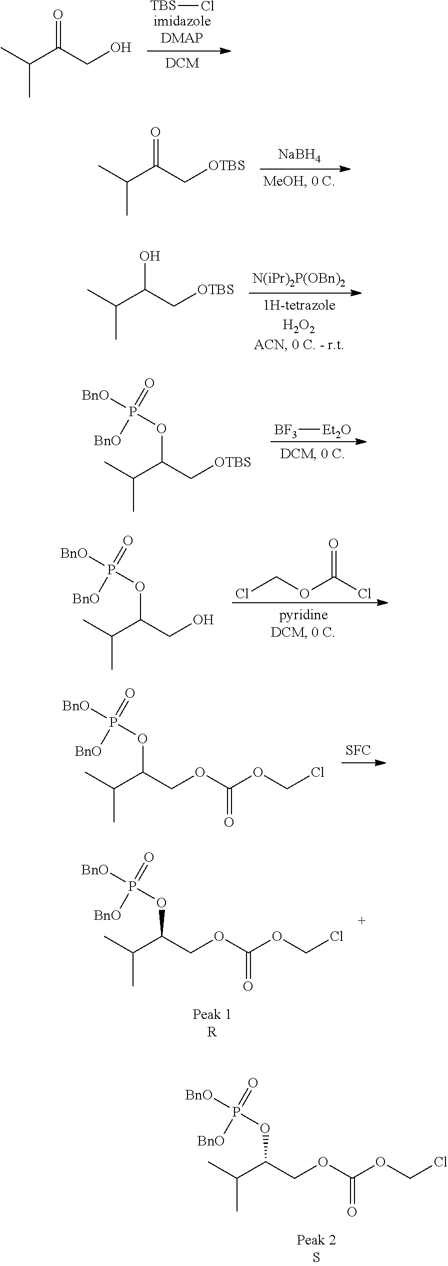

### Step 1: Synthesis of 1-((tert-butyldimethylsilyl)oxy)-3-methylbutan-2-one

To a solution of 1-hydroxy-3-methyl-butan-2-one (1.0 g, 9.79 mmol) in DCM (0.2 M) was added imidazole (800 mg, 11.7 mmol), 4-dimethylaminopyridine (120 mg, 0.97 mmol) and TBS-C₁ (1.77 g, 11.7 mmol). The reaction was stirred for 16 h at room temperature. The reaction mixture was then concentrated and purified by silica gel chromatography to afford the title compound. ¹H NMR (400 MHz, CDCl₃) δ 4.28 (s, 2H), 2.92 (hept, J=6.9 Hz, 1H), 1.12 (d, J=6.9 Hz, 6H), 0.95 (s, 9H), 0.11 (s, 6H).

### Step 2: Synthesis of 1-((tert-butyldimethylsilyl)oxy)-3-methylbutan-2-ol

To a solution of 1-((tert-butyldimethylsilyl)oxy)-3-methylbutan-2-one (1.8 g, 8.3 mmol) was in MeOH (0.2 M) at 0° C. was added sodium borohydride (472 mg, 12 mmol) portionwise. The reaction mixture was stirred for 3 h and then quenched with water, extracted with EtOAc, dried, filtered, concentrated and purified by silica gel chromatography (0-100% EtOAc in Hexanes) to afford the title compound. ¹H NMR (400 MHz, CDCl₃) δ 3.75-3.67 (m, 1H), 3.54-3.45 (m, 1H), 3.43-3.31 (m, 1H), 1.76-1.62 (m, 1H), 1.00 (d, J=6.8 Hz, 3H), 0.97-0.86 (m, 9H), 0.15-0.03 (m, 6H).

### Step 3: Synthesis of dibenzyl (1-((tert-butyldimethylsilyl)oxy)-3-methylbutan-2-yl) phosphate

To a solution of 1-((tert-butyldimethylsilyl)oxy)-3-methylbutan-2-ol (900 mg, 4.12 mmol) in acetonitrile (0.1 M) at 0° C. was added 1H-tetrazole (0.35 g, 4.94 mmol) followed by N-dibenzyloxyphosphanyl-N-isopropyl-propan-2-amine (1.74 g, 4.94 mmol). The reaction mixture was stirred at 0° C. for 1 h. Then hydrogen peroxide (30% in water, 0.61 mL, 8.24 mmol) was added dropwise, and the reaction mixture was stirred for an addition 30 min. The reaction was then quenched with saturated sodium thiosulfate and saturated sodium bicarbonate, extracted with DCM, dried over magnesium sulfate, filtered, dried, concentrated and purified by normal phase silica gel chromatography (0-100% EtOAc in Hexanes) to afford the title compound. MS (m/z): 463 [M+(—CH3)]⁺.

### Step 4: Synthesis of dibenzyl (1-hydroxy-3-methylbutan-2-yl) phosphate

To a solution of dibenzyl (1-((tert-butyldimethylsilyl)oxy)-3-methylbutan-2-yl) phosphate (1.6 g, 3.18 mmol) in DCM (0.3 M) at 0° C. was added boron trifluoride diethyl etherate (1.00 mL, 3.81 mmol) dropwise. The reaction mixture was then stirred at 0° C. for 30 min. The reaction mixture was quenched with saturated sodium bicarbonate, extracted with DCM, dried, concentrated and purified by silica gel chromatography (0-100% EtOAc in Hexanes) to afford the title compound. MS (m/z): 365.0 [M+H]⁺.

### Step 5: Synthesis of 2-((bis(benzyloxy)phosphoryl)oxy)-3-methylbutyl (chloromethyl) carbonate

To a solution of dibenzyl (1-hydroxy-3-methylbutan-2-yl) phosphate (1.3 g, 3.60 mmol) in DCM at 0° C. (0.2 M) was added chloromethyl chloroformate (0.799 mL, 8.99 mmol) followed by pyridine (0.798 mL, 9.89 mmol) dropwise. The reaction mixture was stirred for 30 min, then quenched with water and 1 M HCl. The reaction was extracted with DCM, dried, filtered and concentrated to afford the title compound used directly in the next step without further purification. MS (m/s): 456.9 [M+H]⁺.

### Step 6: Synthesis of (R)-2-((bis(benzyloxy)phosphoryl)oxy)-3-methylbutyl (chloromethyl) carbonate (Intermediate R) and (S)-2-((bis(benzyloxy)phosphoryl)oxy)-3-methylbutyl (chloromethyl) carbonate (Intermediate S)

SFC separation of 2-((bis(benzyloxy)phosphoryl)oxy)-3-methylbutyl (chloromethyl) carbonate (Column: AD-H 5 μm 21×250 mm, 60 mL/min, 10% EtOH—NH₃).

Peak 1: (R)-2-((bis(benzyloxy)phosphoryl)oxy)-3-methylbutyl (chloromethyl) carbonate (Intermediate R): MS (m/z): 456.7 [M+H]⁺.

Peak 2: (S)-2-((bis(benzyloxy)phosphoryl)oxy)-3-methylbutyl (chloromethyl) carbonate (Intermediate S): MS (m/z): 456.3 [M+H]⁺.

### Example 80: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-3-methyl-2-(phosphonooxy)butyl) carbonate (80)

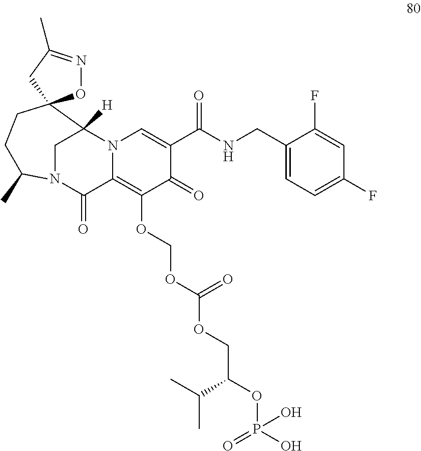 [SMILES: CC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1]

The title compound was prepared in a manner similar to Example 18 except using (R)-2-((bis(benzyloxy)phosphoryl)oxy)-3-methylbutyl (chloromethyl) carbonate (Intermediate R) instead of (R)-1-((bis(benzyloxy)phosphoryl)oxy)propan-2-yl (chloromethyl) carbonate in Step 4. MS (m/z): 726.8 [M+H]⁺. 1H NMR (400 MHz, MeOD) δ 8.58 (d, J=2.0 Hz, 1H), 7.55-7.37 (m, 1H), 7.08-6.88 (m, 3H), 6.08-5.67 (m, 2H), 4.76-4.57 (m, 3H), 4.47 (s, 1H), 4.40-4.18 (m, 1H), 4.09 (s, 1H), 3.94-3.69 (m, 2H), 3.23-3.10 (m, 1H), 3.01 (s, 2H), 2.95-2.81 (m, 2H), 2.81-2.61 (m, 1H), 2.07 (s, 3H), 2.01-1.88 (m, 3H), 1.61-1.49 (m, 1H), 1.29-1.24 (m, 3H), 1.03-0.96 (m, 6H).

### Example 81: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-3-methyl-2-(phosphonooxy)butyl) carbonate (81)

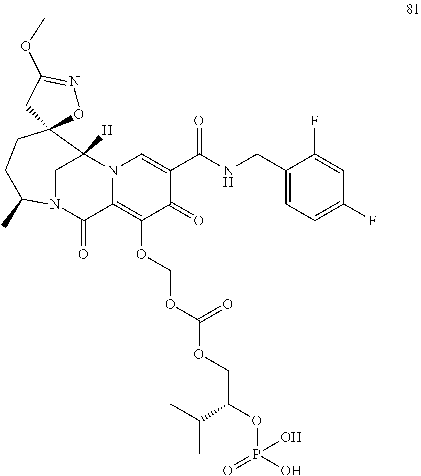 [SMILES: COC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1]

The title compound was prepared in a manner similar to Example 80 except using Intermediate C instead of Intermediate B. in Step 4. MS (m/z): 742.9 [M+H]⁺. 1H NMR (400 MHz, MeOD) δ 8.67-8.46 (m, 1H), 7.55-7.36 (m, 1H), 7.04-6.92 (m, 2H), 6.03-5.68 (m, 1H), 4.73-4.55 (m, 3H), 4.37-3.97 (m, 1H), 3.97-3.70 (m, 3H), 3.21-3.12 (m, 1H), 3.11-2.84 (m, 4H), 2.84-2.65 (m, 1H), 2.26-1.83 (m, 7H), 1.62-1.54 (m, 2H), 1.42 (s, 1H), 1.38-1.15 (m, 3H), 1.09-0.90 (m, 6H).

### Example 82: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-3-methyl-2-(phosphonooxy)butyl) carbonate (82)

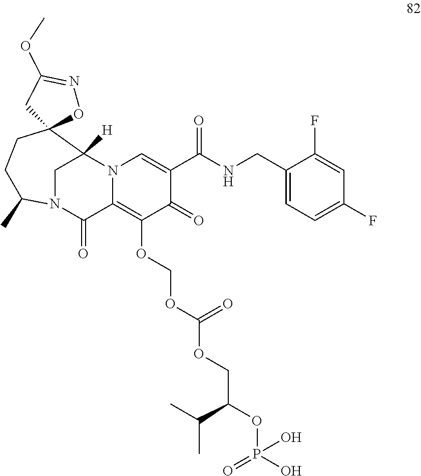 [SMILES: COC1=NO[C@@]2(CC[C@H](C)N3C[C@H]2n2cc(C(=O)NCc4ccc(F)cc4F)c(=O)c(OCOC(=O)OC[C@@H](OP(=O)(O)O)C(C)C)c2C3=O)C1]

The title compound was prepared in a manner similar to Example 80 except using Intermediate C instead of Intermediate B. in Step 4. MS (m/z): 742.8 [M+H]⁺. ¹H NMR (400 MHz, MeOD) δ 8.62 (s, 1H), 7.46 (s, 1H), 7.07-6.85 (m, 2H), 5.90 (d, J=6.2 Hz, 1H), 5.74 (s, 1H), 4.94-4.89 (m, 1H), 4.86-4.76 (m, 1H), 4.72-4.49 (m, 3H), 4.37-4.26 (m, 1H), 4.28-4.12 (m, 2H), 4.01-3.88 (m, 2H), 3.87-3.57 (m, 3H), 2.85-2.54 (m, 2H), 2.24 (s, 1H), 2.17-1.75 (m, 3H), 1.67-1.49 (m, 1H), 1.42 (s, 3H), 1.36-1.19 (m, 4H), 1.08-0.86 (m, 4H).

### Example A: HIV MT-4 Antiviral and Cytotoxicity Assay

### Antiviral Assay in MT-4 Cells

Compounds were tested in a high-throughput 384-well assay format for their ability to inhibit the replication of HIV-1 (IIIB) in MT-4 cells. Compounds were serially diluted (1:3) in DMSO on 384-well polypropylene plates and further diluted 200-fold into complete RPMI media (10% FBS, 1% P/S) using the Biotek Micro Flow and Labcyte ECHO acoustic dispenser. Each plate contained up to 8 test compounds, with negative (No Drug Control) and 5 μM AZT positive controls. MT-4 cells were pre-infected with 10 μL of either RPMI (mock-infected) or a fresh 1:250 dilution of HIV-1 IIIB concentrated virus stock. Infected and uninfected MT-4 cells were further diluted in complete RPMI media and added to each plate using a Micro Flow dispenser. After 5 days incubation in a humidified and temperature controlled incubator (37° C.), Cell Titer Glo (Promega) was added to the assay plates and chemiluminescence read using an Envision plate-reader. EC₅₀ values were defined as the compound concentration that causes a 50% decrease in luminescence signal, and were calculated using a sigmoidal dose-response model to generate curve fits. The EC₅₀ data for exemplary compounds is shown in Table 1.

### Cytotoxicity Assay in MT-4 Cells

Assays were performed as above except uninfected MT-4 cells were added to each well containing test compound. In addition, 10 μM puromycin was added to the last column of each assay plate to assess a base level of cytotoxicity. The CC₅₀ data for exemplary compounds is shown in Table 1.

TABLE 1

| Compound | MT4 EC₅₀ | MT4 CC₅₀ |
|---|---|---|
| No. | (nM) | (nM) |
| 1 | 1.77 | 15429 |
| 2 | 1.37 | 12251 |
| 3 | 0.46 | 13012 |
| 4 | 0.95 | 11556 |
| 5 | 1.96 | 10608 |
| 6 | 2.09 | 15182 |
| 7 | 0.68 | 15305 |
| 8 | 2.04 | 17793 |
| 9 | 2.79 | 12251 |
| 10 | 2.08 | 12408 |
| 11 | 0.53 | 9751.9 |
| 12 | 2.01 | 13975 |
| 13 | 1.98 | 13953 |
| 14 | 1.27 | 17142 |
| 15 | 4.13 | 17332 |
| 16 | 1.25 | 14118 |
| 17 | 2.05 | 13695 |
| 18 | 2.27 | 12105 |
| 19 | 2.35 | 12228 |
| 20 | 2.37 | 20383 |
| 21 | 1.50 | 15152 |
| 22 | 1.93 | 9776 |
| 23 | 2.33 | 14825 |
| 24 | 2.29 | 17094 |
| 25 | 2.21 | 13939 |
| 26 | 2.71 | 13454 |
| 27 | 2.47 | 18747 |
| 28 | 4.85 | 28949 |
| 29 | 4.87 | 28956 |
| 30 | 2.72 | 10251 |
| 31 | 3.50 | 12919 |
| 32 | 2.92 | 17748 |
| 33 | 2.17 | 12319 |
| 34 | 10.58 | 50000 |
| 35 | 1.85 | 9633.8 |
| 36 | 1.22 | 9328.7 |
| 37 | 1.49 | 11097 |
| 38 | 1.45 | 17557 |
| 39 | 1.60 | 16135 |
| 40 | 1.26 | 12570 |
| 41 | 3.09 | 44340 |
| 42 | 1.06 | 10276 |
| 43 | 1.77 | 8455.3 |
| 44 | 1.41 | 11143 |
| 45 | 89.68 | 50000 |
| 46 | 33.66 | 50000 |
| 47 | 100.00 | 50000 |
| 48 | 1.94 | 15272 |
| 49 | 1.63 | 17498 |
| 50 | 1.75 | 17805 |
| 52 | 2.13 | 10580 |
| 53 | 1.96 | 15155 |
| 54 | 4.32 | 15475 |
| 57 | 2.32 | 19940 |
| 58 | 2.11 | 24201 |
| 59 | 2.04 | 18824 |
| 60 | 1.90 | 23786 |
| 61 | 2.35 | 20853 |
| 62 | 2.58 | 28088 |
| 63 | 5.12 | 35409 |
| 64 | 1.24 | 13574 |
| 65 | 1.02 | 14146 |
| 66 | 2.07 | 12766 |
| 67 | 2.41 | 14422 |
| 68 | 2.11 | 16071 |
| 69 | 2.90 | 17317 |
| 70 | 2.86 | 15284 |
| 71 | 5.04 | 21331 |
| 72 | | 27179 |
| 73 | | 30756 |
| 74 | | 26574 |
| 75 | | 34281 |
| 76 | | 24778 |
| 77 | | 36973 |
| 78 | | 28570 |
| 79 | | 45683 |
| 80 | | 27203 |
| 81 | | 36933 |
| 82 | | 39664 |

### Example B: Thermodynamic Solubility Assay

Approximately 7 mg of each test compound as a dry powder was placed in a vial. Aliquots were weighed out for each assay media at 2 hour and 24 hour time points, to be analyzed. The appropriate buffer (FaSSIF or PBS) was added to each vial such that the final dose concentration of 5 mg/mL was achieved. Samples were then vortexed for 5-10 seconds. Following a 2-hour or a 24-hour incubation on a rotary shaker (200 RPM) at ambient temperature (22.3-23.8° C.), the samples were vacuum filtered through a Millipore solubility filter plate with 0.45 μM polycarbonate filter membrane and the filtrates were collected in a 96 well polypropylene plate. The plate was sealed with a pierceable heat seal and analyzed by HPLC-UV.

An Agilent 1290 UHPLC equipped with a micro-well plate autosampler, quaternary HPLC pump, and diode array detector was used for analysis. Each filtrate (1.5 μL) was injected onto the column (AQUASIL C18, 5 μM, 50×2.1 mm) and eluted using a gradient of 10-100% MeCN/water containing 0.1% formic acid. A system QC (0.01 mg/mL Caffeine in DMSO) was injected every 12-15 injections. Data was collected at 214, 254 and 280 nm. Results are reported using data obtained at 280 nm. The resulting peak areas were plotted against the known concentrations from the calibration and the filtrates are quantified with respect to the linear regression using Agilent OpenLab Intelligent Reporting Software. The results are reported electronically in mg/mL.

Solubility data for exemplary compounds is shown in Table 2. Comparative solubility data for Intermediate B and Intermediate C is also included in Table 2.

TABLE 2

| Example | Thermodynamic FaSSIF |
|---|---|
| No. | Solubility @ 24 h (mg/mL) |
| 3 | 6.65 |
| 4 | 4.30 |
| 5 | 0.17 |
| 7 | 3.84 |
| 8 | 3.02 |
| 9 | 2.87 |
| 10 | 4.69 |
| 11 | 5.09 |
| 12 | 5.28 |
| 13 | 4.38 |
| 15 | 4.37 |
| 16 | 2.23 |
| 17 | 4.59 |
| 19 | 5.058 |
| 21 | 5.04 |
| 22 | 4.185 |
| 23 | 4.4 |
| 24 | 5.005 |
| 25 | 5.13 |
| 26 | 4.765 |
| 27 | 4.665 |
| 28 | 4.405 |
| 29 | 4.88 |
| 31 | 4.625 |
| 32 | 4.805 |
| 33 | 5.68 |
| 34 | 4.89 |
| 35 | 4.66 |
| 36 | 5.055 |
| 37 | 4.985 |
| 38 | 2.455 |
| 39 | 2.655 |
| 40 | 5.345 |
| 41 | 5.595 |
| 42 | 5.08 |
| 43 | 5.415 |
| 46 | 4.985 |
| 47 | 0.285 |
| 48 | 3.825 |
| 49 | 1.57 |
| 52 | 5.16 |
| 54 | 4.655 |
| 55 | 4.6 |
| 56 | 4.245 |
| 57 | 4.63 |
| 58 | 4.86 |
| 59 | 5.235 |
| 60 | 4.52 |
| 61 | 4.795 |
| 62 | 5.045 |
| 63 | 4.625 |
| 64 | 4.845 |
| 65 | 3.885 |
| 66 | 4.27 |
| 67 | 5.185 |
| 68 | 4.625 |
| 69 | 4.6 |
| Intermediate B | 0.002 |
| Intermediate C | 0.002 |

FaSSIF = Fasted-state simulated intestinal fluid.

### Example C: Dog PO Pharmacokinetic Studies

Doses are expressed as “mg-eq”, referring to the mass (in mg) of Intermediate B (for Compound 4) or Intermediate C (for Compound 3) equivalents (eq) as a fixed dose or relative to body weight (in kg).

### Dog PK Dosing

Male beagle dogs were fasted overnight. Food was returned approximately 4 hours postdose. Each animal received a single 6 μg/kg intramuscular injection of pentagastrin approximately 30 minutes prior to test article administration to stimulate gastric secretion. The intramuscular dose was administered in a thigh muscle using a needle and syringe.

Solution or suspension oral doses were administered at a dose of 4 mg-eq/kg via gavage and the dosing tube was flushed with ~10 mL water prior to removal. Individual doses were calculated based on body weights recorded on the day of dose administration. Solid or capsule oral doses were administered at a dose of 40 mg-eq fixed, by hand by deep throat deposition. Following each dose, the animals were offered approximately 5 mL of water to assist in swallowing. This was done by depositing ~1-3 mL at a time into the back of the throat and holding the mouth closed until swallowing was observed. This was repeated until all 5 mL of water has been given. Once swallowed, 25 mL water was administered by gavage for a total of 30 mL water administered with each dose. All animals were observed at dosing and each scheduled collection. For tablet formulations, the specified dose was administered orally followed by approximately 10 mL of water via syringe. This was followed by an additional 20 mL of water administered via oral gavage. The total flush volume was approximately 30 mL. Individual doses were administered on a fixed basis. In all cases, food was returned at approximately 4 hours after dosing. All animals were observed at dosing and each scheduled collection.

Serial blood samples (through 168 h) were collected into pre-chilled K₂EDTA with the appropriate volume of 40 mM dichlorvos added to result in a final dichlorvos concentration of 2 mM and stored on wet ice until processed. Whole blood was processed to plasma by centrifugation (3500 rpm for 10 minutes at 5° C.) within 30 minutes of collection. Plasma samples were transferred into Micronic 96 well tubes and stored at 70° C. as soon as possible and remained at 70° C. until shipped for bioanalysis.

### Bioanalysis of Plasma Samples

To a 20 uL aliquot of each plasma sample with exception of the matrix blanks, 120 μL of 100 ng/ml Carbamazepine and Chrysin in acetonitrile (ACN) was added. The matrix blank samples received 120 μL of acetonitrile only. The precipitated proteins were removed by centrifugation and 100 μL of supernatant was transferred into a clean 96-well plate. A 100 μL aliquot of water was added to each sample. An aliquot of 2-2.5 μL was injected into an Applied Biosystems API-6500 LC/MS/MS system, eluting with a gradient of water and acetonitrile (containing 0.1% formic acid).

AUC_{inf} was calculated as area under the plasma concentration vs. time curve from 0 h to infinity.

Bioavailability (% F) was calculated by comparing plasma concentration via PO dose (oral) vs. plasma concentration via IV dose (intravenous) using the following equation

$$\% F = \frac{PO AUCinf}{IV AUCinf} \times \frac{IV Dose}{PO Dose} \times 100$$

### Example D: Dog IV Pharmacokinetic Studies

Three male beagle dogs were fasted overnight. Each animal was administered an intravenous dose of the test compound in the formulation indicated in Table 3 as an approximately 30-minute infusion via an indwelling catheter in a cephalic vein. After dose administration, but prior to removal from the animal, the dose apparatus (i.e., the indwelling catheter) was flushed with approximately 1 mL of saline. Individual doses were calculated based on body weights recorded on the day of dose administration. Food returned at approximately 4 hours after dosing.

Serial blood samples (through 168 h) were collected into pre-chilled K₂EDTA with the appropriate volume of 40 mM dichlorvos added to result in a final dichlorvos concentration of 2 mM and stored on wet ice until processed. Whole blood was processed to plasma by centrifugation (3500 rpm for 10 minutes at 5° C.) within 30 minutes of collection. Plasma samples were transferred into Micronic 96 well tubes and stored at −70° C. as soon as possible and remained at −70° C. until shipped for bioanalysis.

To a 20 uL aliquot of each plasma sample with exception of the matrix blanks, 120 μL of 100 ng/mL Carbamazepine and Chrysin in acetonitrile (ACN) was added. The matrix blank samples received 120 μL of acetonitrile only. The precipitated proteins were removed by centrifugation and 100 μL of supernatant was transferred into a clean 96-well plate. A 100 μL aliquot of water was added to each sample. An aliquot of 2-2.5 μL was injected into an Applied Biosystems API-6500 LC/MS/MS system, eluting with a gradient of water and acetonitrile (containing 0.1% formic acid). AUC_{inf} was calculated as area under the plasma concentration vs. time curve from 0 h to infinity.

AUC_{inf} was calculated as area under the plasma concentration vs. time curve from 0 h to infinity. Bioavailability (% F) was calculated by comparing plasma concentration via PO dose (oral) vs. plasma concentration via IV dose (intravenous) using the following equation

$$\% F = \frac{PO AUCinf}{IV AUCinf} \times \frac{IV Dose}{PO Dose} \times 100$$

Doses expressed as “mg-eq”, refer to the mass (in mg) of parent compound equivalents (eq) as a fixed dose or relative to body weight (in kg).

### PK Data

TABLE 3
Dog IV PK Data of Example D

| | Dog Group | Compound | IV Dose | Compound | AUC_{INF} |
|---|---|---|---|---|---|
| Entry | # | Dosed | (mg/kg) | Measured | (μM · h) |
| 1 | Group 1 | Intermediate B | 1.0 | Intermediate B | 1400 ± |
| | | | | | 226 |
| 2 | Group 2 | Intermediate C | 1.0 | Intermediate C | 707 ± |
| | | | | | 98 |
| 3 | Group 3 | Intermediate C | 0.5 | Intermediate C | 250 ± |
| | | | | | 42 |
| 4 | Group 4 | Intermediate C | 1.0 | Intermediate C | 722 ± |
| | | | | | 98 |

TABLE 4
Dog PO PK Data of Example C

| | Dog | Compound | | Compound | AUC_{INF} | Cmax | |
|---|---|---|---|---|---|---|---|
| Entry | Group # | Dosed | PO Dose | Measured | (μM · h) | (μM) | % F |
| 1 | Group 1 | Intermediate B | 4 mg/kg | Intermediate B | 1940 ± | 39.6 ± | 35.3 ± |
| | | | | | 435 | 3.8 | 10.0 |
| 2 | Group 2 | Intermediate C | 4 mg/kg | Intermediate C | 1040 ± | 28.5 ± | 36.4 ± |
| | | | | | 177 | 2.0 | 1.6 |
| 3 | Group 3 | Compound 3 | 300 mg- | Intermediate C | 1860 ± | 74.7 ± | 12.9 ± |
| | | | eq fixed | | 470 | 21.4 | 3.4 |

TABLE 5
Oral Bioavailability of Intermediate B or Intermediate C in Dog Following
a 4 mg-eq/kg Solution Dose of Test Compound According to Example C.

| | | | Dog % F of Intermediate |
|---|---|---|---|
| | | Compound | B or Intermediate C |
| Entry | Compound Dosed | Measured | (4 mg-eq/kg solution) |
| 1 | 3 | Intermediate C | 11 |
| 2 | 7 | Intermediate B | 22.2 |

TABLE 6
Oral Bioavailability of Intermediate B or Intermediate C in
Dog Following a 40 mg-eq Powder-in-Capsule Fixed Dose
of Test Compound According to Example C.

| | | | Dog % F of Intermediate B |
|---|---|---|---|
| | Compound | Compound | or Intermediate C |
| Entry | Dosed | Measured | (40 mg-eq PIC) |
| 1 | 3 | Intermediate C | 31 |
| 2 | 7 | Intermediate B | 23.7 |
| 3 | 10 | Intermediate B | 18.6 |
| 4 | 12 | Intermediate B | 19.1 |

TABLE 7
Oral Bioavailability of Intermediate B or Intermediate C in
Dog Following a 175 mg-eq Tablet Fixed Dose of
Test Compound According to Example C.

| | | | Dog % F of Intermediate B |
|---|---|---|---|
| | Compound | Compound | or Intermediate C |
| Entry | Dosed | Measured | (175 mg-eq Tablet) |
| 1 | 3 | Intermediate C | 20.7 |
| 2 | 12 | Intermediate B | 36.1 |
| 3 | 17 | Intermediate B | 29.3 |
| 4 | 18 | Intermediate B | 33.2 |
| 5 | 19 | Intermediate B | 28.5 |
| 6 | 22 | Intermediate B | 25.5 |
| 7 | 23 | Intermediate C | 37.3 |
| 8 | 28 | Intermediate B | 38.8 |
| 9 | 29 | Intermediate C | 30.5 |
| 10 | 32 | Intermediate C | 28.6 |
| 11 | 42 | Intermediate B | 9.1 |
| 12 | 50 | Intermediate B | 4.6 |

TABLE 8
Oral Bioavailability of Intermediate B or Intermediate C in Dog
Following a 300 mg-eq Powder-in-Capsule or Tablet Fixed
Dose of Test Compound According to Example C.

| | | | Dog % F of Intermediate B or |
|---|---|---|---|
| | Compound | Compound | Intermediate C |
| Entry | Dosed | Measured | (300 mg-eq PIC or Tablet) |
| 1 | 3 | Intermediate C | 12.9 |
| 2 | 4 | Intermediate B | 13.1 |
| 3 | 8 | Intermediate B | 8.3 |

TABLE 9
Oral Bioavailability of Intermediate B or Intermediate
C in Dog Following a 350 mg-eq Tablet
Fixed Dose of Test Compound According to Example C.

| | | | Dog % F of Intermediate B or |
|---|---|---|---|
| | Compound | Compound | Intermediate C |
| Entry | Dosed | Measured | (300 mg-eq PIC or Tablet) |
| 1 | 17 | Intermediate B | 31.7 |
| 2 | 23 | Intermediate C | 24.7 |
| 3 | 28 | Intermediate B | 35.7 |
| 4 | 29 | Intermediate C | 38 |

TABLE 10
Oral Bioavailability of Intermediate B or Intermediate C in Dog
Following a 175 mg Tablet Fixed Dose According to Example C.

| Entry | Compound Dosed | Dog % F (175 mg Tablet) |
|---|---|---|
| 1 | Intermediate B | 9.2 |
| 2 | Intermediate C | 2.9 |

The compounds disclosed herein generally show improved oral bioavailability of Intermediate B or Intermediate C in dogs relative to when Intermediate B or Intermediate C is dosed.

### Example E: Rat PO Pharmacokinetic Studies

Six male Sprague-Dawley rats were fasted overnight. Three animals were administered a 5 mg-eq/kg oral dose of Compound 1′ and the other three animals were administered a 5 mg-eq/kg oral dose of lenacapavir. In all cases, food was returned at approximately 4 hours after dosing. All animals were observed at dosing and each scheduled collection. The delivery vehicle for the oral dose of Compound 1′ and lenacapavir is described in Table 11.

TABLE 11
Delivery Vehicle for Oral Dose of Compound 1′ and Lenacapavir

| | | | Dose | |
|---|---|---|---|---|
| Test | Dose Level | Conc. | Volume | |
| Article | (mg-eq./kg) | (mg/mL) | (mL/kg) | Vehicle |
| Compound | 5 | 1 | 5 | 0.5% hydroxypropyl |
| 1′ | | | | methylcellulose, |
| | | | | high viscosity; |
| | | | | 99.5% water; final |
| | | | | formulation pH 2.25 |
| Lenacapavir | 5 | 5 | 1 | 0.5% hydroxypropyl |
| | | | | methylcellulose, |
| | | | | high viscosity; |
| | | | | 99.5% water; final |
| | | | | formulation pH 2.6 |

Serial blood samples (through 168 h) were collected into pre-chilled K₂EDTA with the appropriate volume of 40 mM dichlorvos added to result in a final dichlorvos concentration of 2 mM and stored on wet ice until processed. Whole blood was processed to plasma by centrifugation (3500 rpm for 10 minutes at 5° C.) within 30 minutes of collection. Plasma samples were transferred into Micronic 96 well tubes and stored at 70° C. as soon as possible and remained at 70° C. until shipped for bioanalysis. Bioanalysis was carried out according to Example C.

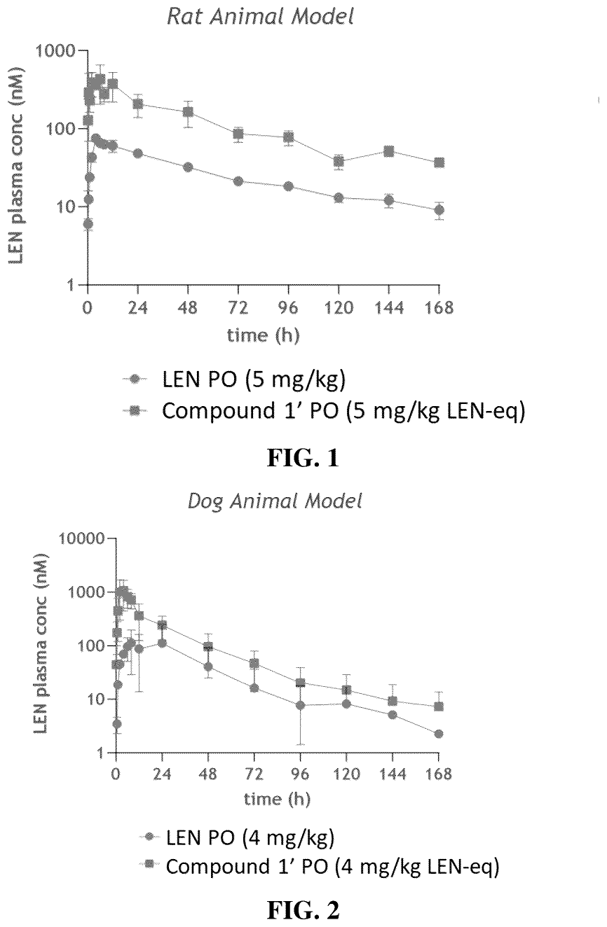

The plasma concentration of Compound 1′ and lenacapavir are also illustrated in the Figures. In particular, FIG. 1 shows that the plasma concentration of Compound 1′ is higher than that of lenacapavir over the monitored time period.

### Example F: Dog PO Pharmacokinetic Studies

Six male beagle dogs were fasted overnight. Three animals were administered a 4 mg-eq/kg oral dose of Compound 1′ and the other three animals were administered a 4 mg-eq/kg oral dose of lenacapavir. In all cases, food was returned at approximately 4 hours after dosing. All animals were observed at dosing and each scheduled collection. The delivery vehicle for the oral dose of Compound 1′ and lenacapavir is described in Table 12.

TABLE 12
Delivery Vehicle for Oral Dose of Compound 1′ and Lenacapavir

| | | | Dose | |
|---|---|---|---|---|
| | Dose Level | Conc. | Volume | |
| Test Article | (mg-eq./kg) | (mg/mL) | (mL/kg) | Vehicle |
| Compound | 4 | 0.8 | 5 | 5% ethanol; 20% |
| 1′ | | | | propylene glycol; |
| | | | | 75% water |
| Lenacapavir | 4 | 4 | 1 | 0.5% hydroxypropyl |
| | | | | methylcellulose, |
| | | | | high viscosity; |
| | | | | 99.5% water; final |
| | | | | formulation pH 2.9 |

Serial blood samples (through 168 h) were collected into pre-chilled K₂EDTA with the appropriate volume of 40 mM dichlorvos added to result in a final dichlorvos concentration of 2 mM and stored on wet ice until processed. Whole blood was processed to plasma by centrifugation (3500 rpm for 10 minutes at 5° C.) within 30 minutes of collection. Plasma samples were transferred into Micronic 96 well tubes and stored at 70° C. as soon as possible and remained at 70° C. until shipped for bioanalysis. Bioanalysis was carried out according to Example C.

The plasma concentration of Compound 1′ and lenacapavir are also illustrated in the Figures. In particular, FIG. 2 shows that the plasma concentration of Compound 1′ is higher than that of lenacapavir over the monitored time period.

All of the U.S. patents, U.S. patent application publications, U.S. patent applications, foreign patents, foreign patent applications and non-patent publications referred to in this specification are incorporated herein by reference, in their entirety to the extent non inconsistent with the present description.

From the forgoing it will be appreciated that, although specific embodiments have been described herein for purposes of illustration, various modifications may be made without deviating from the spirit and scope of this disclosure. Accordingly, this disclosure is not limited except as by the appended claims.

### Example G: Dog PO Pharmacokinetic Studies with Compound 28

Six male beagle dogs were fasted overnight. Three animals were administered a 350 mg-eq/kg oral dose of Compound 28 and the other three animals were administered a 350 mg-eq/kg oral dose of Compound 28 and a 350 mg dose of Compound 1′. In all cases, food was returned at approximately 4 hours after dosing. All animals were observed at dosing and each scheduled collection. The delivery vehicle for the oral dose of Compound 1′ and Compound 28 is described in Table 13. Doses expressed as “mg-eq”, refer to the mass (in mg) of parent compound equivalents (eq) as a fixed dose or relative to body weight (in kg).

TABLE 13
Delivery Vehicle for Oral Dose of Compound 28 and Compound 1′

| | Dose | | |
|---|---|---|---|
| Test Article | Level | Conc. | Vehicle |
| Compound 28 | 350 mg- | 175 | 7% Crospovidone; |
| | eq/animal | (mg/tablet) | 4% Hydroxypropyl cellulose; |
| | | | 22.5% Lactose, monohydrate; |
| | | | 1% Magnesium |
| | | | stearate; 22.5% Microcrystalline |
| | | | cellulose; 3% |
| | | | Poloxamer 407 |
| Compound 1′ | 350 | 350 | 10% Crospovidone; |
| | mg/ | (mg/tablet) | 2% Magnesium stearate; |
| | animal | | 28% Microcrystalline cell |

Serial blood samples (through 336 h) were collected into pre-chilled K₂EDTA with 26 μL of 80 mM dichlorvos and maintained on wet ice, in chilled cryoracks, or at approximately 5° C. prior to blood collection centrifugation to obtain plasma (the final concentration of dichlorvos in blood was approximately 2 mM). Centrifugation began within 1 hour of collection. Individual plasma samples were harvested, placed into 1.1 mL micronic tubes with barcoded labels, and maintained on dry ice prior to storage at approximately −70° C. until bioanalysis was carried out.

Bioanalysis of plasma samples was carried out according to the following procedure: To a 20 μL aliquot of each plasma sample with exception of the matrix blanks, 120 μL of 100 ng/mL Carbamazepine and Chrysin in acetonitrile (ACN) was added. The matrix blank samples received 120 μL of acetonitrile only. The precipitated proteins were removed by centrifugation and 50 μL of supernatant was transferred into two sets of clean 96 well plates for samples to run on two instruments. A 50 μL aliquot of water was added to each sample. An aliquot of 3-5 μL was injected into a Sciex API-5500 LC/MS/MS system.

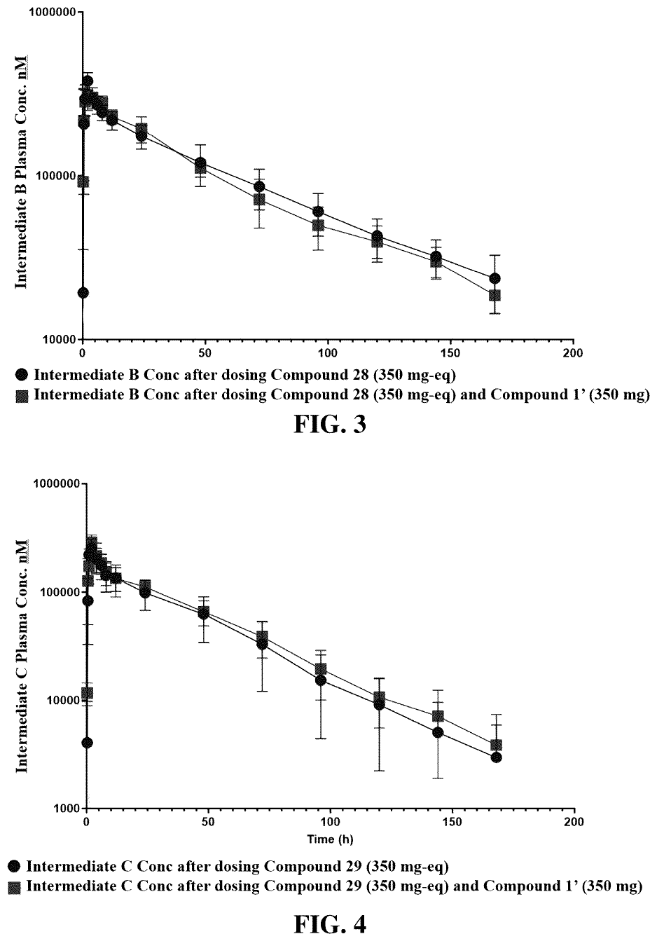

The bioavailability of Intermediate B after dosing Compound 28 with and without Compound 1′ is shown in Table 14 below, and the plasma concentration of Intermediate B for each run (i.e., Compound 28 and Compound 28+Compound 1′) can be found in FIG. 3.

This data shows that the bioavailability of Intermediate B stays high even when Compound 28 is coadministered with Compound 1′. Note that both intestinal ALP and absorption capacity were not saturated, and that these prodrugs were dosed with the dog-equivalent of the human efficacious dose.

TABLE 14
Bioavailability of Intermediate B

| Intermediate B % F | Dog 1 | Dog 2 | Dog 3 | Avg |
|---|---|---|---|---|
| Compound 28 only | 50.0 | 43.0 | 34.3 | 42.4 ± 7.8 |
| Compound 28 + | 48.2 | 41.8 | 33.5 | 41.2 ± 7.4 |
| Compound 1′ | | | | |

### Example H: Dog PO Pharmacokinetic Studies with Compound 29

Six male beagle dogs were fasted overnight. Three animals were administered a 350 mg-eq/kg oral dose of Compound 29 and the other three animals were administered a 350 mg-eq/kg oral dose of Compound 29 and a 350 mg dose of Compound 1′. In all cases, food was returned at approximately 4 hours after dosing. All animals were observed at dosing and each scheduled collection. The delivery vehicle for the oral dose of Compound 1′ and Compound 29 is described in Table 15. Doses expressed as “mg-eq”, refer to the mass (in mg) of parent compound equivalents (eq) as a fixed dose or relative to body weight (in kg).

TABLE 15
Delivery Vehicle for Oral Dose of Compound 29 and Compound 1'

| | Dose | | |
|---|---|---|---|
| Test Article | Level | Conc. | Vehicle |
| Compound 29 | 350 mg- | 254 | 7% Crospovidone; |
| | eq/animal | (mg/tablet) | 4% Hydroxypropyl cellulose; |
| | | | 22.5% Lactose, monohydrate; |
| | | | 1% Magnesium |
| | | | stearate; 22.5% Microcrystalline |
| | | | cellulose; 3% |
| | | | Poloxamer 407 |
| Compound 1′ | 350 | 350 | 10% Crospovidone; |
| | mg/ | (mg/tablet) | 2% Magnesium stearate; |
| | animal | | 28% Microcrystalline cell |

Serial blood samples (through 336 h) were collected into pre-chilled K₂EDTA with 26 μL of 80 mM dichlorvos and maintained on wet ice, in chilled cryoracks, or at approximately 5° C. prior to blood collection centrifugation to obtain plasma (the final concentration of dichlorvos in blood was approximately 2 mM). Centrifugation began within 1 hour of collection. Individual plasma samples were harvested, placed into 1.1 mL micronic tubes with barcoded labels, and maintained on dry ice prior to storage at approximately −70° C. until bioanalysis was carried out. Bioanalysis of plasma samples was carried out according to the procedure in Example G.

The bioavailability of Intermediate C after dosing Compound 29 with and without Compound 1′ is shown in Table 16 below, and the plasma concentration of Intermediate C for each run (i.e., Compound 29 and Compound 29+Compound 1′) can be found in FIG. 4.

This data shows that the bioavailability of Intermediate C stays high even when Compound 29 is coadministered with Compound 1′. Note that both intestinal ALP and absorption capacity were not saturated, and that these prodrugs were dosed with the dog-equivalent of the human efficacious dose.

TABLE 16
Bioavailability of Intermediate C

| Intermediate C % F | Avg |
|---|---|
| Compound 29 only | 38.0 ± 8.4 (n = 5) |
| Compound 29 + Compound 1′ | 40.5 ± 13.5 (n = 3) |

### EMBODIMENTS

Embodiment 1. A method for therapeutic treatment of an HIV infection in a subject, the method comprising administering to the subject (a) Compound 1

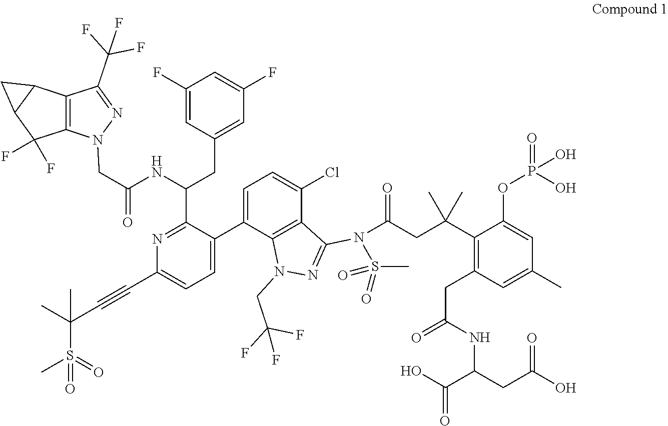 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, and (b) a compound of Formula I

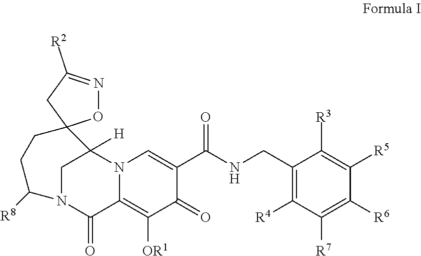

or a pharmaceutically acceptable salt thereof, wherein:
R¹ is —(CR^{1A}R^{1B}O)ₐ(Y)_{b}(CR^{1C}R^{1D})_{d}X;
   wherein a is 0 or 1;
   b is 0 or 1;
   d is 0, 1, 2, 3, 4 or 5;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl; or
   optionally R^{1C} and R^{1D} on the same carbon atom are joined to form a spiro cyclopropyl group;
   Y is —C(O)—, —C(O)O—, —C(O)NH— or —C(O)NR^{1H}—;
   R^{1H} is C₁₋₄alkyl optionally substituted with one or two substituents independently selected from the group consisting of —COOH, —OH, —NH₂, —CONH₂, —P(O)(OH)₂, and —S(O)₂(OH);
   X is selected from the group consisting of:
   (a) —O—P(O)(OR^{1E})₂,
   wherein each R^{1E} is independently H or phenyl;
   (b) —N(R^{1F})₂,
      wherein each R^{1F} is independently H, COO(CR^{1I}R^{1J})ₑOPO(OH)₂, or C₁₋₄alkyl; wherein the C₁₋₄ alkyl is optionally substituted with one or two substituents independently selected from the group consisting of —COOH, —S(O)₂(OH), —P(O)(OH)₂, —OH, —NH₂, and —CONH₂;
      e is 1, 2, or 3;
      each R^{1I} is independently H or C₁₋₃alkyl;
      each R^{1J} is independently H or C₁₋₃alkyl; or
      optionally R^{1I} and R^{1J} on the same carbon atom are joined to form a spiro cyclopropyl group; and
   (c) —N⁺(R^{1G})₃Z⁻,
      wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂;
      Z⁻ is a counterion;
   R² is C₁₋₃ alkyl or C₁₋₃ alkoxy;
   each R³, R⁴, R⁵, R⁶ and R⁷ is independently H or halo; and
R⁸ is H or C₁₋₃alkyl.

Embodiment 2. The method of embodiment 1, wherein Z-is selected from the group consisting of acetate, ascorbate, aspartate, besylate, benzoate, bromide, bicarbonate, carbonate, cinnamate, citrate, chloride, formate, fumarate, gluconate, glutamate, glycolate, lactate, malate, maleate, malonate, mandelate, mesylate, nicotinate, nitrate, oxalate, dihydrogen phosphate, hydrogen phosphate, phosphate, propionate, tosylate, pyroglutamate, salicylate, succinate, bisulfate, sulfate, tartrate, thiocyanate, triflate, and trifluoroacetate.

Embodiment 3. The method of embodiment 1 or 2, wherein X is —O—P(O)(OR^{1E})₂.

Embodiment 4. The method of any one of embodiments 1-3, wherein each R^{1E} is phenyl.

Embodiment 5. The method of any one of embodiments 1-3, wherein each R^{1E} is H.

Embodiment 6. The method of embodiment 1 or 2, wherein X is —N(R^{1F})₂.

Embodiment 7. The method of embodiment 6, wherein each R^{1F} is independently —COO(CR^{1I}R^{1J})ₑOPO(OH)₂ or C₁₋₄alkyl; wherein the C₁₋₄ alkyl is optionally substituted with one or two-COOH.

Embodiment 8. The method of embodiment 6 or 7, wherein each R^{1F} is independently —COO(CR^{1I}R^{1J})ₑOPO(OH)₂ or C₁₋₄alkyl; wherein the C₁₋₄ alkyl is optionally substituted with one —COOH.

Embodiment 9. The method of any one of embodiments 1, 2, and 6-8, wherein e is 1 or 2.

Embodiment 10. The method of any one of embodiments 1, 2, and 6-9, wherein each R^{1I} and each R^{1J} is H.

Embodiment 11. The method of any one of embodiments 1, 2, and 6-10, wherein each R^{1F} is independently —COOCH₂OPO(OH)₂ or C₁₋₄alkyl; wherein the C₁₋₄ alkyl is optionally substituted with one or two-COOH.

Embodiment 12. The method of any one of embodiments 1, 2, and 6-11, wherein each R^{1F} is independently —COOCH₂OPO(OH)₂ or C₁₋₄alkyl; wherein the C₁₋₄ alkyl is optionally substituted with one —COOH.

Embodiment 13. The method of any one of embodiments 1, 2, and 6-12, wherein each R^{1F} is independently —COO(CH₂)₂OPO(OH)₂, —COOCH₂OPO(OH)₂, —CH₃, or —CH₂COOH.

Embodiment 14. The method of any one of embodiments 1, 2, and 6-13, wherein each R^{1F} is independently —COOCH₂OPO(OH)₂, —CH₃, or —CH₂COOH.

Embodiment 15. The method of embodiment 1 or 2, wherein X is —N⁺(R^{1G})₃Z⁻.

Embodiment 16. The method of embodiment 1, 2, and 15, wherein R^{1G} is —CH₃.

Embodiment 17. The method of any one of embodiments 1-16, wherein a is 0.

Embodiment 18. The method of any one of embodiments 1-16, wherein a is 1.

Embodiment 19. The method of any one of embodiments 1-18, wherein b is 0.

Embodiment 20. The method of any one of embodiments 1-18, wherein b is 1.

Embodiment 21. The method of any one of embodiments 1-20, wherein Y is —C(O)—, —C(O)O—, —C(O)NH— or —C(O)NCH₃—.

Embodiment 22. The method of any one of embodiments 1-21, wherein Y is —C(O)—, —C(O)O— or —C(O)NCH₃—.

Embodiment 23. The method of any one of embodiments 1-22, wherein Y is —C(O)—.

Embodiment 24. The method of any one of embodiments 1-22, wherein Y is —C(O)O—.

Embodiment 25. The method of any one of embodiments 1-22, wherein Y is —C(O)NCH₃—.

Embodiment 26. The method of any one of embodiments 1-20, wherein Y is —C(O)NR^{1H}—.

Embodiment 27. The method of embodiment 26, wherein R^{1H} is C₁₋₂alkyl optionally substituted with —COOH, —P(O)(OH)₂, or —S(O)₂(OH).

Embodiment 28. The method of any one of embodiments 1-27, wherein d is 1, 2, or 3.

Embodiment 29. The method of any one of embodiments 1-28, wherein d is 1.

Embodiment 30. The method of any one of embodiments 1-28, wherein d is 2.

Embodiment 31. The method of any one of embodiments 1-28, wherein d is 3.

Embodiment 32. The method of any one of embodiments 1-31, wherein R⁴, R⁵ and R⁷ are each H.

Embodiment 33. The method of any one of embodiments 1-32, wherein R³ and R⁶ are each independently a halo.

Embodiment 34. The method of any one of embodiments 1-33, wherein R³ and R⁶ are each F.

Embodiment 35. The method of any one of embodiments 1-34, wherein R⁸ is C₁₋₃alkyl.

Embodiment 36. The method of any one of embodiments 1-35, wherein R⁸ is methyl.

Embodiment 37. The method of any one of embodiments 1-36, wherein R² is methyl or methoxy.

Embodiment 38. The method of any one of embodiments 1-37, wherein R² is methyl.

Embodiment 39. The method of any one of embodiments 1-37, wherein R² is methoxy.

Embodiment 40. The method of any one of embodiments 1-39, wherein R¹ is selected from the group consisting of

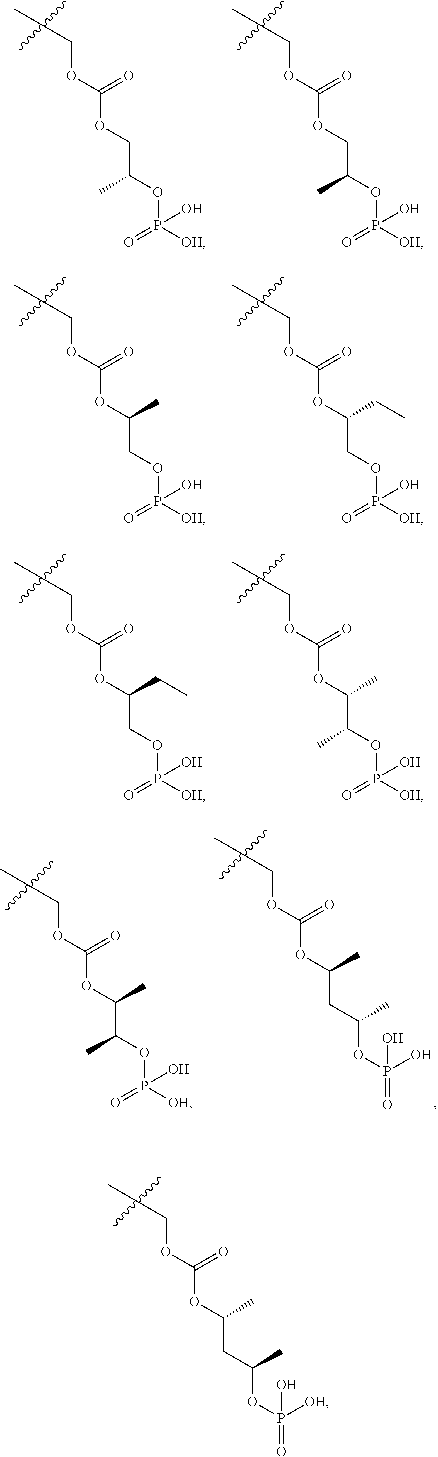

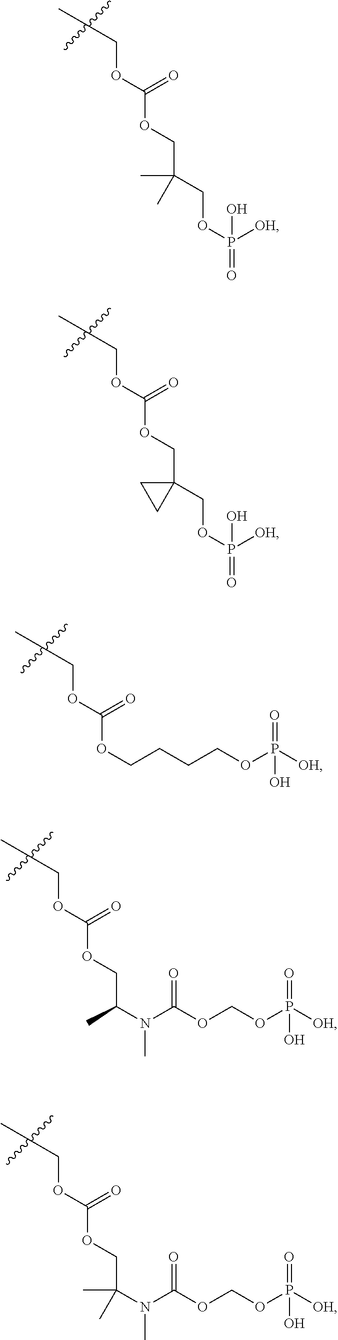 [SMILES: *COC(=O)OCC(C)(C)COP(=O)(O)O ; *COC(=O)OCC1(COP(=O)(O)O)CC1 ; *COC(=O)OCCCCOP(=O)(O)O ; *COC(=O)OC[C@H](C)N(C)C(=O)OCOP(=O)(O)O ; *COC(=O)OCC(C)(C)N(C)C(=O)OCOP(=O)(O)O]

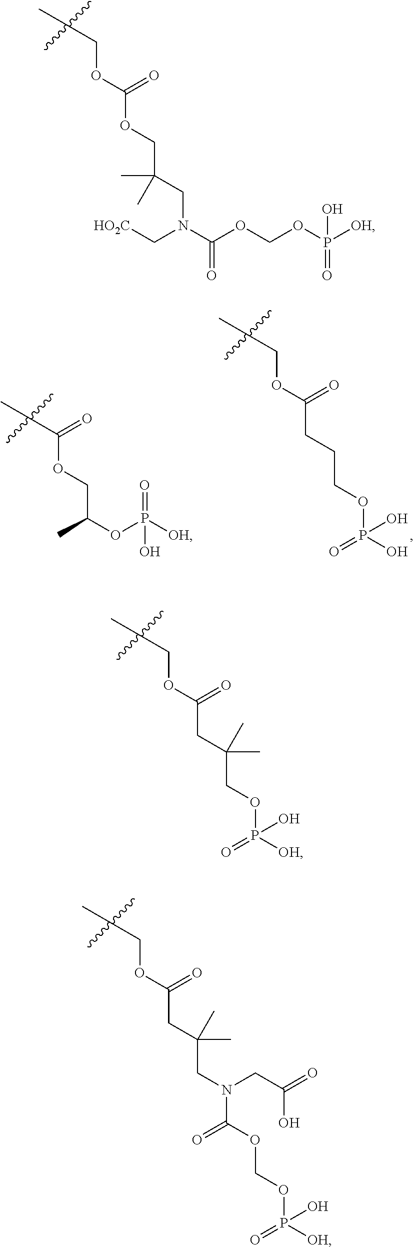 [SMILES: *COC(=O)OCC(C)(C)CN(CC(=O)O)C(=O)OCOP(=O)(O)O ; *C(=O)OC[C@H](C)OP(=O)(O)O ; *COC(=O)CCCOP(=O)(O)O ; *COC(=O)CC(C)(C)COP(=O)(O)O ; *COC(=O)CC(C)(C)CN(CC(=O)O)C(=O)OCOP(=O)(O)O]

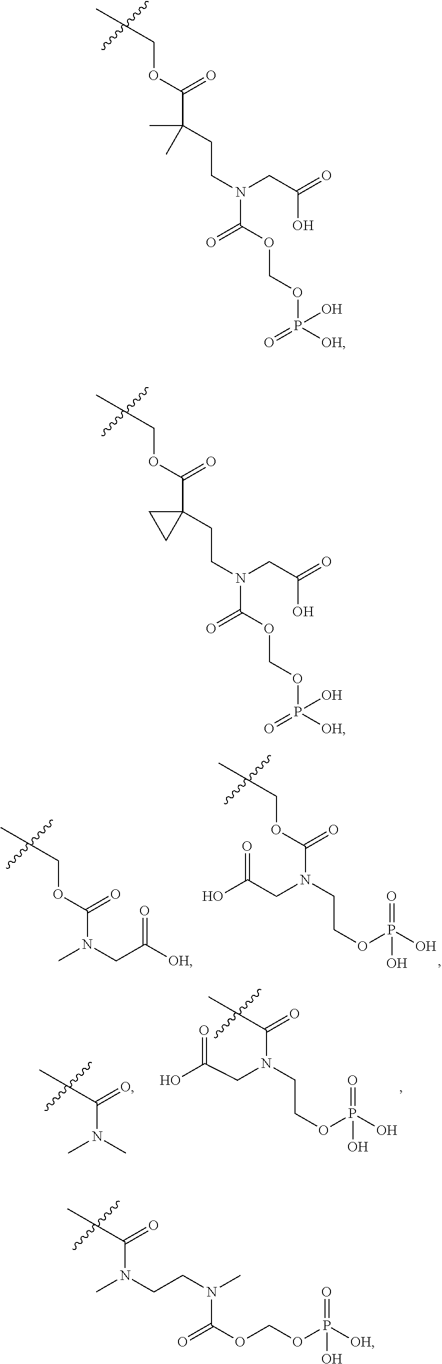

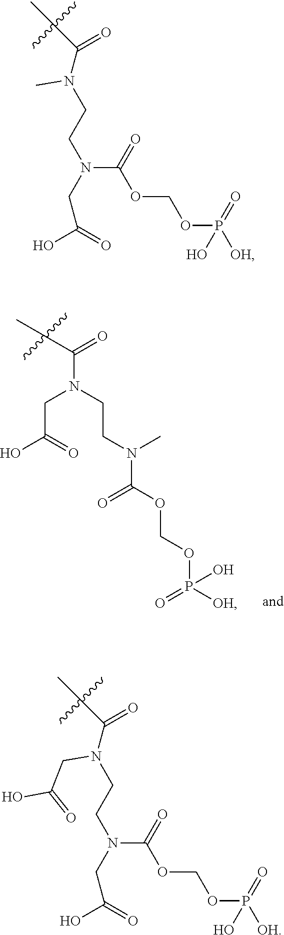 [SMILES: *C(=O)N(C)CCN(CC(=O)O)C(=O)OCOP(=O)(O)O ; *C(=O)N(CCN(C)C(=O)OCOP(=O)(O)O)CC(=O)O ; *C(=O)N(CCN(CC(=O)O)C(=O)OCOP(=O)(O)O)CC(=O)O]

Embodiment 41. The method of any one of embodiments 1-39, wherein R¹ is selected from the group consisting of

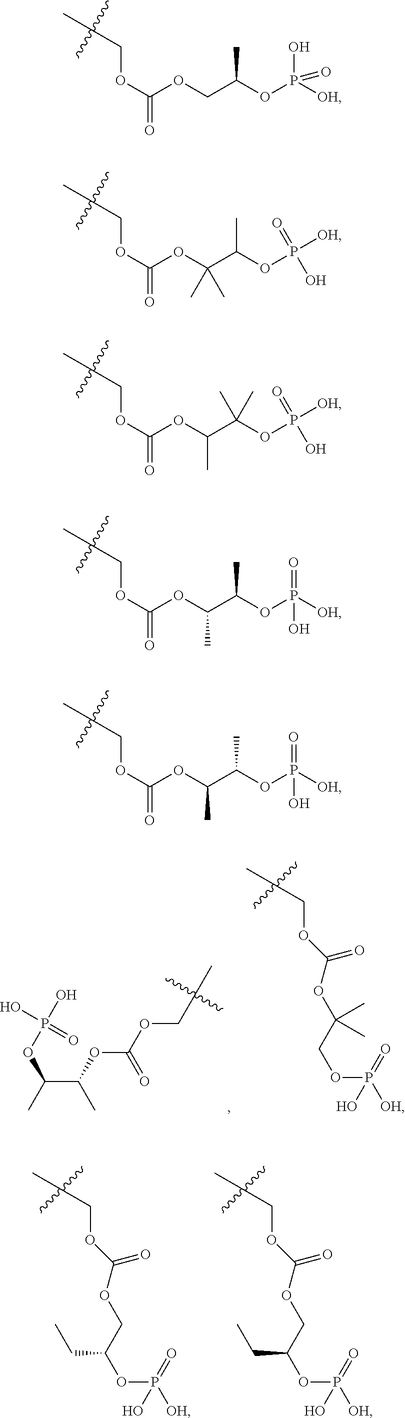

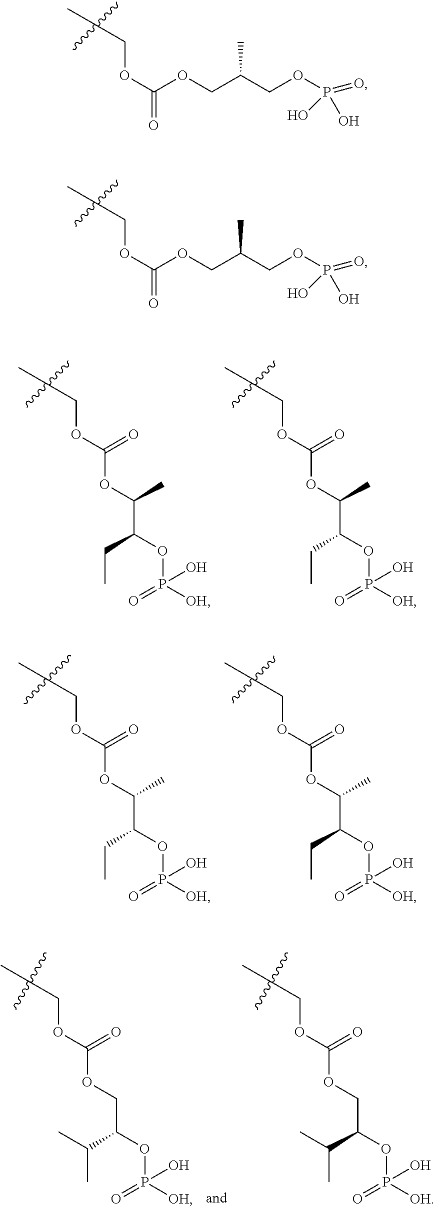

Embodiment 42. The method of any one of embodiments 1-39, wherein R¹ is selected from the group consisting of

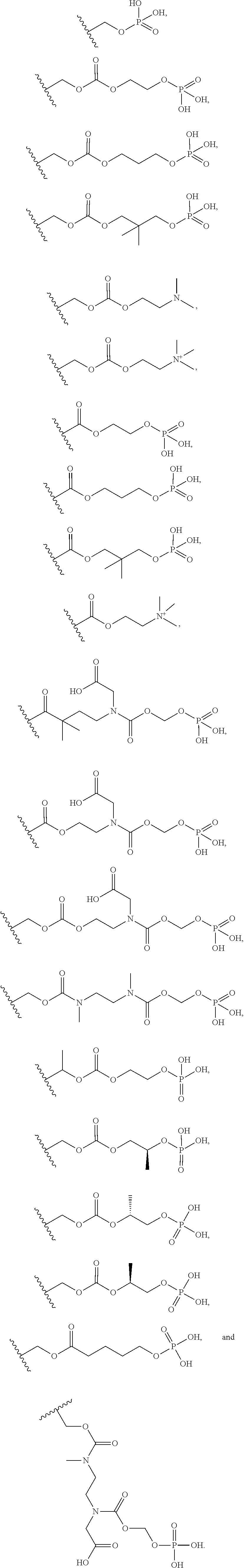

Embodiment 43. The method of any one of embodiments 1-42, wherein the compound is selected from the group consisting of

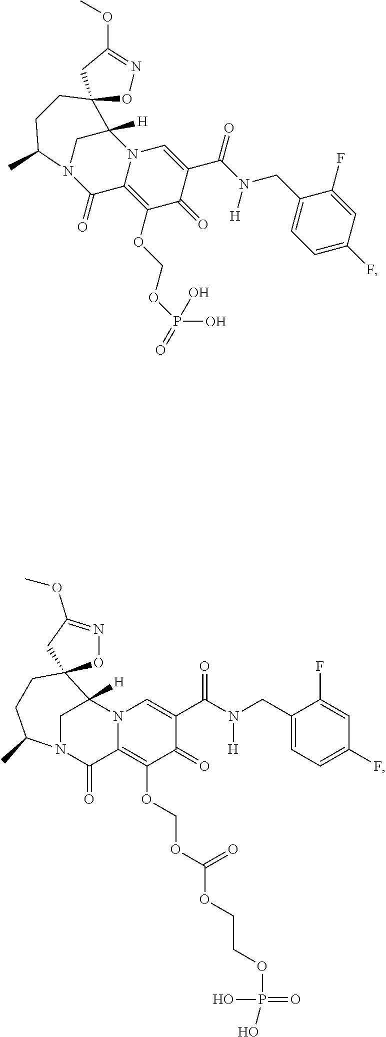 [SMILES: COC1=NO[C@@]2(CC[C@H](C)N3C[C@H]2n2cc(C(=O)NCc4ccc(F)cc4F)c(=O)c(OCOP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1]

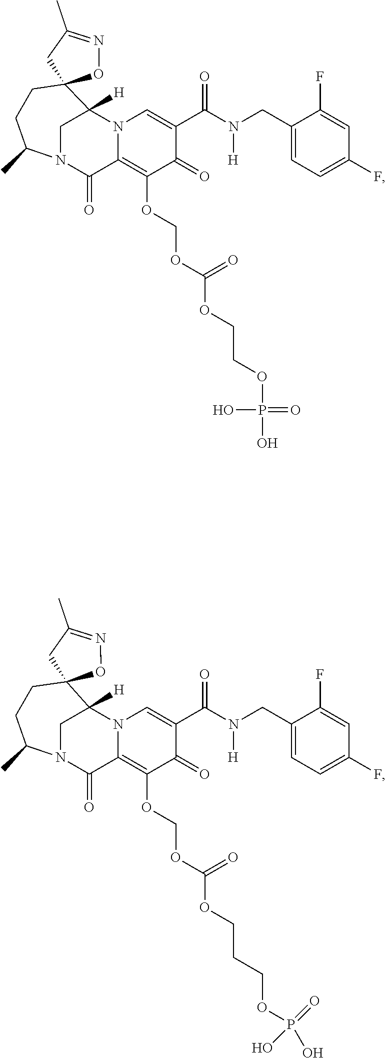 [SMILES: CC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCCOP(=O)(O)O)c2C3=O)C1]

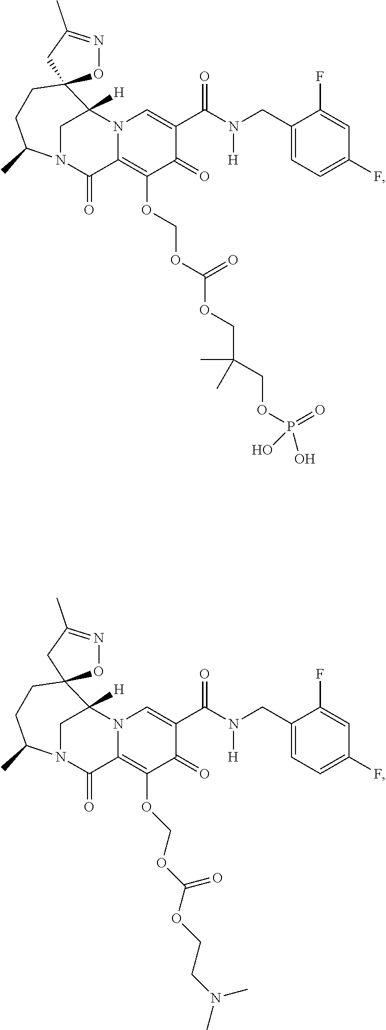 [SMILES: CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCN(C)C)c2C3=O)C1]

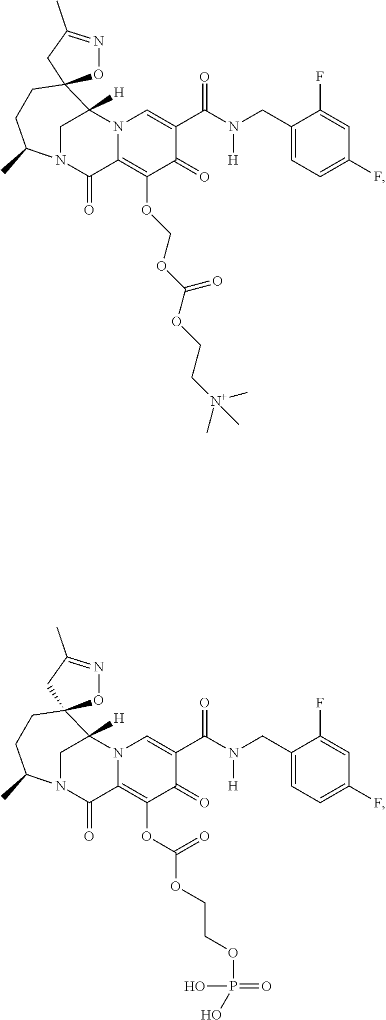 [SMILES: CC1=NO[C@@]2(CC[C@H](C)N3C[C@H]2n2cc(C(=O)NCc4ccc(F)cc4F)c(=O)c(OCOC(=O)OCC[N+](C)(C)C)c2C3=O)C1 ; CC1=NO[C@@]2(CC[C@H](C)N3C[C@H]2n2cc(C(=O)NCc4ccc(F)cc4F)c(=O)c(OC(=O)OCCOP(=O)(O)O)c2C3=O)C1]

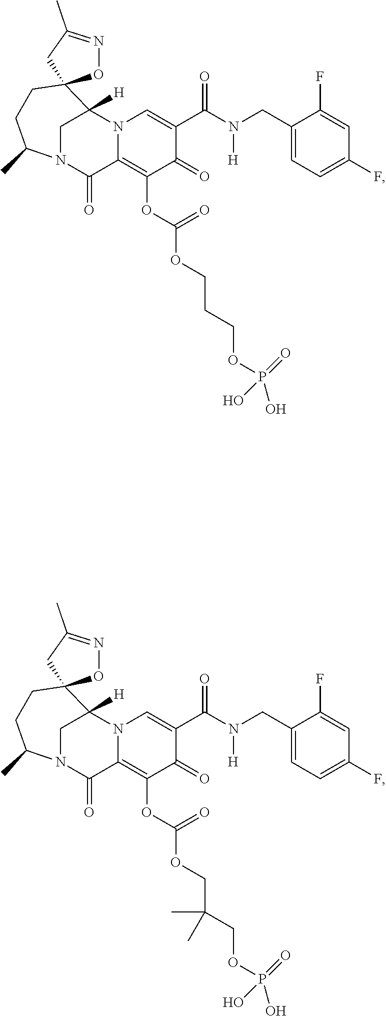 [SMILES: CC1=NO[C@@]2(CC[C@H](C)N3C[C@H]2n2cc(C(=O)NCc4ccc(F)cc4F)c(=O)c(OC(=O)OCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCC(C)(C)COP(=O)(O)O)c2C3=O)C1]

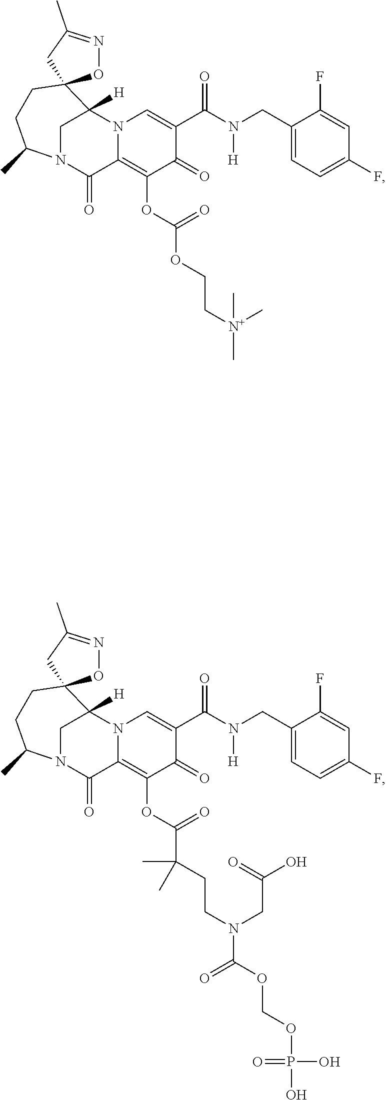 [SMILES: CC1=NO[C@@]2(CC[C@H](C)N3C[C@H]2n2cc(C(=O)NCc4ccc(F)cc4F)c(=O)c(OC(=O)OCC[N+](C)(C)C)c2C3=O)C1 ; CC1=NO[C@@]2(CC[C@H](C)N3C[C@H]2n2cc(C(=O)NCc4ccc(F)cc4F)c(=O)c(OC(=O)C(C)(C)CCN(CC(=O)O)C(=O)OCOP(=O)(O)O)c2C3=O)C1]

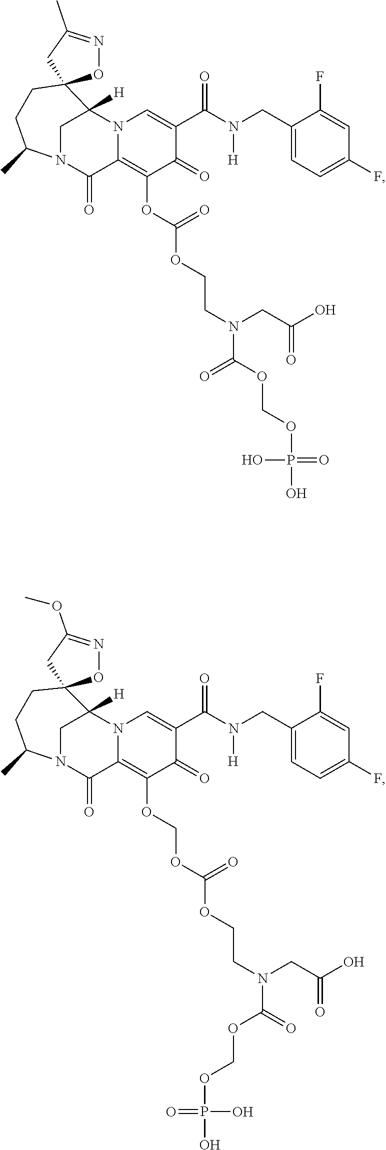 [SMILES: CC1=NO[C@@]2(CC[C@H](C)N3C[C@H]2n2cc(C(=O)NCc4ccc(F)cc4F)c(=O)c(OC(=O)OCCN(CC(=O)O)C(=O)OCOP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCCN(CC(=O)O)C(=O)OCOP(=O)(O)O)c2C3=O)C1]

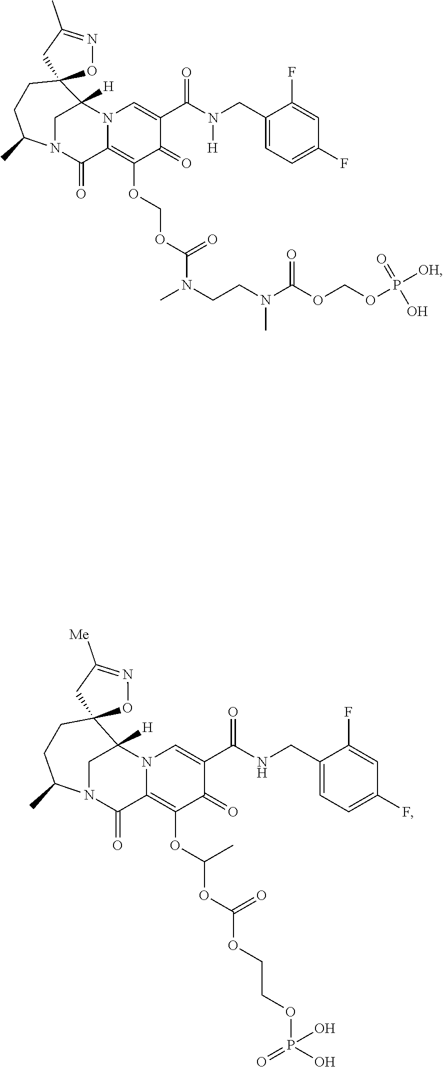 [SMILES: CC1=NO[C@@]2(CC[C@H](C)N3C[C@H]2n2cc(C(=O)NCc4ccc(F)cc4F)c(=O)c(OCOC(=O)N(C)CCN(C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(C)OC(=O)OCCOP(=O)(O)O)c2C3=O)C1]

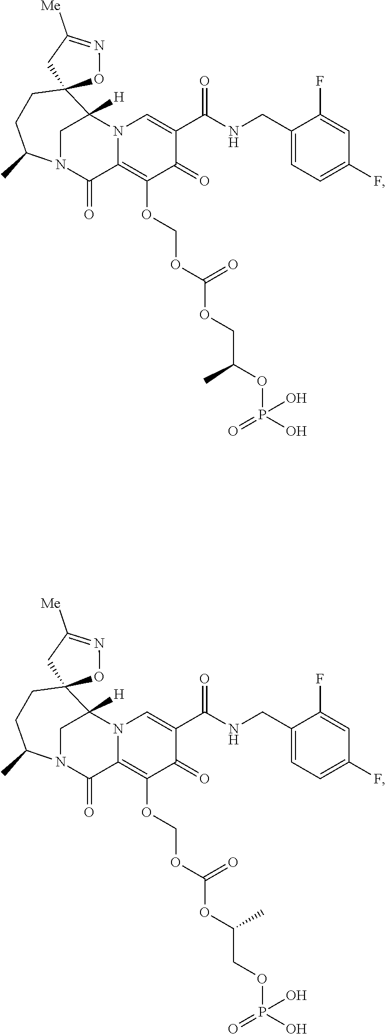 [SMILES: CC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1]

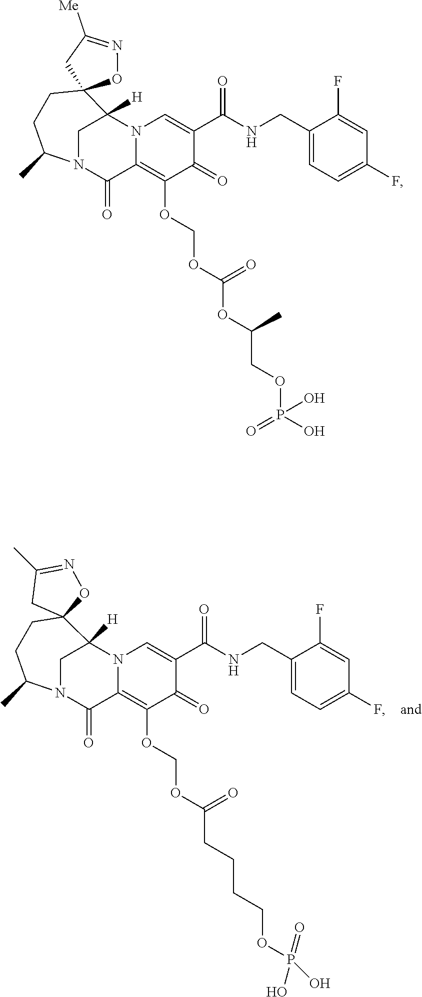 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CCCCOP(=O)(O)O)c2C3=O)C1]

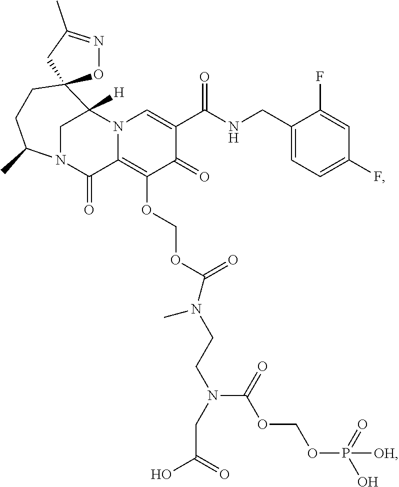 [SMILES: CC1=NO[C@@]2(CC[C@H](C)N3C[C@H]2n2cc(C(=O)NCc4ccc(F)cc4F)c(=O)c(OCOC(=O)N(C)CCN(CC(=O)O)C(=O)OCOP(=O)(O)O)c2C3=O)C1]

or a pharmaceutically acceptable salt thereof.

Embodiment 44. The method of any one of embodiments 1-42, wherein the compound is selected from the group consisting of

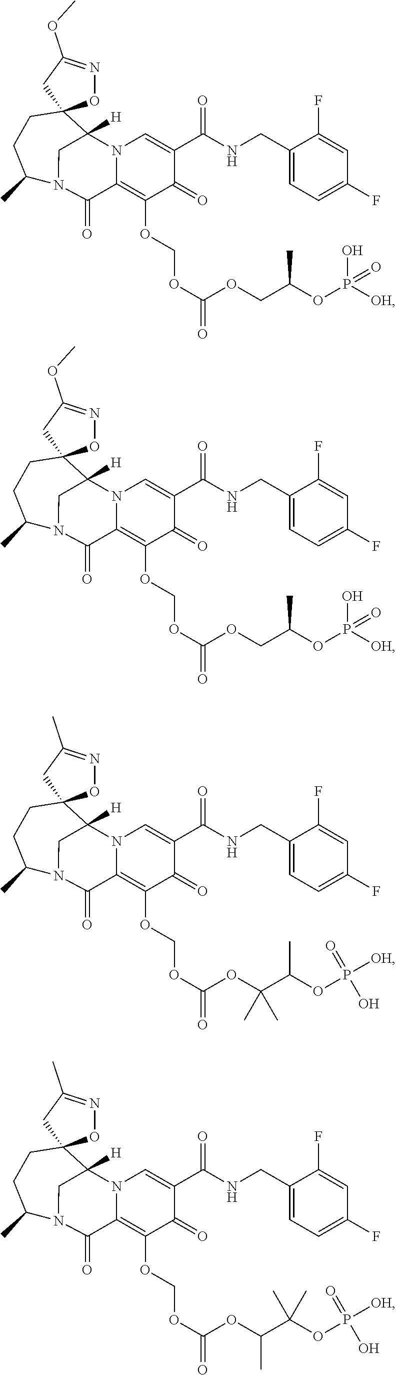 [SMILES: COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC(C)(C)C(C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC(C)C(C)(C)OP(=O)(O)O)c2C3=O)C1]

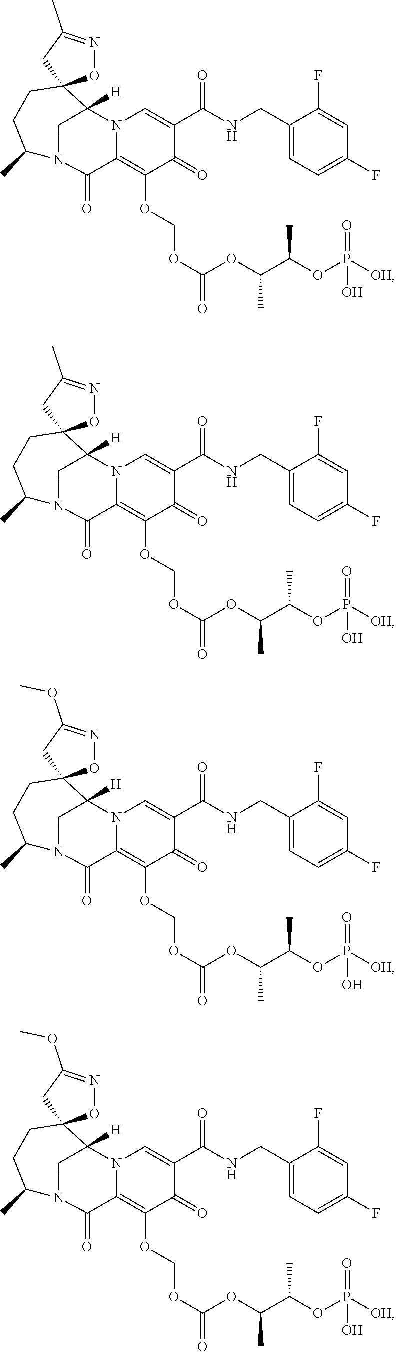 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1]

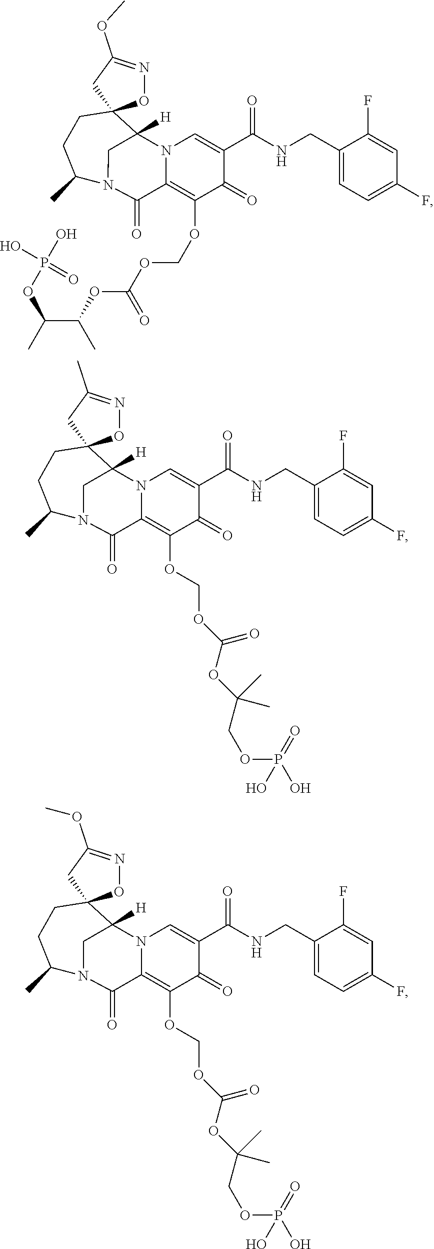

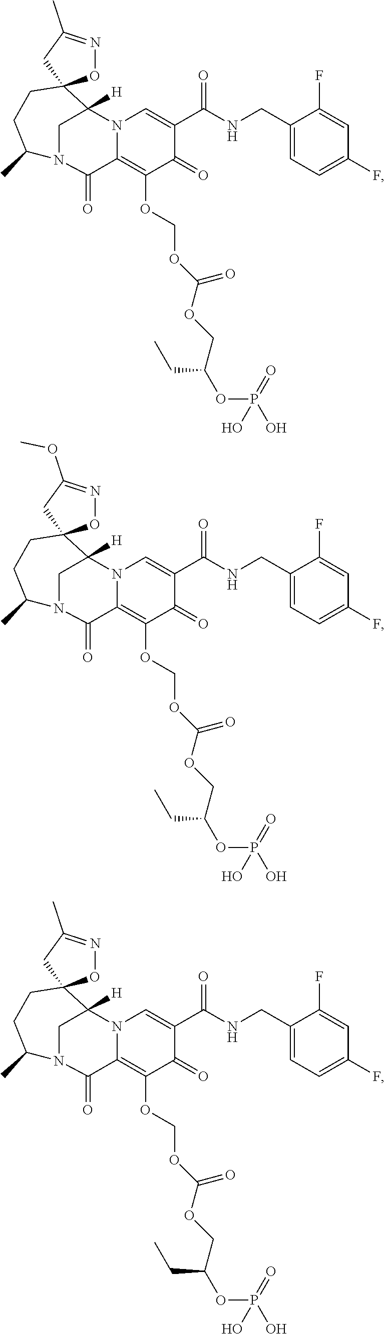 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O ; CC[C@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O ; CC[C@@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O]

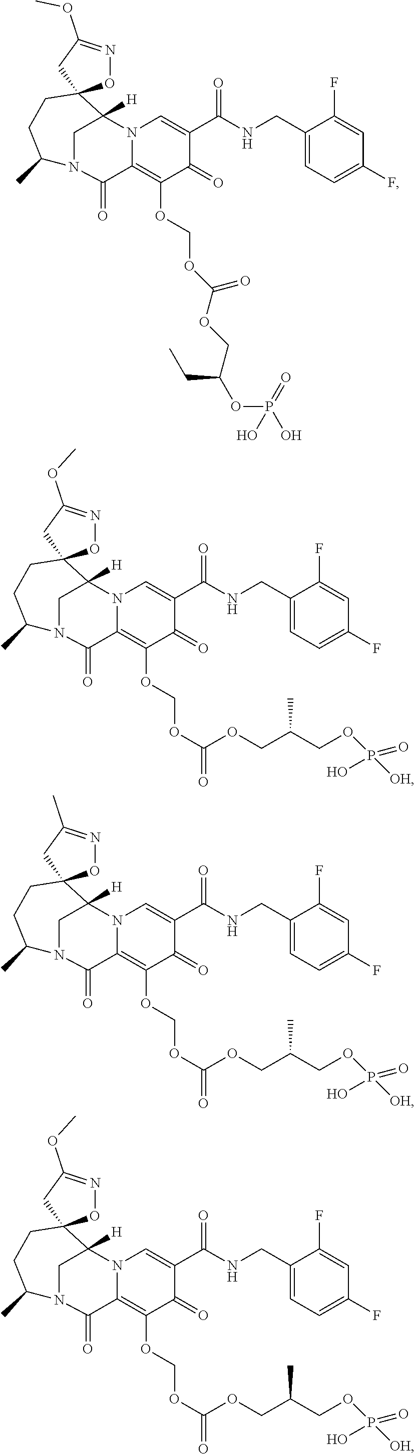 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O ; COC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1]

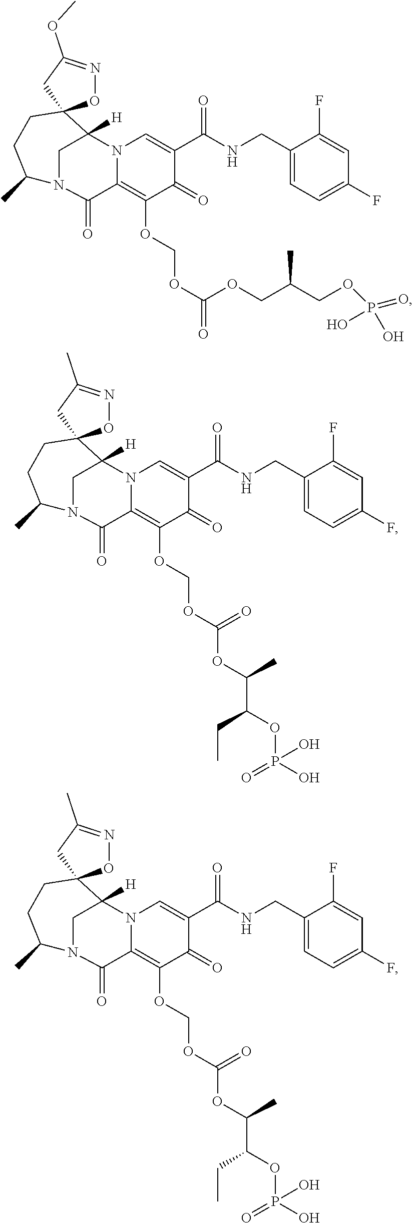 [SMILES: COC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1 ; CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

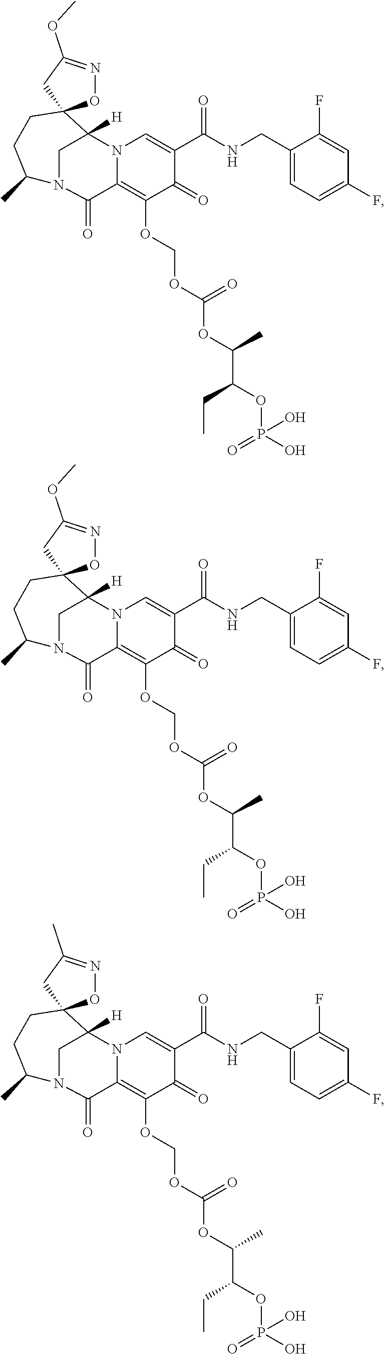 [SMILES: CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2]

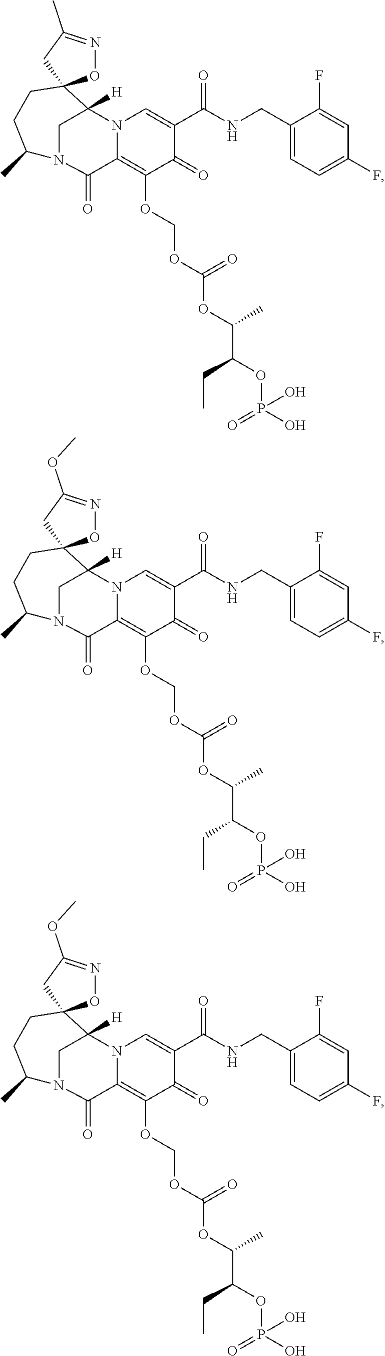 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

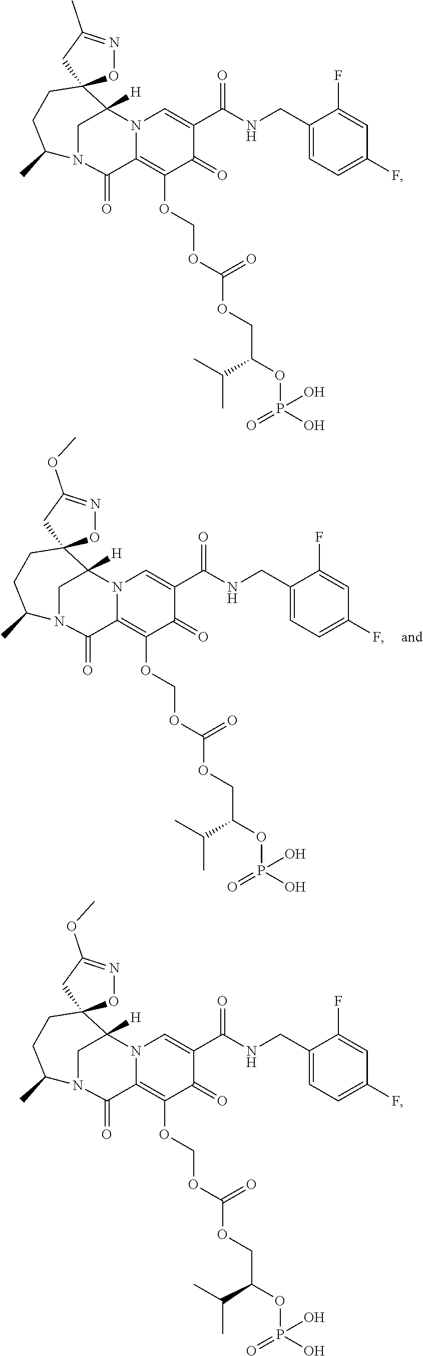 [SMILES: CC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1 ; COC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1 ; COC1=NO[C@@]2(CC[C@H](C)N3C[C@H]2n2cc(C(=O)NCc4ccc(F)cc4F)c(=O)c(OCOC(=O)OC[C@@H](OP(=O)(O)O)C(C)C)c2C3=O)C1]

or a pharmaceutically acceptable salt thereof.

Embodiment 45. The method of any one of embodiments 0-42, wherein the compound is selected from the group consisting of

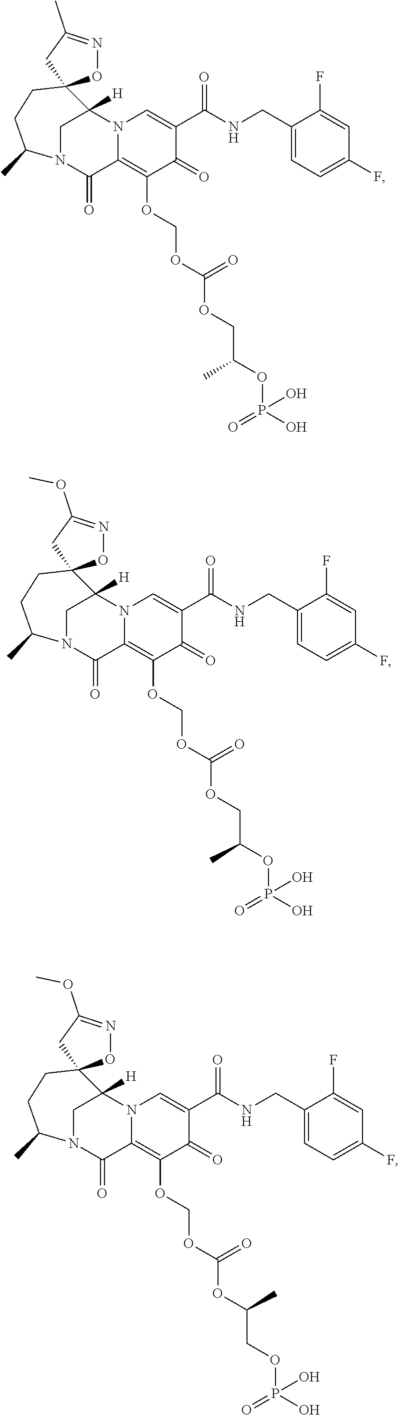 [SMILES: CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

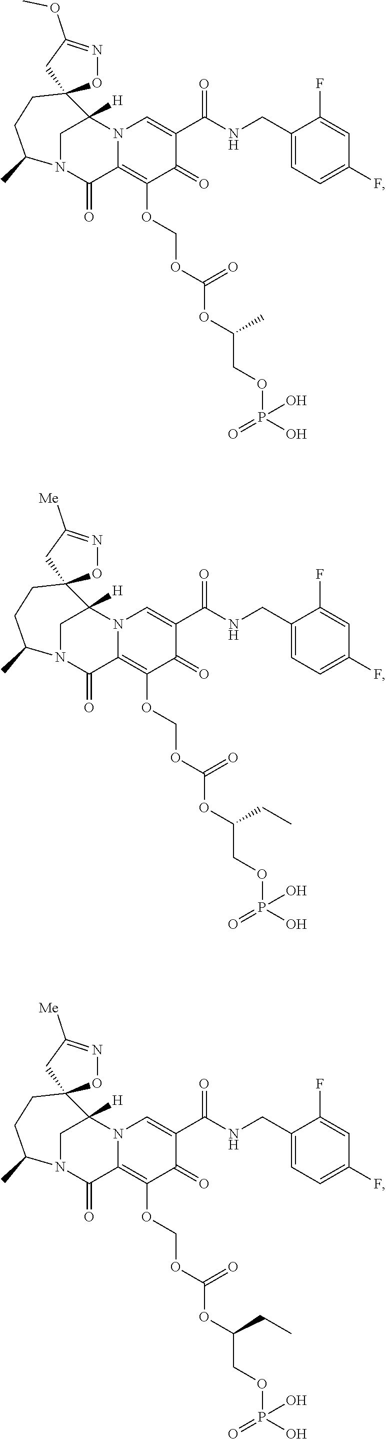 [SMILES: COC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1 ; CC[C@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2]

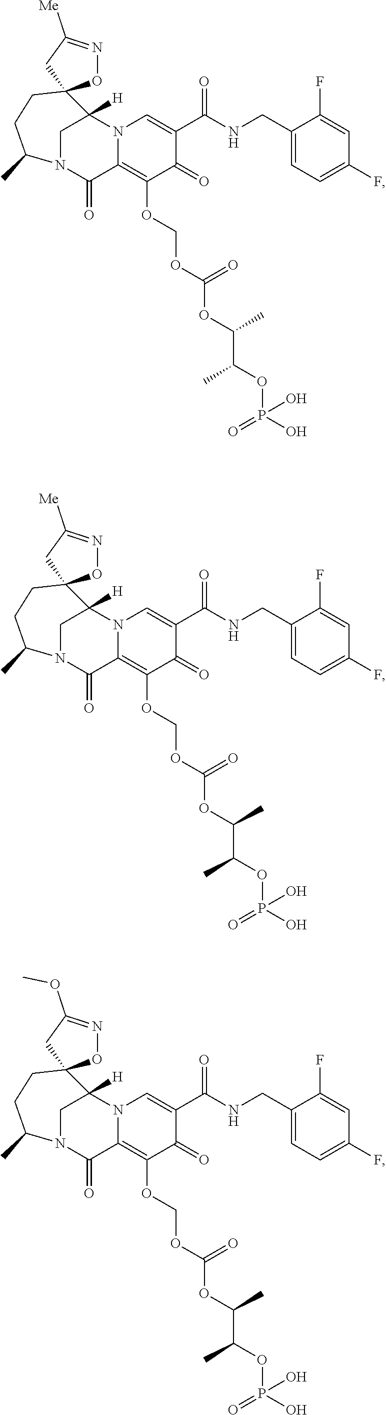 [SMILES: CC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

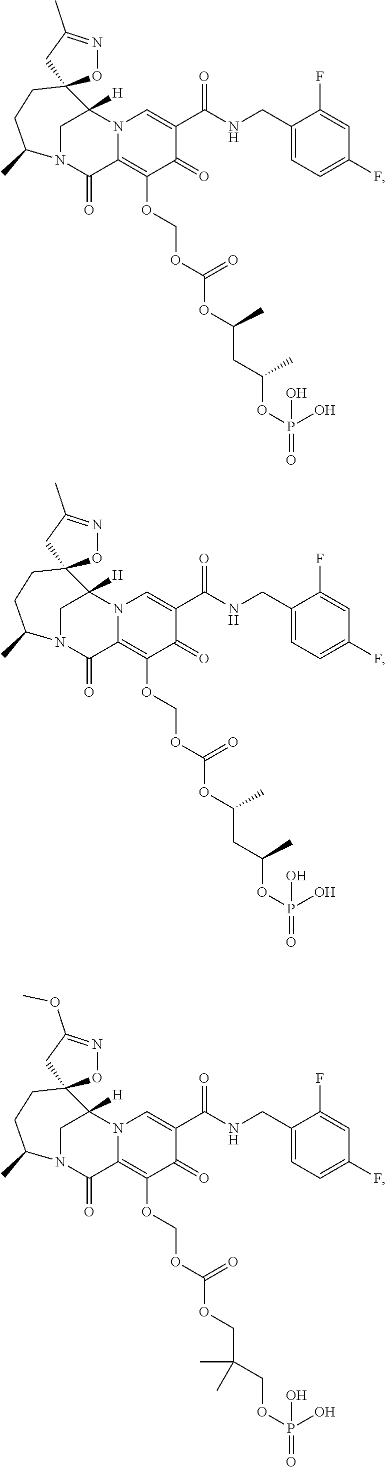 [SMILES: CC1=NO[C@@]2(CC[C@H](C)N3C[C@H]2n2cc(C(=O)NCc4ccc(F)cc4F)c(=O)c(OCOC(=O)O[C@@H](C)C[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)C[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

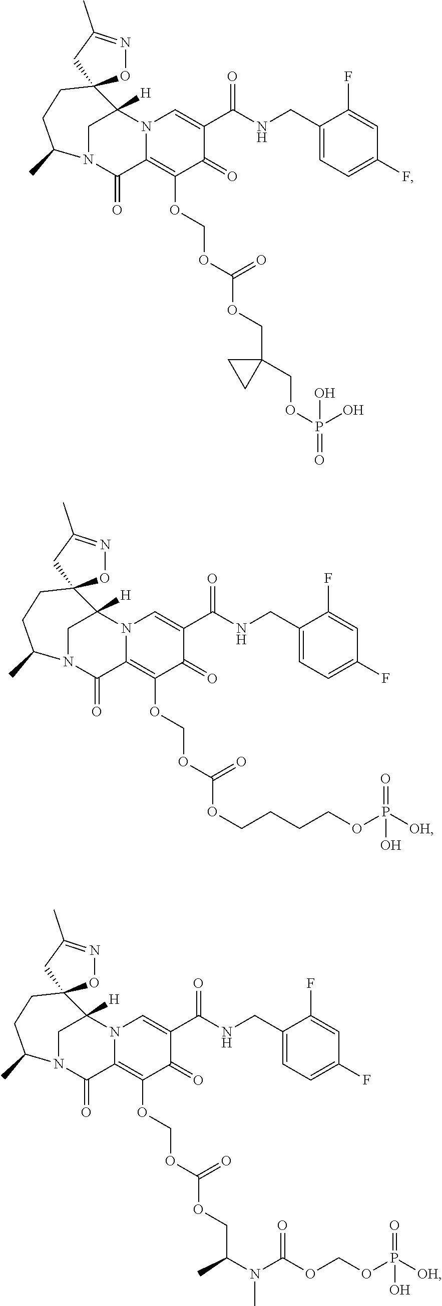 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)CC4)c2C3=O)C1 ; CC1=NO[C@@]2(CC[C@H](C)N3C[C@H]2n2cc(C(=O)NCc4ccc(F)cc4F)c(=O)c(OCOC(=O)OCCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)N(C)C(=O)OCOP(=O)(O)O)c2C3=O)C1]

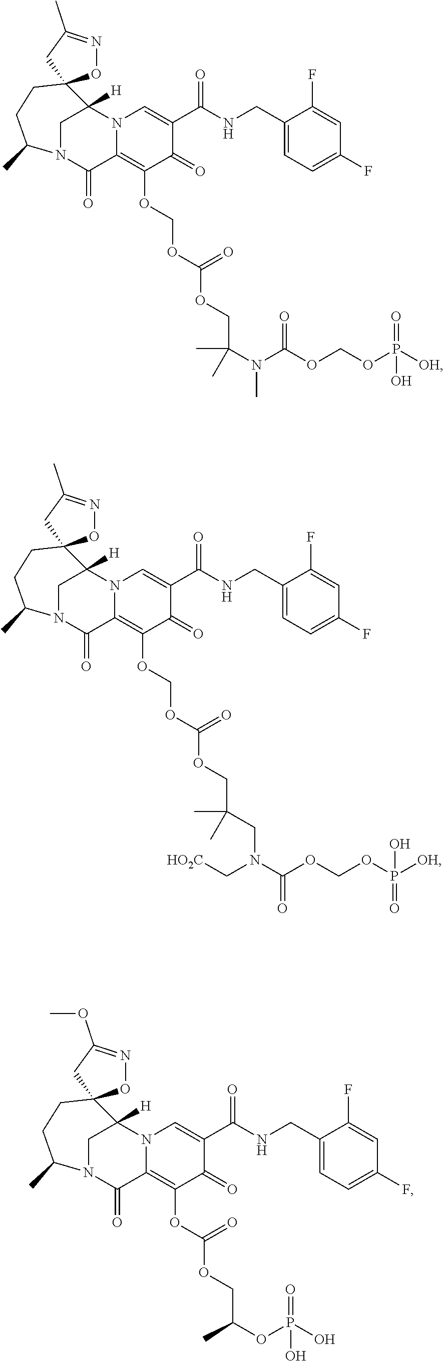 [SMILES: CC1=NO[C@@]2(CC[C@H](C)N3C[C@H]2n2cc(C(=O)NCc4ccc(F)cc4F)c(=O)c(OCOC(=O)OCC(C)(C)N(C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCC(C)(C)CN(CC(=O)O)C(=O)OCOP(=O)(O)O)c2C3=O)C1 ; COC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1]

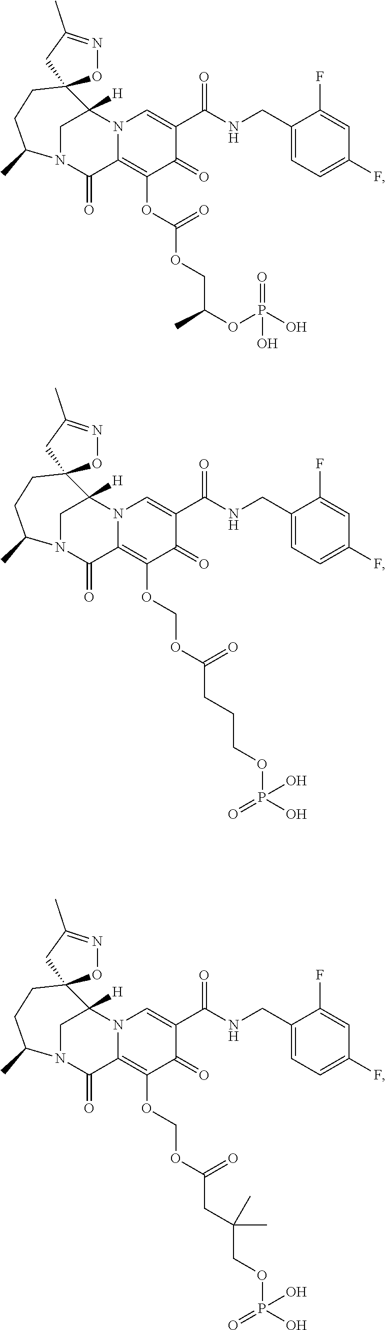 [SMILES: CC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)COP(=O)(O)O)c2C3=O)C1]

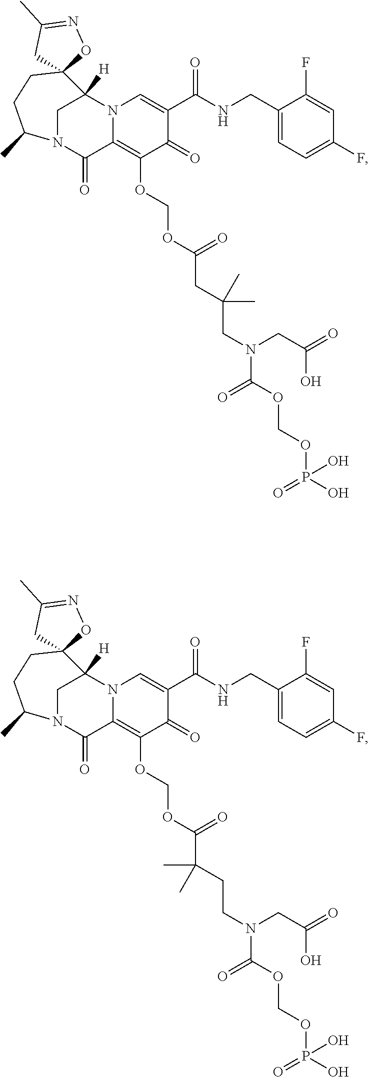 [SMILES: CC1=NO[C@@]2(CC[C@H](C)N3C[C@H]2n2cc(C(=O)NCc4ccc(F)cc4F)c(=O)c(OCOC(=O)CC(C)(C)CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)C(C)(C)CCN(CC(=O)O)C(=O)OCOP(=O)(O)O)c2C3=O)C1]

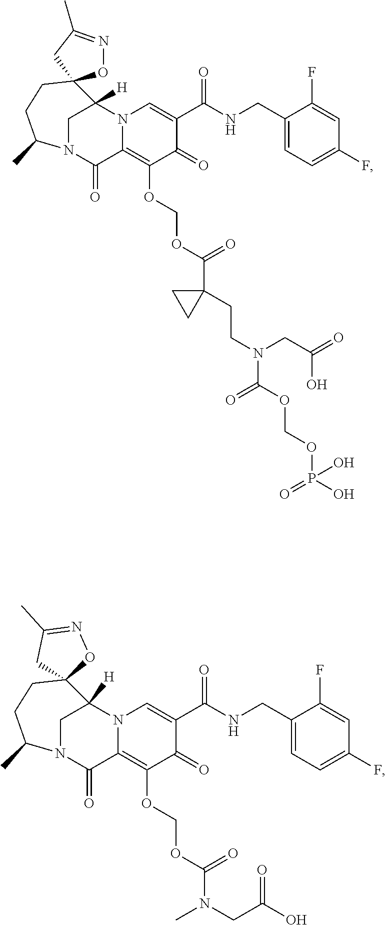 [SMILES: CC1=NO[C@@]2(CC[C@H](C)N3C[C@H]2n2cc(C(=O)NCc4ccc(F)cc4F)c(=O)c(OCOC(=O)C4(CCN(CC(=O)O)C(=O)OCOP(=O)(O)O)CC4)c2C3=O)C1 ; CC1=NO[C@@]2(CC[C@H](C)N3C[C@H]2n2cc(C(=O)NCc4ccc(F)cc4F)c(=O)c(OCOC(=O)N(C)CC(=O)O)c2C3=O)C1]

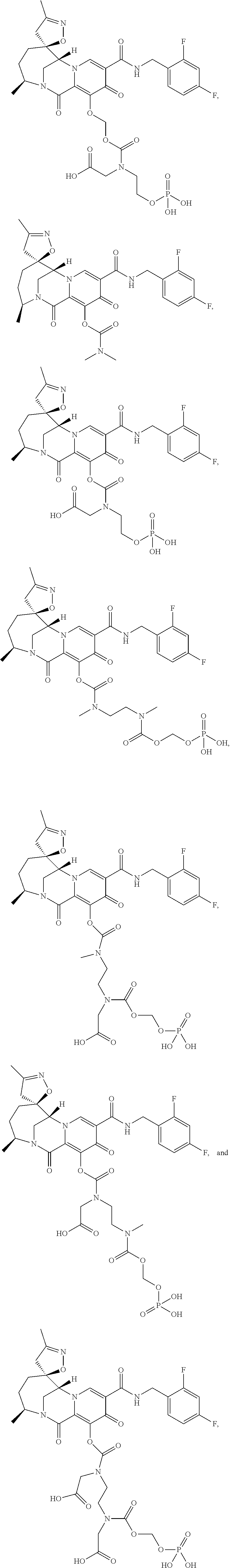

or a pharmaceutically acceptable salt thereof.

Embodiment 46. The method of any one of embodiments 1-45, wherein the subject is virologically suppressed.

Embodiment 47. The method of any one of embodiments 1-46, wherein in the subject is treatment experienced.

Embodiment 48. The method of any one of embodiments 1-47, wherein in the subject is treatment naive.

Embodiment 49. The method of any one of embodiments 1-48, wherein the Compound 1, or the pharmaceutically acceptable salt thereof, and the compound of Formula I, or the pharmaceutically acceptable salt thereof, are administered separately.

Embodiment 50. The method of any one of embodiments 1-48, wherein the Compound 1, or the pharmaceutically acceptable salt thereof, and the compound of Formula I, or the pharmaceutically acceptable salt thereof, are co-administered.

Embodiment 51. The method of any one of embodiments 1-48 and 50, wherein the Compound 1, or the pharmaceutically acceptable salt thereof, and the compound of Formula I, or the pharmaceutically acceptable salt thereof, are administered as a co-formulation.

Embodiment 52. The method of any one of embodiments 1-51, wherein the Compound 1, or the pharmaceutically acceptable salt thereof, and the compound of Formula I, or the pharmaceutically acceptable salt thereof, are administered once monthly, twice monthly, three times monthly, or four times monthly.

Embodiment 53. The method of any one of embodiments 1-52, wherein the Compound 1, or the pharmaceutically acceptable salt thereof, and the compound of Formula I, or the pharmaceutically acceptable salt thereof, are administered once monthly.

Embodiment 54. The method of any one of embodiments 1-53, wherein the Compound 1, or the pharmaceutically acceptable salt thereof, is administered orally, intravenously, subcutaneously, or intramuscularly.

Embodiment 55. The method of any one of embodiments 1-53, wherein the compound of Formula I, or the pharmaceutically acceptable salt thereof, is administered orally, intravenously, subcutaneously, or intramuscularly.

Embodiment 56. The method of any one of embodiments 1-55, wherein both the Compound 1, or the pharmaceutically acceptable salt thereof, and the compound of Formula I, or the pharmaceutically acceptable salt thereof, are administered orally, intravenously, subcutaneously, or intramuscularly.

Embodiment 57. The method of any one of embodiments 1-56, wherein the Compound 1, or the pharmaceutically acceptable salt thereof, is administered orally.

Embodiment 58. The method of any one of embodiments 1-57, wherein the compound of Formula I, or the pharmaceutically acceptable salt thereof, is administered orally.

Embodiment 59. The method of any one of embodiments 1-58, wherein both the Compound 1, or the pharmaceutically acceptable salt thereof, and the compound of Formula I, or the pharmaceutically acceptable salt thereof, are administered orally.

Embodiment 60. The method of any one of embodiments 1-58, wherein both the Compound 1, or the pharmaceutically acceptable salt thereof, and the compound of Formula I, or the pharmaceutically acceptable salt thereof, are administered orally once monthly.

Embodiment 61. The method of any one of embodiments 1-60, comprising administering the Compound 1 free acid.

Embodiment 62. The method of any one of embodiments 1-61, comprising administering the compound of Formula I free acid.

Embodiment 63. The method of any one of embodiments 1-62, comprising administering the Compound 1 free acid and the compound of Formula I free acid.

Embodiment 64. Use of Compound 1

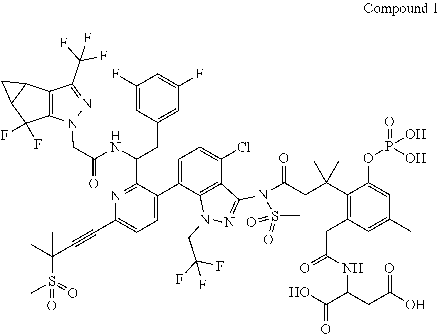 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, and a compound of Formula I of any one of embodiments 1-45, or a pharmaceutically acceptable salt thereof, for therapeutic treatment of an HIV infection in a subject.

Embodiment 65. A Compound 1

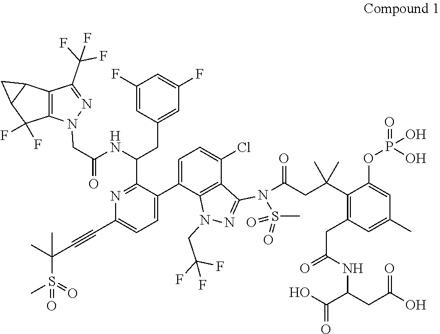 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, and a compound of Formula I of any one of embodiments 1-45, or a pharmaceutically acceptable salt thereof, for use in a therapeutic treatment of an HIV infection in a subject.

Embodiment 66. A pharmaceutical composition comprising a therapeutically effective amount of (a) Compound 1

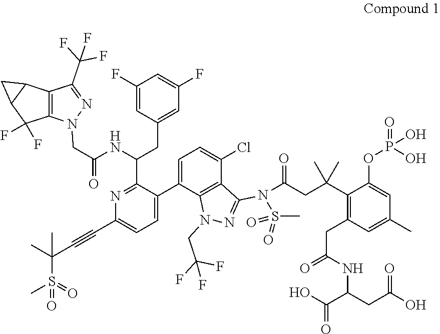 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, and (b) a therapeutically effective amount of a compound of Formula I of any one of embodiments 1-45, or a pharmaceutically acceptable salt thereof, and a pharmaceutically acceptable excipient.

Embodiment 67. A kit comprising (a) Compound 1

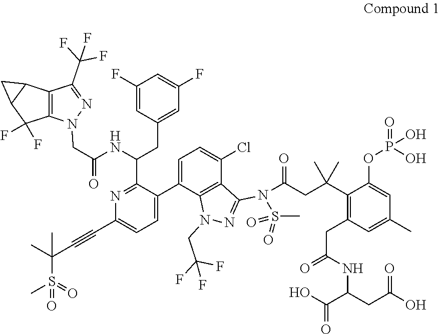 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, and (b) a compound of Formula I of any one of embodiments 1-45, or a pharmaceutically acceptable salt thereof, and instructions for use.

Embodiment 68. A combination comprising (a) Compound 1

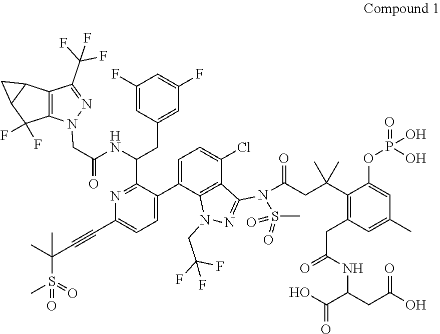 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, and (b) a compound of Formula I of any one of embodiments 1-45, or a pharmaceutically acceptable salt thereof.

Embodiment 69. The method, use, Compound 1 and compound of Formula I for use, pharmaceutical composition, kit, and combination of any one of embodiments 1-68, wherein the subject is human.

Embodiment 70. The method, use, Compound 1 and compound of Formula I for use, pharmaceutical composition, kit, and combination of any one of embodiments 1-69, wherein Compound 1 is Compound 1′

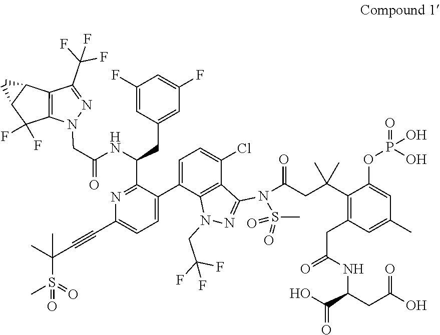 [SMILES: Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof.

Embodiment 71. The method, use, Compound 1 and compound of Formula I for use, pharmaceutical composition, kit, and combination of any one of embodiments 1-70, wherein the compound of Formula I is a compound of Formula I′:

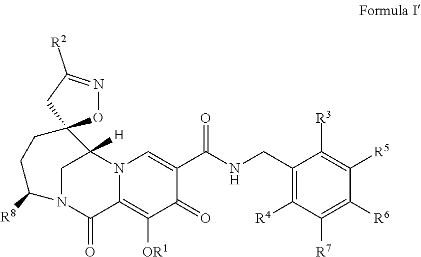

or a pharmaceutically acceptable salt thereof.

1. A method for therapeutic treatment of an HIV infection in a subject in need thereof, the method comprising administering to the subject:
(1) Compound 1, of the formula 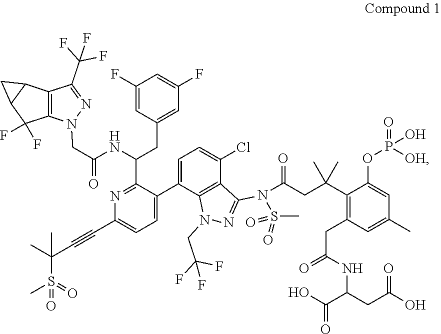 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]
or a pharmaceutically acceptable salt thereof, and
(2) a compound of Formula I 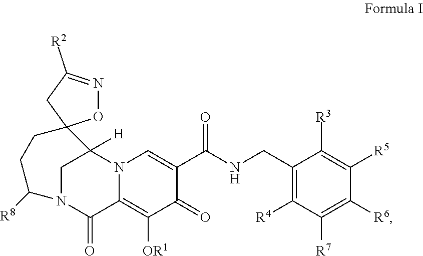
or a pharmaceutically acceptable salt thereof, wherein:
R¹ is —(CR^{1A}R^{1B}O)ₐ(Y)_{b}(CR^{1C}R^{1D})_{d}X;
wherein a is 0 or 1;
b is 0 or 1;
d is 0, 1, 2, 3, 4 or 5;
R^{1A} is H or C₁₋₃alkyl;
R^{1B} is H or C₁₋₃alkyl;
each R^{1C} is independently H or C₁₋₃alkyl;
each R^{1D} is independently H or C₁₋₃alkyl; or
optionally R^{1C} and R^{1D} on the same carbon atom are joined to form a spiro cyclopropyl group;
Y is —C(O)—, —C(O)O—, —C(O)NH— or —C(O)NR^{1H}—;
R^{1H} is C₁₋₄alkyl optionally substituted with one or two substituents independently selected from the group consisting of —COOH, —OH, —NH₂, —CONH₂, —P(O)(OH)₂, and —S(O)₂(OH);
X is selected from the group consisting of:
(a) —O—P(O)(OR^{1E})₂,
wherein each R^{1E} is independently H or phenyl;
(b) —N(R^{1F})₂,
wherein each R^{1F} is independently H, COO(CR^{1I}R^{1J})ₑOPO(OH)₂, or C₁₋₄alkyl; wherein the C₁₋₄ alkyl is optionally substituted with one or two substituents independently selected from the group consisting of —COOH, —S(O)₂(OH), —P(O)(OH)₂, —OH, —NH₂, and —CONH₂;
e is 1, 2, or 3;
each R^{1I} is independently H or C₁₋₃alkyl;
each R^{1J} is independently H or C₁₋₃alkyl; or
optionally R^{1I} and R^{1J} on the same carbon atom are joined to form a spiro cyclopropyl group; and
(c) —N⁺(R^{1G})₃Z″,
wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂;
Z⁻ is a counterion;
R² is C₁₋₃ alkyl or C₁₋₃ alkoxy;
each R³, R⁴, R⁵, R⁶ and R⁷ is independently H or halo, and
R⁸ is H or C₁₋₃alkyl.

2-3. (canceled)

4. A pharmaceutical composition comprising a therapeutically effective amount of
(1) Compound 1, of the formula 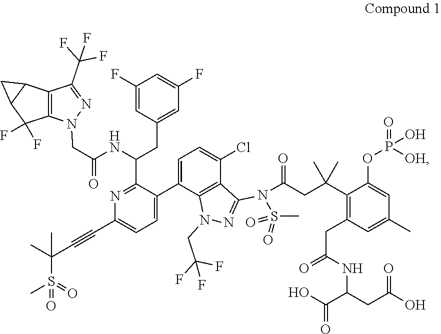 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]
or a pharmaceutically acceptable salt thereof; and
(2) a therapeutically effective amount of a compound of Formula I 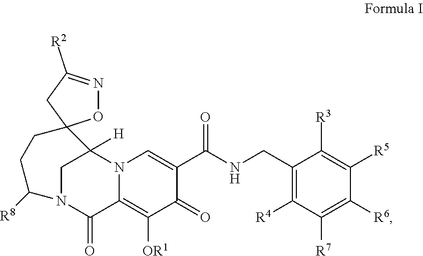
or a pharmaceutically acceptable salt thereof, wherein:
R¹ is —(CR^{1A}R^{1B}O)ₐ(Y)_{b}(C^{1I}CR^{1D})_{d}X;
wherein a is 0 or 1;
b is 0 or 1;
d is 0, 1, 2, 3, 4 or 5;
R^{1A} is H or C₁₋₃alkyl;
R^{1B} is H or C₁₋₃alkyl;
each R^{1C} is independently H or C₁₋₃alkyl;
each R^{1D} is independently H or C₁₋₃alkyl; or
optionally R^{1C} and R^{1D} on the same carbon atom are joined to form a spiro cyclopropyl group;
Y is —C(O)—, —C(O)O—, —C(O)NH— or —C(O)NR^{1H}—;
R^{1H} is C₁₋₄alkyl optionally substituted with one or two substituents independently selected from the group consisting of —COOH, —OH, —NH₂, —CONH₂, —P(O)(OH)₂, and —S(O)₂(OH);
X is selected from the group consisting of:
(c) —O—P(O)(OR^{1E})₂,
wherein each R^{1E} is independently H or phenyl;
(d) —N(R^{1F})₂,
wherein each R^{1F} is independently H, COO(CR^{1I}R^{1J})ₑOPO(OH)₂, or C₁₋₄alkyl; wherein the C₁₋₄ alkyl is optionally substituted with one or two substituents independently selected from the group consisting of —COOH, —S(O)₂(OH), —P(O)(OH)₂, —OH, —NH₂, and —CONH₂;
e is 1, 2, or 3;
each R^{1I} is independently H or C₁₋₃alkyl;
each R^{1J} is independently H or C₁₋₃alkyl; or
optionally R^{1I} and R^{1J} on the same carbon atom are joined to form a spiro cyclopropyl group; and
(c) —N⁺(R^{1G})₃Z⁻,
wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂;
Z⁻ is a counterion;
R² is C₁₋₃ alkyl or C₁₋₃ alkoxy;
each R³, R⁴, R⁵, R⁶ and R⁷ is independently H or halo, and
R⁸ is H or C₁₋₃alkyl; and
(3) a pharmaceutically acceptable excipient.

5. A kit comprising:
(1) Compound 1, of the formula 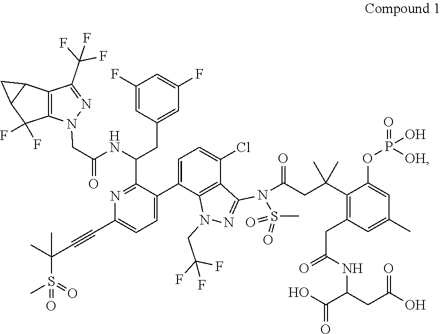 [SMILES: Cc1cc(CC(=O)NC(CC(=O)O)C(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]
or a pharmaceutically acceptable salt thereof; and
(2) a compound of Formula I 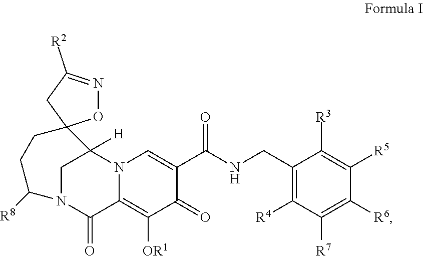
or a pharmaceutically acceptable salt thereof, wherein:
R¹ is —(CR^{1A}R^{1B}O)ₐ(Y)_{b}(C^{1C}R^{1D})_{d}X;
wherein a is 0 or 1;
b is 0 or 1;
d is 0, 1, 2, 3, 4 or 5;
R^{1A} is H or C₁₋₃alkyl;
R^{1B} is H or C₁₋₃alkyl;
each R^{1C} is independently H or C₁₋₃alkyl;
each R^{1D} is independently H or C₁₋₃alkyl; or
optionally R^{1C} and R^{1D} on the same carbon atom are joined to form a spiro cyclopropyl group;
Y is —C(O)—, —C(O)O—, —C(O)NH— or —C(O)NR^{1H}—;
R^{1H} is C₁₋₄alkyl optionally substituted with one or two substituents independently selected from the group consisting of —COOH, —OH, —NH₂, —CONH₂, —P(O)(OH)₂, and —S(O)₂(OH);
X is selected from the group consisting of:
(e) —O—P(O)(OR^{1E})₂,
wherein each R^{1E} is independently H or phenyl;
(f) —N(R^{1F})₂,
wherein each R^{1F} is independently H, COO(CR^{1I}R^{1J})ₑOPO(OH)₂, or C₁₋₄alkyl; wherein the C₁₋₄ alkyl is optionally substituted with one or two substituents independently selected from the group consisting of —COOH, —S(O)₂(OH), —P(O)(OH)₂, —OH, —NH₂, and —CONH₂;
e is 1, 2, or 3;
each R^{1I} is independently H or C₁₋₃alkyl;
each R^{1J} is independently H or C₁₋₃alkyl; or
optionally R^{1I} and R^{1J} on the same carbon atom are joined to form a spiro cyclopropyl group; and
(c) —N⁺(R^{1G})₃Z⁻,
wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂;
Z⁻ is a counterion;
R² is C₁₋₃ alkyl or C₁₋₃ alkoxy;
each R³, R⁴, R⁵, R⁶ and R⁷ is independently H or halo, and
R⁸ is H or C₁₋₃alkyl; and
instructions for use.

6-7. (canceled)

8. The method of claim 1, wherein X is —O—P(O)(OR^{1E})₂.

9. (canceled)

10. The method of claim 1, wherein each R^{1E} is H.

11-22. (canceled)

23. The method of claim 1, wherein a is 1.

24. (canceled)

25. The method of claim 1, wherein b is 1.

26. The method of claim 1, wherein Y is —C(O)—, —C(O)O—, —C(O) NH—, or —C(O)NCH₃—.

27-32. (canceled)

33. The method of claim 1, wherein d is 1, 2, or 3.

34-36. (canceled)

37. The method of claim 1, wherein R⁴, R⁵, and R⁷ are each H.

38. The method of claim 1, wherein R³ and R⁶ are each independently a halo.

39. (canceled)

40. The method of claim 1, wherein R⁸ is C₁₋₃alkyl.

41. (canceled)

42. The method of claim 1, wherein R² is methyl or methoxy.

43-44. (canceled)

45. The method of claim 1, wherein R¹ is selected from the group consisting of:

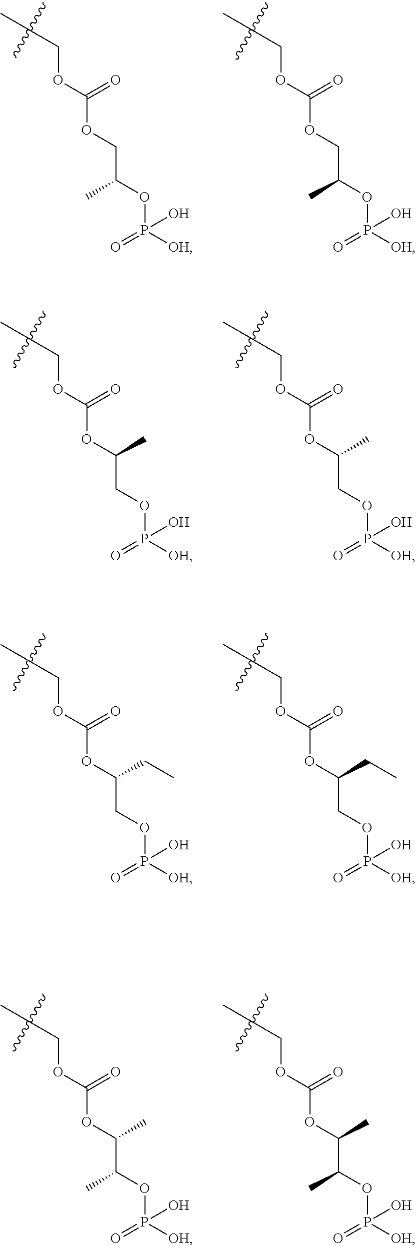

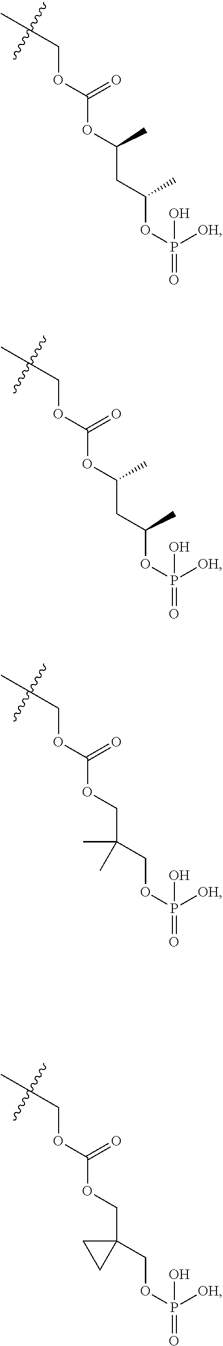 [SMILES: *COC(=O)O[C@@H](C)C[C@H](C)OP(=O)(O)O ; *COC(=O)O[C@H](C)C[C@@H](C)OP(=O)(O)O ; *COC(=O)OCC(C)(C)COP(=O)(O)O ; *COC(=O)OCC1(COP(=O)(O)O)CC1]

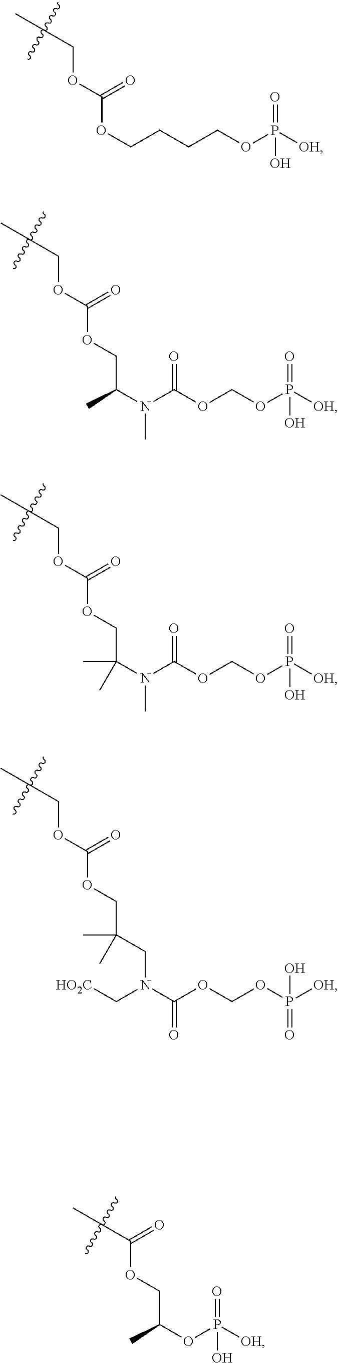 [SMILES: *COC(=O)OCCCCOP(=O)(O)O ; *COC(=O)OC[C@H](C)N(C)C(=O)OCOP(=O)(O)O ; *COC(=O)OCC(C)(C)N(C)C(=O)OCOP(=O)(O)O ; *COC(=O)OCC(C)(C)CN(CC(=O)O)C(=O)OCOP(=O)(O)O ; *C(=O)OC[C@H](C)OP(=O)(O)O]

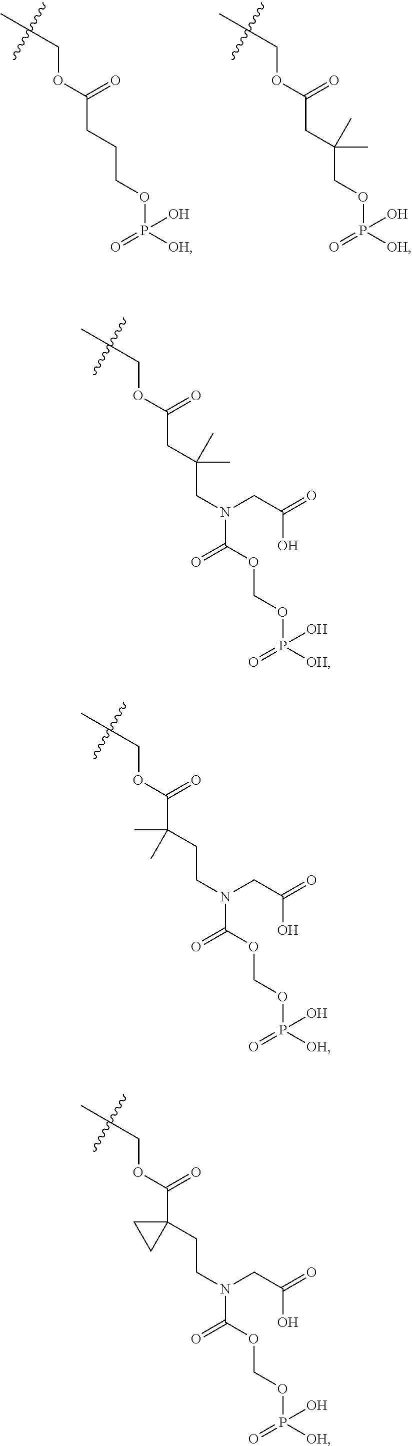 [SMILES: *COC(=O)CCCOP(=O)(O)O ; *COC(=O)CC(C)(C)COP(=O)(O)O ; *COC(=O)CC(C)(C)CN(CC(=O)O)C(=O)OCOP(=O)(O)O ; *COC(=O)C(C)(C)CCN(CC(=O)O)C(=O)OCOP(=O)(O)O ; *COC(=O)C1(CCN(CC(=O)O)C(=O)OCOP(=O)(O)O)CC1]

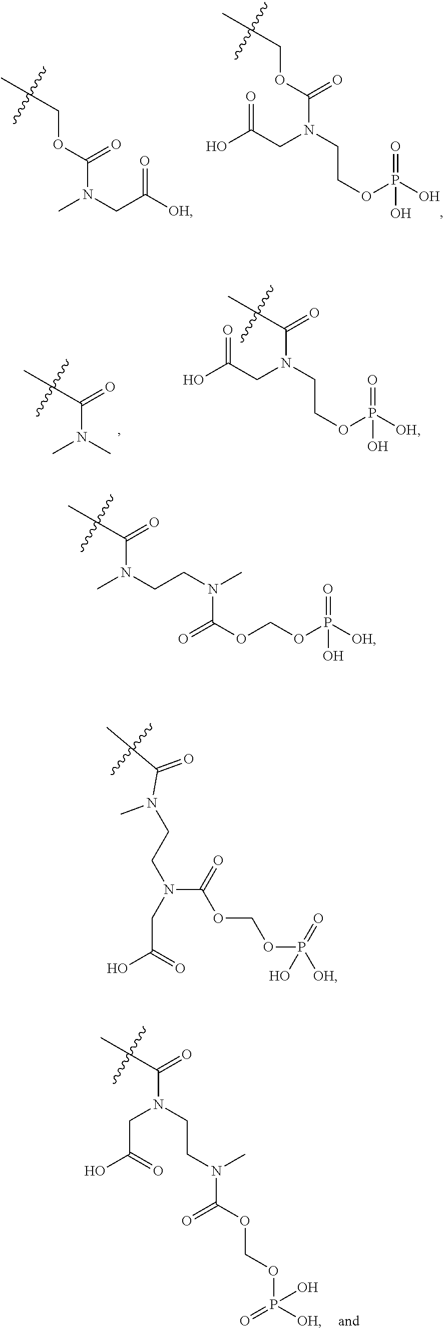

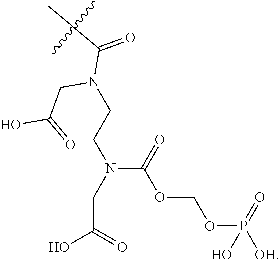

46-47. (canceled)

48. The method of claim 1, wherein the compound of Formula I is selected from the group consisting of 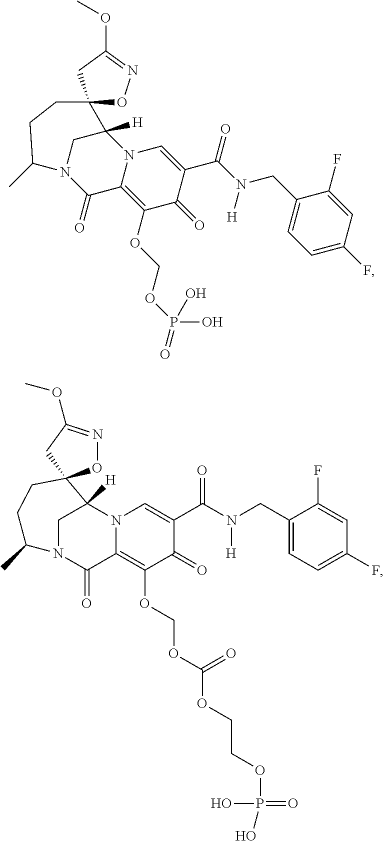 [SMILES: COC1=NO[C@@]2(CCC(C)N3C[C@H]2n2cc(C(=O)NCc4ccc(F)cc4F)c(=O)c(OCOP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1] 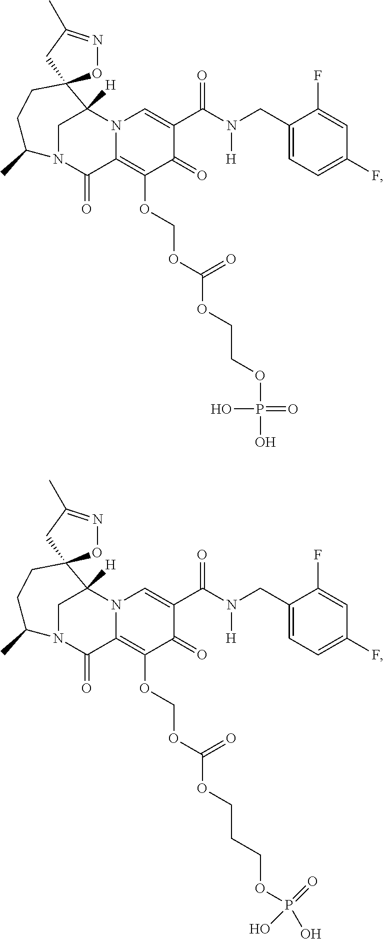 [SMILES: CC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCCOP(=O)(O)O)c2C3=O)C1] 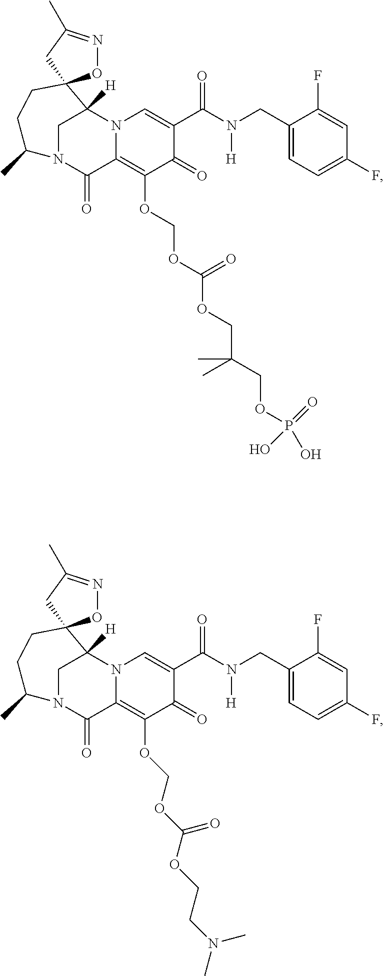 [SMILES: CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCN(C)C)c2C3=O)C1] 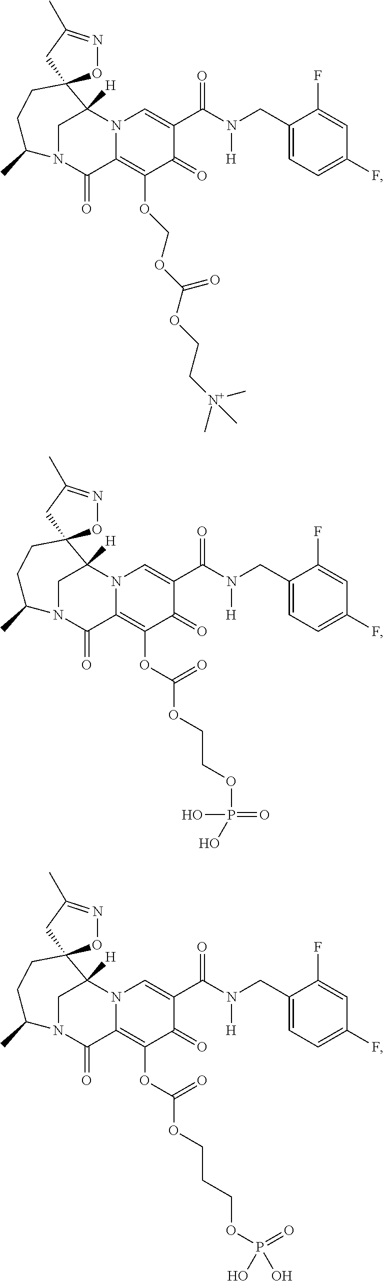 [SMILES: CC1=NO[C@@]2(CC[C@H](C)N3C[C@H]2n2cc(C(=O)NCc4ccc(F)cc4F)c(=O)c(OCOC(=O)OCC[N+](C)(C)C)c2C3=O)C1 ; CC1=NO[C@@]2(CC[C@H](C)N3C[C@H]2n2cc(C(=O)NCc4ccc(F)cc4F)c(=O)c(OC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCCCOP(=O)(O)O)c2C3=O)C1] 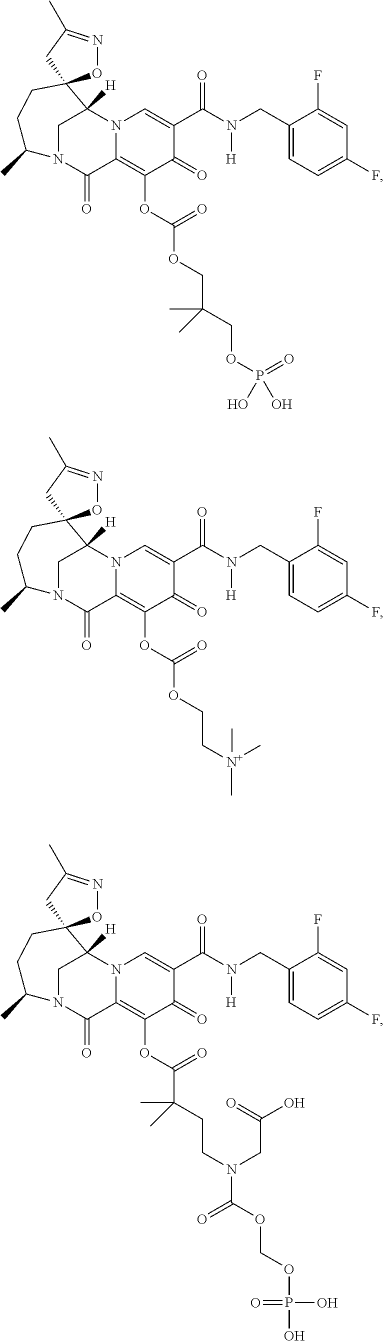 [SMILES: CC1=NO[C@@]2(CC[C@H](C)N3C[C@H]2n2cc(C(=O)NCc4ccc(F)cc4F)c(=O)c(OC(=O)OCC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCC[N+](C)(C)C)c2C3=O)C1 ; CC1=NO[C@@]2(CC[C@H](C)N3C[C@H]2n2cc(C(=O)NCc4ccc(F)cc4F)c(=O)c(OC(=O)C(C)(C)CCN(CC(=O)O)C(=O)OCOP(=O)(O)O)c2C3=O)C1] 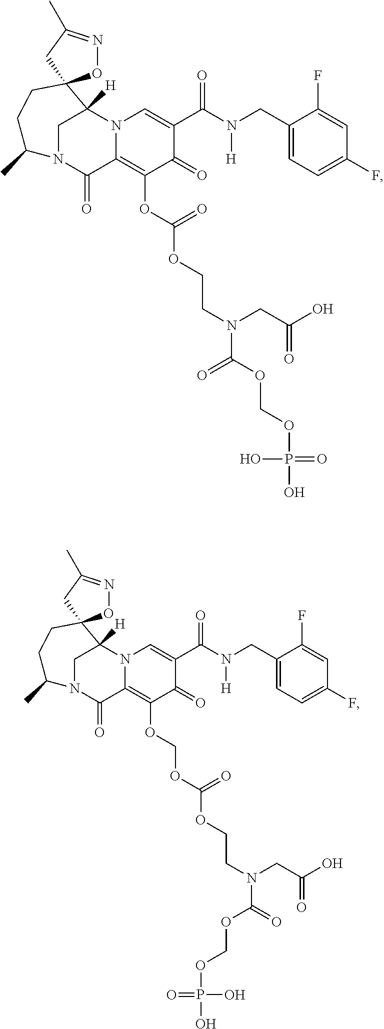 [SMILES: CC1=NO[C@@]2(CC[C@H](C)N3C[C@H]2n2cc(C(=O)NCc4ccc(F)cc4F)c(=O)c(OC(=O)OCCN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCN(CC(=O)O)C(=O)OCOP(=O)(O)O)c2C3=O)C1] 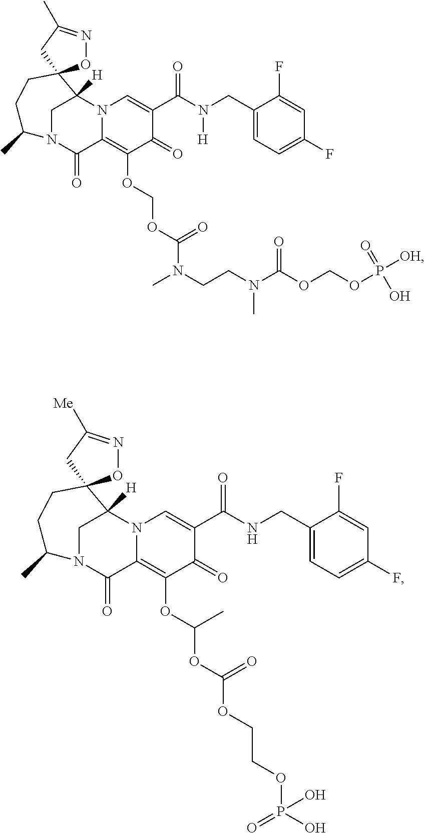 [SMILES: CC1=NO[C@@]2(CC[C@H](C)N3C[C@H]2n2cc(C(=O)NCc4ccc(F)cc4F)c(=O)c(OCOC(=O)N(C)CCN(C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(C)OC(=O)OCCOP(=O)(O)O)c2C3=O)C1] 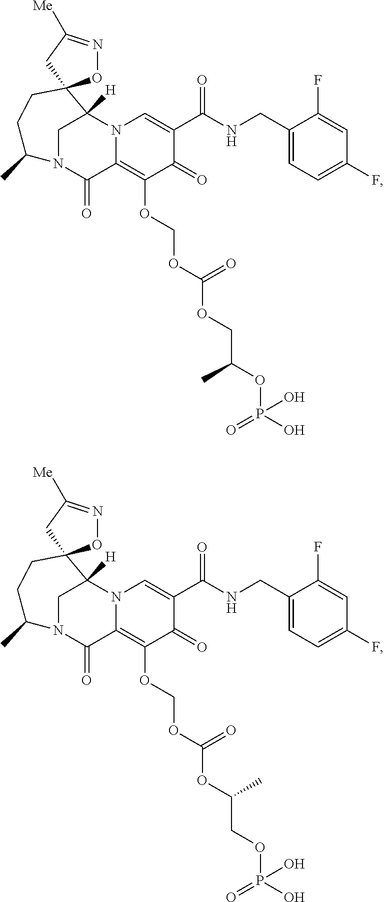 [SMILES: CC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1] 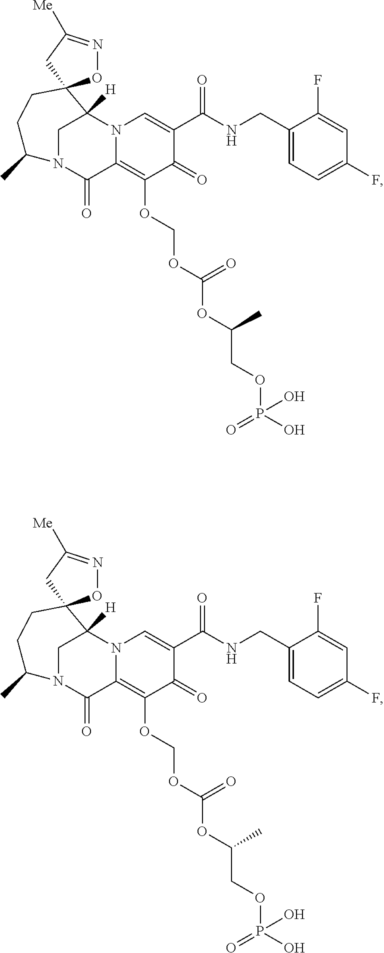 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1] 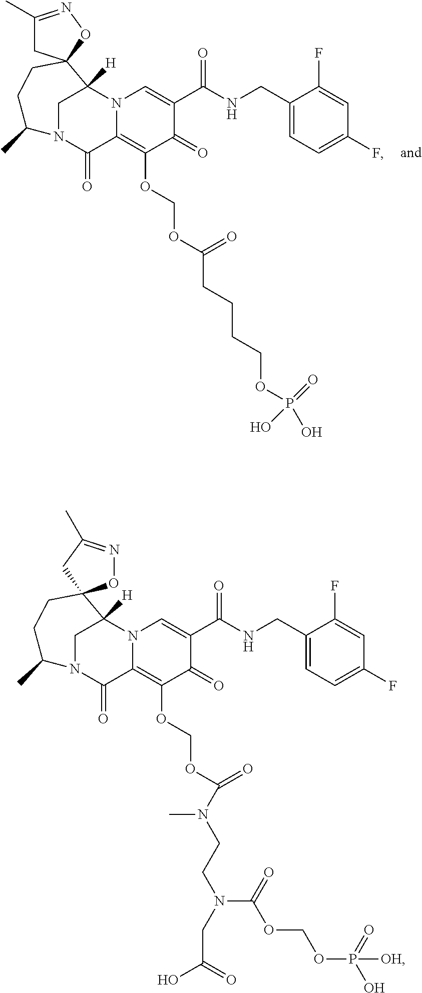 [SMILES: CC1=NO[C@@]2(CC[C@H](C)N3C[C@H]2n2cc(C(=O)NCc4ccc(F)cc4F)c(=O)c(OCOC(=O)CCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)CCN(CC(=O)O)C(=O)OCOP(=O)(O)O)c2C3=O)C1]
or a pharmaceutically acceptable salt thereof.

49. The method of claim 1, wherein the compound of Formula I is selected from the group consisting of 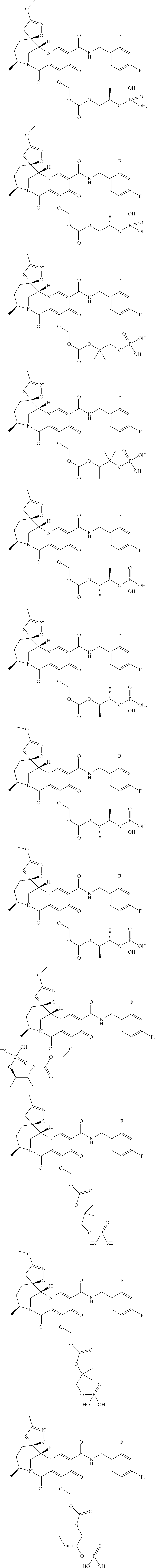 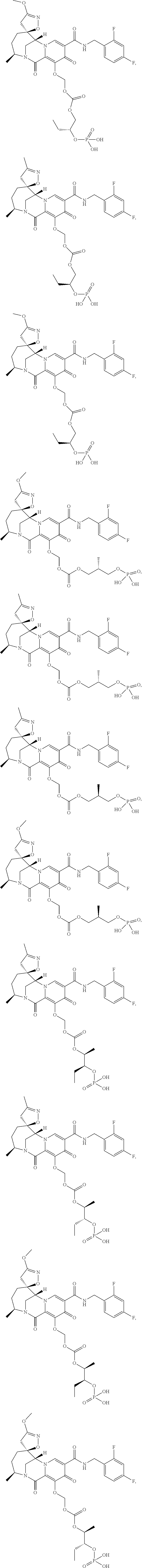 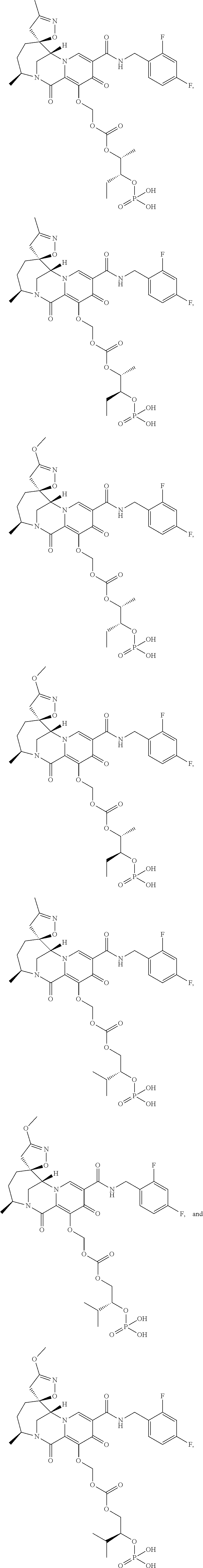
or a pharmaceutically acceptable salt thereof.

50. The method of claim 1, wherein the compound of Formula I is selected from the group consisting of: 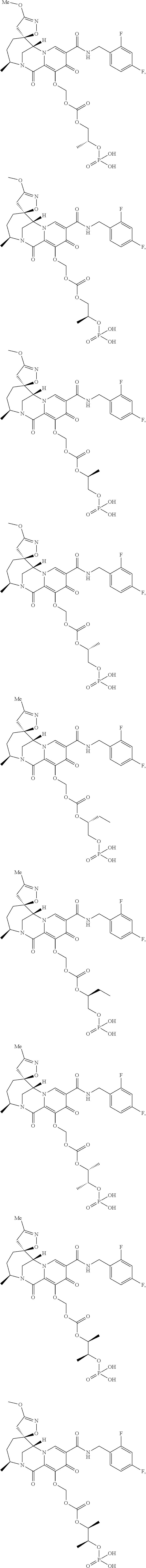 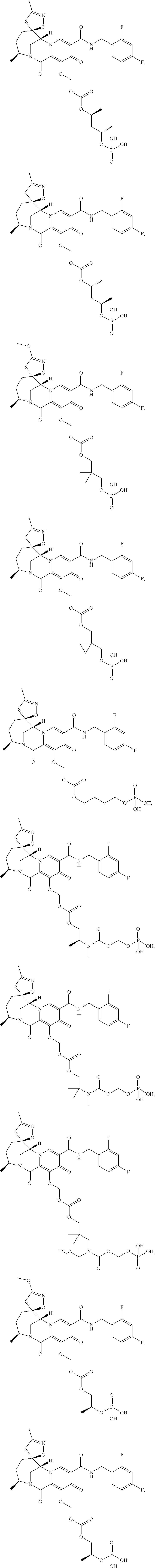 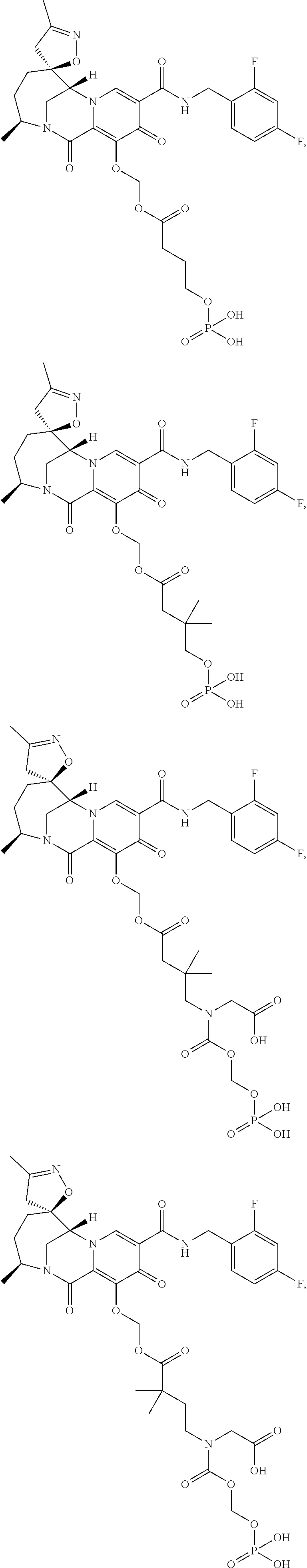 [SMILES: CC1=NO[C@@]2(CC[C@H](C)N3C[C@H]2n2cc(C(=O)NCc4ccc(F)cc4F)c(=O)c(OCOC(=O)CCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)C(C)(C)CCN(CC(=O)O)C(=O)OCOP(=O)(O)O)c2C3=O)C1] 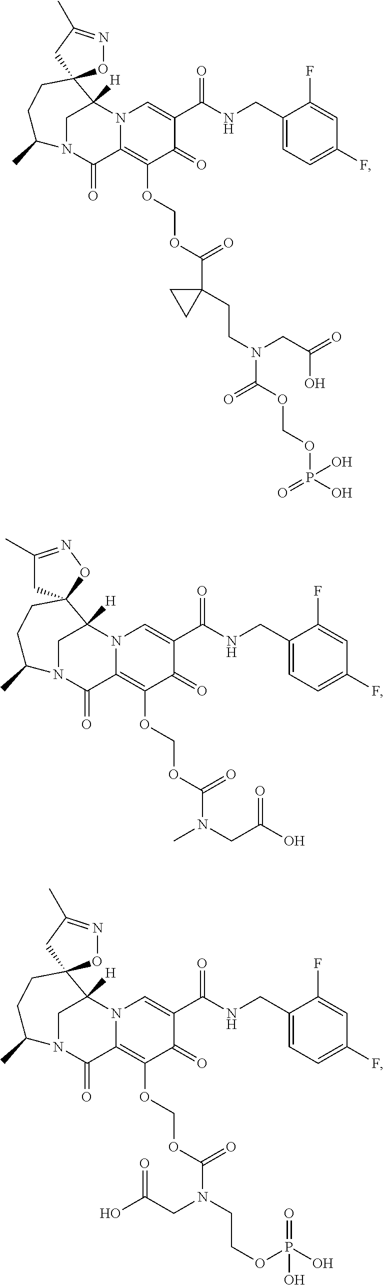 [SMILES: CC1=NO[C@@]2(CC[C@H](C)N3C[C@H]2n2cc(C(=O)NCc4ccc(F)cc4F)c(=O)c(OCOC(=O)C4(CCN(CC(=O)O)C(=O)OCOP(=O)(O)O)CC4)c2C3=O)C1 ; CC1=NO[C@@]2(CC[C@H](C)N3C[C@H]2n2cc(C(=O)NCc4ccc(F)cc4F)c(=O)c(OCOC(=O)N(C)CC(=O)O)c2C3=O)C1 ; CC1=NO[C@@]2(CC[C@H](C)N3C[C@H]2n2cc(C(=O)NCc4ccc(F)cc4F)c(=O)c(OCOC(=O)N(CCOP(=O)(O)O)CC(=O)O)c2C3=O)C1] 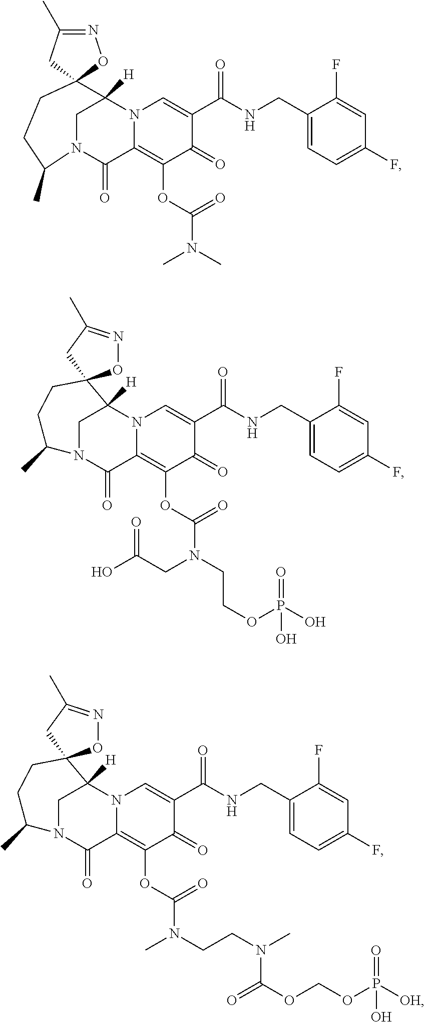 [SMILES: CC1=NO[C@@]2(CC[C@H](C)N3C[C@H]2n2cc(C(=O)NCc4ccc(F)cc4F)c(=O)c(OC(=O)N(C)C)c2C3=O)C1 ; CC1=NO[C@@]2(CC[C@H](C)N3C[C@H]2n2cc(C(=O)NCc4ccc(F)cc4F)c(=O)c(OC(=O)N(CCOP(=O)(O)O)CC(=O)O)c2C3=O)C1 ; CC1=NO[C@@]2(CC[C@H](C)N3C[C@H]2n2cc(C(=O)NCc4ccc(F)cc4F)c(=O)c(OC(=O)N(C)CCN(C)C(=O)OCOP(=O)(O)O)c2C3=O)C1] 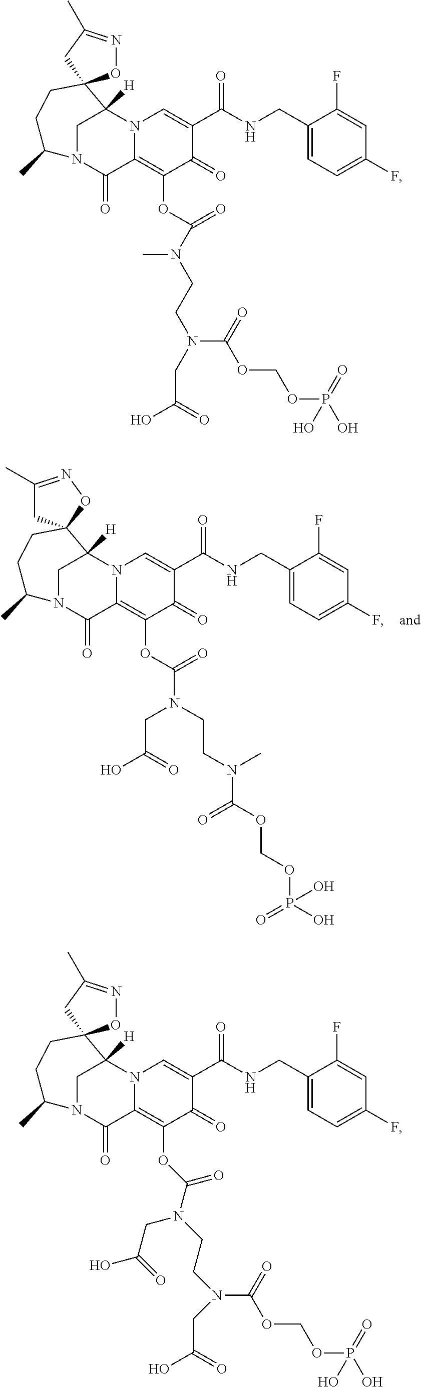 [SMILES: CC1=NO[C@@]2(CC[C@H](C)N3C[C@H]2n2cc(C(=O)NCc4ccc(F)cc4F)c(=O)c(OC(=O)N(C)CCN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(CCN(C)C(=O)OCOP(=O)(O)O)CC(=O)O)c2C3=O)C1 ; CC1=NO[C@@]2(CC[C@H](C)N3C[C@H]2n2cc(C(=O)NCc4ccc(F)cc4F)c(=O)c(OC(=O)N(CCN(CC(=O)O)C(=O)OCOP(=O)(O)O)CC(=O)O)c2C3=O)C1]
or a pharmaceutically acceptable salt thereof.

51. The method of claim 1, wherein the compound of Formula I is selected from: 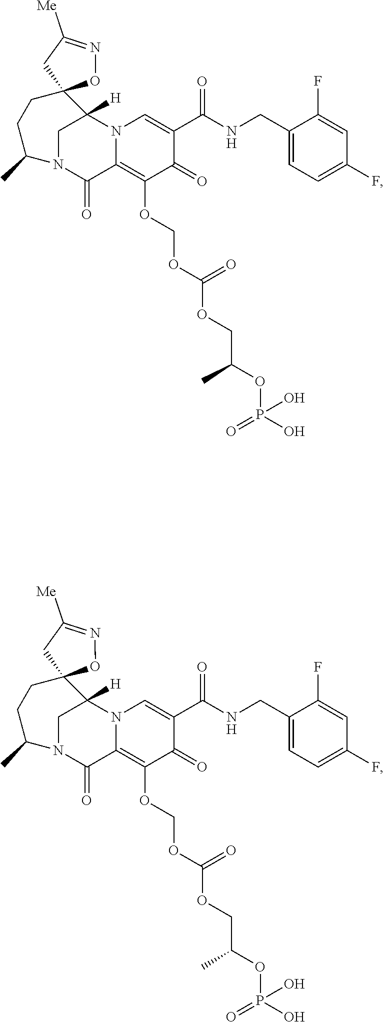 [SMILES: CC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1] 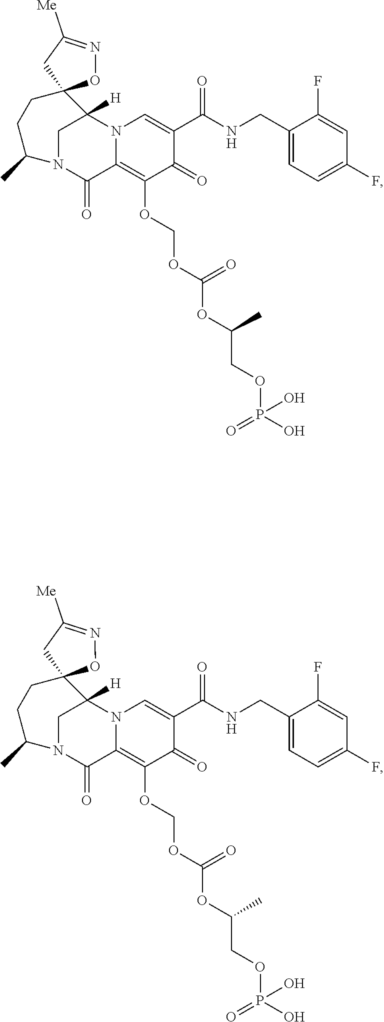 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1] 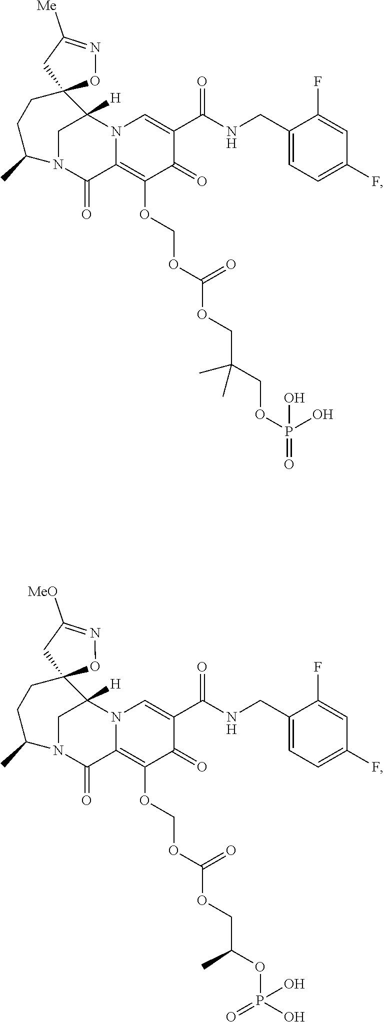 [SMILES: CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1] 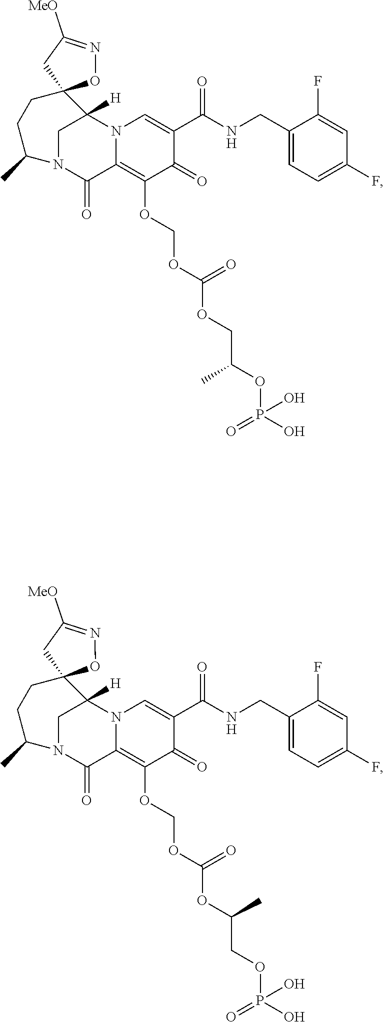 [SMILES: COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1] 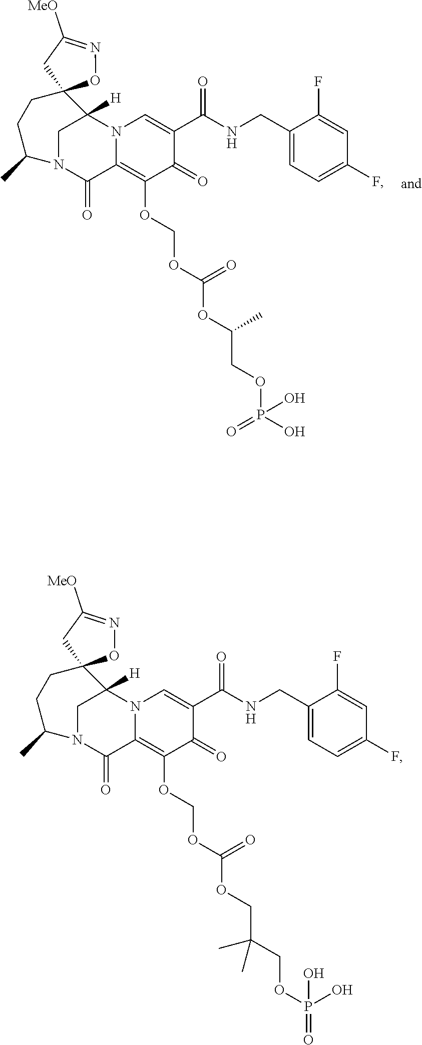 [SMILES: COC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]
or a pharmaceutically acceptable salt thereof.

52-77. (canceled)

78. The method of claim 1, comprising administering the Compound 1 free acid and the compound of Formula I free acid.

79. The method of claim 1, wherein the subject is human.

80. The method of claim 1, wherein Compound 1 is Compound 1′: 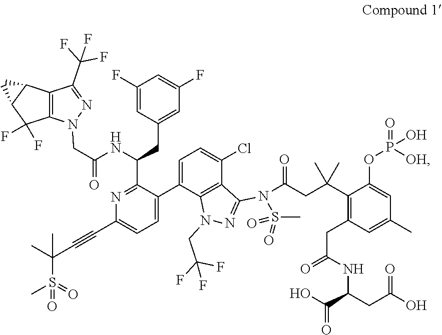 [SMILES: Cc1cc(CC(=O)N[C@@H](CC(=O)O)C(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]
or a pharmaceutically acceptable salt thereof.
